(19) United States
(12) Patent Application Publication
(10) Pub. No.: US 2005/0107423 A1
(43) Pub. Date: May 19, 2005

(54) Quinolone antibacterial agents

(76) Inventors: Edmund Lee Ellsworth, Pinckney, MI (US); Richard J. Sciotti, Saline, MI (US); Jeremy Tyson Starr, Ann Arbor, MI (US)

Correspondence Address: WARNER-LAMBERT COMPANY, 2800 PLYMOUTH RD, ANN ARBOR, MI, 48105, US

(21) Appl. No.: 10/936,441

(22) Filed: Sep. 8, 2004

Related U.S. Application Data
(60) Provisional application No. 60/502,330, filed on Sep. 12, 2003.

(51) Int. Cl. C07D 471/02; C07D 215/16; A61K 31/47; A61K 31/4709; A61K 31/4745

(52) U.S. Cl. 514/300; 514/312; 546/123; 546/156

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

### FIELD OF THE INVENTION

The invention relates to compounds bearing a quinolone core structure which exhibit antibacterial activity, methods for their preparation, as well as pharmaceutically acceptable compositions comprising such compounds.

### BACKGROUND OF THE INVENTION

Antibacterial resistance is a global clinical and public health problem that has emerged with alarming rapidity in recent years. Resistance is a problem in the community as well as in health care settings, where transmission of bacteria is greatly amplified. Because multiple drug resistance is a growing problem, physicians are now confronted with infections for which there is no effective therapy. The morbidity, mortality, and financial costs of such infections pose an increasing burden for health care systems worldwide. As a result, alternative and improved agents are needed for the treatment of bacterial infections, particularly for the treatment of infections caused by resistant strains of bacteria.

### SUMMARY OF THE INVENTION

These and other needs are met by the present invention, which is directed to a compound of formula I: 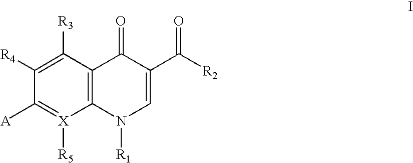
or a pharmaceutically acceptable salt thereof, wherein:
X is N or C, provided that when X is N, R₅ is absent at that position;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 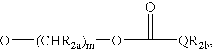
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 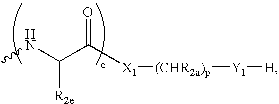
   wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R₁ and R₅, together with the carbons to which they are attached, form a substituted or unsubstituted 6-membered ring containing an additional heteroatom selected from O, S, NH, or N(C₁-C₆)alkyl;
   with the proviso that when R₁ is (C₃-C₆)cycloalkyl or halo(C₃-C₆)cycloalkyl, R₅ is H, halo, NH₂, (C₁-C₆)alkoxy, or halo(C₁-C₆)alkoxy. 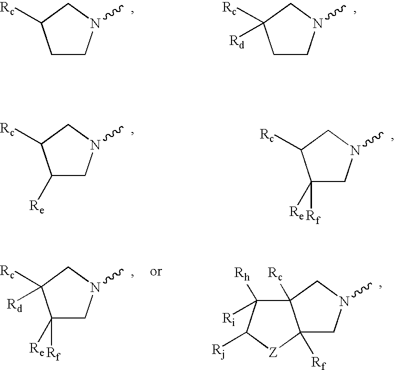
   wherein R_{c} is OH,
   OPO(OH)₂,
   OPO(O(C₁-C₆)alkyl)₂, 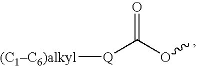
   wherein “ [symbol] ” indicates the point of attachment and Q is 0 or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(Cl -C₆)haloalkyl-O—,
   RᵢᵢO(C₃-C₆)cycloalkyl-O—, 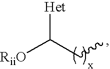
   wherein “ [symbol] ” indicates the point of attachment, het is a 5- or 6-membered heterocyclo or heteroaryl group, and x is an integer of from 0 to 10; 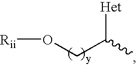
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, or 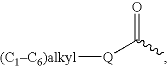
         as defined above; and
R_{d}, Rₑ, and R_{f} are each independently (C₁-C₆)alkyl, 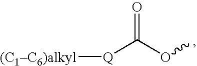
   wherein “ [symbol] ” indicates the point of attachment and Q is 0 or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 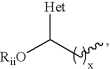
   wherein “ [symbol] ” indicates the point of attachment, het is a 5- or 6-membered heterocyclo or heteroaryl group, and x is an integer of from 0 to 10; 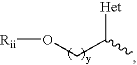
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, or 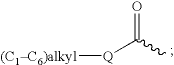
Z is absent or is a linker containing1, 2, or 3 substituted or unsubstituted carbon atoms;
Rₕ, Rᵢ, and Rⱼ are each independently H,
   OH,
   OPO(OH)₂,
   OPO(O(C₁-C₆)alkyl)₂, 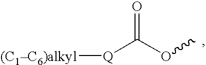
   wherein “ [symbol] ” indicates the point of attachment and Q is 0 or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 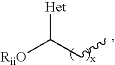
   wherein “ [symbol] ” indicates the point of attachment, het is a 5- or 6-membered heterocyclo or heteroaryl group, and x is an integer of from 0 to 10; 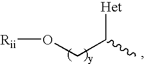
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 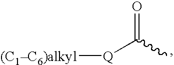
         as defined above;
   provided that not all of Rₕ Rᵢ, and Rⱼ are H.

What is also provided is a compound of formula II 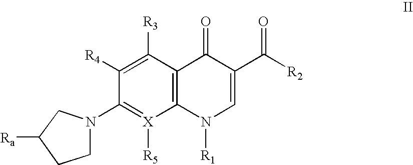
or a pharmaceutically acceptable salt thereof, wherein:
X is N or C, provided that when X is N, R₅ is absent at that position;
R₁ is (C₃-C₆)cycloalkyl,
   halo(C₃-C₆)cycloalkyl,
   (C₁-C₆)alkyl,
   halo(C₁-C₆)alkyl,
   aryl, and
   heteroaryl;
R₂ is OH,
   OBF₂,
   O(C₁-C₆)alkyl,
   O(C₃-C₆)cycloalkyl, 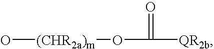
   wherein m is an integer of from 1 to 10, Q is O or is absent, and R₂a is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 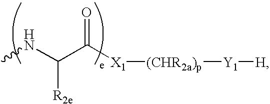
   wherein “ [symbol] ” indicates the point of attachment, 2a is as defined above, R₂ₑ is H or (C₁-C)alkyl, e is an integer of from 1 to 10, p is an integer of from 2 to 10, and X₁ and Y₁ are each independently NH or O;
R₃ and R₄ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R₅ is H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is OPO(OH)₂,
   OPO(O(C₁-C₆)alkyl)₂, 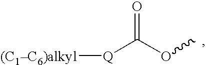
      wherein “ [symbol] ” indicates the point of attachment and Q is 0 or is absent,
   RᵢᵢO(C₂-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 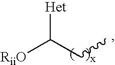
   wherein “ [symbol] ” indicates the point of attachment, het is a 5- or 6-membered heterocyclo or heteroaryl group, and x is an integer of from 0 to 10; 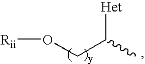
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, or 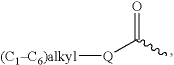
         as defined above; or
R₁ and R₅ , together with the carbons to which they are attached, form a substituted or unsubstituted 6-membered ring containing an additional heteroatom selected from O, S, NH, or N(C₁-C₆)alkyl;
   with the proviso that when R₁ is (C₃-C₆)cycloalkyl or halo(C₃-C₆)cycloalkyl, R₅ is H, halo, NH₂,(C₁-C₆)alkoxy, or halo(C₁-C₆)alkoxy.

What is also provided is a compound which is 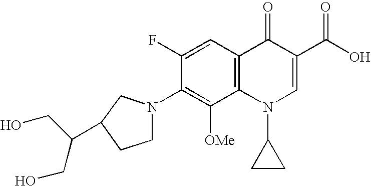
1-Cyclopropyl-6-fluoro-7-[3-(2-hydroxy-1-hydroxymethyl-ethyl)-pyrrolidin-1-yl]-8-methoxy-4-oxo-1,4-dihydro-quinoline-3-carboxylic acid; 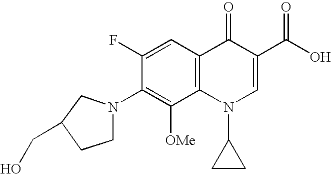
1-Cyclopropyl-6-fluoro-7-(3-hydroxymethyl-pyrrolidin-1-yl)-8-methoxy-4-oxo-1,4-dihydro-quinoline-3-carboxylic acid; 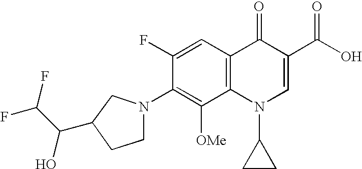
1-Cyclopropyl-7-[3-(2,2-difluoro-1-hydroxy-ethyl)-pyrrolidin-1-yl]-6-fluoro-8-methoxy-4-oxo-1,4-dihydro-quinoline-3-carboxylic acid; 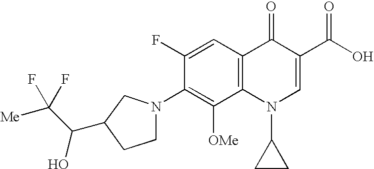
1-Cyclopropyl-7-[3-(2,2-difluoro-1-hydroxy-propyl)-pyrrolidin-1-yl]-6-fluoro-8-methoxy-4-oxo-1,4-dihydro-quinoline-3-carboxylic acid; 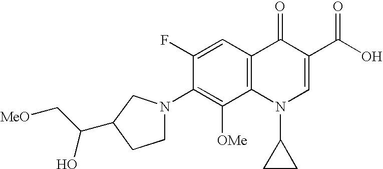
1-Cyclopropyl-6-fluoro-7-[3-(1-hydroxy-2-methoxy-ethyl)-pyrrolidin-1-yl]-8-methoxy-4-oxo-1,4-dihydro-quinoline-3-carboxylic acid; 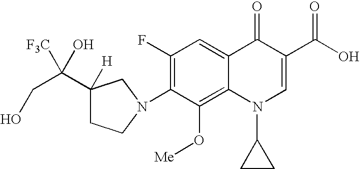
1-Cyclopropyl-6-fluoro-8-methoxy-4-oxo-7-[3-(2,2,2-trifluoro-1-hydroxy-1-hydroxymethyl-ethyl)-pyrrolidin-1-yl]-1,4-dihydro-quinoline-3-carboxylic acid; 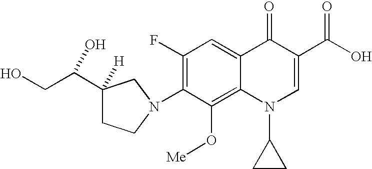
1-Cyclopropyl-7-[3R-(1R,2-dihydroxy-ethyl)-pyrrolidin-1-yl]-6-fluoro-8-methoxy-4-oxo-1,4-dihydro-quinoline-3-carboxylic acid; 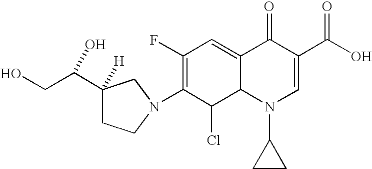
8-Chloro-1-cyclopropyl-7-[3R-(1R,2-dihydroxy-ethyl)-pyrrolidin-1-yl]-6-fluoro-4-oxo-1,4,8,8a-tetrahydro-quinoline-3-carboxylic acid; 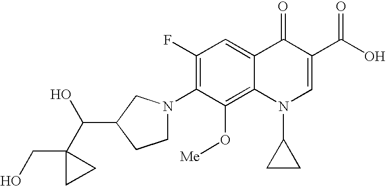
1-Cyclopropyl-6-fluoro-7-{3-[hydroxy-(1-hydroxymethyl-cyclopropyl)-methyl]-pyrrolidin-1-yl}-8-methoxy-4-oxo-1,4-dihydro-quinoline-3-carboxylic acid; 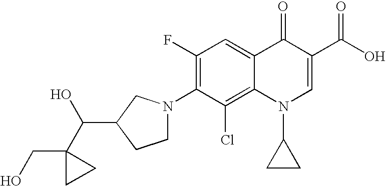
8-Chloro-1-cyclopropyl-6-fluoro-7-{3-[hydroxy-(1-hydroxymethyl-cyclopropyl)-methyl]-pyrrolidin-1-yl}-4-oxo-1,4-dihydro-quinoline-3-carboxylic acid; 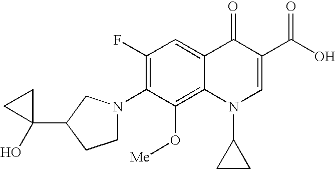
1-Cyclopropyl-6-fluoro-7-[3-(1-hydroxy-cyclopropyl)-pyrrolidin-1-yl]-8-methoxy-4-oxo-1,4-dihydro-quinoline-3-carboxylic acid; 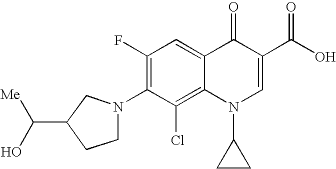
1-Cyclopropyl-6-fluoro-8-chloro-4-oxo-7-[3-(1-hydroxy-ethyl)-pyrrolidin-1-yl]-1,4-dihydro-quinoline-3-carboxylic acid; 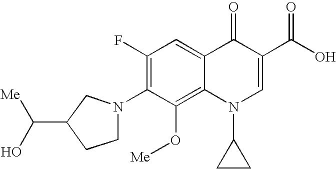
1-Cyclopropyl-6-fluoro-8-methoxy-4-oxo-7-[3-(1-hydroxy-ethyl)-pyrrolidin-1-yl]-1,4-dihydro-quinoline-3-carboxylic acid; 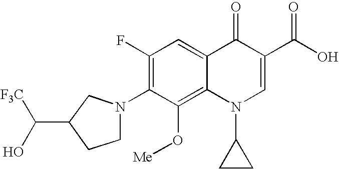
1-Cyclopropyl-6-fluoro-8-methoxy-4-oxo-7-[3-(2,2,2-trifluoro-1-hydroxy-ethyl)-pyrrolidin-1-yl]-1,4-dihydro-quinoline-3-carboxylic acid; 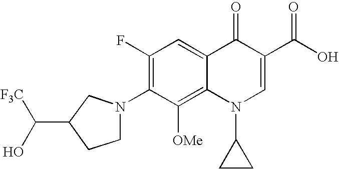
1-Cyclopropyl-6-fluoro-8-methoxy-4-oxo-7-[3-(2,2,2-trifluoro-1-hydroxy-ethyl)-pyrrolidin-1-yl]-1,4-dihydro-quinoline-3-carboxylic acid; 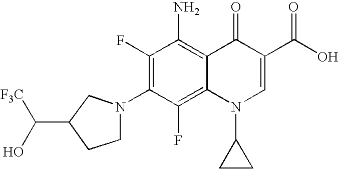
5-Amino-1-cyclopropyl-6,8-difluoro-4-oxo-7-[3-(2,2,2-trifluoro-1-hydroxy-ethyl)-pyrrolidin-1-yl]-1,4-dihydro-quinoline-3-carboxylic acid; 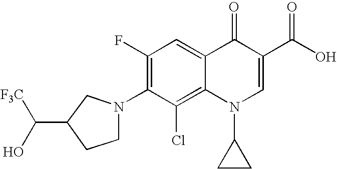
8-Chloro-1-cyclopropyl-6-fluoro-4-oxo-7-[3-(2,2,2-trifluoro-1-hydroxy-ethyl)-pyrrolidin-1-yl]-1,4-dihydro-quinoline-3-carboxylic acid; 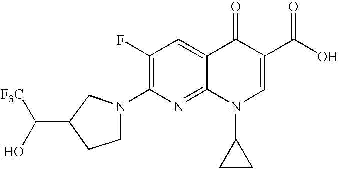
1-Cyclopropyl-6-fluoro-4-oxo-7-[3-(2,2,2-trifluoro-1-hydroxy-ethyl)-pyrrolidin-1-yl]-1,4-dihydro-[1,8]naphthyridine-3-carboxylic acid; 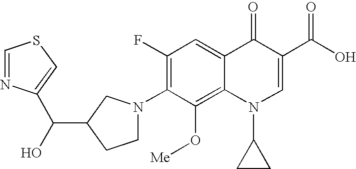
1-Cyclopropyl-6-fluoro-7-[3-(hydroxy-thiazol-4-yl-methyl)-pyrrolidin-1-yl]-8-methoxy-4-oxo-1,4-dihydro-quinoline-3-carboxylic acid; 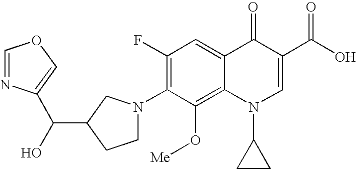
1-Cyclopropyl-6-fluoro-7-[3-(hydroxy-thiazol-4-yl-methyl)-pyrrolidin-1-yl]-8-methoxy-4-oxo-1,4-dihydro-quinoline-3-carboxylic acid; 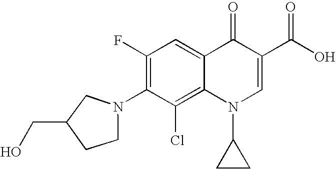
8-Chloro-1-cyclopropyl-6-fluoro-7-(3-hydroxymethyl-pyrrolidin-1-yl)-4-oxo-1,4-dihydro-quinoline-3-carboxylic acid; 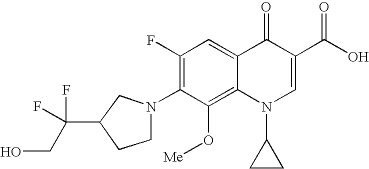
1-Cyclopropyl-7-[3-(1,1-difluoro-2-hydroxy-ethyl)-pyrrolidin-1-yl]-6-fluoro-8-methoxy-4-oxo-1,4-dihydro-quinoline-3-carboxylic acid; 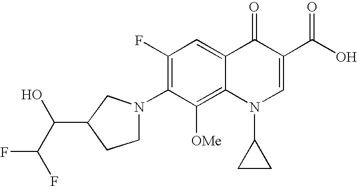
1-Cyclopropyl-7-[3-(2,2-difluoro-1-hydroxy-ethyl)-pyrrolidin-1-yl]-6-fluoro-8-methoxy-4-oxo-1,4-dihydro-quinoline-3-carboxylic acid; 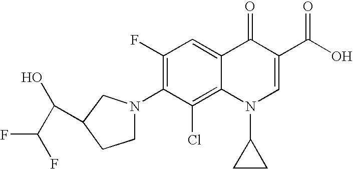
1-Cyclopropyl-7-[3-(2,2-difluoro-1-hydroxy-ethyl)-pyrrolidin-1-yl]-6-fluoro-8-chloro-4-oxo-1,4-dihydro-quinoline-3-carboxylic acid; 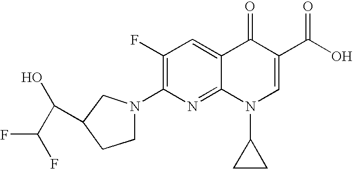
1-Cyclopropyl-7-[3-(2,2-difluoro-1-hydroxy-ethyl)-pyrrolidin-1-yl]-6-fluoro-4-oxo-1,4-dihydro-[1,8]naphthyridine-3-carboxylic acid; 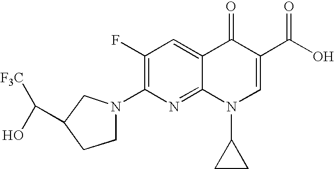
1-Cyclopropyl-6-fluoro-4-oxo-7-[3-(2,2,2-trifluoro-1-hydroxy-ethyl)-pyrrolidin-1-yl]-1,4-dihydro-[1,8]naphthyridine-3-carboxylic acid; 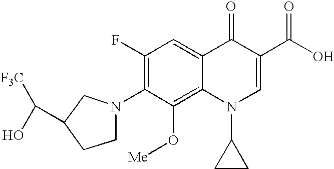
1-Cyclopropyl-6-fluoro-8-methoxy-4-oxo-7-[3-(2,2,2-trifluoro-1-hydroxy-ethyl)-pyrrolidin-1-yl]-1,4-dihydro-quinoline-3-carboxylic acid; 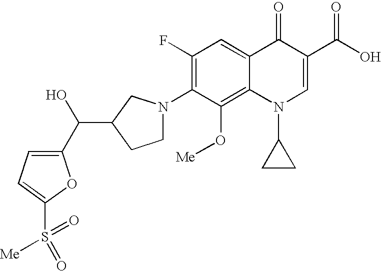
1-Cyclopropyl-6-fluoro-7-{3-[hydroxy-(5-methanesulfonyl-furan-2-yl)-methyl]-pyrrolidin-1-yl}-8-methoxy-4-oxo-1,4-dihydro-quinoline-3-carboxylic acid; 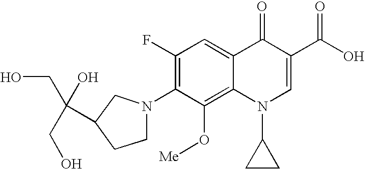
1-Cyclopropyl-7-[3-(1,2-dihydroxy-1-hydroxymethyl-ethyl)-pyrrolidin-1-yl]-6-fluoro-8-methoxy-4-oxo-1,4-dihydro-quinoline-3-carboxylic acid; 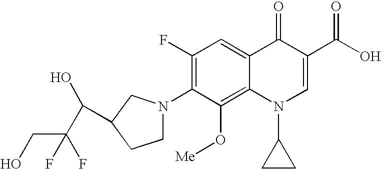
1-Cyclopropyl-7-[3-(2,2-difluoro-1,3-dihydroxy-propyl)-pyrrolidin-1-yl]-6-fluoro-8-methoxy-4-oxo-1,4-dihydro-quinoline-3-carboxylic acid; 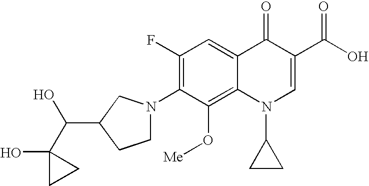
1-Cyclopropyl-6-fluoro-7-{3-[hydroxy-(1-hydroxy-cyclopropyl)-methyl]-pyrrolidin-1-yl}-8-methoxy-4-oxo-1,4-dihydro-quinoline-3-carboxylic acid; 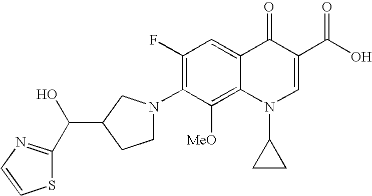
1-Cyclopropyl-6-fluoro-7-[3-(hydroxy-thiazol-2-yl-methyl)-pyrrolidin-1-yl]-8-methoxy-4-oxo-1,4-dihydro-quinoline-3-carboxylic acid; 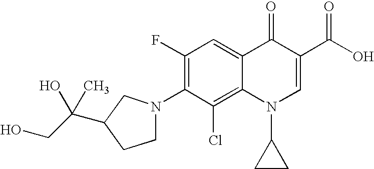
8-Chloro-1-cyclopropyl-7-[3-(1,2-dihydroxy-1-methyl-ethyl)-pyrrolidin-1-yl]-6-fluoro-4-oxo-1,4-dihydro-quinoline-3-carboxylic acid; 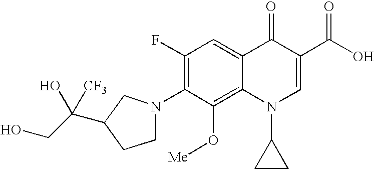
1-Cyclopropyl-6-fluoro-8-methoxy-4-oxo-7-[3-(2,2,2-trifluoro-1-hydroxy-1-hydroxymethyl-ethyl)-pyrrolidin-1-yl]-1,4-dihydro-quinoline-3-carboxylic acid; 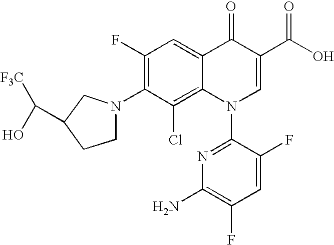
1-(6-Amino-3,5-difluoro-pyridin-2-yl)-8-chloro-6-fluoro-4-oxo-7-[3-(2,2,2-trifluoro-1-hydroxy-ethyl)-pyrrolidin-1-yl]-1,4-dihydro-quinoline-3-carboxylic acid; 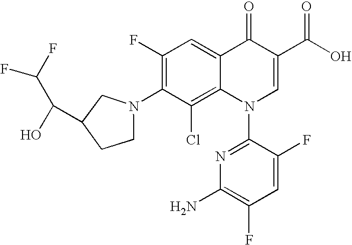
1-(6-Amino-3,5-difluoro-pyridin-2-yl)-8-chloro-7-[3-(2,2-difluoro-1-hydroxy-ethyl)-pyrrolidin-1-yl]-6-fluoro-4-oxo-1,4-dihydro-quinoline-3-carboxylic acid; or 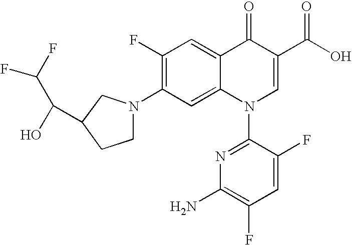
1-(6-Amino-3,5-difluoro-pyridin-2-yl)-7-[3-(2,2-difluoro-1-hydroxy-ethyl)-pyrrolidin-1-yl]-6-fluoro-4-oxo-1,4-dihydro-quinoline-3-carboxylic acid.

What is also provided is a compound of formula III 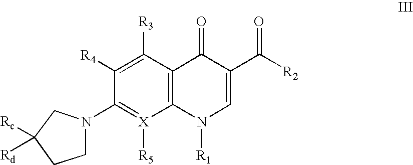
or a pharmaceutically acceptable salt thereof, wherein:
X is C or N, provided that when X is N, R₅ is absent;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 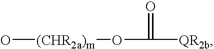
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 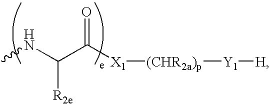
   wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R_{c} is OH,
   OPO(OH)₂,
   OPO(O(C₁-C₆)alkyl)₂, 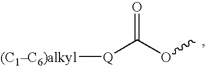
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 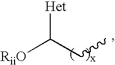
   wherein “ [symbol] ” indicates the point of attachment, het is a 5- or 6-membered heterocyclo or heteroaryl group, and x is an integer of from 0 to 10; 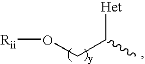
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 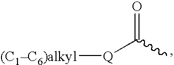
         as defined above; and
R_{d} is (C₁-C₆)alkyl, 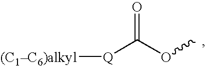
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 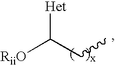
wherein “ [symbol] ” indicates the point of attachment, het is a 5- or 6-membered heterocyclo or heteroaryl group, and x is an integer of from 0 to 10; 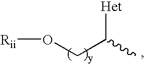
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂,

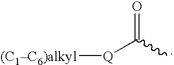

What is also provided is a compound which is 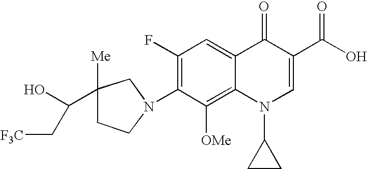
1-Cyclopropyl-6-fluoro-8-methoxy-7-[3-methyl-3-(3,3,3-trifluoro-1-hydroxy-propyl)-pyrrolidin-1-yl]-4-oxo-1,4-dihydro-quinoline-3-carboxylic acid; 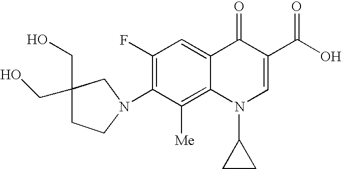
7-(3,3-Bis-hydroxymethyl-pyrrolidin-1-yl)-1-cyclopropyl-6-fluoro-8-methyl-4-oxo-1,4-dihydro-quinoline-3-carboxylic acid; or 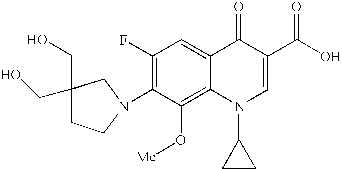
7-(3,3-Bis-hydroxymethyl-pyrrolidin-1-yl)-1-cyclopropyl-6-fluoro-8-methoxy-4-oxo-1,4-dihydro-quinoline-3-carboxylic acid.

What is also provided is a compound of formual IV 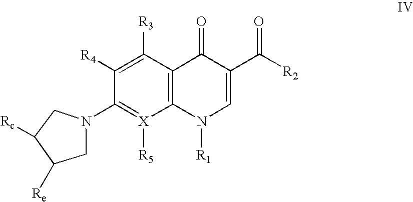
or a pharmaceutically acceptable salt thereof, wherein:
X is C or N, provided that when X is N, R₅ is absent;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 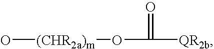
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 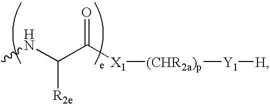
   wherein “ [symbol] ” indicates the point of attachment, 2a is as defined above, R₂e is H or (C₁-C₆)alkyl, e is an integer of from 1 to 10, p is an integer of from 2 to 10, and X₁ and Y₁ are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R_{c} is OH,
   OPO(OH)₂,
   OPO(O(C₁-C₆)alkyl)₂, 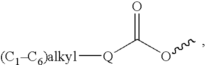
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 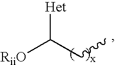
   wherein “ [symbol] ” indicates the point of attachment, het is a 5- or 6-membered heterocyclo or heteroaryl group, and x is an integer of from 0 to 10; 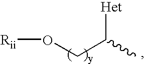
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 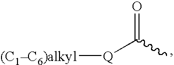
         as defined above;
      provided that 3 or fewer of R_{c} R_{d}, Rₑ, and R_{f} are H; and
Rₑ is OPO(OH)₂,
   OPO(O(C₁-C₆)alkyl)₂, 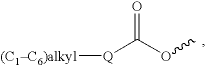
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 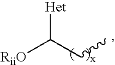
   wherein “ [symbol] ” indicates the point of attachment, het is a 5- or 6-membered heterocyclo or heteroaryl group, and x is an integer of from 0 to 10; 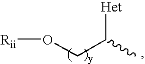
   wherein “ [symbol] ” indicates the point of attachment, het is as defined above, and y is an integer of from 1 to 10, wherein Rᵢᵢ is H,
   (C₁-C₆)alkyl,
   PO(OH)₂,
   PO(O(C₁-C₆)alkyl)₂, or 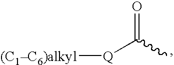
   as defined above.

What is also provided is a compound which is 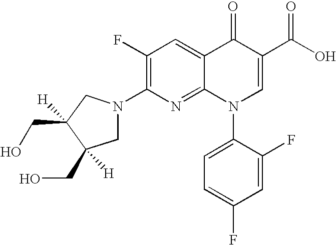
7-(3,4-Bis-hydroxymethyl-pyrrolidin-1-yl)-1-(2,4-difluoro-phenyl)-6-fluoro-4-oxo-1,4-dihydro-[1,8]naphthyridine-3-carboxylic acid; 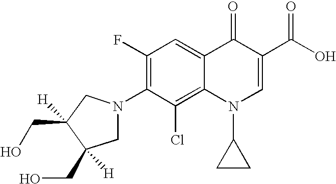
7-(3R,4S-bis-hydroxymethyl-pyrrolidin-1-yl)-8-chloro-1-cyclopropyl-6-fluoro-4-oxo-1,4-dihydro-quinoline-3-carboxylic acid; 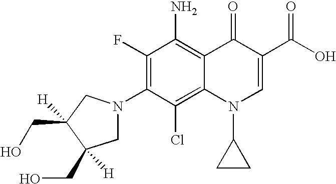
5-Amino-7-(3R,4S-bis-hydroxymethyl-pyrrolidin-1-yl)-8-chloro-1-cyclopropyl-6-fluoro-4-oxo-1,4-dihydro-quinoline-3-carboxylic acid; 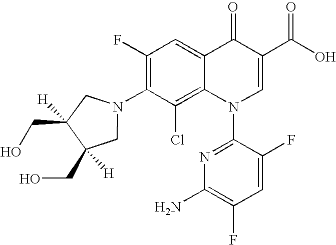
1-(6-Amino-3,5-difluoro-pyridin-2-yl)-7-(3,4-bis-hydroxymethyl-pyrrolidin-1-yl)-8-chloro-6-fluoro-4-oxo-1,4-dihydro-quinoline-3-carboxylic acid; 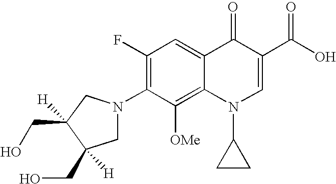
7-(3,4-Bis-hydroxymethyl-pyrrolidin-1-yl)-1-cyclopropyl-6-fluoro-8-methoxy-4-oxo-1,4-dihydro-quinoline-3-carboxylic acid; 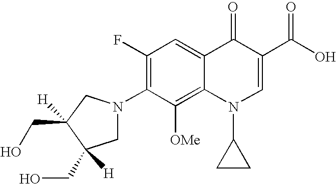
7-(3,4-Bis-hydroxymethyl-pyrrolidin-1-yl)-1-cyclopropyl-6-fluoro-8-methoxy-4-oxo-1,4-dihydro-quinoline-3-carboxylic acid; 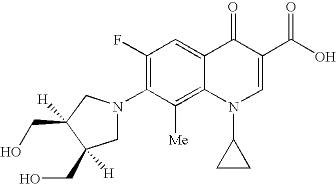
7-(3,4-Bis-hydroxymethyl-pyrrolidin-1-yl)-1-cyclopropyl-6-fluoro-8-methyl-4-oxo-1,4-dihydro-quinoline-3-carboxylic acid; 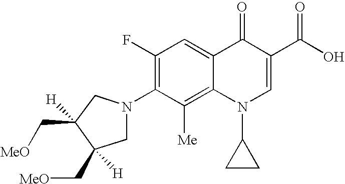
7-(3,4-Bis-methoxymethyl-pyrrolidin-1-yl)-1-cyclopropyl-6-fluoro-8-methyl-4-oxo-1,4-dihydro-quinoline-3-carboxylic acid; 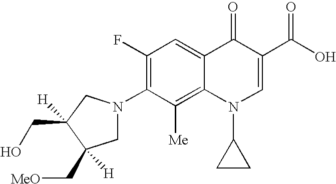
1-Cyclopropyl-7-(3-methoxymethyl-4-hydroxymethyl-pyrrolidin-1-yl)-6-fluoro-8-methyl-4-oxo-1,4-dihydro-quinoline-3-carboxylic acid; 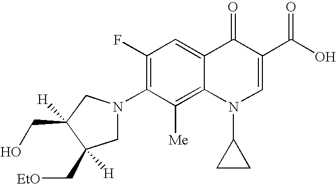
1-Cyclopropyl-7-(3-ethoxymethyl-4-hydroxymethyl-pyrrolidin-1-yl)-6-fluoro-8-methyl-4-oxo-1,4-dihydro-quinoline-3-carboxylic acid; 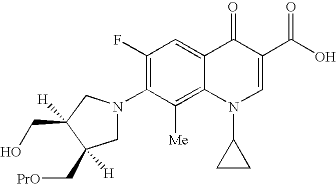
1-Cyclopropyl-7-(3-propoxymethyl-4-hydroxymethyl-pyrrolidin-1-yl)-6-fluoro-8-methyl-4-oxo-1,4-dihydro-quinoline-3-carboxylic acid; 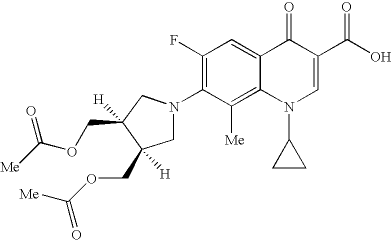
7-(3,4-Bis-acetoxymethyl-pyrrolidin-1-yl)-1-cyclopropyl-6-fluoro-8-methyl-4-oxo-1,4-dihydro-quinoline-3-carboxylic acid; 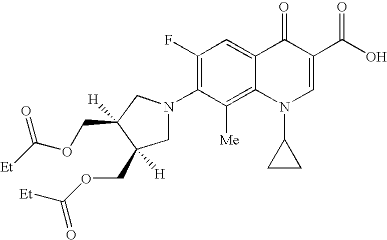
7-(3,4-Bis-propionyloxymethyl-pyrrolidin-1-yl)-1-cyclopropyl-6-fluoro-8-methyl-4-oxo-1,4-dihydro-quinoline-3-carboxylic acid; 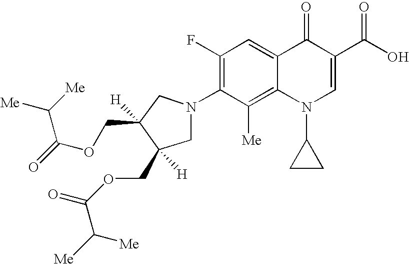
7-(3,4-Bis-isobutyryloxymethyl-pyrrolidin-1-yl)-1-cyclopropyl-6-fluoro-8-methyl-4-oxo-1,4-dihydro-quinoline-3-carboxylic acid; 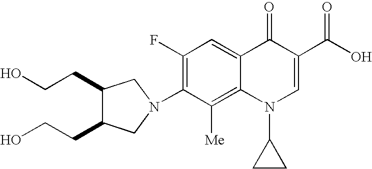
7-[3S,4R-Bis-(2-hydroxy-ethyl)-pyrrolidin-1-yl]-1-cyclopropyl-6-fluoro-8-methyl-4-oxo-1,4-dihydro-quinoline-3-carboxylic acid; 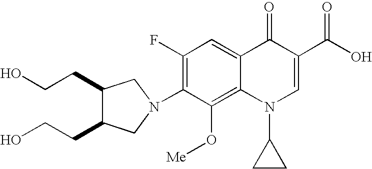
7-[3S,4R-Bis-(2-hydroxy-ethyl)-pyrrolidin-1-yl]-1-cyclopropyl-6-fluoro-8-methoxy-4-oxo-1,4-dihydro-quinoline-3-carboxylic acid; or 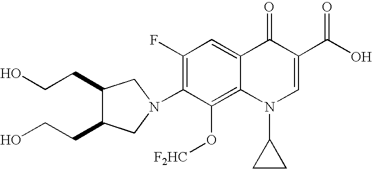
7-[3,4-Bis-(2-hydroxy-ethyl)-pyrrolidin-1-yl]-1-cyclopropyl-8-difluoromethoxy-6-fluoro-4-oxo-1,4-dihydro-quinoline-3-carboxylic acid.

What is also provided is a compound of formula V 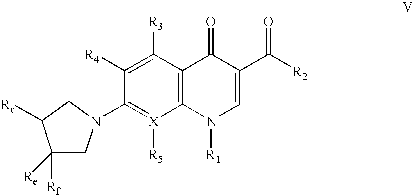
or a pharmaceutically acceptable salt thereof, wherein:
X is C or N, provided that when X is N, R₅ is absent;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 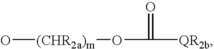
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 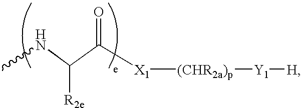
   wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R_{c} is
   OPO(OH)₂,
   OPO(O(C₁-C₆)alkyl)₂, 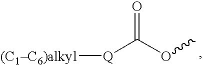
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 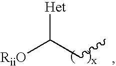
   wherein “ [symbol] ” indicates the point of attachment, het is a 5- or 6-membered heterocyclo or heteroaryl group, and x is an integer of from 0 to 10; 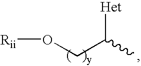
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 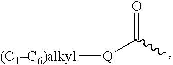
         as defined above; and
Rₑ and R_{f} are each independently (C₁-C₆)alkyl or together with the carbon to which they are attached, form a substituted or unsubstituted 3, 4, 5, or 6-membered ring containing 0, 1, 2, or 3 heteroatoms selected from NH, N(C₁-C₆)alkyl, S, or O.

What is also provided is a compound which is 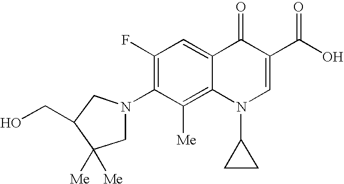
8-Methyl-1-cyclopropyl-6-fluoro-7-(4-hydroxymethyl-3,3-dimethyl-pyrrolidin-1-yl)-4-oxo-1,4-dihydro-quinoline-3-carboxylic acid; 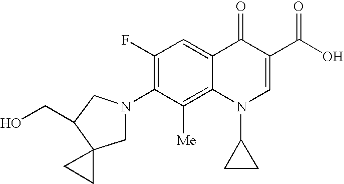
8-Methyl-1-cyclopropyl-6-fluoro-7-(7-hydroxymethyl-5-aza-spiro[2.4]hept-5-yl)-4-oxo-1,4-dihydro-quinoline-3-carboxylic acid; 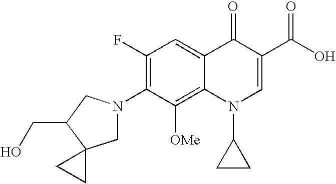
8-Methoxy-1-cyclopropyl-6-fluoro-7-(7-hydroxymethyl-5-aza-spiro[2.4]hept-5-yl)-4-oxo-1,4-dihydro-quinoline-3-carboxylic acid; 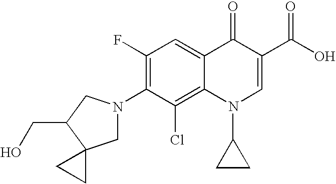
8-Chloro-1-cyclopropyl-6-fluoro-7-(7-hydroxymethyl-5-aza-spiro[2.4] hept-5-yl)-4-oxo-1,4-dihydro-quinoline-3-carboxylic acid; 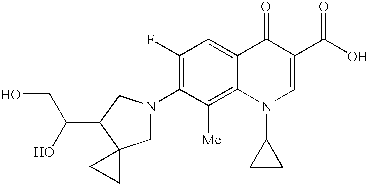
1-Cyclopropyl-7-[7-(1,2-dihydroxy-ethyl)-5-aza-spiro[2.4]hept-5-yl]-6-fluoro-8-methyl-4-oxo-1,4-dihydro-quinoline-3-carboxylic acid; 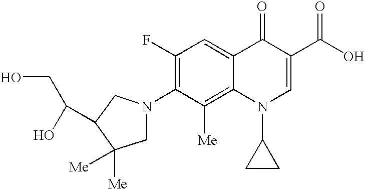
1-Cyclopropyl-6-fluoro-7-[4-(1-hydroxy-ethyl)-3,3-dimethyl-pyrrolidin-1-yl]-8-methyl-4-oxo-1,4-dihydro-quinoline-3-carboxylic acid; 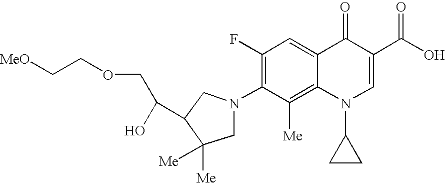
1-Cyclopropyl-6-fluoro-7-{4-[1-hydroxy-2-(2-methoxy-ethoxy)-ethyl]-3,3-dimethyl-pyrrolidin-1-yl}-8-methyl-4-oxo-1,4-dihydro-quinoline-3-carboxylic acid; 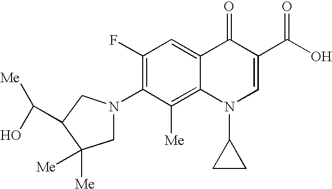
1-Cyclopropyl-6-fluoro-7-[4-(1-hydroxy-ethyl)-3,3-dimethyl-pyrrolidin-1-yl]-8-methyl-4-oxo-1,4-dihydro-quinoline-3-carboxylic acid; or 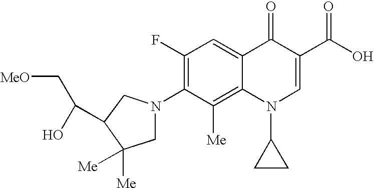
1-Cyclopropyl-6-fluoro-7-[4-(1-hydroxy-2-methoxy-ethyl)-3,3-dimethyl-pyrrolidin-1-yl]-8-methyl-4-oxo-1,4-dihydro-quinoline-3-carboxylic acid.

What is also provided is a compound of formula VI 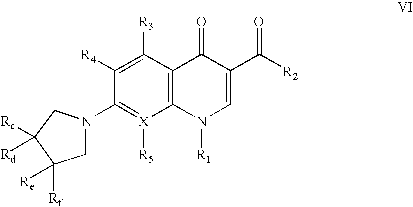
or a pharmaceutically acceptable salt thereof, wherein:
X is C or N, provided that whne X is N, R₅ is absent;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 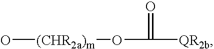
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR₂d, wherein R_{2d} is as defined above, 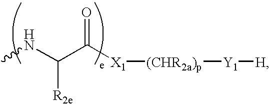
   wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(CX-C₆)alkoxy;
R_{c} and Rₑ are OH,
   OPO(OH)₂,
   OPO(O(C₁-C₆)alkyl)₂, 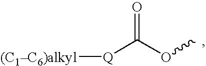
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 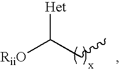
   wherein “ [symbol] ” indicates the point of attachment, het is a 5- or 6-membered heterocyclo or heteroaryl group, that does not contain an NH, and x is an integer of from 0 to 10; 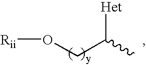
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 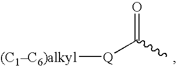
         as defined above; and
R_{d} and R_{f} are each independently (C₁-C₆)alkyl.

What is also provided is a compound which is 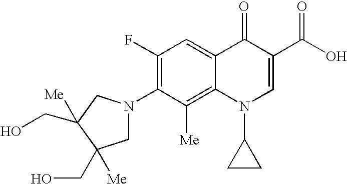
7-(3,4-Bis-hydroxymethyl-3,4-dimethyl-pyrrolidin-1-yl)-1-cyclopropyl-6-fluoro-8-methyl-4-oxo-1,4-dihydro-quinoline-3-carboxylic acid; or 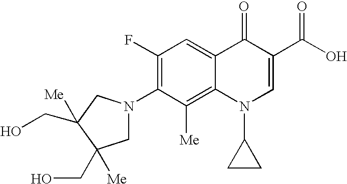
7-(3,4-Bis-hydroxymethyl-3,4-dimethyl-pyrrolidin-1-yl)-1-cyclopropyl-6-fluoro-8-methoxy-4-oxo-1,4-dihydro-quinoline-3-carboxylic acid.

What is also provided is a compound of formula VII 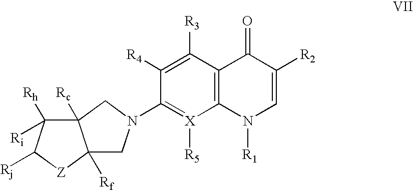
or a pharmaceutically acceptable salt thereof, wherein:
X is C or N, provided that whne X is N, R₅ is absent;
Z is absent or is a linker containing1, 2, or 3 substituted or unsubstituted carbon atoms;
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is OH,
   OBF₂,
   O(C₁-C₆)alkyl,
   O(C₃-C₆)cycloalkyl, 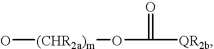
   wherein m is an integer of from 1 to 10, Q is O or is absent, and R₂ₐ is H or (C₁-C₆)alkyl and R_{2b} is (C₁-C₆)alkyl, aryl, or heteroaryl,
   O—(CHR₂ₐ)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
   NR_{2d}, wherein R_{2d} is as defined above, 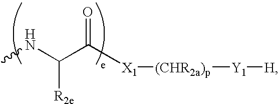
   wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y, are each independently NH or O;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
R_{c} and R_{f} are each independently H, (C₁-C₆)alkyl, or HO—(C₁-C₆)alkyl;
Rₕ, Rᵢ, and Rⱼ are each independently H,
   OH,
   OPO(OH)₂,
   OPO(O(C₁-C₆)alkyl)₂, 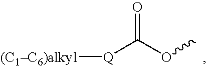
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 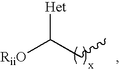
   wherein “ [symbol] ” indicates the point of attachment, het is a 5- or 6-membered heterocyclo or heteroaryl group that does not contain an NH, and x is an integer of from 0 to 10; 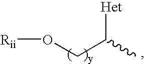
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 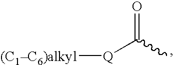
         as defined above;
         provided that not all of Rₕ Rᵢ, and Rⱼ are H.

What is also provided is a compound which is 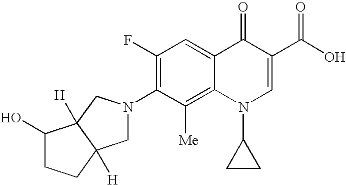
1-Cyclopropyl-6-fluoro-7-(4-hydroxy-hexahydro-cyclopenta[c]pyrrol-2-yl)-8-methyl-4-oxo-1,4-dihydro-quinoline-3-carboxylic acid; 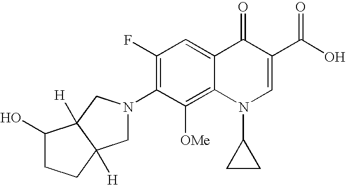
1-Cyclopropyl-6-fluoro-7-(4-hydroxy-hexahydro-cyclopenta[c]pyrrol-2-yl)-8-methoxy-4-oxo-1,4-dihydro-quinoline-3-carboxylic acid; 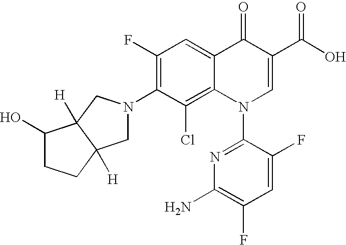
1-(6-Amino-3,5-difluoro-pyridin-2-yl)-8-chloro-6-fluoro-7-(4-hydroxy-hexahydro-cyclopenta[c]pyrrol-2-yl)-4-oxo-1,4-dihydro-quinoline-3-carboxylic acid; 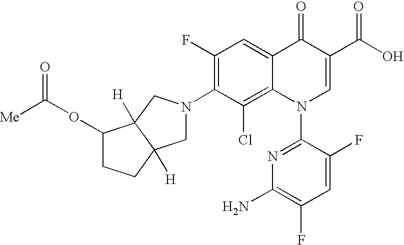
7-(4-Acetoxy-hexahydro-cyclopenta[c]pyrrol-2-yl)-1-(6-amino-3,5-difluoro-pyridin-2-yl)-8-chloro-6-fluoro-4-oxo-1,4-dihydro-quinoline-3-carboxylic acid; 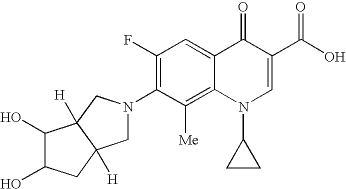
1-Cyclopropyl-7-(4,5-dihydroxy-hexahydro-cyclopenta[cpyrrol-2-yl)-6-fluoro-8-methyl-4-oxo-1,4-dihydro-quinoline-3-carboxylic acid; 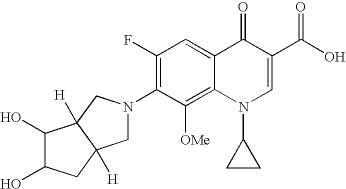
1-Cyclopropyl-7-(4,5-dihydroxy-hexahydro-cyclopenta[c]pyrrol-2-yl)-6-fluoro-8-methoxy-4-oxo-1,4-dihydro-quinoline-3-carboxylic acid; 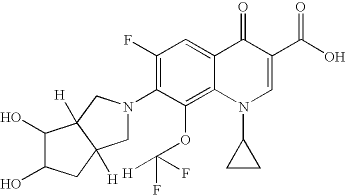
1-Cyclopropyl-8-difluoromethoxy-7-(4,5-dihydroxy-hexahydro-cyclopenta [c]pyrrol-2-yl)-6-fluoro-4-oxo-1,4-dihydro-quinoline-3-carboxylic acid; 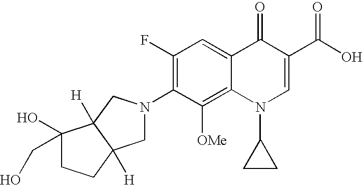
1-Cyclopropyl -6-fluoro-7-(4-hydroxy-4-hydroxymethyl-hexahydro-cyclopenta [c]pyrrol-2-yl)-8-methoxy-4-oxo-1,4-dihydro-quinoline-3-carboxylic acid; 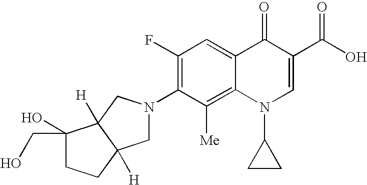
1-Cyclopropyl-6-fluoro-7-(4-hydroxy-4-hydroxymethyl-hexahydro-cyclopenta [c]pyrrol-2-yl)-8-methyl-4-oxo-1,4-dihydro-quinoline-3-carboxylic acid; 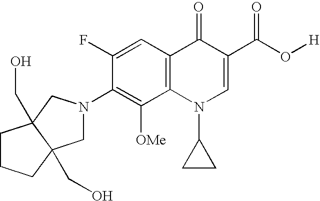
7-(3aR,7aS-Bis-hydroxymethyl-octahydro-isoindol-2-yl)-1-cyclopropyl-6-fluoro-8-methoxy-4-oxo-1,4-dihydro-quinoline-3-carboxylic acid; or 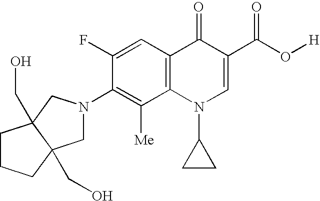
7-(3aR,7aS-Bis-hydroxymethyl-octahydro-isoindol-2-yl)-1-cyclopropyl-6-fluoro-8-methyl-4-oxo-1,4-dihydro-quinoline-3-carboxylic acid.

What is also provided is a pharmaceutical formulation comprising a compound of one of formulas I-V admixed with a pharmaceutically acceptable diluent, carrier, or excipient.

What is also provided is a method of treating a bacterial infection in a mammal, comprising administering to a mammal in need thereof an effective amount of a compound of formula I.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a straight or branched hydrocarbon of from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower (C₁-C₆)alkoxy, (C₁-C₆)thioalkoxy, halogen, oxo, thio, —OH, —SH, —F, —CF₃, —OCF₃, —NO₂, —CO₂H, —CO₂(C₁-C₆)alkyl, or

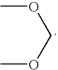

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substituents selected from alkyl, alkoxy, thioalkoxy, hydroxy, thiol, halogen, formyl, carboxyl, —CO₂(C₁-C₆)alkyl, —CO(C₁-C₆)alkyl, aryl, heteroaryl, wherein alkyl, aryl, and heteroaryl are as defined herein, or as indicated above for alkyl. Examples of substituted cycloalkyl groups include fluorocyclopropyl.

The term “halo” includes chlorine, fluorine, bromine, and iodine.

The term “aryl” means a cyclic or polycyclic aromatic ring having from 5 to 12 carbon atoms, and being unsubstituted or substituted with one or more of the substituent groups recited above for alkyl groups including, halogen, nitro, cyano —OH, —SH, —F, —CF₃, —OCF₃, 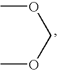
—CO₂H, —CO₂(C₁-C₆)alkyl, or —SO₂alkyl. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5- 1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

When a bond is represented by a symbol such as “- - - ” this is meant to represent that the bond may be absent or present provided that the resultant compound is stable and of satisfactory valency.

When a bond is represented by a line such as “ [symbol] ” this is meant to represent that the bond is the point of attachment between two molecular subunits.

The term “patient” means all mammals, including humans. Other examples of patients include cows, dogs, cats, goats, sheep, pigs, and rabbits.

A “therapeutically effective amount” is an amount of a compound of the present invention that, when administered to a patient, provides the desired effect; i.e., lessening in the severity of the symptoms associated with a bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain compounds of Formula I are also useful as intermediates for preparing other compounds of Formula I. Thus, a compound wherein R₂ is NR₂, can be metabolized to form another compound of the invention wherein R₂ is H. This conversion can occur under physiological conditions. To that end, both the non-metabolized compound of the invention and the metabolized compound of the invention—that is, the compound wherein R₂ is NR₂ and the compound wherein R₂ is H—can have antibacterial activity.

Some of the compounds of Formula I are capable of further forming pharmaceutically acceptable acid-addition and/or base salts. All of these forms are within the scope of the present invention. Thus, pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, S. M. et. al., “Pharmaceutical Salts,” Journal of Phannaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

A “prodrug” is an inactive derivative of a drug molecule that requires a chemical or an enzymatic biotransformation in order to release the active parent drug in the body.

Specific and preferred values for the compounds of the present invention are listed below for radicals, substituents, and ranges are for illustration purposes only, and they do not exclude other defined values or other values within defined ranges for the radicals and substituents.

Thus, we turn now to a compound of formula I, which has the structure:

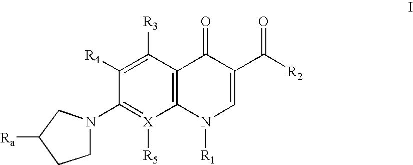

A specific value for R₁ is (C₁-C₆)cycloalkyl and halo(C₁-C₆)cycloalkyl, aryl, or heteroaryl. A specifc value for R₃ is H or NH₂. A specific value for R₄ is H or halo. A specific value for R₅ is halo, methyl, trifluoromethyl, methoxy, fluoromethoxy, difluoromethoxy, or trifluoromethoxy.

In another embodiment of a compound of formula I, a specific value for R₁ is cyclopropyl or fluorocyclopropyl. A specific value for R₃ is H or NH₂. A specific value for R₄ is H or F. A specific value for X is C or N. A specific value for R₅ is halo or methoxy.

In another embodiment of a compound of formula I, R₁, R₃, R₄, and R₅ are as provided in the following structures, wherein R₂ is OH, OBF₂, or O(C₁-C₆)alkyl, R₄ is H or F, and wherein A is

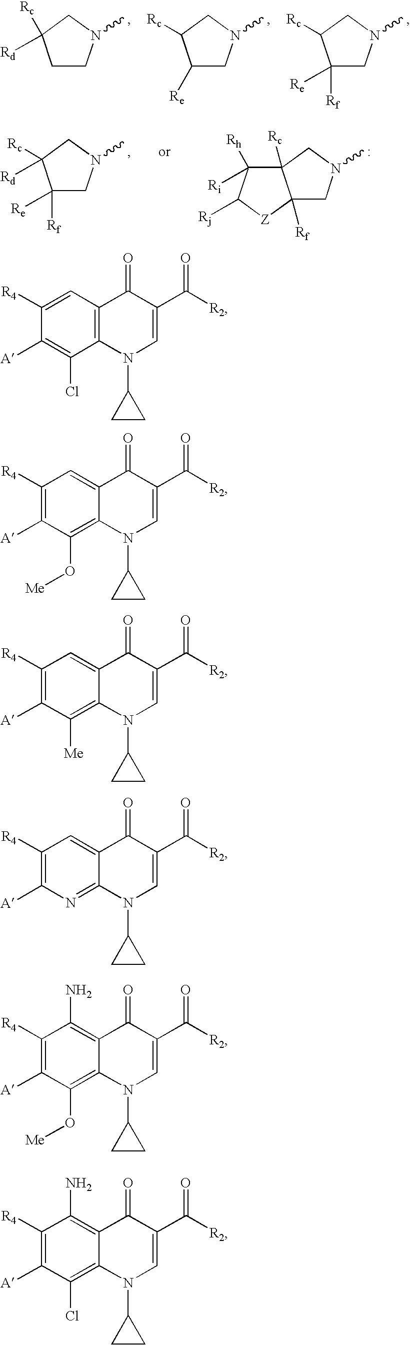

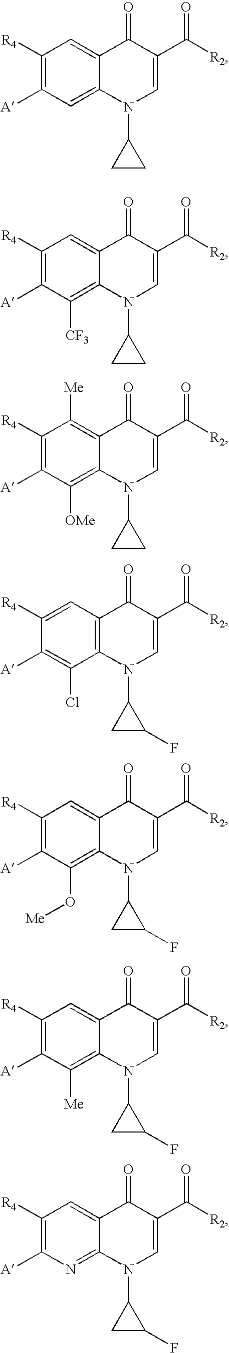

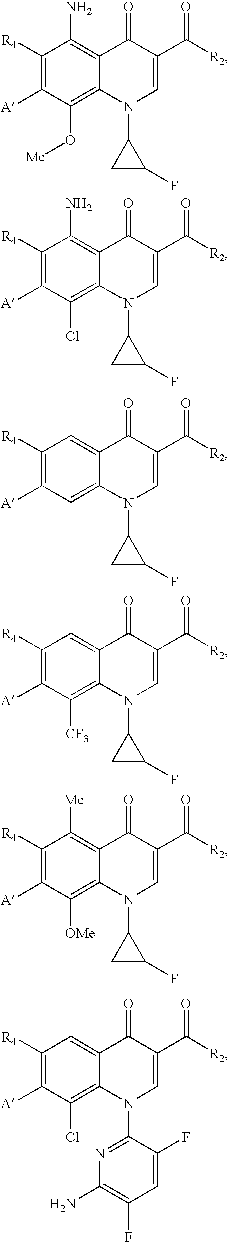

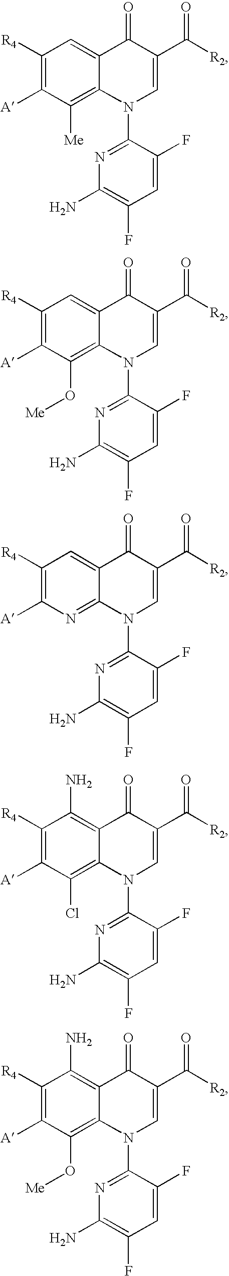

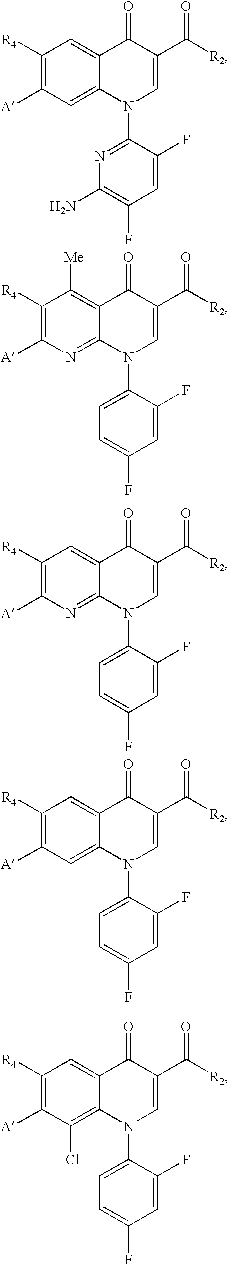

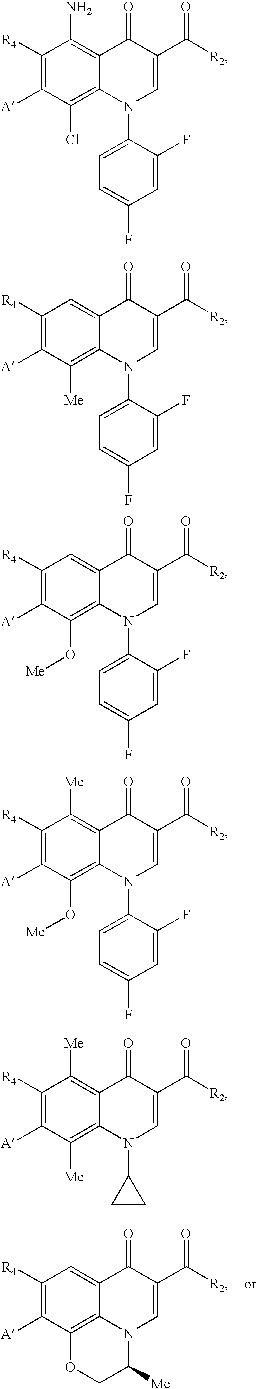

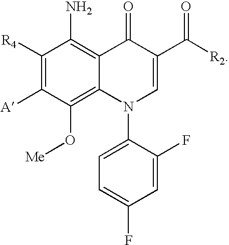

More particularly, compounds of the invention have the following core structures, wherein R₂ is OH and wherein A+ is

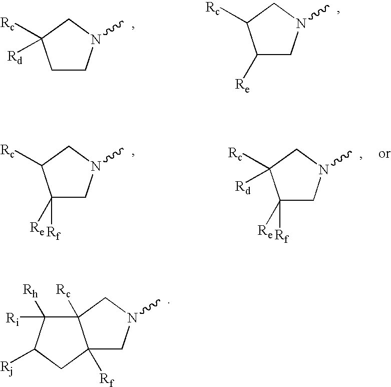

In another embodiment of a compound of formula I as depicted in the previous paragraph, A′ is 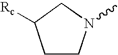
wherein R_{c} is OPO(OH)₂,
   OPO(O(C₂-C₆)alkyl)₂, 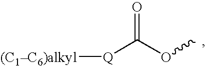
   wherein “ [symbol] ” indicates the point of attachment and Q is O or is absent,
   RᵢᵢO(C₂-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(Cc -C₆)haloalkyl-O—,
   RᵢᵢO(C₃-C₆)cycloalkyl-O—, 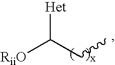
   wherein “ [symbol] ” indicates the point of attachment, het is a 5- or 6-membered heterocyclo or heteroaryl group, and x is an integer of from 0 to 10; 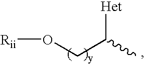
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₂-C₆)alkyl)₂, or 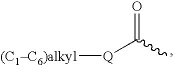
         as defined above.

Specific values for 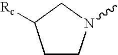
include:

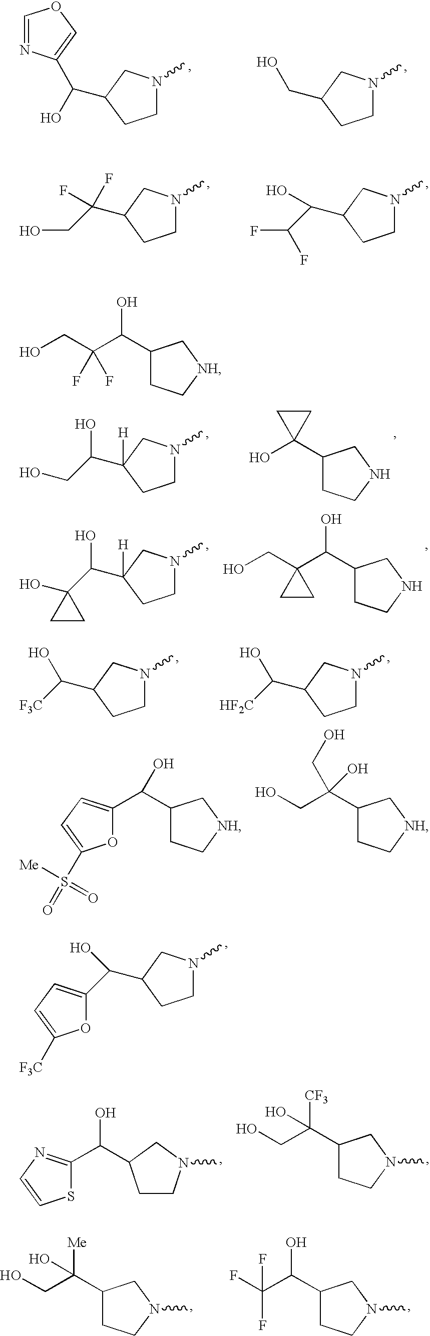

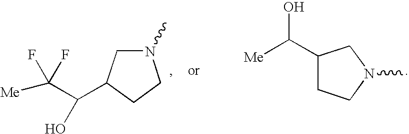

In another embodiment of the invention compound A′ is 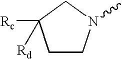
wherein R_{c} is OH,
   OPO(OH)₂)
   OPO(O(C₁-C₆)alkyl)₂, 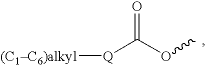
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 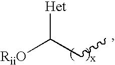
   wherein “ [symbol] ” indicates the point of attachment, het is a 5- or 6-membered heterocyclo or heteroaryl group, and x is an integer of from 0 to 10; 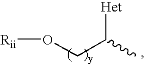
   wherein “ [symbol] ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, or 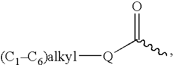
         as defined above; and
R_{d} is halo,
   (C₁-C₆)alkyl,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, or 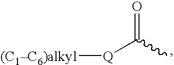
   wherein “ [symbol] ” indicates the point of attachment and Q is O or is absent.

A specific value for

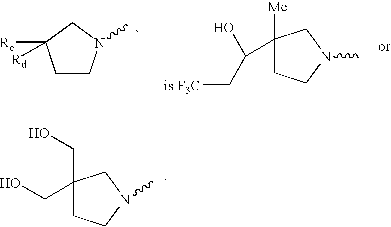

In another embodiment of a compound of formula I, A′ is 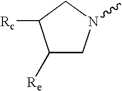
wherein R_{c} is OH,
   OPO(OH)₂,
   OPO(O(C₁-C₆)alkyl)₂, 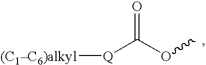
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 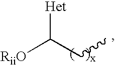
   wherein “ [symbol] ” indicates the point of attachment, het is a 5- or 6-membered heterocyclo or heteroaryl group, and x is an integer of from 0 to 10; 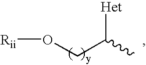
   wherein “ [symbol] ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 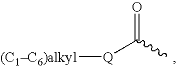
         as defined above; and
Rₑ is halo 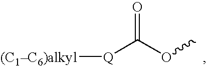
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 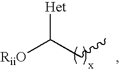
   wherein “ [symbol] ” indicates the point of attachment, het is a 5- or 6-membered heterocyclo or heteroaryl group, and x is an integer of from 0 to 10; 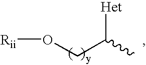
   wherein “ [symbol] ” indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, or 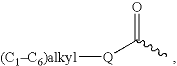
         as defined above.

Specific values for 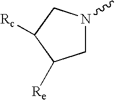
include

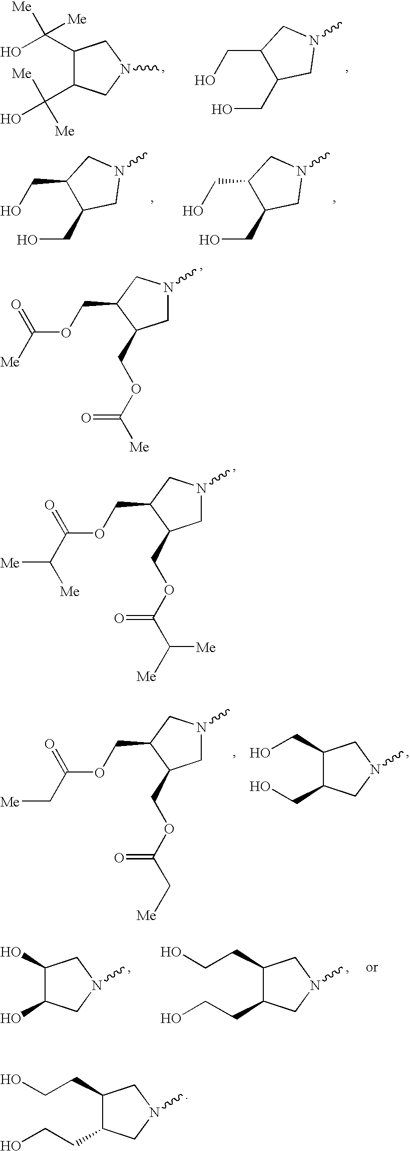

In another embodiment of the invention compound, A′ is 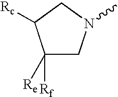
wherein R_{c} is
   OPO(OH)₂,
   OPO(O(C₁-C₆)alkyl)₂, 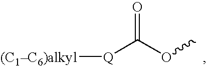
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 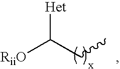
   wherein “ [symbol] ” indicates the point of attachment, het is a 5- or 6-membered heterocyclo or heteroaryl group, and x is an integer of from 0 to 10; 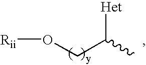
   wherein “ [symbol] ” indicates the point of attachment, het is as defined above, and y is an integer of from 1 to 10,
      wherein Rᵢᵢ is H,
         (C₁-C₆)alkyl,
         PO(OH)₂,
         PO(O(C₁-C₆)alkyl)₂, 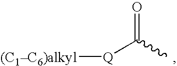
         as defined above; and
Rₑ and R_{f} are each independently (C₁-C₆)alkyl or together with the carbon to which they are attached, form a substituted or unsubstituted 3, 4, 5, or 6-membered ring containing 0, 1, 2, or 3 heteroatoms selected from NH, N(C₁-C₆)alkyl, S, or O.

Specific values for 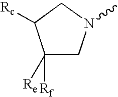
include

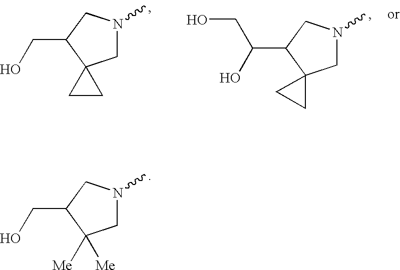

In another embodiment of the invention compound, A′ is 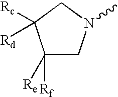
wherein R_{c} and Rₑ are each independently H,
   OH,
   OPO(OH)₂, 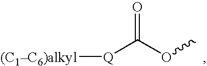
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 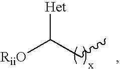
   wherein “ [symbol] ” indicates the point of attachment, het 5 is a 5- or 6-membered heterocyclo or heteroaryl group, and x is an integer of from 0 to 10; 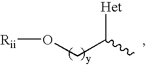
   wherein “ [symbol] ” indicates the point of attachment, het is as defined above, and y is an integer of from 1 to 10,
      wherein Rᵢᵢ is H,
         (C₁-C₆)alkyl,
         PO(OH)₂, 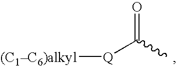
         as defined above; and
R_{d} and R_{f} are each independently (C₁-C₆)alkyl, or taken together with the carbons to which they are attached form a substituted or unsubstituted 4, 5, or 6 membered ring, optionally containing one heteroatom selected from NH, N(C₁-C₆)alkyl, S, or O.

A specific value for

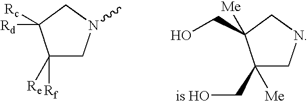

In another embodiment of the invention compound, A′ is 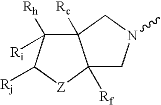
wherein
Z is absent or is a linker containing1, 2, or 3 substituted or unsubstituted carbon atoms;
R_{c} and R_{f} are each independently H, (C₁-C₆)alkyl, or HO—(C₁-C₆)alkyl;
Rₕ, Rᵢ, and Rⱼ are each independently H,
   OH,
   OPO(OH)₂, 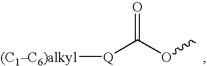
   wherein “ [symbol] ” indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 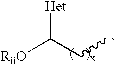
   wherein “ [symbol] ” indicates the point of attachment, het is a 5- or 6-membered heterocyclo or heteroaryl group, and x is an integer of from 0 to 10; 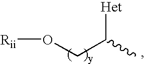
   wherein “ [symbol] ” indicates the point of attachment, het is as defined above, and y is an integer of from 1 to 10,
      wherein Rᵢᵢ is H,
         (C₁-C₆)alkyl,
         PO(OH)₂, 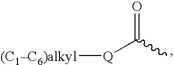
         as defined above;
provided that not all of Rₕ Rᵢ, and Rⱼ are H.

Specific values for 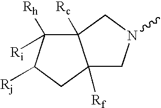
include

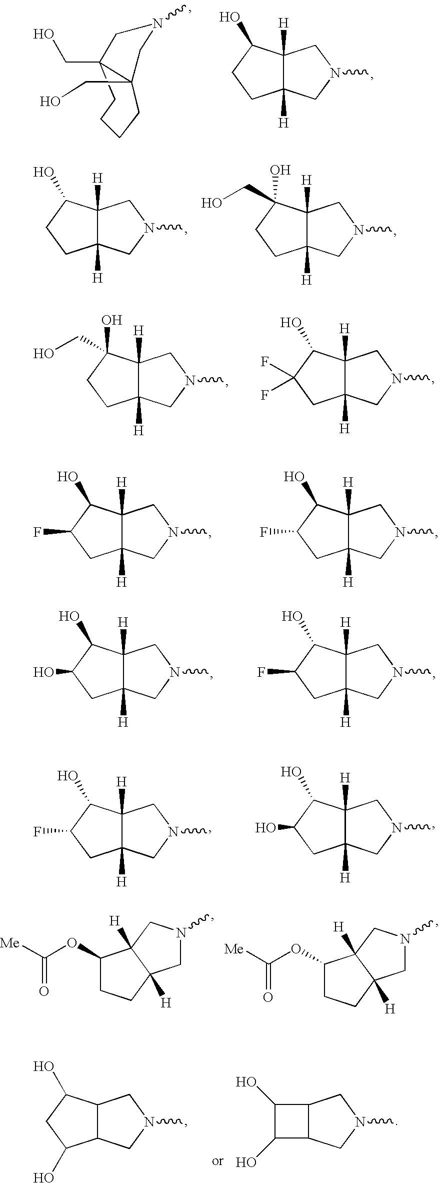

Turning now to a compound of formula II, specific values for R₁, R₂, R₃, R₄, and R₅ and for 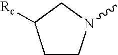
are as provided for a compound of formula I.

Turning now to a compound of formula III, specific values for R₁, R₂, R₃, R₄, and R₅ and, 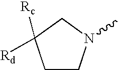
for are as provided for a compound of formula I.

Turning now to a compound of formula IV, specific values for R₁, R₂, R₃, R₄, and R₅ and 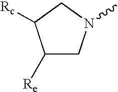
are as provided for a compound of formula I.

Turning now to a compound of formula V, specific values for R₁, R₂, R₃, R₄, and R₅ and 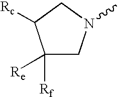
are as provided for a compound of formula I.

Turning now to a compound of formula VI, specific values for R₁, R₂, R₃, R₄, and R₅ and 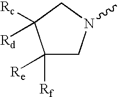
are as provided for a compound of formula I.

Turning now to a compound of formula VII, specific values for R₁, R₂, R₃, R₄, and R₅ and 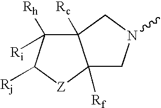
are as provided for a compound of formula I.

### Preparation of Invention Compounds

Strategies for the preparation of invention compounds are depicted in in the following Schemes.

As is readily apparent from this disclosure, compounds of the present invention are characterized by a quinolone core, covalently bound to an hydroxylated pyrollidinyl C-7 sidechain. As retrosynthetically depicted in Scheme I, the invention compounds can be prepared via coupling of a suitably C-7 substituted quinolone core precursor, wherein X is halo, triflate, or a similar reactive group known to the skilled artisan, and an appropriately substituted pyrollidine.

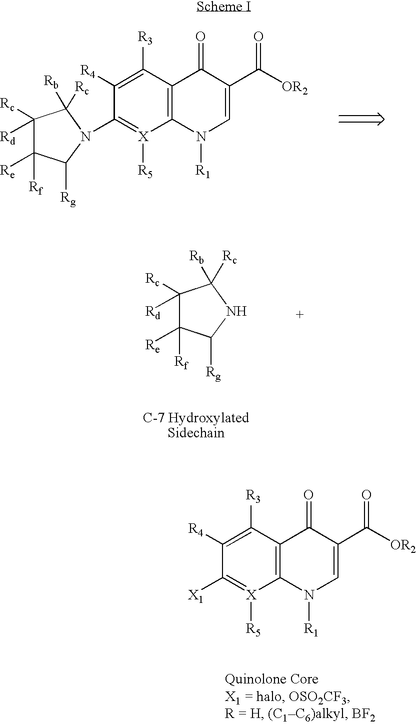

Reflecting the synthetic strategy summarized in Scheme I, the following section describing the preparation of the invention compounds has several parts. The first part describes the synthesis of the requisite quinolone core precursors. The second part describes the synthesis of the requisite C-7 sidechain precursors. The final part describes the coupling of the C-7 sidechain and quinolone core precursors to provide the invention compounds, and details any further chemical elaboration of invention compounds to produce other invention compounds.

A. Synthesis of Aminoquinazolinedione Core Precurors

The quinolone core precursors that are used to prepare the invention compounds are generally known to the skilled artisan and can be commercially obtained, or alternatively, can be prepared using routine synthetic methods. The following sections provide relevant citations that describe the preparation of the quinolone core precursors used to practice the invention disclosed herein.

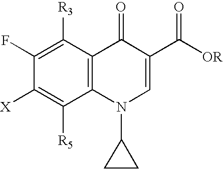

1. Preparation of Quinolone Core Precursors

| | | |
|---|---|---|
| a. | 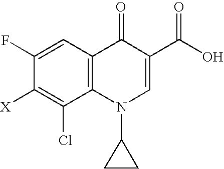 | EP 0357047; EP 0167763; EP 0195841 |
| b. | 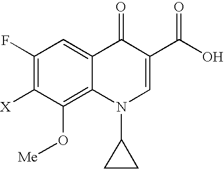 | JP8-9958 US 5869991 |
| c. | 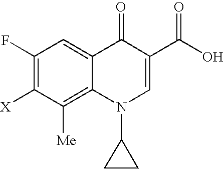 | EP 0357047 |
| d. | 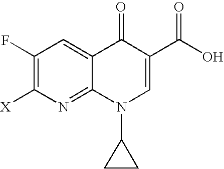 | EP 0172651 |
| e. | 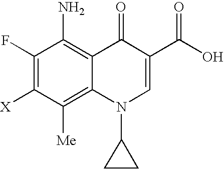 | US 5859,026 |
| f. | 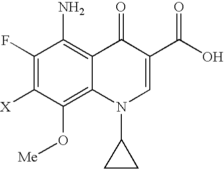 | EP 0610958 |
| g. | 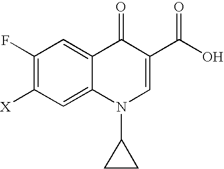 | US 5639886 |
| | 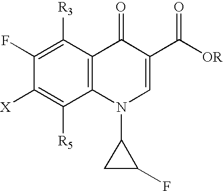 | |

2. Preparation of Quinolone Core Precursors

| | | |
|---|---|---|
| a. | 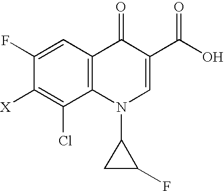 | As provided for 1A, above, except fluorocyclopropyl amine is used instead of cyclopropyl amine |
| b. | 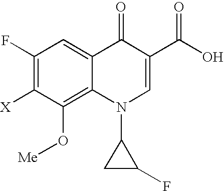 | As provided for 1A, above, except fluorocyclopropyl amine is used instead of cyclopropyl amine |
| c. | 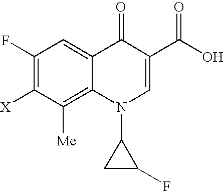 | As provided for 1C, above, except fluorocyclopropyl amine is used instead of cyclopropyl amine |
| d. | 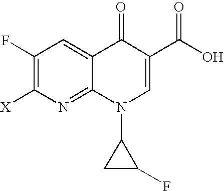 | As provided for 1D, above, except fluorocyclopropyl amine is used instead of cyclopropyl amine |
| e. | 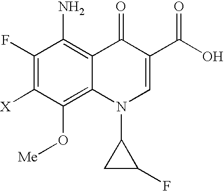 | As provided for 1F, above, except fluorocyclopropyl amine is used instead of cyclopropyl amine |
| f. | 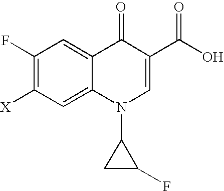 | As provided for 1H, above, except fluorocyclopropyl amine is used instead of cyclopropyl amine |

| | |
|---|---|
| 3. Preparation of | 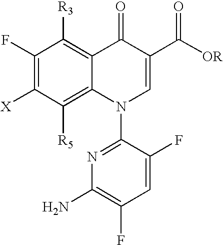 |

| | | |
|---|---|---|
| a. | 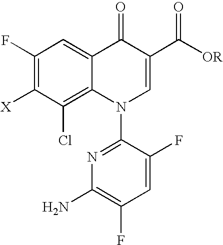 | U.S. Pat. No. 5998436 |
| b. | 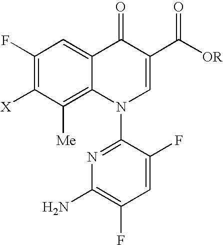 | U.S. Pat. No. 5998436 |
| c. | 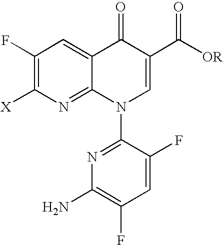 | U.S. Pat. No. 5998436 |
| d. | 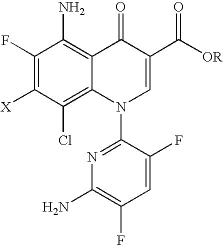 | U.S. Pat. No. 5998436 |
| e. | 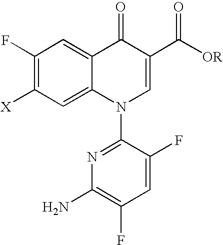 | U.S. Pat. No. 5998436 |

| | |
|---|---|
| 4. Preparation of | 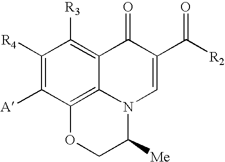 |

| | | |
|---|---|---|
| a. | 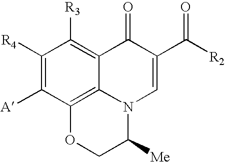 | U.S. Pat. No. 5053407 |

B. Synthesis of Hydroxylated C-7 Sidechain Precurors

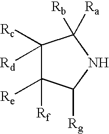

1. Preparation of

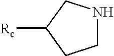

The sidechain precursor 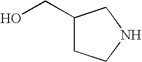
(1-3) as the S stereoisomer was prepared asymmetrically as depicted in Scheme 1. S-1 phenyl ethyl amine was used as a chiral protecting group in the aldehyde reduction. Deprotection 1-2 of provded the requisite sidechain precursor as a 1:1 mixture of diastereomers which were chromatographically separated.

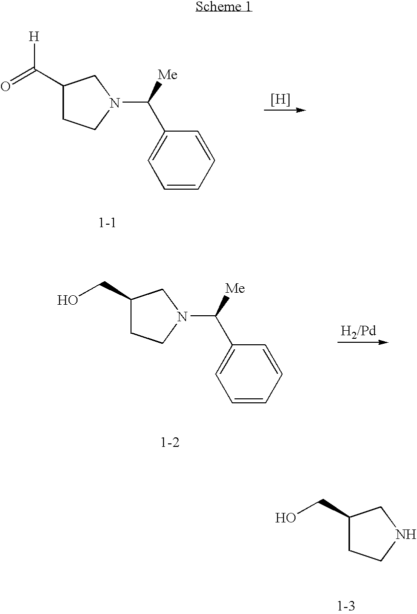

The sidechain precursor 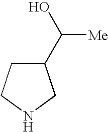
(2-5) was prepared as depicted in Scheme 2. In the racemic variant, 1-benzyl-pyrrolidine-3-carboxylic acid ethyl ester 2-1 was converted to the Weinreb amide 2-2. After a protecting group exchange, ketone formation, reduction, and deprotection provided the target compound.

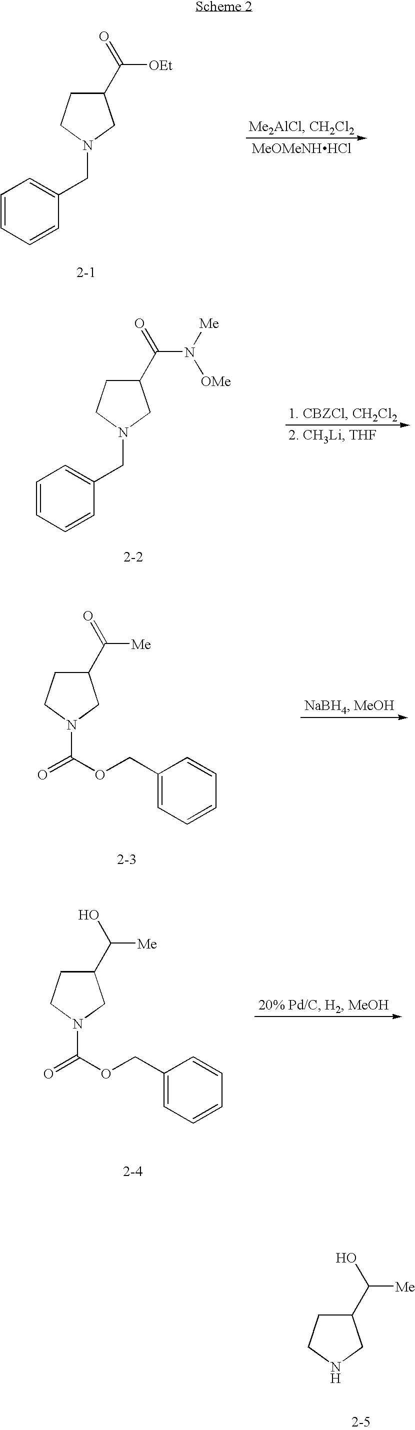

The sidechain precursor 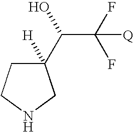
(3-7), wherein Q is H or F, was prepared as depicted in Scheme 3. S-1-phenyl-ethylamine was converted to the pyrrolidin-2-one upon treatment with 4-chloro-butyryl chloride in the presence of base. Alkylation of 3-2 using lithium diisopropylamide (LDA) and difluoro-acetic acid ethyl ester provided ketone 3-3. Zinc borohydride reduction of the ketone moiety in 3-3, provided the alcohol 3-4. N,N′-Dicyclohexylcarbodiimide (DCC)-mediated coupling of 3-4 to BOC-alanine, provided 3-5. Alane reduction of the amide moiety, followed by deprotection and hydrogenation, provided the requisite sidechain precursor.

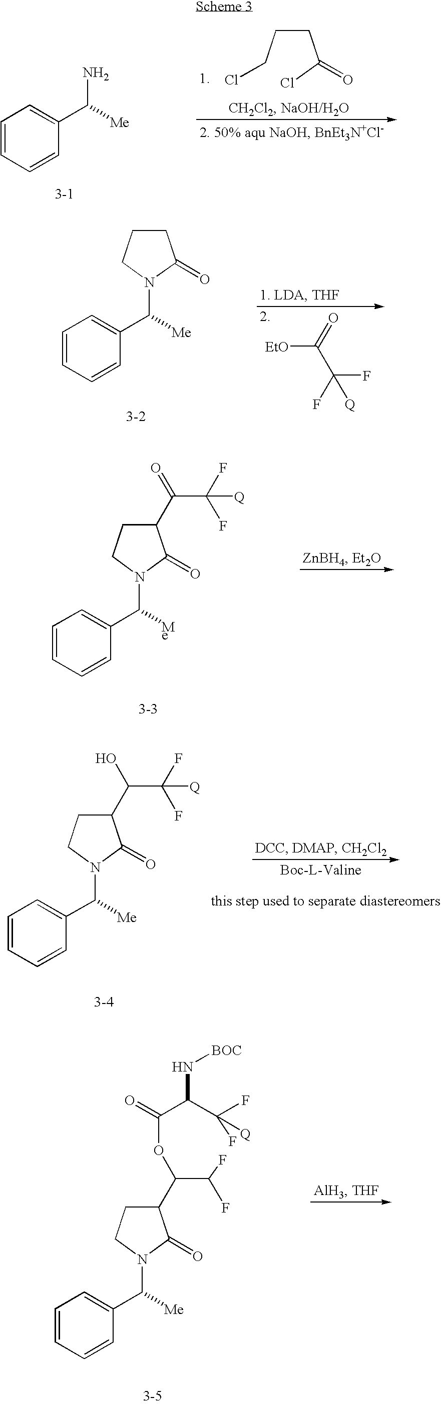

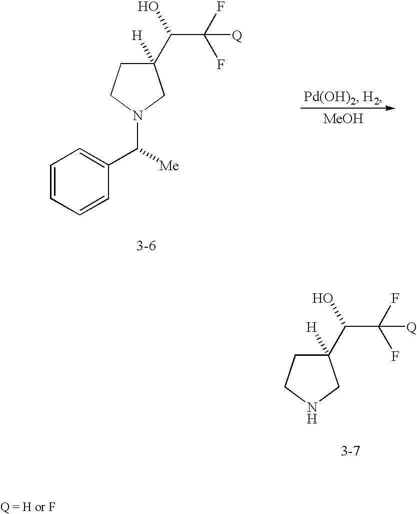

The sidechain precursor 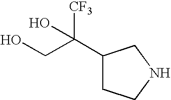
(4-7) was prepared as depicted in Scheme 4. Base mediated benzylation of but-3-ene-1,2-diol provided compound 4-2. Oxidation of the hydroyl moiety in 4-2 using the Dess-Martin reagent (triacetoxyperiodinane) provided the vinyl ketone 4-3. Compound 4-3 was converted to pyrrolidine 4-5 via [3+2] cycloadditon of the vinyl ketone moiety with the azomethine ylide derived from benzyl-methoxymethyl-trimethylsilanylmethyl-amine. CsF-catalyzed trifluoromethylation of the ketone moiety in 4-5 with TMS-CF₃ followed by deprotection provided the requisite sidechain in two additional steps, see, e.g., Singh, R. P.; Cao, G.; Kirchmeier, R. L.; Shreeve, J. M. J. Org. Chem. 1999, 64, 2873-2876.

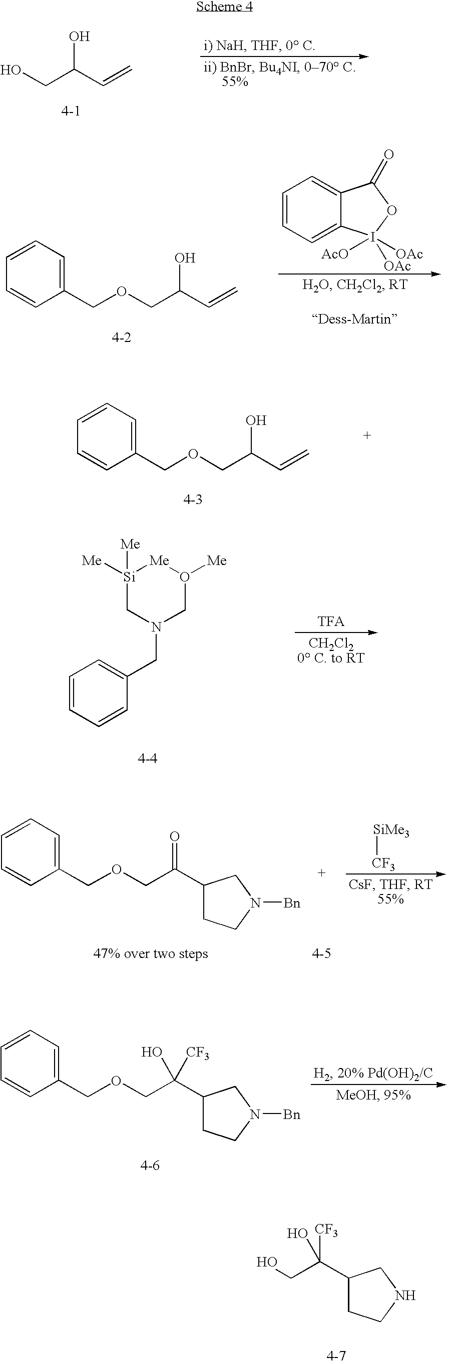

The sidechain precursor 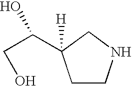
(5-11) was prepared as depicted in Scheme 5. Thus, 5-hydroxymethyl-5H-furan-2-one was prepared according to Nagaoka, Iwashima, Abe, Yamada Tet. Lett. (1989), 30, 5911-5914 and was subsequently protected as the triisopropyl silyl ether. 5-Triisopropylsilanyl oxymethyl-5H-furan-2-one was converted to 5-Triisopropylsilanyloxymethyl-4-nitromethyl-dihydro-furan-2-one under conventional conditions. 5-Triisopropylsilanyl oxymethyl-4-nitromethyl-dihydro-furan-2-one was hydrogenated to provide 4R-Aminomethyl-5S-triisopropylsilanyloxymethyl-dihydro-furan-2-one. Treatment of 4R-Aminomethyl-5S-triisopropylsilanyloxymethyl-dihydro-furan-2-one with base provided 4R-(1-hydroxy-2-triisopropylsilanyloxymethyl-ethyl)-pyrrolidin-2-one, which was converted to the target compound in a straightforward manner.

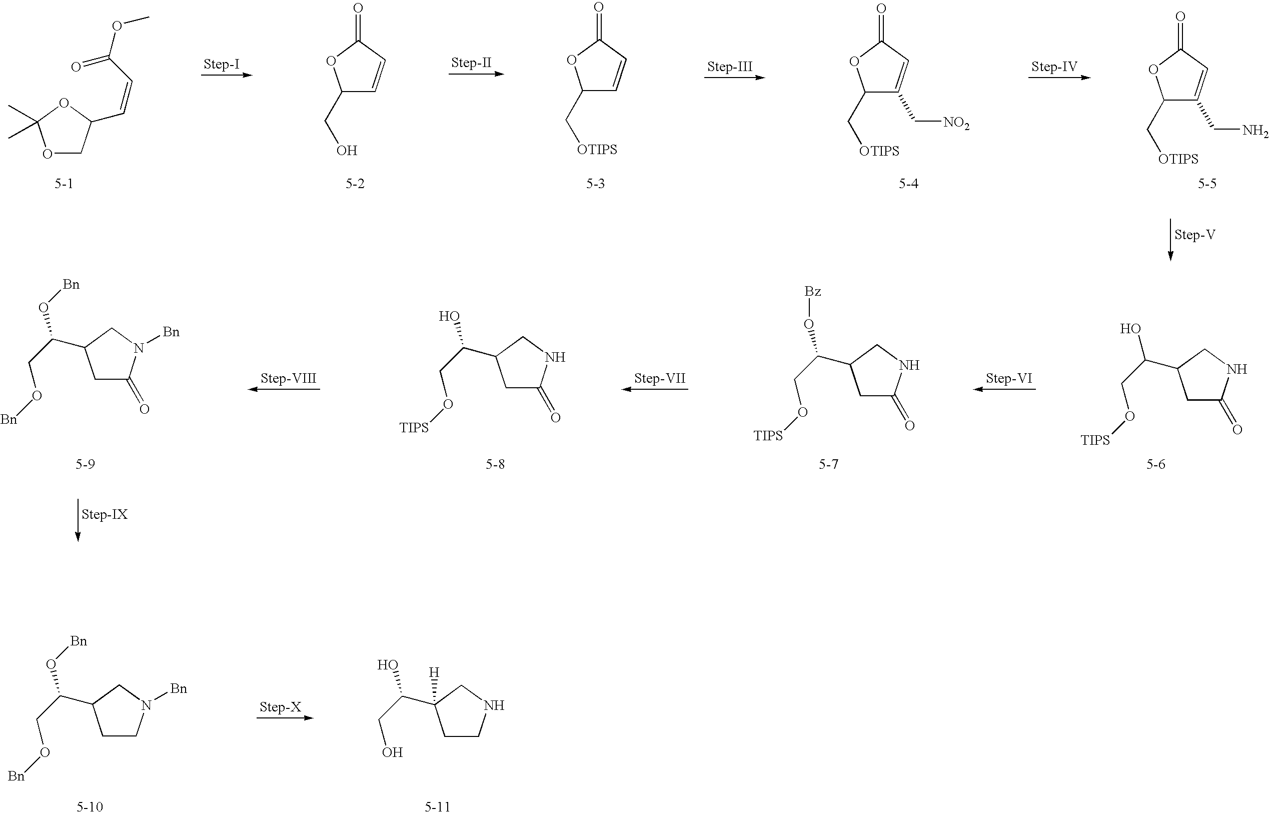

The sidechain precursor 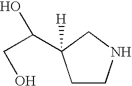
was prepared as indicated in Scheme 6. Steps 1-5 are identical to steps 1-5 in Scheme 5. Reduction provided the requisite sidechan precursor.

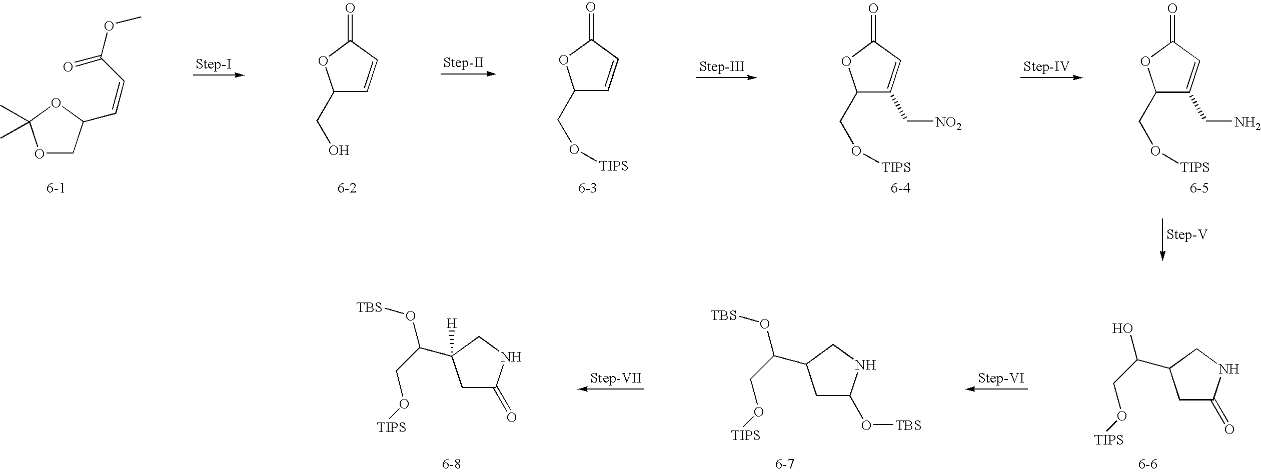

The sidechain precursor 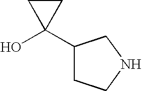
(7-5) was prepared as indicated in Scheme 7. Similar to Scheme 4, ethyl acrylate 7-1 was converted to pyrrolidine 7-3 via [3+2] cycloadditon with the azomethine ylide derived from benzyl-methoxymethyl-trimethylsilanylmethyl-amine. Compound 7-3 was converted to the cyclopropanol 7-4 upon treatment with ethylmagnesium bromide in the presence of Ti(O-iPr)₄. Deprotection provided the requisite compound.

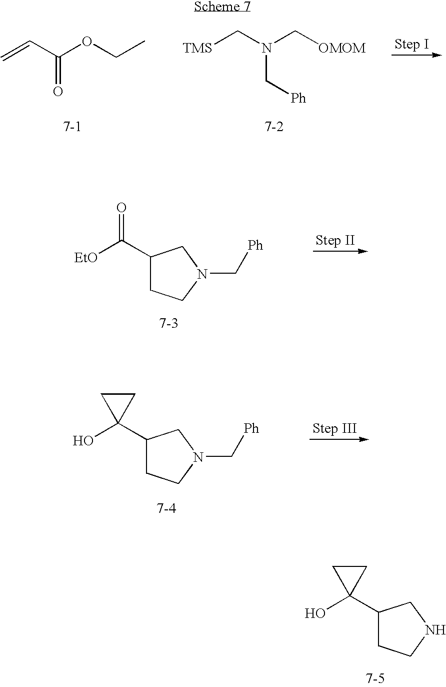

The sidechain precursor 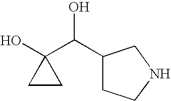
(8-7) was prepared as indicated in Scheme 8. Thus, 1-hydroxy-cyclopropanecarboxylic acid ethyl ester was protected as the tertbutyldimethylsilyl (TBDMS) ether, then underwent reaction with the anion of lactam 8-3 to provide the alkylation product 8-4. Reduction of the carbonyl moieties in 8-4 followed by a sequence of protection and deprotection reactions, provided the requisite sidechain precursor.

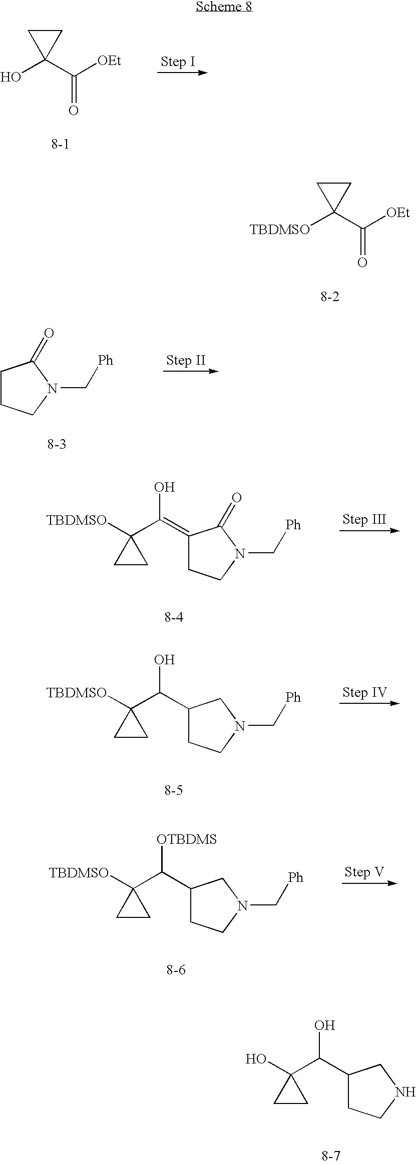

The sidechain precursor 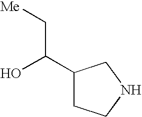
(9-2) was prepared upon hydrogenation of compound 9-1 as indicated in Scheme 9.

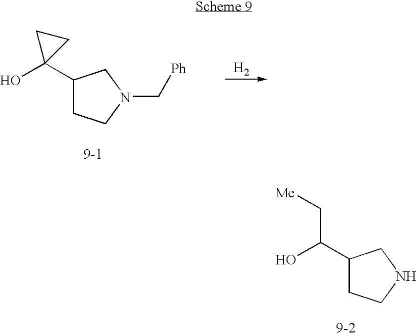

The sidechain precursor 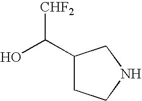
was prepared as indicated in Scheme 10. Thus, lithium aluminum hydride (LAH) reduction of compound 10-1 provided alcohol 10-2, which underwent hydrogenation to provided the requisite sidechain precursor.

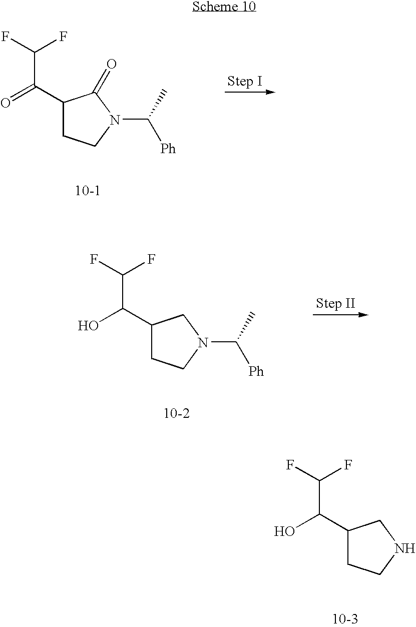

The sidechain precursor 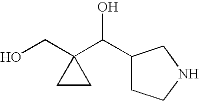
was prepared as indicated in Scheme 11. Thus, LDA-mediated alkylation of lactam 11-1 with cyclopropane-1,1-dicarboxylic acid diethyl ester provided diketoester 11-3. Reduction of compound 11-3 using LAH and aluminum trichloride (AlCi₃) provided alcohol 11-4, which underwent hydrogenation to provided the requisite sidechain precursor.

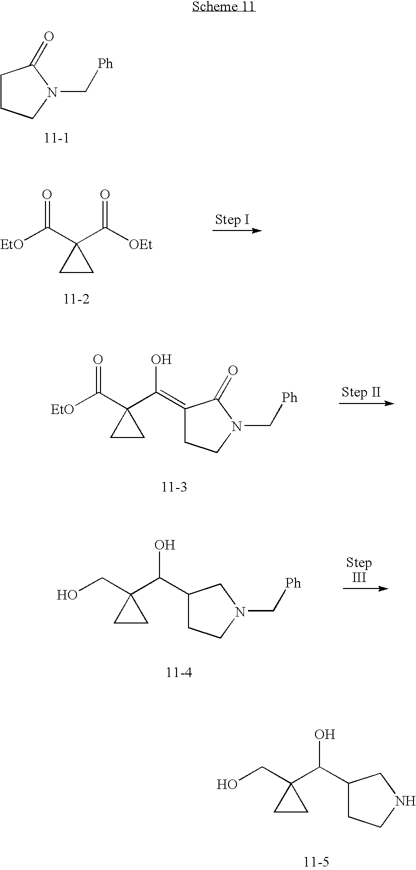

The sidechain precursor 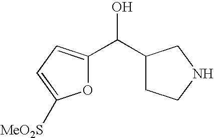
(12-9) was prepared as indicated in Scheme 12. Thus, 5-nitro-furan-2-carboxylic acid 12-1 was converted to the ethyl ester 12-2 under conventional conditions. Compound 12-2 was converted to the thioether 12-3 upon heating in the presence of NaSMe. Thioether 12-3 was oxidized to the sulfone 12-4 using m-chloroperbenozic acid (mCPBA). LDA-mediated alkylation of lactam 11-1 with sulfone 12-4 provided the requisite sidechain precursor 12-5 which was converted to to12-9 under standard conditions.

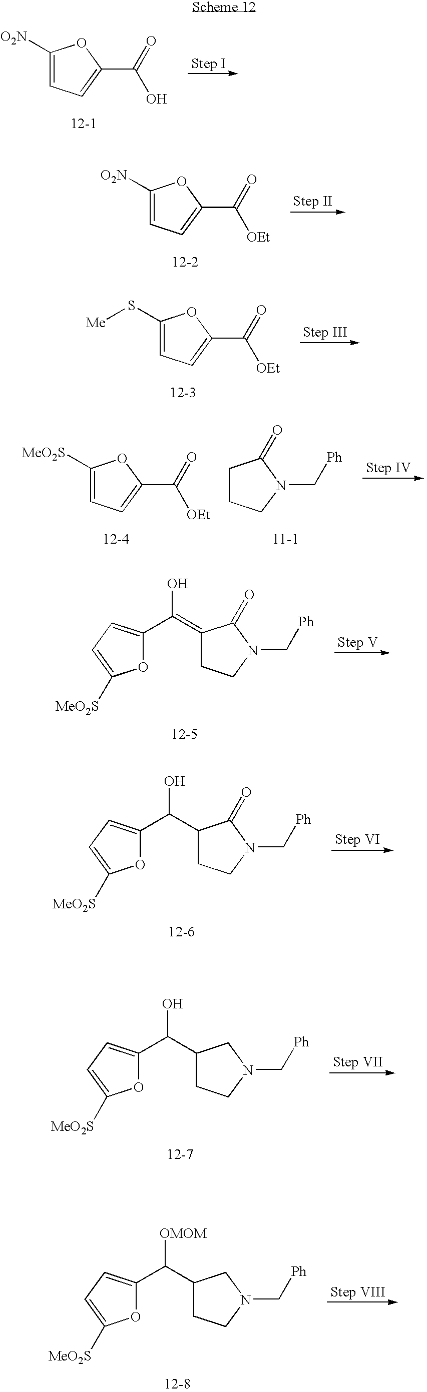

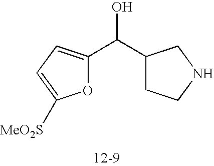

The sidechain precursor 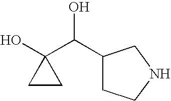
(13-6) was prepared as indicated in Scheme 13. Thus, 1-hydroxy-cyclopropanecarboxylic acid ethyl ester 13-1 was protected as the TBDMS ether 13-2, and then allowed to undergo reaction with the anion of lactam 11-1 to provide the alkylation product 13-3. Reduction of the carbonyl moieties in 13-3, followed by a sequence of protection and deprotection reactions, provided the requisite sidechain precursor.

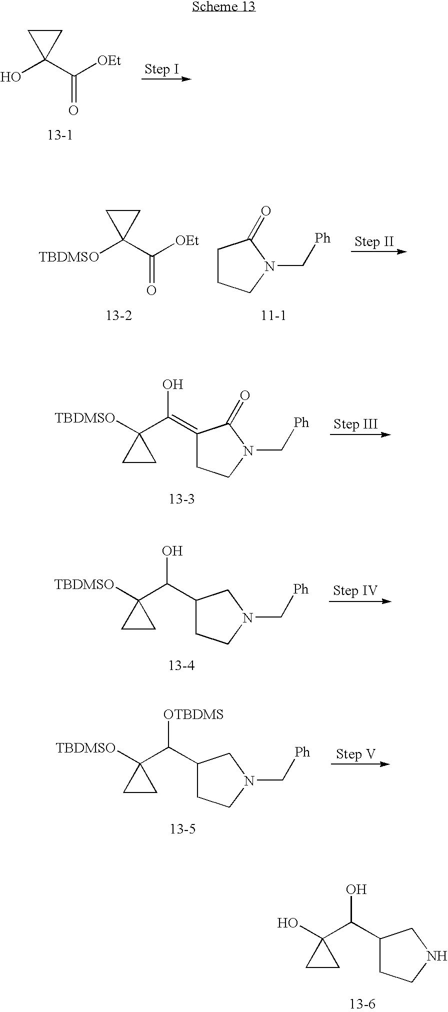

The sidechain precursur 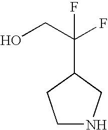
(14-3) was prepared as indicated in Scheme 14. Thus, ketone 14-1 was converted to the gem-difluoride 14-2 upon treatment with diethylaminosulfur trifluoride. Hydrogenation of compound 14-2 under standard conditions provided the requisite sidechain.

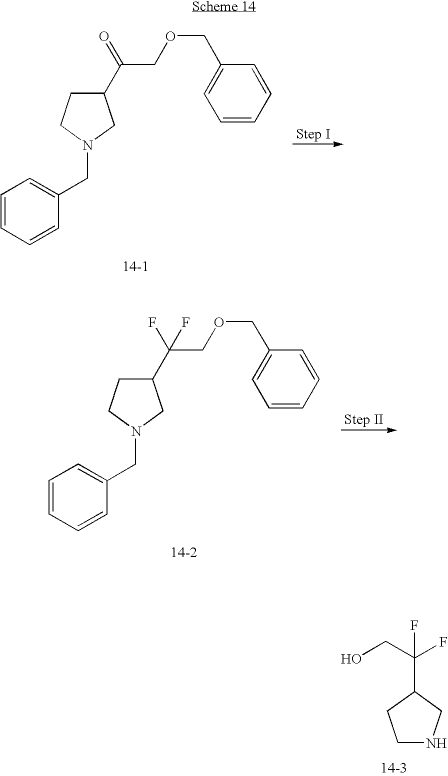

2. Preparation of

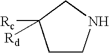

The sidechain precursor 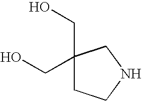
(15-5) was prepared as indicated in Scheme 15. Thus, compound 15-1 was prepared according to Org. Syn. Coll. Vol. IV, p. 298. Similar to Scheme 7, acrylate 15-3 was converted to pyrrolidine 15-4 via [3+2] cycloadditon with the azomethine ylide derived from benzyl-methoxymethyl-trimethylsilanylmethyl-amine, followed by reduction of the resulting diester. Compound 15-4 was converted to the requisite sidechain precursor upon deprotection.

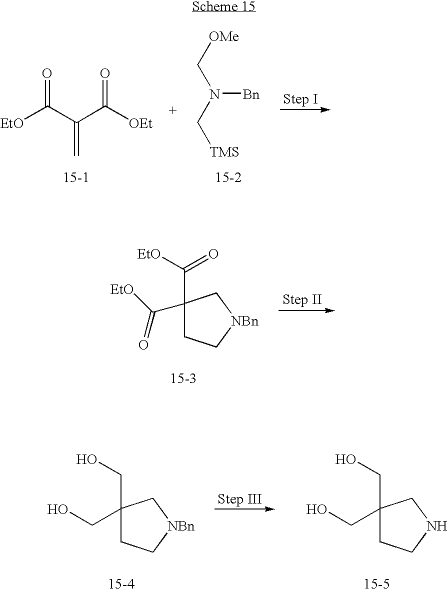

3. Preparation of

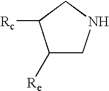

The sidechain precursor 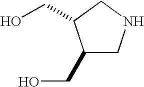
(16-3) was prepared as indicated in Scheme 16. Thus, diester 16-1 was reduced to the diol 16-2, which was hydrogenated under conventional condtions to provide the requisite sidechain precursor.

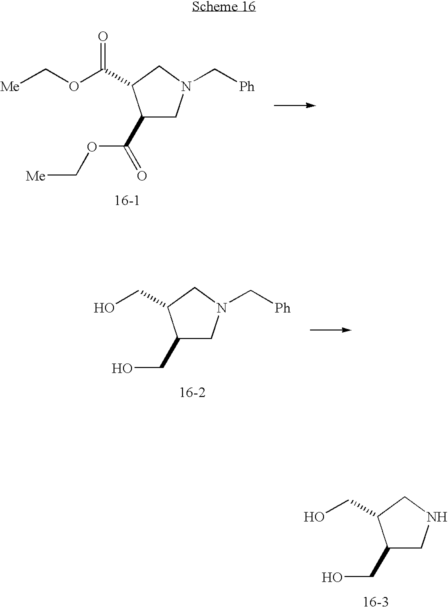

The sidechain precursor 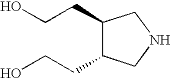
(17-6) can be prepared as indicated in Scheme 17. Thus, the isoindole 17-1 can be benzylated to provide 17-2. Oxidative cleavage of 17-2 using a reagent known to the skilled artisan such as permanganate (MnO₄) or by ozonlysis can provide dialdehyde 17-3, which is readily reduced to the diol 17-4. Reduction of the imide moiety in 17-4 and deprotection provides the requisite sidechain precursor.

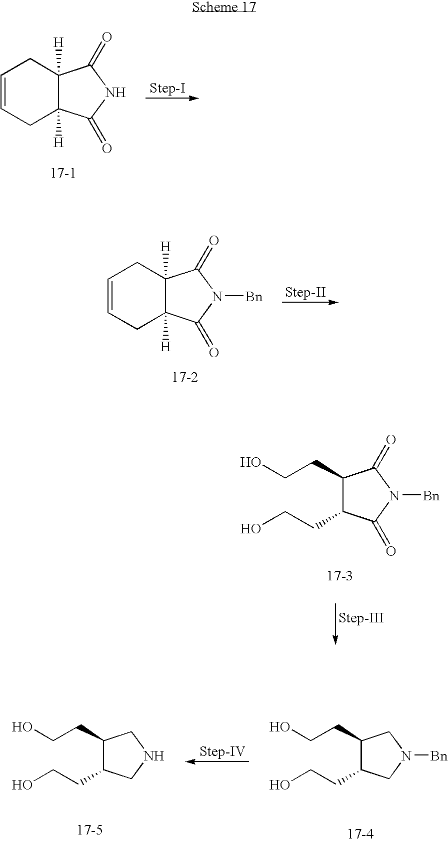

The sidechain precursor 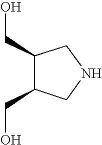
(18-4) was prepared as indicated in Scheme 18. Thus, [3+2] cycloaddition of the cis maleate 18-1 with the azomethine ylide derived form compound 15-2 provided pyrrolidine 18-2. Reduction of the ester moieties in 18-2, followed by deprotection, provided the requisite sidechain precursor.

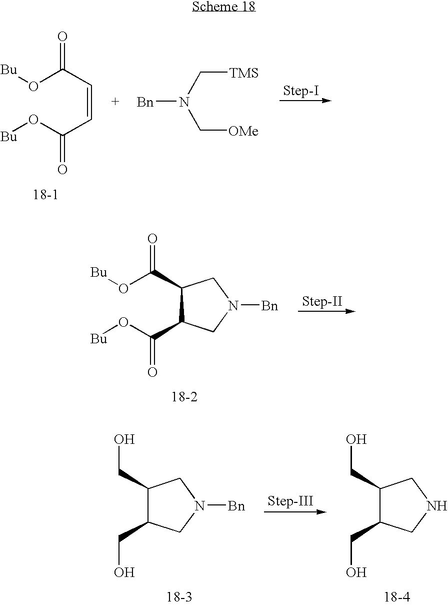

The sidechain precursor 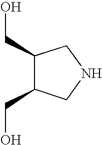
(19-6) was prepared as indicated in Scheme 19. Thus, similar to Scheme 18, [3+2] cycloaddition of the trans maleate 19-1 with the azomethine ylide derived from compound 15-2 provided pyrrolidine 19-2. Reduction of the ester moieties in 19-2, followed by silylation of the resulting diol and deprotection, provided 19-5, which can be converted to the free diol upon deprotection.

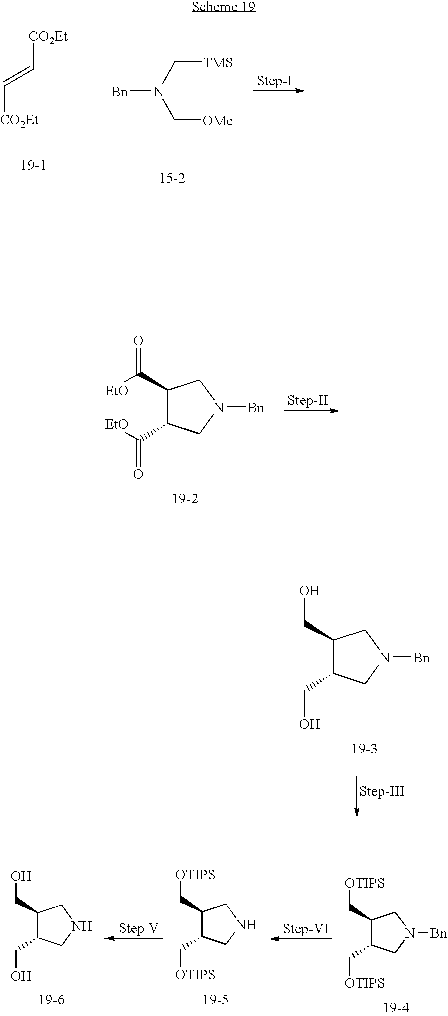

The sidechain precursor 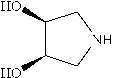
(20-3) was prepared as indicated in Scheme 20. Thus, osmium tetroxide (OsO₄)-catalyzed hydroxylation of the double bond in compound 20-1 provided the cis diol 20-2, which was converted to the requisite sidechain precursor upon deprotection.

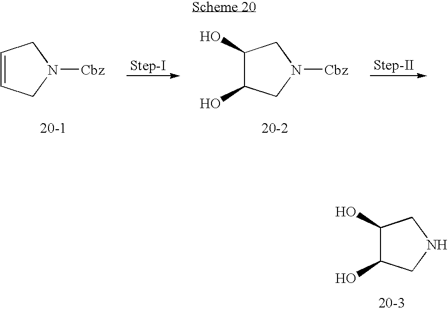

4. Preparation of

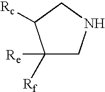

The sidechain precursor 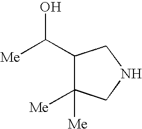
(21-5) was prepared as indicated in Scheme 21. Thus, similar to Scheme 18, [3+2] cycloaddition of the 4-Methyl-pent-3-en-2-one with the azomethine ylide derived form compound 21-1 provided pyrrolidine 21-2. Compound 21-2 was converted to the CBZ amide 21-3. Reduction of the ketone moiety in 21-4, followed by deprotection, provided the requisite sidechain precursor.

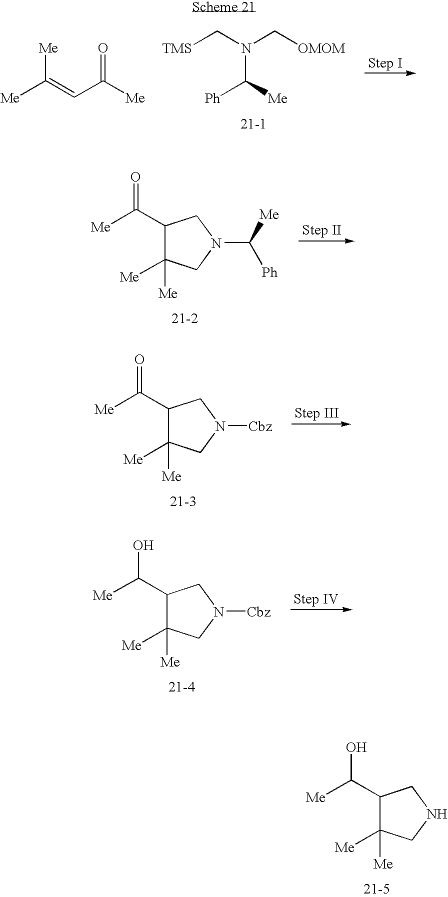

5. Preparation of

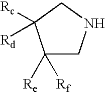

The sidechain precursor 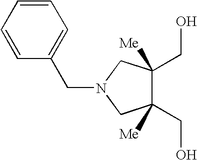
(22-2) was prepared as provided in Scheme 22. Thus [3+2] cycloaddtion of 3,4-dimethyl-furan-2,5-dione with the azomethine ylide derived form compound 15-2 provided pyrrolidine 22-1. Hydrogenation of 22-1 under standard conditions afforded the requisite sidechain precursor.

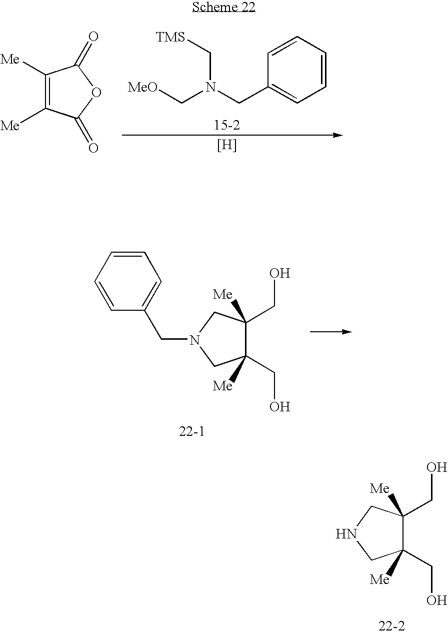

6. Preparation of

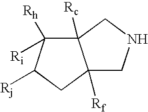

The sidechain precursor 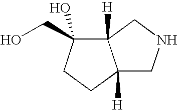
(23-4) was prepared as indicated in Scheme 23. Thus, [3+2] cycloaddition of cyclopent-2-enone with the azomethine ylide derived from compound 15-2 provided pyrrolidine 23-1. Compound 23-1 was deprotected, then reporotected as the CBZ amide 23-2. SmI₂-catalyzed hydroxymethylation of 23-2 provided hydroxy ether 23-3, see, e.g., Imamoto, T.; Takeyama, T.; Yokoyama, M. Tetrahedron Letters, 1984, 25, 3225-3226. Removal of the protecting groups by hydrogenation provided the requisite sidechain precursor.

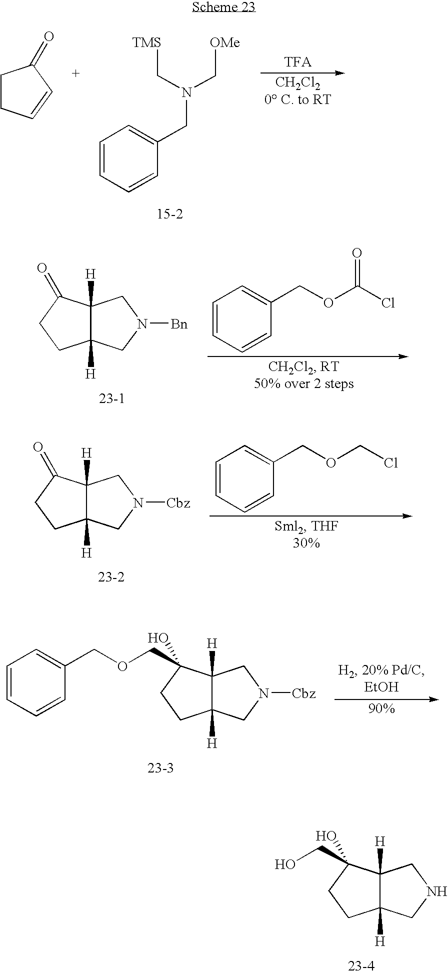

The sidechain precursor 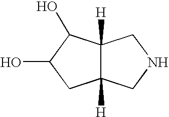
(24-4) was prepared as indicated in Scheme 24. Compound 23-2 was prepared as indicated in Scheme 23. L-Selectride reduction of the ketone moiety in compound 23-2 provided alcohol 24-1, see, e.g., Ogata, M.; Matsumoto, H.; Shimizu, S.; Kida, S.; Nakai, H.; Motokawa, K.; Miwa, H.; Matsuura, S.; Yoshida, T. Eur. J. Med. Chem. 1991, 26, 889-906. Syn elimination of the hydroxy moiety in compound 24-1 using the Burgess reagent provided compound 24-2 see, e.g., Campbell, E.; Martin, J. J.; Bordner, J.; Kleinman, E. F. J. Org. Chem. 1996, 61, 4806-4809. Yamada, O.; Ogasawara, K. Tetrahedron Letters 1998, 39, 7747-7750. OsO₄-catalyzed hydroxylation of the double bond in compound 24-2 provided the cis diol 24-3, which was converted to the requisite sidechain precursor upon deprotection.

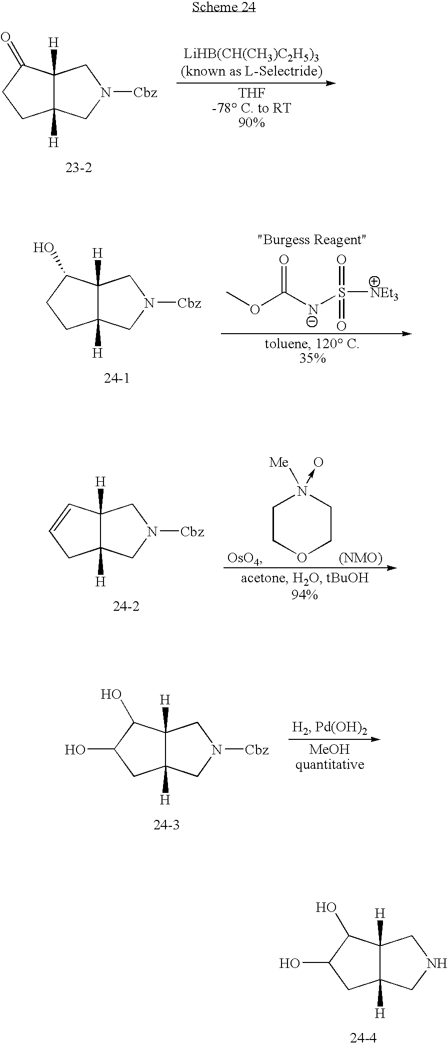

The sidechain precursor 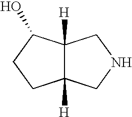
(25-1) was prepared as indicated in Scheme 25. Compound 24-1 was prepared as indicated in Scheme 24. Deprotection of compound 24-1 provided the requisite sidechain precursor.

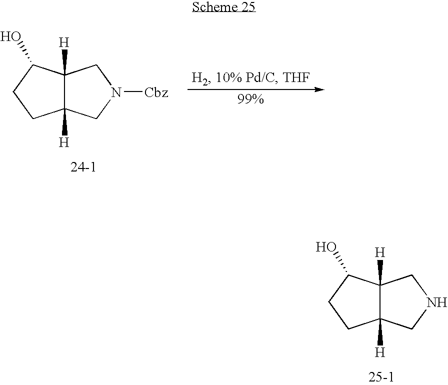

The sidechain precursor 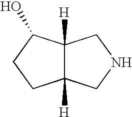
(26-3) was prepared as indicated in Scheme 26. Compound 24-1 was prepared as indicated in Scheme 24. Mitsunobu reaction of the alcohol moiety in compound 24-1 provided the ester 26-1, see, e.g., Jeong, L. S.; Yoo, S. J.; Moon, H. R.; Kim, Y. H.; Chun, M. W. J. Chem. Soc., Perkin Trans. 1 1998, 3325-3326. Ester 26-1 was saponified under conventional conditions to provided compound 26-2. Deprotection of compound 26-2 provided the requisite sidechain precursor.

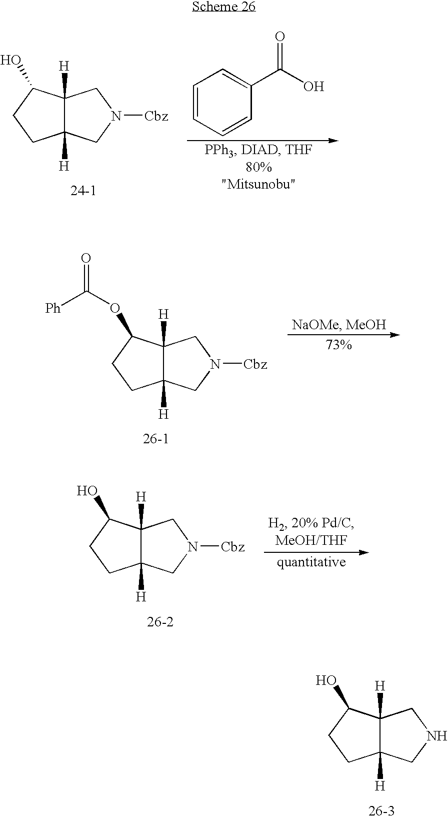

The sidechain precursors 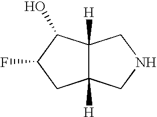
(27-4a) and 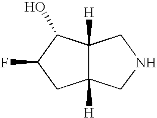
(27-4b) were prepared as indicated in Scheme 27. Compound 23-2, prepared as indicatedin Scheme 23, was converted to the silyl enol ether 27-1 under conventional conditions. Concomittant desilylation and fluorination provided fluorides 27-2a and 27-2b as a separatable mixture of diastereomers. Diastereomers 27-2a and 27-2b were reduced using L-Selectride to provide alcohols 27-3a and 27-3b, which were deprotected to provided the requisite sidechains.

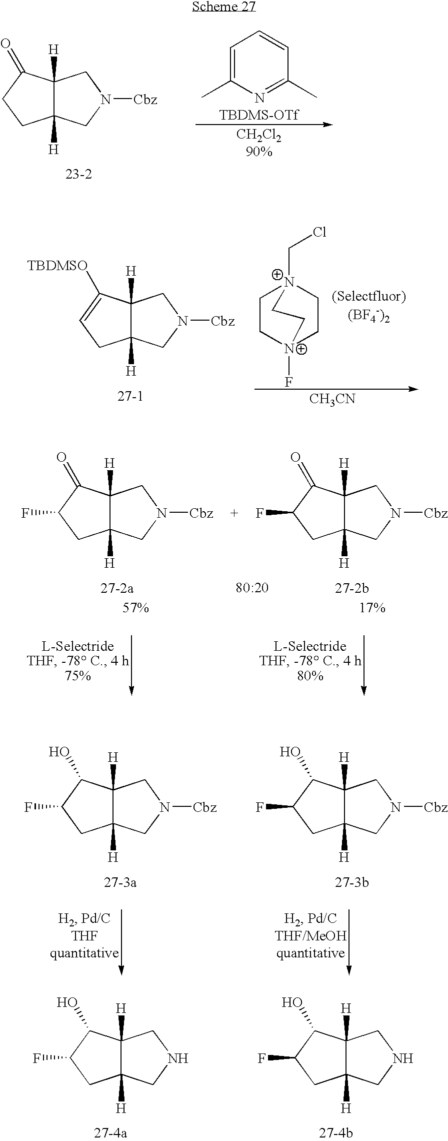

The sidechain precursor 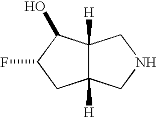
was prepared as indicated in Scheme 28. Compound 28-1 underwent Mitsunobu reaction to provide ester 28-2, which underwent transesterification and deprotection to provide the requisite sidechain.

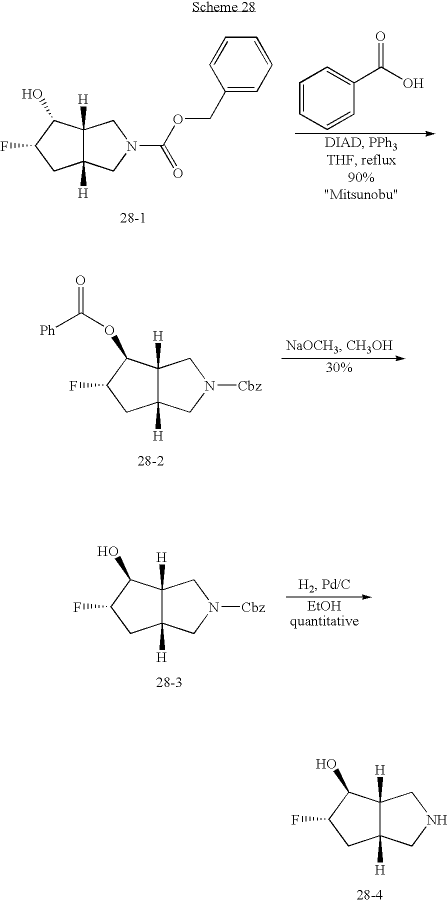

Similarly, as indicated in Scheme 29, the sidechain precursor 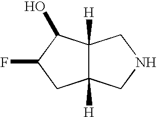
(29-4) was prepared via the same sequence of reactions as provided in Schemes 27 and 29. Compound 29-1 underwent Mitsunobu reaction to provide ester 29-2, which underwent transesterification and deprotection to provide the requisite sidechain.

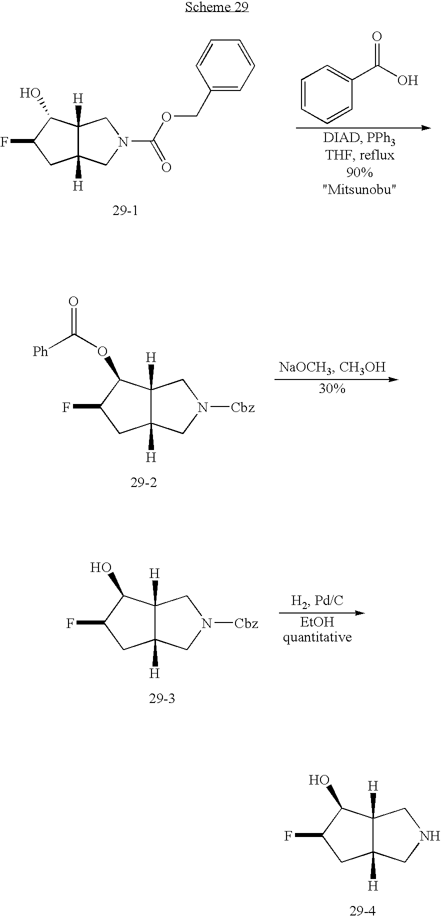

Sidechain precursor 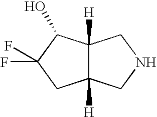
(31-2) was prepared as provided in Scheme 31 from the diasteromeric mixture of alpha fluoro ketones obtained in Scheme 27, or from either diastereomer alone. Thus deprotonation and fluorination, see, e.g., Thomas, M. G.; Suckling, C. J.; Pitt, A. R.; Suckling, K. E. J. Chem. Soc., Perkin Trans. 1 1999, 3191-3198, provided difluoroketone 31-1. Reduction of compound 31-1, followed by deprotection, provided the requisite sidechain.

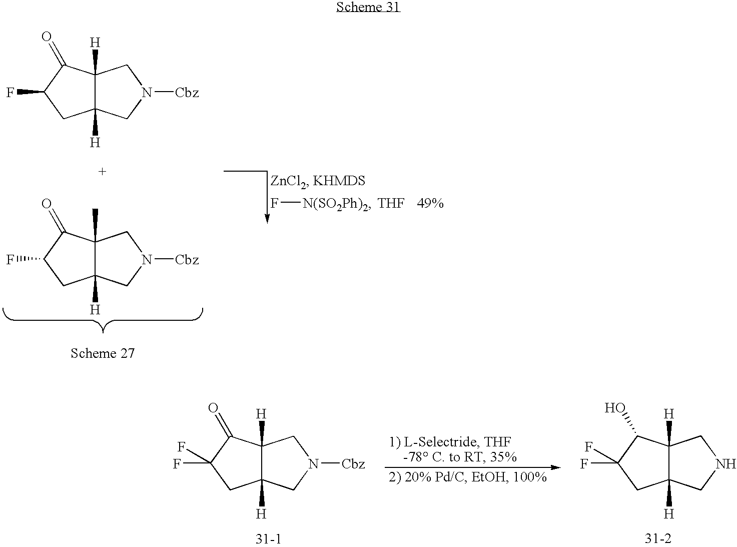

The sidechain precursor 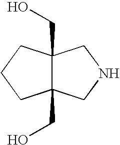
(15-2) was prepared as indiciated in Scheme 32. Thus, [3+2] cycloaddtion of 5,6-dihydro-4H-cyclopenta[c]furan-1,3-dione with the azomethine ylide derived from compound 15-2 provided compound 15-1. Hydrogenation of 15-1 under standard conditions afforded the requisite sidechain precursor. 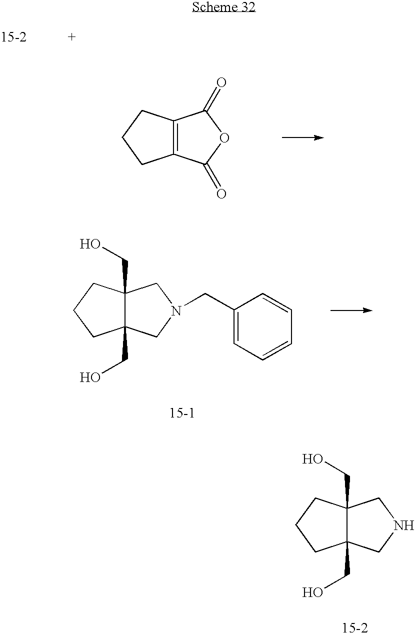
C. Coupling of Hydroxylated C-7 Sidechain and Quinolone Core Precurors to Provide Invention Compounds

Coupling of the sidechain precursor to the quinolone core precursor to provide the compounds of the present invention can occur from either the core precursor as the free acid, alkyl ester, or borate ester, as depicted in Scheme II.

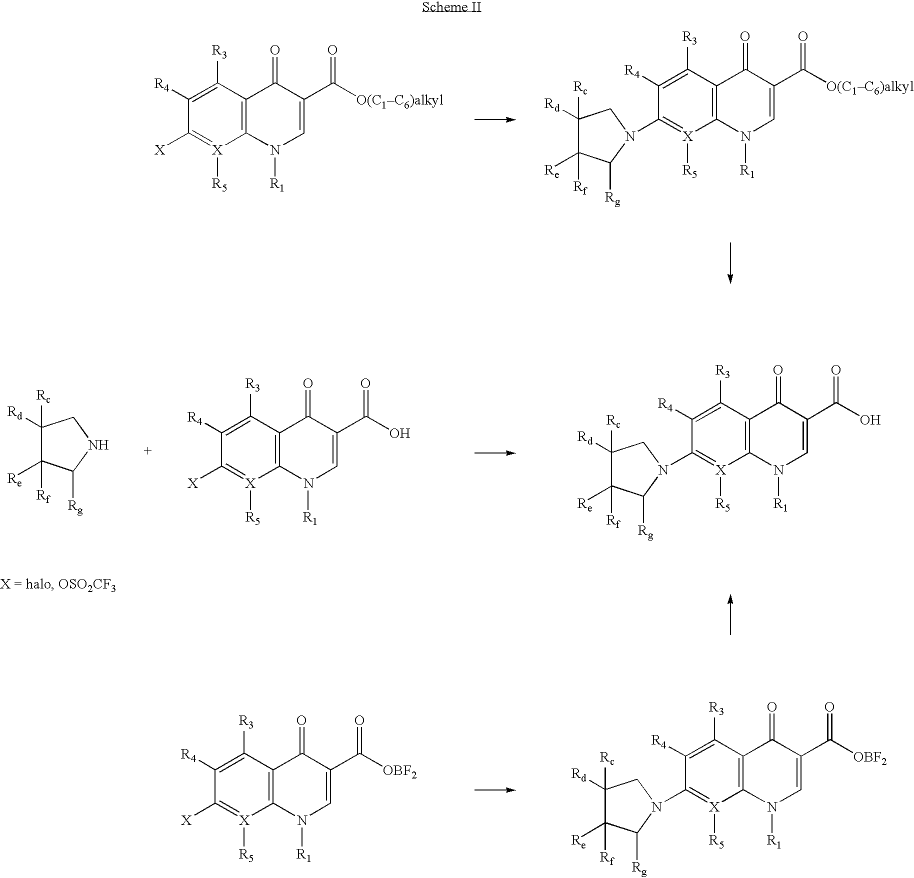

Typically, when a free acid is used in the coupling reaction, a molar excess of the side chain precursor is combined with the quinolone core in a polar solvent such as acetonitrile (6 mL). A molar excess of an amine base such as triethylamine is added, and the reaction mixture is heated to about 80° C. Typically, the reaction mixtures becomes homogenous. The mixture is heated for sufficient time to drive the raction to completion, typically from about 3 to about 12 hours. The mixture is then worked up according to procedures widely uused by the skilled artisan to provide a compound of the invention.

When an alkyl ester is used in the coupling reaction,the quinolone core, sidechain, and triethylamine are combined in a solvent such as acetonitrile. The resulting reaction mixture is heated to 80° C. and stirred for 12 hours. is heated to about 80° C. Typically, the reaction mixtures becomes homogenous. The mixture is heated for sufficient time to drive the raction to completion, typically from about 3 to about 12 hours. The mixture is then worked up according to procedures widely uused by the skilled artisan to provide a compound of the invention.

When a borate ester is used in the coupling reaction, the requisite borate ester is typically prepared from the free acid upon reaction with BF₃ according to conditions available to the skilled artisan. Theborater ester is typically combined with the side chain in a solvent such as acetonitrile and treated with an amine base such as triethylamine. The resulting reaction mixture is typically stirred at room temperature for sufficient time to drive the reaction to completion, typically from about 24 to about 96 hours. The mixture is then worked up according to procedures widely used by the skilled artisan to provide a compound of the invention.

### Pharmaceutical Formulations

The present invention also provides pharmaceutical compositions which comprise a bioactive invention compound or a salt such or a pharmaceutically acceptable salt thereof and optionally a pharmaceutically acceptable carrier. The compositions include those in a form adapted for oral, topical or parenteral use and can be used for the treatment of bacterial infection in mammals including humans.

The compounds, such as antibiotic compounds, also referred to herein as antimicrobial compounds, according to the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

### Biological Activity

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

In one embodiment, the invention provides methods of treating or preventing a bacterial infection in a subject, such as a human or other animal subject, comprising administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985;28:766; Heifetz, et. al., Antimicrob., 1974;6:124). The results of the evaluation are shown in Tables 1A and B, wherein “-” means no data.

TABLE 1A

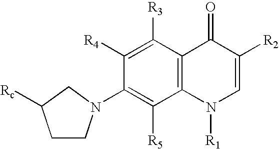

| | Minimum Inhibitory Concentrations μg/mL Gram Negative Bacteria | | |
|---|---|---|---|
| Compound | H. influenzae HI-3542 | M. catarrhalis BC-3531 | E. coli EC-2549 |
| 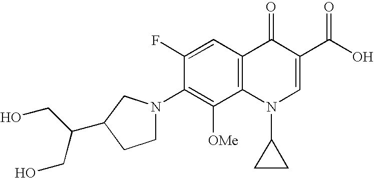 | 0.008 | 0.015 | 0.06 |
| 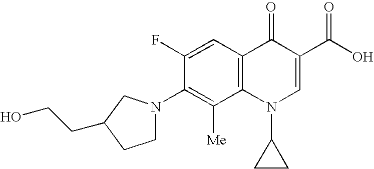 | 0.030 | 0.015 | 0.06 |
| 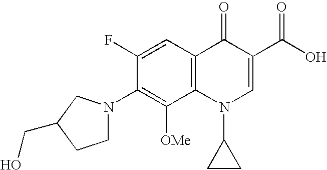 | 0.002 | 0.004 | 0.03 |
| 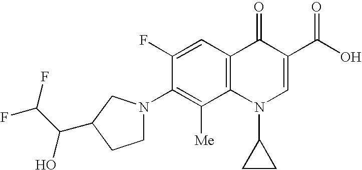 | 0.008 | 0.008 | 0.06 |
| 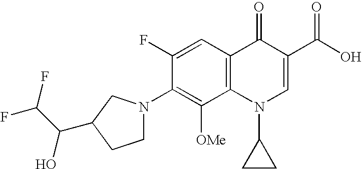 | 0.015 | 0.03 | 0.125 |
| 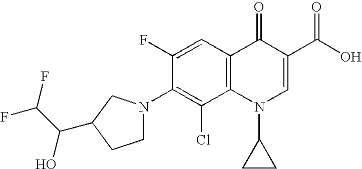 | 0.004 | 0.008 | 0.06 |
| 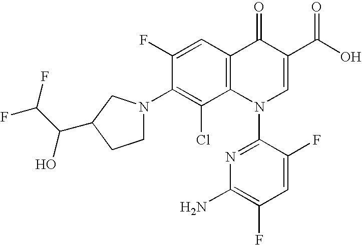 | 0.008 | 0.015 | 0.25 |
| 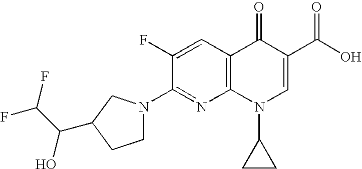 | 0.008 | 0.016 | 0.125 |
| 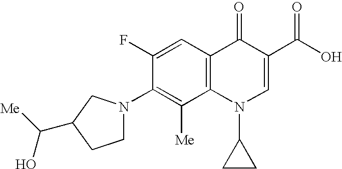 | 0.002 | 0.002 | 0.06 |
| 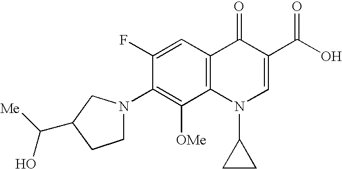 | 0.008 | 0.015 | 0.125 |
| 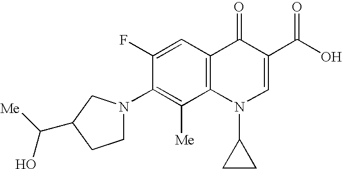 | 0.004 | 0.008 | 0.06 |
| 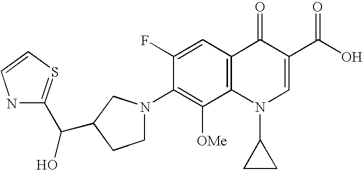 | 0.03 | 0.03 | 0.125 |
| 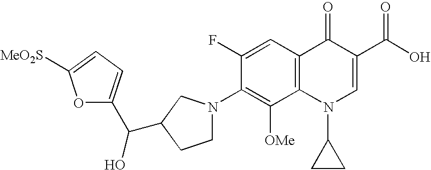 | 0.06 | 0.125 | 2 |
| 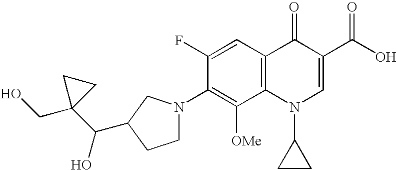 | 0.03 | — | 0.25 |
| 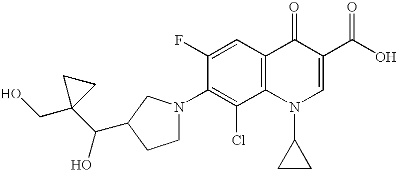 | 0.008 | 0.016 | 0.06 |
| 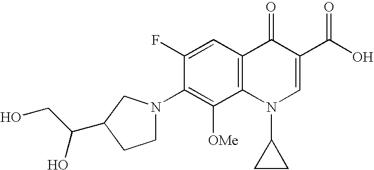 | 0.015 | 0.04 | 0.03 |
| 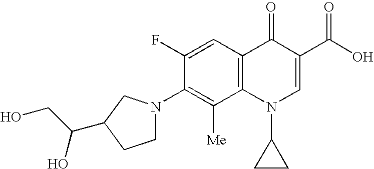 | 0.5 | 1 | 4 |
| 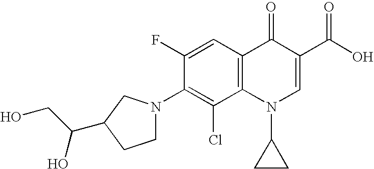 | 0.004 | 0.016 | 0.06 |
| 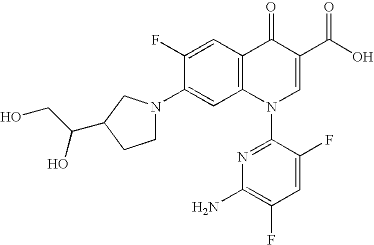 | 0.004 | 0.015 | 0.06 |
| 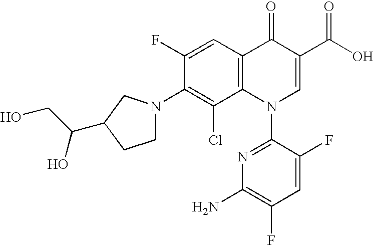 | 0.008 | 0.125 | 0.25 |
| 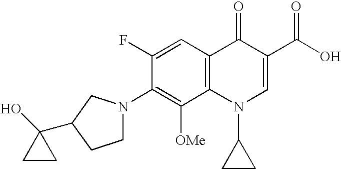 | 0.03 | 0.03 | 0.25 |
| 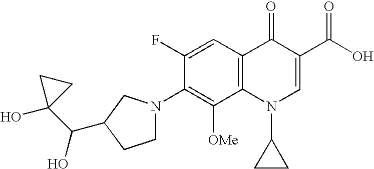 | 0.015 | 0.125 | 0.5 |
| 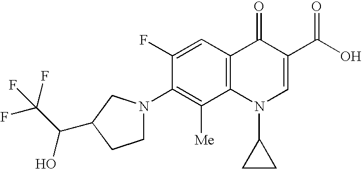 | 0.06 | 0.03 | 0.5 |
| 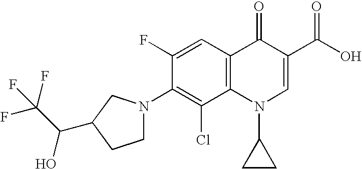 | 0.004 | 0.004 | 0.06 |
| 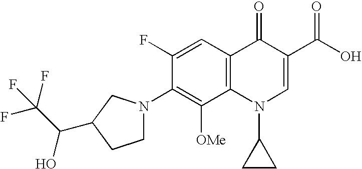 | 0.015 | 0.008 | 0.125 |
| 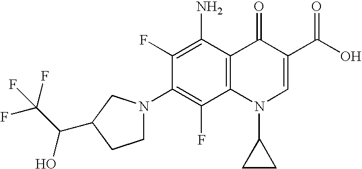 | 0.004 | 0.008 | 0.06 |
| 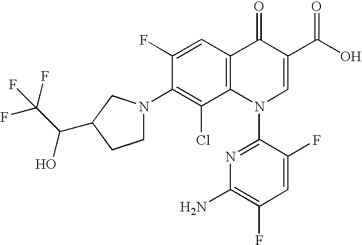 | 0.06 | 0.125 | 1 |
| 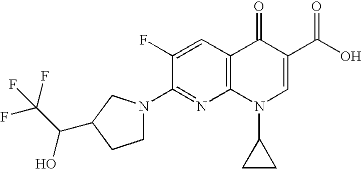 | 0.016 | 0.016 | 0.125 |
| 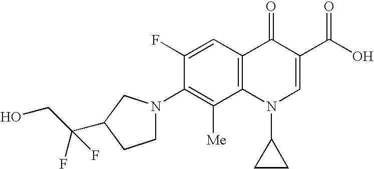 | 0.015 | 0.03 | 0.125 |
| 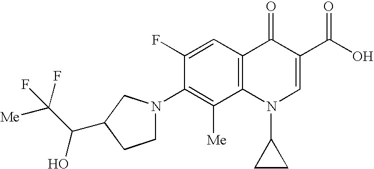 | 0.03 | 0.03 | 0.125 |
| 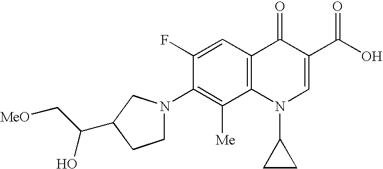 | 0.125 | 0.125 | 1 |
| 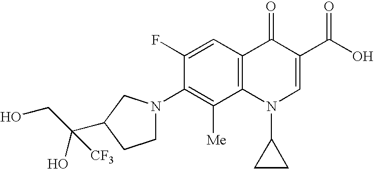 | 0.06 | 0.06 | 0.5 |
| 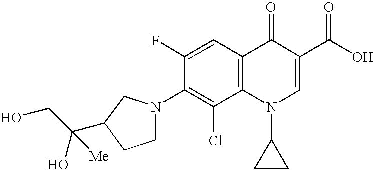 | 0.16 | 0.03 | 0.125 |

TABLE 1B

| | Minimum Inhibitory Concentrations μg/mL Gram Positive Bacteria | | | |
|---|---|---|---|---|
| Compound | E. faecalis MGH-2 | S. pneumo SV-1 | S. aureus UC-76 | S pyogenes C203 |
| 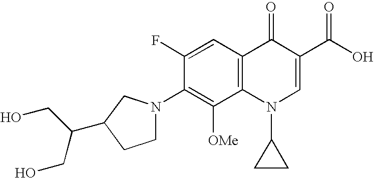 | 0.03 | 0.03 | 0.004 | 0.06 |
| 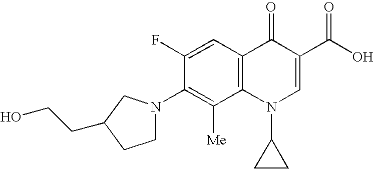 | 2 | 0.03 | 0.125 | 0.06 |
| 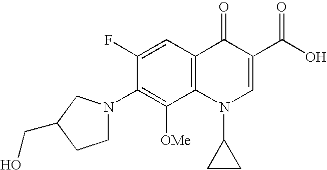 | 0.06 | 0.06 | 0.00403 | 0.06 |
| 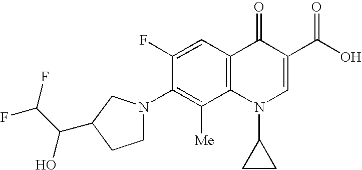 | 0.06 | 0.06 | 0.004 | 0.125 |
| 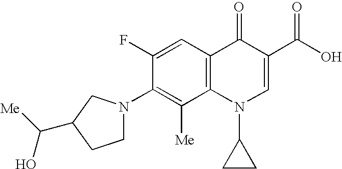 | 0.06 | 0.03 | — | 0.06 |
| 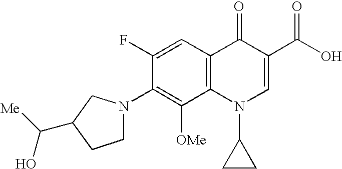 | 0.125 | 0.06 | — | 0.125 |
| 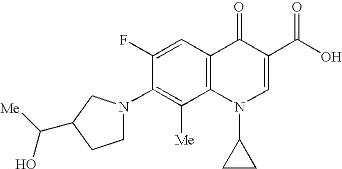 | 0.06 | 0.06 | — | 0.06 |
| 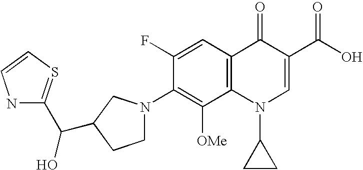 | 0.125 | 0.06 | — | 0.125 |
| 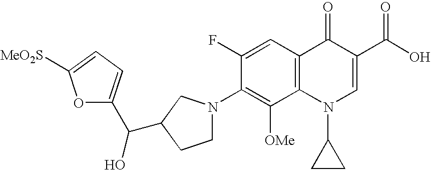 | 0.5 | 0.5 | — | 0.5 |
| 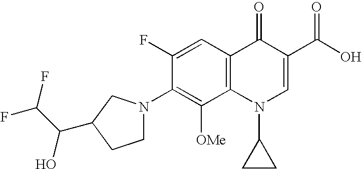 | 0.125 | 0.06 | 0.008 | 0.25 |
| 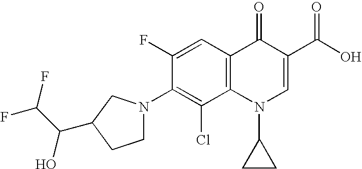 | 0.125 | 0.125 | — | 0.25 |
| 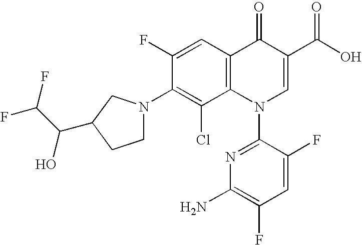 | 0.25 | 0.06 | — | 0.125 |
| 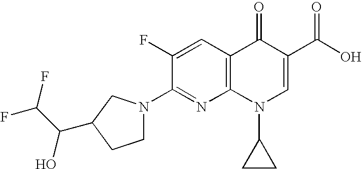 | 0.125 | 0.25 | — | 0.25 |
| 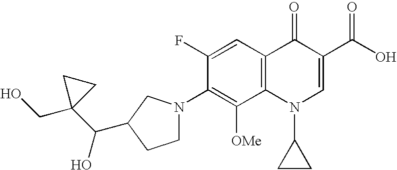 | 0.25 | 0.125 | — | 0.25 |
| 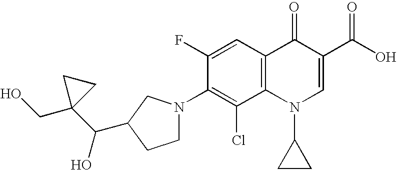 | 0.125 | 0.125 | — | 0.125 |
| 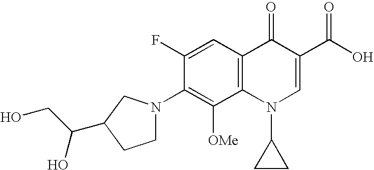 | 0.03 | 0.03 | 0.002 | 0.03 |
| 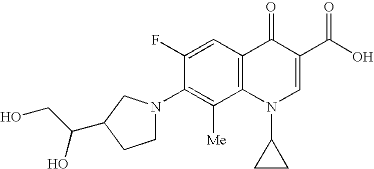 | 2 | 1 | — | 1 |
| 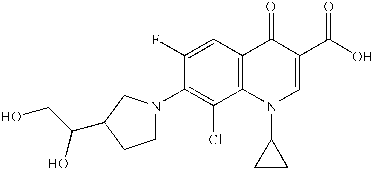 | 0.06 | 0.06 | — | 0.03 |
| 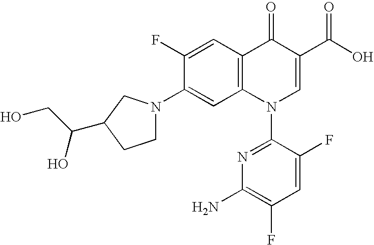 | 0.125 | 0.06 | — | 0.125 |
| 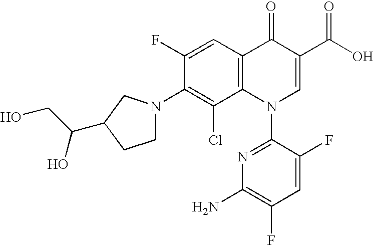 | 0.5 | 0.5 | — | 0.5 |
| 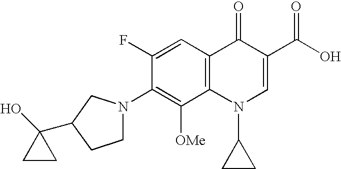 | 0.125 | 0.125 | 0.015 | 0.25 |
| 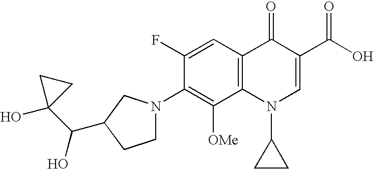 | 0.5 | 0.25 | 0.015 | 0.25 |
| 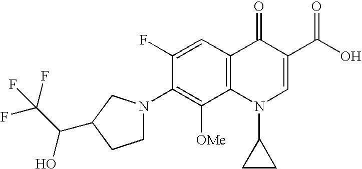 | 0.06 | 0.06 | — | 0.125 |
| 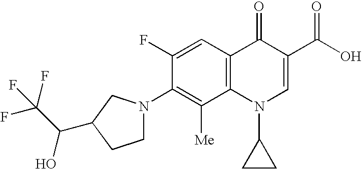 | 0.25 | 0.25 | 0.0015 | 0.5 |
| 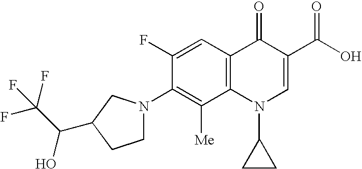 | 0.06 | 0.125 | — | 0.5 |
| 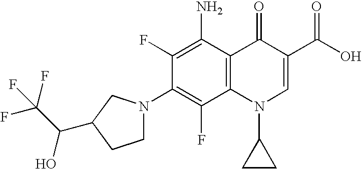 | 0.06 | 0.125 | — | 0.5 |
| 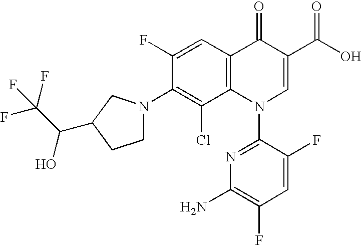 | 0.5 | 0.5 | — | 1 |
| 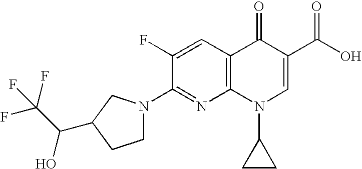 | 0.25 | 0.5 | — | 1 |
| 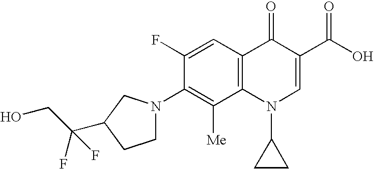 | 0.125 | 0.06 | 0.008 | 0.25 |
| 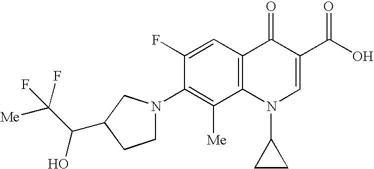 | 0.25 | 0.125 | 0.004 | 0.25 |
| 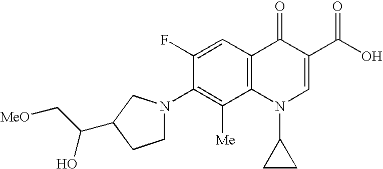 | 1 | 0.5 | 0.06 | 1 |
| 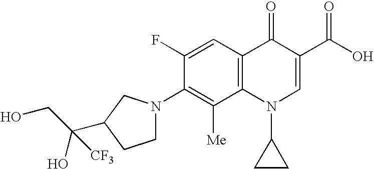 | 0.25 | 0.25 | — | 0.25 |
| 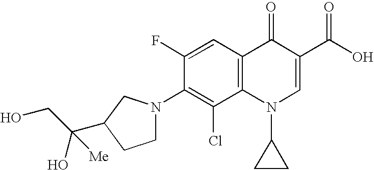 | 0.25 | 0.25 | — | 0.25 |

TABLE 2A

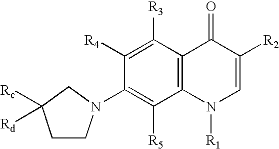

| | Minimum Inhibitory Concentrations μg/mL Gram Negative Bacteria | | |
|---|---|---|---|
| Compound | H. influenzae HI-3542 | M. catarrhalis BC-3531 | E. coli EC-2549 |
| 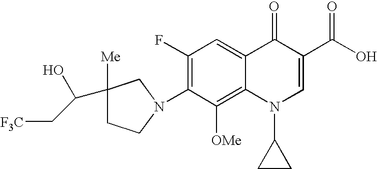 | 0.03 | 0.03 | 0.125 |
| 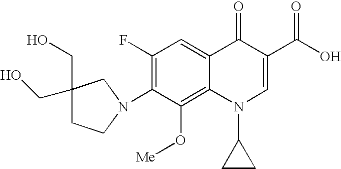 | 0.15 | 0.0125 | 0.5 |

TABLE 2B

| | Minimum Inhibitory Concentrations μg/mL Gram Positive Bacteria | | | |
|---|---|---|---|---|
| Compound | E. faecalis MGH-2 | S. pneumo SV-1 | S. aureus UC-76 | S pyogenes C203 |
| 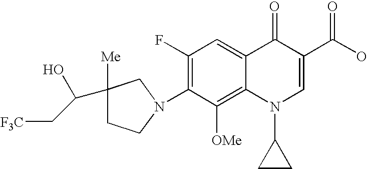 | 0.125 | 0.125 | — | 0.25 |
| 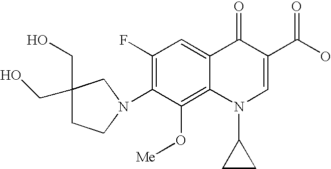 | 0.5 | 0.25 | 0.06 | 0.25 |

TABLE 3A

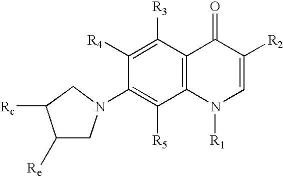

| | Minimum Inhibitory Concentrations μg/mL Gram Negative Bacteria | | |
|---|---|---|---|
| Compound | H. influenzae HI-3542 | M. catarrhalis BC-3531 | E. coli EC-2549 |
| 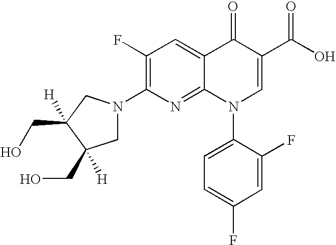 | 0.03 | 0.25 | 0.5 |
| 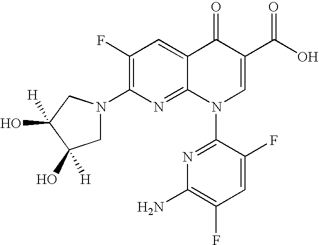 | 0.008 | 0.125 | 0.25 |
| 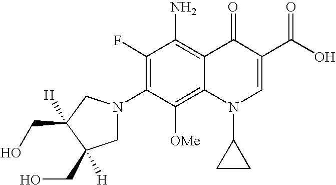 | 0.03 | 0.125 | 0.25 |
| 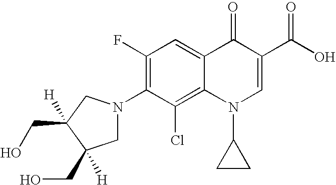 | 0.004 | 0.03 | 0.06 |
| 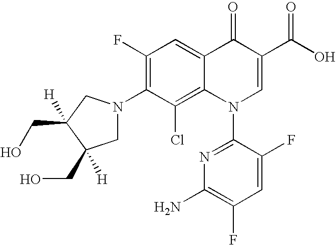 | 0.008 | 0.25 | 0.5 |
| 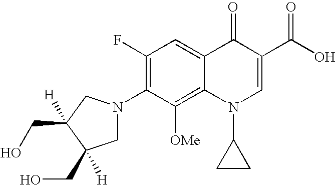 | 0.004 | 0.015 | 0.125 |
| 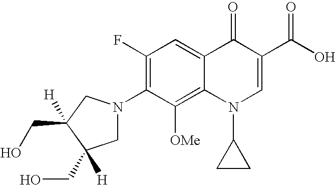 | 0.008 | 0.06 | 0.25 |
| 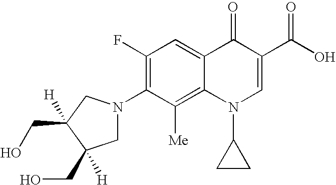 | 0.001 | 0.015 | 0.06 |
| 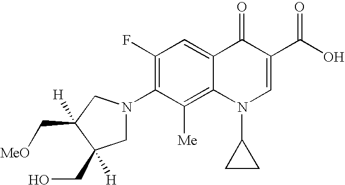 | 0.008 | 0.015 | 0.06 |
| 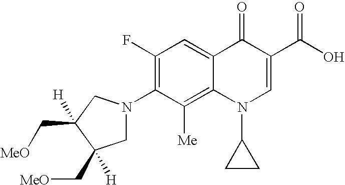 | 0.25 | 0.125 | 2 |
| 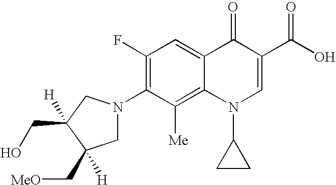 | 0.008 | 0.015 | 0.06 |
| 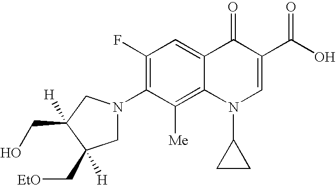 | 0.03 | 0.03 | 0.125 |
| 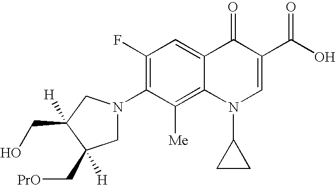 | 0.06 | 0.03 | 0.25 |
| 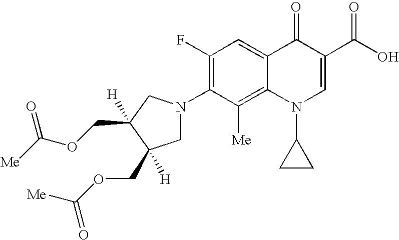 | 0.125 | 0.06 | 0.5 |
| 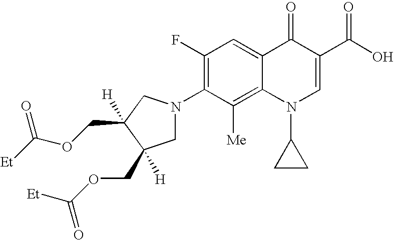 | 0.5 | 0.03 | 1 |
| 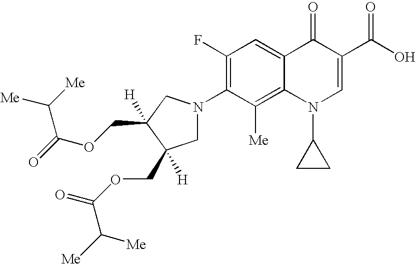 | 2 | 2 | 8 |
| 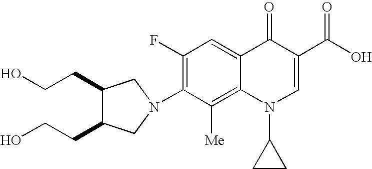 | 0.015 | 0.06 | 0.125 |
| 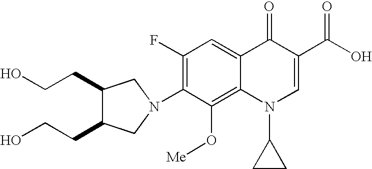 | 0.06 | 0.125 | 1 |
| 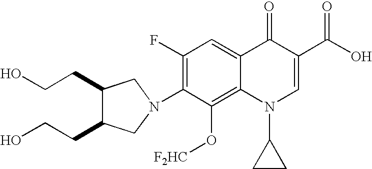 | 0.06 | 0.125 | 1 |

TABLE 3B

| | Minimum Inhibitory Concentrations μg/mL Gram Positive Bacteria | | | |
|---|---|---|---|---|
| Compound | E. faecalis MGH-2 | S. pneumo SV-1 | S. aureus UC-76 | S pyogenes C203 |
| 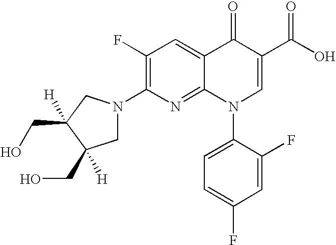 | 0.5 | 0.5 | 0.06 | 0.5 |
| 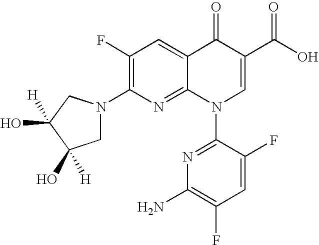 | 0.5 | 0.5 | 0.125 | 0.25 |
| 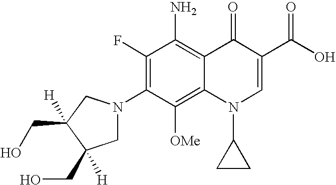 | 0.25 | 0.125 | 0.008 | 0.25 |
| 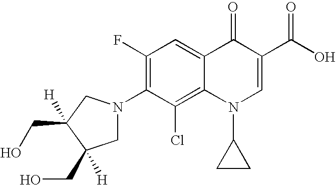 | 0.125 | 0.125 | 0.004 | 0.125 |
| 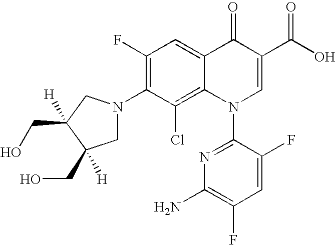 | 2 | 0.5 | 0.125 | 0.5 |
| 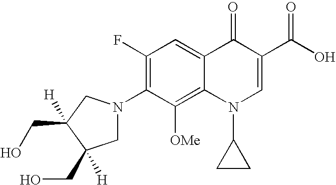 | 0.06 | 0.06 | 0.002 | 0.06 |
| 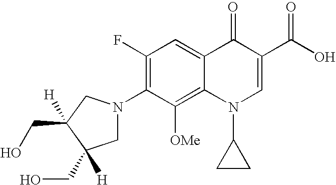 | 0.125 | 0.125 | 0.008 | 0.125 |
| 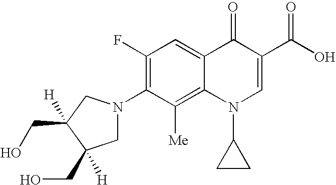 | 0.06 | 0.06 | 0.004 | 0.06 |
| 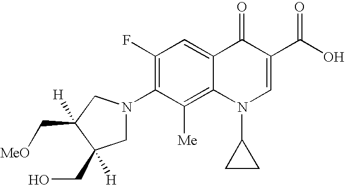 | 0.125 | 0.03 | 0.004 | 0.06 |
| 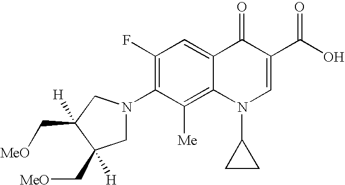 | 0.5 | 0.25 | 0.03 | 0.25 |
| 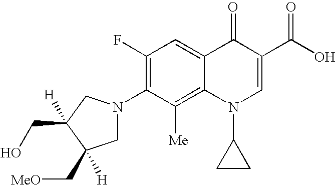 | 0.06 | 0.03 | 0.004 | 0.06 |
| 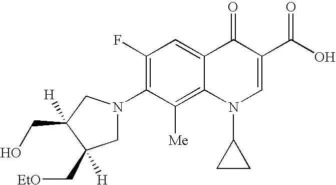 | 0.125 | 0.06 | 0.004 | 0.125 |
| 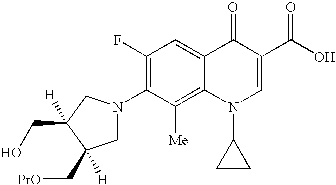 | 0.125 | 0.06 | 0.004 | 0.125 |
| 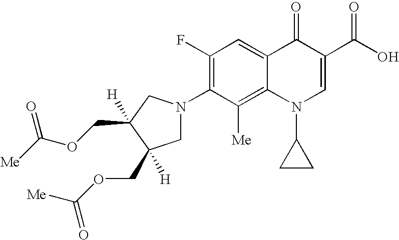 | 0.125 | 0.06 | 0.015 | 0.125 |
| 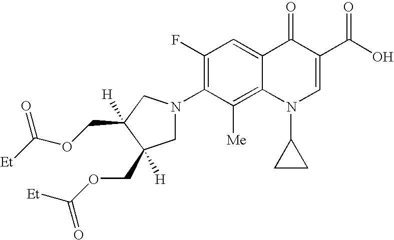 | 0.5 | 0.06 | 0.03 | 0.125 |
| 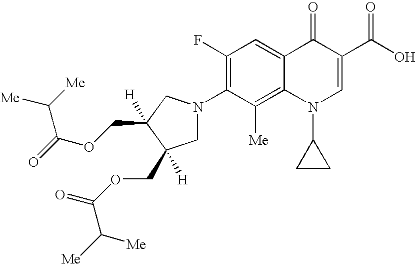 | 4 | 0.125 | 1 | 0.25 |
| 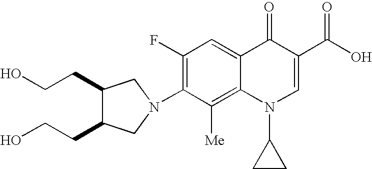 | 0.06 | 0.03 | 0.002 | 0.03 |
| 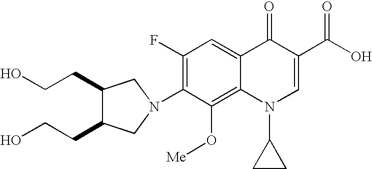 | 0.125 | 0.06 | 0.004 | 0.06 |
| 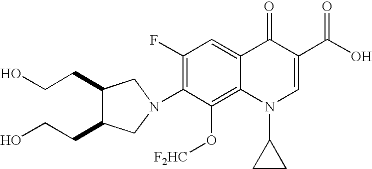 | 0.125 | 0.125 | 0.008 | 0.125 |

TABLE 4A

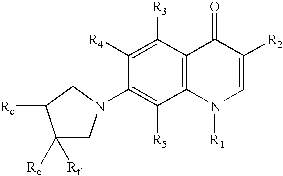

| | Minimum Inhibitory Concentrations μg/mL Gram Negative Bacteria | | |
|---|---|---|---|
| Compound | H. influenzae HI-3542 | M. catarrhalis BC-3531 | E. coli EC-2549 |
| 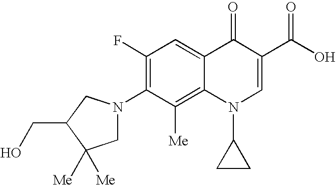 | 0.015 | 0.008 | 0.06 |
| 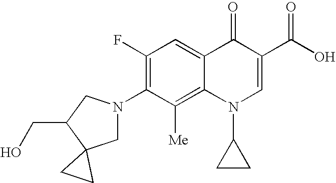 | 0.015 | 0.008 | 0.125 |
| 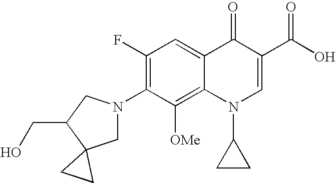 | 0.008 | 0.008 | 0.06 |
| 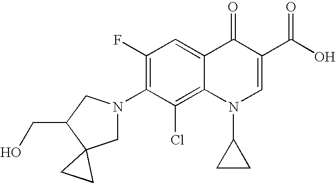 | 0.004 | 0.008 | 0.06 |
| 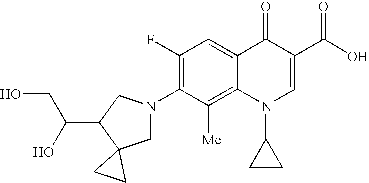 | 0.015 | 0.03 | 0.125 |
| 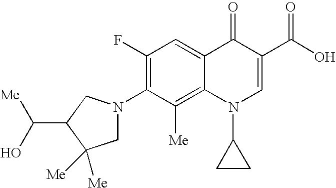 | 0.06 | 0.015 | 0.25 |
| 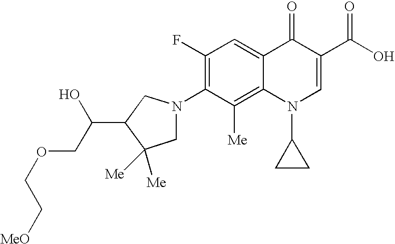 | 0.5 | 0.25 | 2 |
| 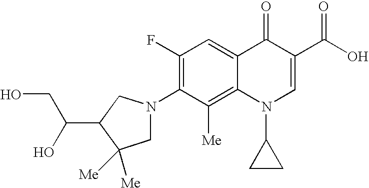 | 0.06 | 0.06 | 0.5 |
| 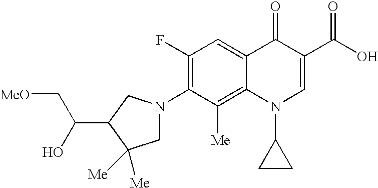 | 0.25 | 0.125 | 1 |

TABLE 4B

| | Minimum Inhibitory Concentrations μg/mL | | | |
|---|---|---|---|---|
| | Gram Positive Bacteria | | | |
| | E. | | | |
| | faecalis | S. pneumo | S. aureus | S pyogenes |
| Compound | MGH-2 | SV-1 | UC-76 | C203 |
| 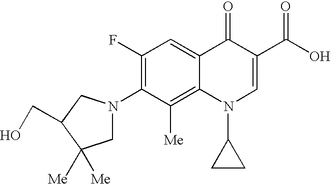 | 0.06 | 0.03 | 0.002 | 0.06 |
| 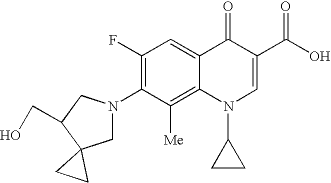 | 0.06 | 0.06 | 0.004 | 0.06 |
| 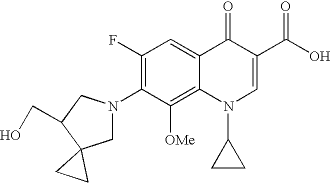 | 0.06 | 0.03 | — | 0.03 |
| 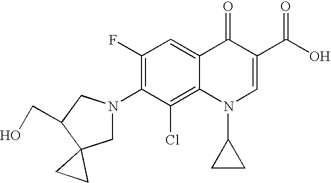 | 0.06 | 0.03 | — | 0.06 |
| 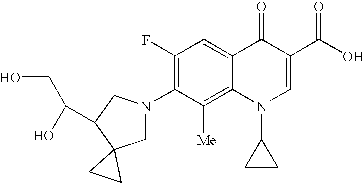 | 0.125 | 0.125 | 0.008 | 0.125 |
| 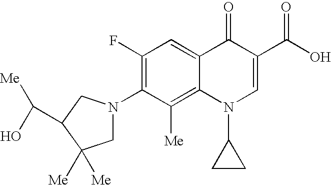 | 0.06 | 0.06 | 0.008 | 0.125 |
| 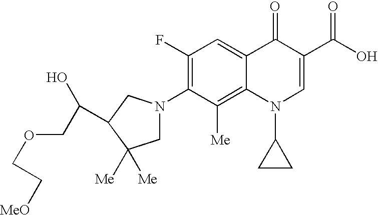 | 0.5 | 1 | 0.03 | 1 |
| 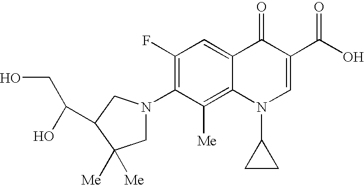 | 0.125 | 0.125 | 0.008 | 0.125 |
| 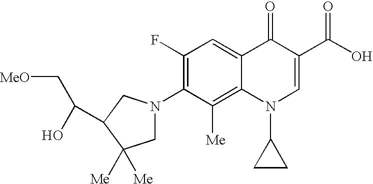 | 0.25 | 0.25 | 0.015 | 0.5 |

TABLE 5A

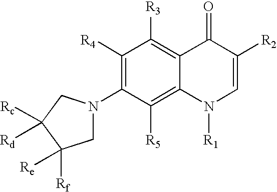

| | Minimum Inhibitory Concentrations | | |
|---|---|---|---|
| | μg/mL Gram Negative Bacteria | | |
| | H. | | |
| | influenzae | M. catarrhalis | E. coli |
| Compound | HI-3542 | BC-3531 | EC-2549 |
| 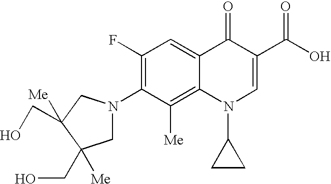 | 0.06 | 0.06 | 0.5 |
| 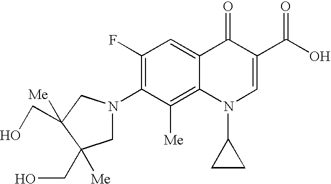 | 0.125 | 0.25 | 2 |

TABLE 5B

| | Minimum Inhibitory Concentrations μg/mL | | | |
|---|---|---|---|---|
| | Gram Positive Bacteria | | | |
| | E. | | | |
| | faecalis | S. pneumo | S. aureus | S pyogenes |
| Compound | MGH-2 | SV-1 | UC-76 | C203 |
| 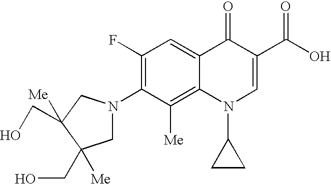 | 0.25 | 0.25 | 0.15 | 0.25 |
| 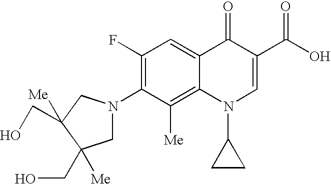 | 1 | 0.5 | 0.015 | 1 |

TABLE 6A
VII

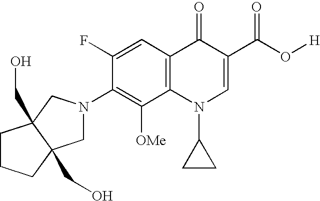

| | Minimum Inhibitory Concentrations | | |
|---|---|---|---|
| | μg/mL Gram Negative Bacteria | | |
| | H. | | |
| | influenzae | M. catarrhalis | E. coli |
| Compound | HI-3542 | BC-3531 | EC-2549 |
| 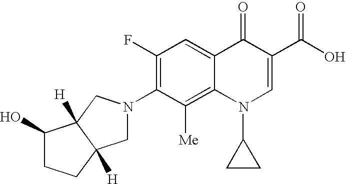 | 0.008 | 0.008 | 0.03 |
| 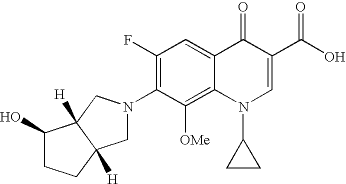 | 0.03 | 0.03 | 0.25 |
| 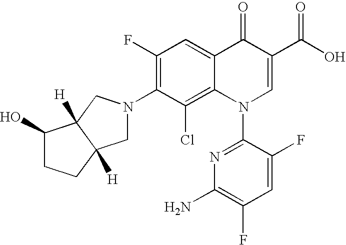 | 0.03 | 0.03 | 0.25 |
| 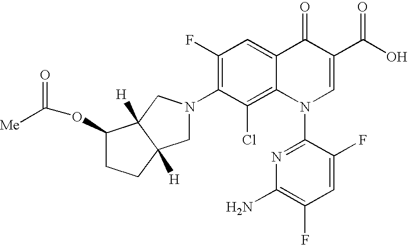 | 0.03 | 0.06 | 0.5 |
| 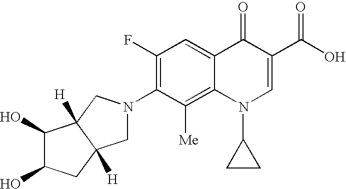 | 0.008 | 0.03 | 0.125 |
| 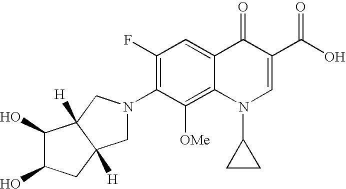 | 0.015 | 0.06 | 0.25 |
| 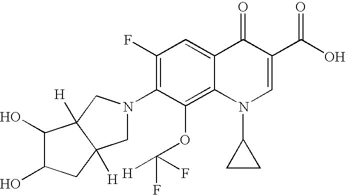 | 0.03 | 0.06 | 0.5 |
| 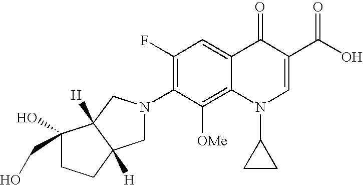 | 0.06 | 0.06 | 0.5 |
| 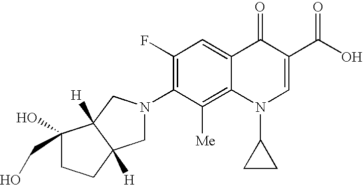 | 0.015 | 0.03 | 0.125 |
| 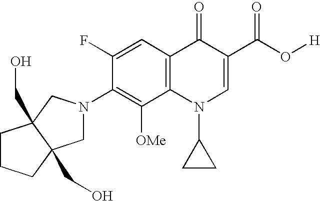 | 0.125 | 0.25 | 2 |
| 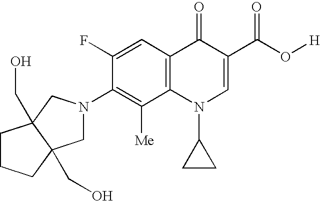 | 0.015 | 0.03 | 0.125 |

TABLE 6B

| | Minimum Inhibitory Concentrations μg/mL | | | |
|---|---|---|---|---|
| | Gram Positive Bacteria | | | |
| | E. faecalis | S. pneumo | S. aureus | S pyogenes |
| Compound | MGH-2 | SV-1 | UC-76 | C203 |
| 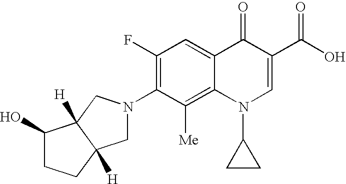 | 0.06 | 0.03 | 0.004 | 0.06 |
| 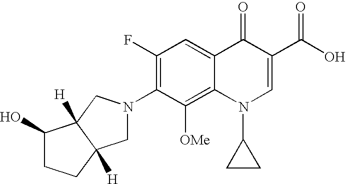 | 0.125 | 0.125 | — | 0.125 |
| 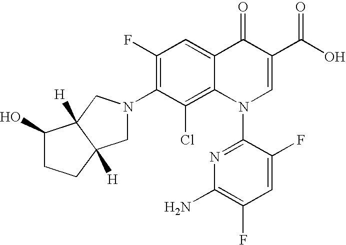 | 0.25 | 0.125 | — | 0.25 |
| 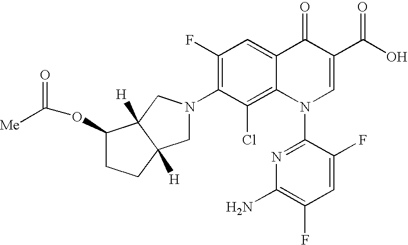 | 0.5 | 0.125 | — | 0.5 |
| 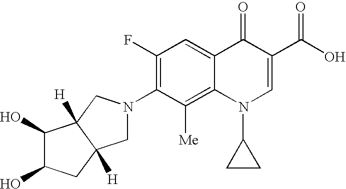 | 0.125 | 0.125 | 0.008 | 0.125 |
| 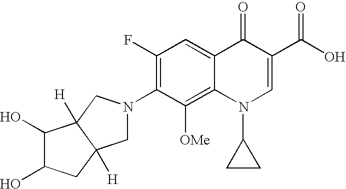 | 0.25 | 0.25 | 0.008 | 0.25 |
| 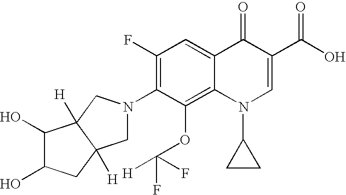 | 0.25 | 0.25 | 0.008 | 0.5 |
| 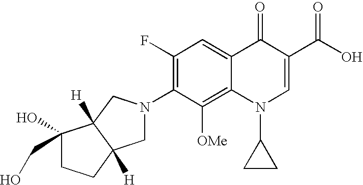 | 0.25 | 0.25 | 0.015 | 0.5 |
| 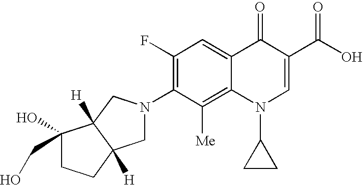 | 0.125 | 0.06 | 0.008 | 0.25 |
| 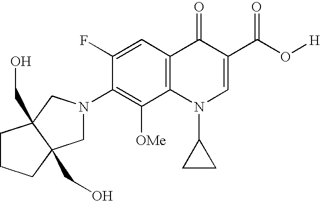 | 0.5 | 0.25 | 0.15 | 1 |
| 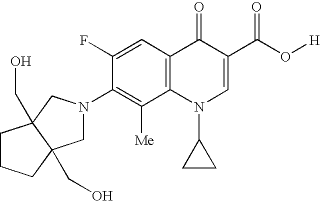 | 0.125 | 0.06 | 0.004 | 0.125 |

The following examples are provided to illustrate but not limit the claimed invention.

A. Synthesis of Sidechain Precursors

### EXAMPLE 1

### Preparation of 3,3,3-Trifluoro-2-pyrrolidin-3-yl-propane-1,2-diol

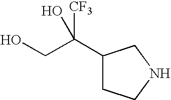

### Step 1: Preparation of 1-Benzyloxy-but-3-en-2-ol

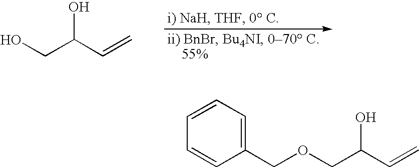

To a cooled (0° C.) solution of 3,4-dihydroxy-1-butene (9.9 mL, 118 mmol) in tetrahydrofuran (400 mL) was added sodium hydride (NAH) (5.7 g, 142 mmol, 60% dispersion in mineral oil) portionwise over 20 minutes. The resulting white slurry was allowed to stir for 30 minutes, and benzyl bromide (16.8 mL, 142 mmol) was added, followed by tetrabutylammonium iodide (8.7 g, 24 mmol). The reaction mixture was warmed to room temperature and was then heated at 70° C. for 24 hours. The mixture was concentrated in vacuo, and the resulting residue was partitioned between saturated aqueous ammonium chloride and dichloromethane. The aqueous layer was extracted three times with dichloromethane, and the combined organics were dried over magnesium sulfate, filtered, and concentrated in vacuo. The resulting residue was purified on a 65M Biotage column using an ethyl acetate/hexanes gradient to afford the title compound (11.6 g, 55%) as a clear oil. ¹H NMR (CDCl₃): δ 2.45 (m, 1H), 3.38 (dd, J=9.5, 7.9 Hz, 1H), 3.54 (dd, J=9.5, 3.4 Hz, 1H), 4.35 (m, 1H), 4.58 (s, 2H), 5.34 (m, 1H), 5.38 (m, 1H), 5.82 (m, 1H), 7.38-7.29 (m, 5H).

### Step 2: Preparation of 1-Benzyloxy-but-3-en-2-one

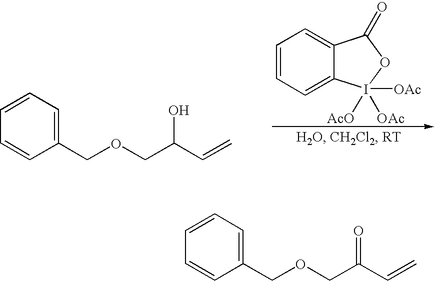

To a solution of 1-benzyloxy-but-3-en-2-ol product (8.0 g, 45 mmol) and Dess-Martin periodinane (23 g, 54 mmol) in dichloromethane (140 mL) was added water (0.89 mL, 49 mmol) in dichloromethane (30 mL) over 30 minutes. After 1.5 hours, the white slurry was diluted with diethyl ether and filtered through a pad of diatomaceous earth (Celite®). The filtrate was washed with a 1:1 mixture of saturated aqueous sodium bicarbonate and 0.1N sodium thiosulfate, dried over magnesium sulfate, filtered, and concentrated in vacuo to give the title compound (7.6 g, 96%) as a yellow liquid which was used without further purification. ¹H NMR (CDCl₃): δ 4.27 (s, 2H), 4.61 (s, 2H), 5.82 (dd, J=10.7, 1.5 Hz, 1H), 6.34 (dd, J=17.6, 1.5 Hz, 1H), 6.55 (dd, J=17.6, 10.7 Hz, 1H), 7.33 (m, 5H).

### Step 3: Preparation of 2-Benzyloxy-1-(1-benzyl-pyrrolidin-3-yl)-ethanone

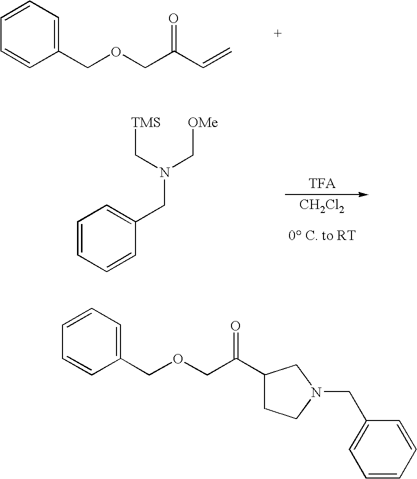

To a cooled (0° C.) solution of 1-(phenylmethoxy)-3-butene-2-one (7.6 g, 43 mmol) and benzyl-1-methoxymethyl-1-trimethylsilylmethyl amine (15 g, 65 mmol) in dichloromethane (105 mL) was added trifluoroacetic acid (3 mL, 1.0 M in dichloromethane). The reaction mixture was allowed to slowly warm to room temperature over 18 hours. The mixture was diluted with dichloromethane and saturated aqueous sodium bicarbonate. The aqueous layer was extracted two times with dichloromethane, and the combined organics were washed with saturated aqueous sodium bicarbonate, brine, dried over magnesium sulfate, filtered, and concentrated in vacuo. The residue was purified on a 40L Biotage column using an ethyl acetate/dichloromethane gradient to afford the title compound (5.0 g, 37%) as a yellow oil. MS(APCI+): m/z 310.1 (M+H)⁺.

### Step 4: Preparation of 3-Benzyloxy-2-(1-benzyl-pyrrolidin-3-yl)-1,1,1-trifluoro-propan-2-ol

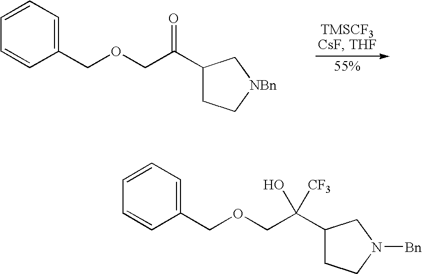

To a mixture of 2-benzyloxy-1-(1-benzyl-pyrrolidin-3-yl)-ethanone (5.0 g, 16 mmol) and 4 angstrom molecular sieves was added trimethyl(trifluoromethyl) silane (65 mL, 32 mmol, 0.5 M in tetrahydrofuran), and the slurry was stirred at room temperature for 10 minutes. Cesium fluoride (1.2 g, 8.1 mmol) was added, and the reaction mixture was stirred for 56 hours. Tetrabutylammonium fluoride (32 mL, 32 mmol, 1 M in tetrahydrofuran) was added and after 5 hours, the mixture was concentrated in vacuo, and the resulting residue was taken up in dichloromethane and filtered through a pad of diatomaceous earth (Celite®). The organic layers were combined, washed with saturated aqueous sodium bicarbonate, brine, dried over magnesium sulfate, filtered, and concentrated in vacuo. The residue was purified on a 65M Biotage column using an ethyl acetate/dichloromethane gradient to afford the title compound (3.3 g, 55%) as an orange oil. MS(APCI+): m/z 380.2 (M+H)⁺, 378.1 (M−H)⁺.

### Step 5: Preparation of 3,3,3-Trifluoro-2-pyrrolidin-3-yl-propane-1,2-diol

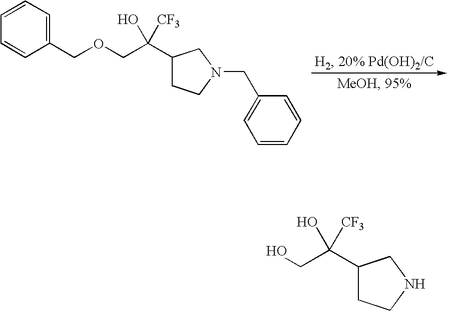

To a solution of 3-Benzyloxy-2-(1-benzyl-pyrrolidin-3-yl)-1,1,1-trifluoro-propan-2-ol (3.3 g, 8.7 mmol) in tetrahydrofuran (50 mL) and methanol (50 mL) in a Parr shaker was added 20% palladium hydroxide on carbon (2.5 g), and hydrogen gas was introduced at 37 psi for 18 hours. The reaction mixture was diluted with methanol, filtered through diatomaceous earth (Celite®), and concentrated in vacuo to afford the title compound (1.5 g, 87%) as an orange oil which was used without further purification. MS(APCI+): m/z 200.0 (M+H)⁺.

### EXAMPLE 2

### Preparation of 2-Pyrrolidin-3-yl-propane-1,2-diol

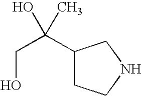

### Step 1: Preparation of 1-Benzyl-pyrrolidine-3-carboxylic acid methoxy-methyl-amide

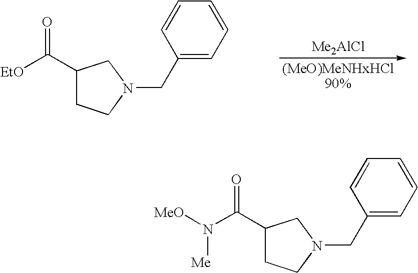

To a cooled (0° C.) solution of N,O-dimethylhydroxylamine hydrochloride (3.7 g, 38 mmol) in dichloromethane (120 mL) was added dimethylaluminum chloride (38 mL, 38 mmol, 1.0 M in hexanes) dropwise. The reaction mixture was warmed to room temperature, and after 1 hour, 1-benzylpyrrolidine-3-carboxylic acid ethyl ester (3.0 g, 13 mmol) in dichloromethane (40 mL) was added. The transfer was completed with 10 mL of dichloromethane. After 18 hours, the reaction mixture was poured into 150 mL of cold (0° C.) 1 M potassium carbonate (pH 13). The slurry was stirred at 0° C. for 1 hour, room temperature for 2 hours, and filtered through a pad of diatomaceous earth (Celite®) using chloroform as the eluent. The aqueous layer was removed, and the organics were dried over magnesium sulfate, filtered, and concentrated in vacuo to afford the title compound (2.9 g, 91%) as an orange oil which was used without further purification. MS(APCI+): m/z 249.2 (M+H)⁺.

### Step 2: Preparation of 3-(Methoxy-methyl-carbamoyl)-pyrrolidine-1-carboxylic acid benzyl ester

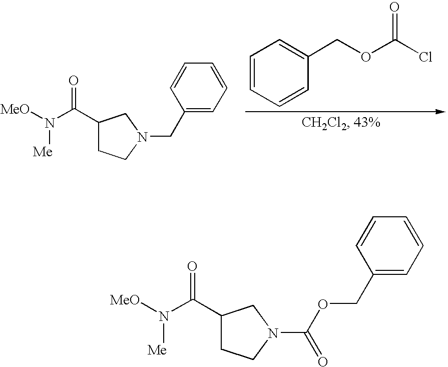

To a solution of 1-benzyl-pyrrolidine-3-carboxylic acid methoxy-methyl-amide (1.1 g, 4.4 mmol) in dichloromethane (40 mL) was added benzyl chloroformate (1.1 mL, 7.5 mmol). The reaction mixture was stirred at room temperature for 4 hours and was then concentrated in vacuo. The resulting residue was purified on a 40L Biotage column using an ethyl acetate/hexanes gradient to afford the title compound (0.91 g, 71%) as a clear liquid. MS(APCI+): m/z 293.1 (M+H)⁺.

### Step 3: Preparation of 3-Acetyl-pyrrolidine-1-carboxylic acid benzyl ester

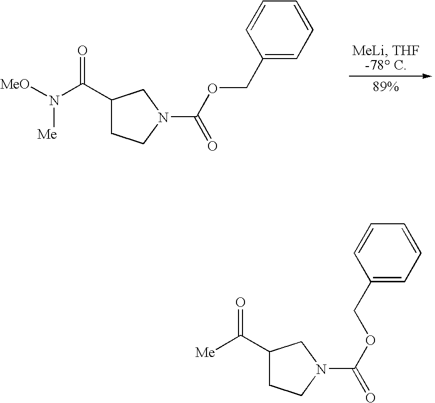

To a cooled (−78° C.) solution of 3-(methoxy-methyl-carbamoyl)-pyrrolidine-1-carboxylic acid benzyl ester (2.8 g, 9.6 mmol) in tetrahydrofuran (50 mL) was added methyllithium (9.0 mL, 14 mmol, 1.6 M in diethyl ether) dropwise. After 1 hour, the reaction mixture was warmed to room temperature and diluted with saturated aqueous ammonium chloride and ethyl acetate. The aqueous layer was extracted two times with ethyl acetate, and the combined organics were washed with brine, dried over magnesium sulfate, filtered, and concentrated in vacuo. The residue was purified on a 40S Biotage column using an ethyl acetate/hexanes gradient to afford the title compound (2.1 g, 89%) as a yellow oil. MS(APCI+): m/z 248.1 (M+H)⁺.

### Step 4: Preparation of 3-Acetyl-pyrrolidine-1-carboxylic acid benzyl ester

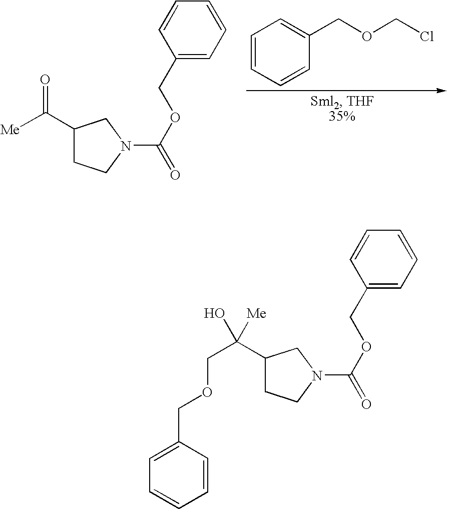

To a mixture of 3-acetyl-pyrrolidine-1-carboxylic acid benzyl ester (2.3 g, 9.3 mmol) and benzyl chloromethyl ether (2.6 mL, 11 mmol, 60% technical grade) was added samarium iodide (280 mL, 28.0 mmol, 0.1 M in tetrahydrofuran). The dark blue reaction mixture was stirred at room temperature for 3 hours, during which time it turned bright yellow. The mixture was diluted with saturated aqueous ammonium chloride and ethyl acetate. The aqueous layer was extracted two times with ethyl acetate, and the combined organics were washed with brine, dried over magnesium sulfate, filtered, and concentrated in vacuo. The residue was purified on a 40M Biotage column using an ethyl acetate/hexanes gradient to afford the title compound (1.2 g, 35%) as a pale yellow oil. MS(APCI+): m/z 370.1 (M+H)⁺.

### Step 5: Preparation of 3-Acetyl-pyrrolidine-1-carboxylic acid benzyl ester

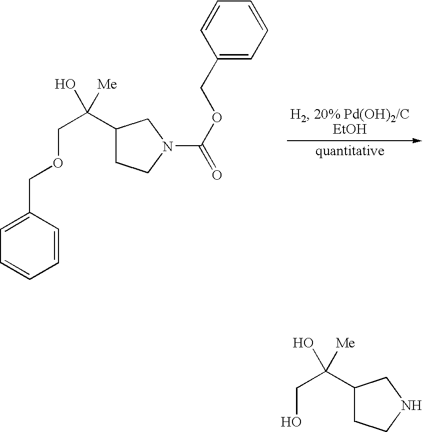

To a solution of 3-acetyl-pyrrolidine-1-carboxylic acid benzyl ester (0.60 g, 1.6 mmol) in ethanol (16 mL) in a Parr shaker was added 20% palladium hydroxide on carbon (0.66 g), and hydrogen gas was introduced at 37 psi for 130 hours. The reaction mixture was diluted with methanol, filtered through diatomaceous earth (Celite®), and concentrated in vacuo to afford the title compound (0.24 g, 100%) as an orange oil which was used without further purification. MS(APCI+): m/z 146.1 (M+H)⁺.

### EXAMPLE 3

### 1-Pyrrolidin-3-yl-ethanol

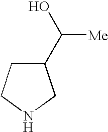

### Step 1: Preparation of 1-Benzyl-pyrrolidine-3-carboxylic acid methoxy-methyl-amide

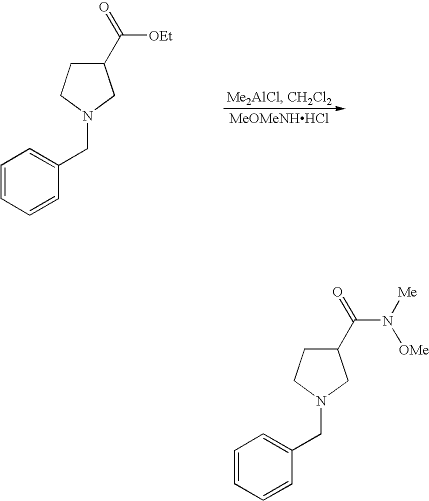

To methoxymethylamine hydrochloride (3.74 g, 38 mmol) in dichloromethane (120 mL) at 0° C. was added dimethylaluminum chloride (38 mL, 38 mmol) via addition funnel. The reaction was warmed to room temperature and stirred for 1 hour. 1-benzyl-pyrrolidine-3-carboxylic acid ethyl ester (3.0 g, 12.8 mmol) in dichloromethane (40 mL) was added and after 12 hours, the mixture was poured into 1M potassium carbonate solution (150 mL) at 0° C., stirred at 0° C. for 1 hour, then at room temperature for 2 hours. The mixture was filtered through celite, and extracted with chloroform. The organic layer was dried over magnesium sulfate, filtered, and concentrated in vacuo to give the title compound (Yield: 2.9 g) which was used in the next step without further purification.

### Step 2: Preparation of 3-Acetyl-pyrrolidine-1-carboxylic acid benzyl ester

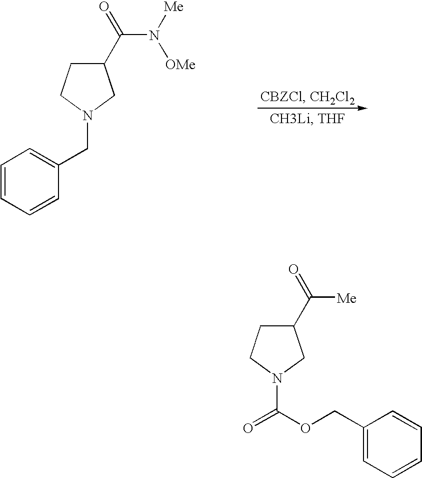

To a solution of 1-benzyl-pyrrolidine-3-carboxylic acid methoxy-methyl-amide (240 mg, 0.97 mmol) in dichloromethane (10 mL) was added carboxybenzyloxychloride (0.23 mL, 1.64 mmol). After stirring at room temperature for 1.5 hours, the reaction mixture was concentrated and the crude residue was purified by column chromatography (0 to 100% ethyl acetate in dichloromethane) to afford the requisite CBZ intermediate (Yield: 120 mg, 43%) which was used in the next step without further purification.

To a solution of the CBZ intermediate (2.8 g, 9.6 mmol) in tetrahydrofuran (50 mL) at −78° C. was added methyl lithium (9 mL of a 1.6M solution in ether) dropwise by syringe. After 1 hour, the reaction mixture was warmed to room temperature, quenched with saturated aqueous ammonium chloride solution and extracted with ethyl acetate. The organic layer was washed with brine, dried over magnesium sulfate, filtered, and concentrated to afford a crude residue which was purified by column chromatography (0 to 100% ethyl acetate in hexanes) to afford the title compound (Yield: 2.1 g, 89%) which was used without further purification in the next step.

### Step 3: Preparation of 3-(1-Hydroxy-ethyl)-pyrrolidine-1-carboxylic acid benzyl ester

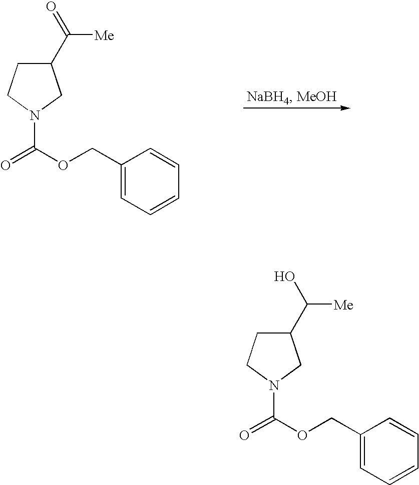

To a solution of 3-Acetyl-pyrrolidine-1-carboxylic acid benzyl ester (1.4 g, 5.7 mmol) in methanol (30 mL) at 0° C. was added sodium borohydride (0.32 g, 8.5 mmol). After 2 hours, the reaction was quenched with saturated ammonium chloride solution and extracted with dichloromethane. The organic layer was dried over magnesium sulfate, filtered, and concentrated. The crude residue was purified by column chromatography (30 to 50% ethyl acetate in hexanes) to afford the title compound as a 2:1 mixture of diastereomers (Yield: 1.0 g, 71%). MS(APCI+): m/z 250 (M+H)+.

### Step 4: Preparation of 1-Pyrrolidin-3-yl-ethanol

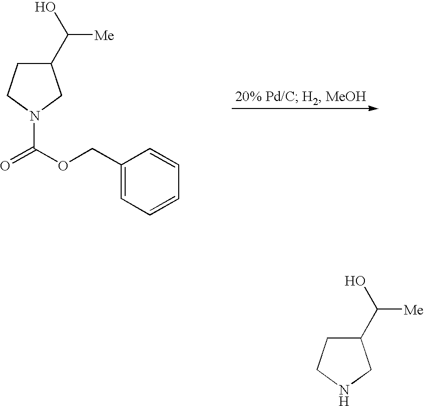

To a solution of 3-(1-hydroxy-ethyl)-pyrrolidine-1-carboxylic acid benzyl ester (1.0 g, 4.0 mmol) in methanol (50 mL) was added 20% Pd/C. The reaction was stirred under an atmosphere of hydrogen for 12 hours. The reaction was filtered and concentrated to give the title compound (Yield: 426 mg, 92%). MS(APCI+): m/z 116 (M+H)+.

### EXAMPLE 4

### 2,2-Difluoro-1S-pyrrolidin-3R-yl-ethanol

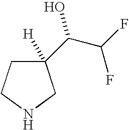

### Step 1: Preparation of 1-(1R-Phenyl-ethyl)-pyrrolidin-2-one

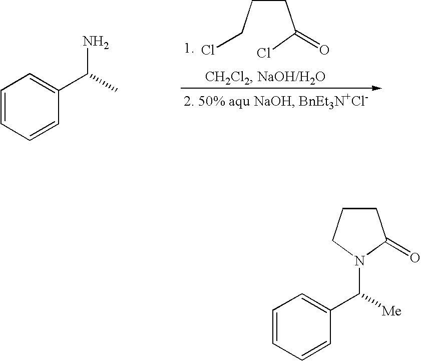

R-(+)-α-methylbenzylamine (100 g, 825 mmol), methylene chloride (330 mL), and sodium hydroxide (26 g, 907 mmol) in water (412 mL) were introduced into a 3 liter 3-necked round bottom flask which was mechanically stirred and equipped with a 250 mL dropping funnel and thermometer. The mixture was cooled in an ice bath while stirring vigorously. 4-Chlorobutyryl chloride (92 mL, 825 mmol) in dichloromethane (80 mL) was added dropwise keeping the temperature under 10° C. The reaction mixture was stirred vigorously for 15 minutes, and transferred to a 2 liter separatory funnel. The dichloromethane layer was transferred into a 2 liter 3-necked round bottom flask containing benzyltriethylammonium chloride (9.4 g, 41 mmol) to which a 50% solution of sodium hydroxide in water (330 mL) was added rapidly. The mixture was stirred vigorously then heated at reflux for 2 hours. Water was added and the dichloromethane layer was drawn off. The aqueous layer was back extracted 2× and the combined organic layers were washed with 1N HCl followed by water. The organic layer was dried over magnesium sulfate, filtered and concentrated to give the title compound (Yield: 155 g, 99%) which was used in the next step without further purification.

### Step 2: Preparation of 3-(2,2-Difluoro-acetyl)-1-(1R-phenyl-ethyl)-pyrrolidin-2-one

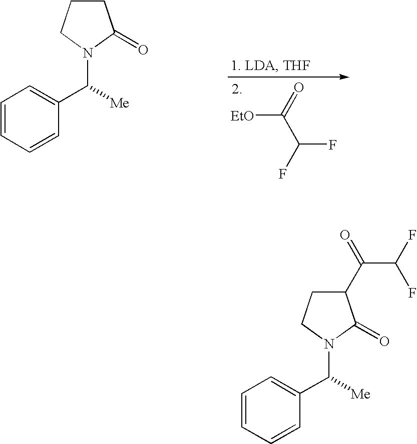

To a solution of 1-(1R-phenyl-ethyl)-pyrrolidin-2-one (23.2 g, 123 mmol) in tetrahydrofuran (300 mL) at −78° C. was added lithium diisopropylamide (74 mL of a 2M solution in heptane/tetrahydrofuran/ethyl benzene). The reaction stirred for 1 hour 40 minutes. This solution was added via cannula to a solution of ethyl difluoroacetate (16 mL, 160) in tetrahydrofuran (100 mL). After 1 hour, the reaction was quenched by the addition of saturated aqueous ammonium chloride solution. The reaction was warmed to room temperature and concentrated to remove the tetrahydrofuran. The mixture was diluted with water and extracted with ethyl acetate. The organic layer was dried over magnesium sulfate, filtered and concentrated. The crude residue was purified by column chromatography (20% ethyl acetate in hexanes) to afford the title compound (Yield: 20.9 g, 64%) which was used in the next step without further purification.

### Step 3: Preparation of 3-(2,2-Difluoro-1-hydroxy-ethyl)-1-(1R-phenyl-ethyl)-pyrrolidin-2-one

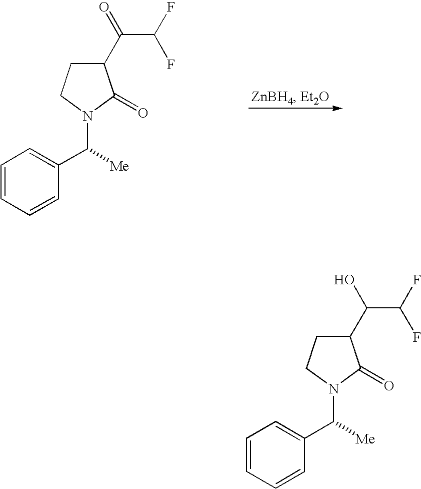

To a solution of 3-(2,2-Difluoro-acetyl)-1-(1R-phenyl-ethyl)-pyrrolidin-2-one (19.6 g, 73.4 mmol) in ether (200 mL) at 0° C. was added a solution of zinc borohydride (100 mmol) in ether (200 mL) via cannula. The reaction warmed to room temperature overnight. The reaction was quenched by the dropwise addition of saturated aqueous ammonium chloride solution. The mixture was extracted with ethyl acetate and the organic layer was dried over magnesium sulfate, filtered and concentrated. The crude residue was purified by column chromatography (30 to 40% ethyl acetate in hexanes) to give a mixture of diastereomers 1 and 2 (Yield: 8.7 g, 44%) and a mixture of diastereomers 3 and 4 (Yield 4.7 g, 24%). Diastereomers 1 and 2 were carried into the next step without further purification.

### Step 4: Preparation of BOC Valine-Protected 3-(2,2-Difluoro-1-hydroxy-ethyl)-1-(1R-phenyl-ethyl)-pyrrolidin-2-one

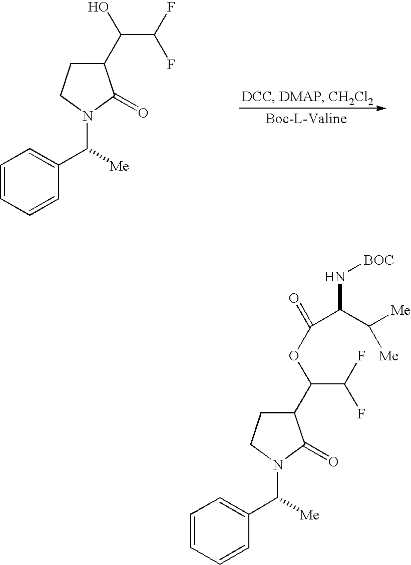

To a solution of 3-(2,2-difluoro-1-hydroxy-ethyl)-1-(1R-phenyl-ethyl)-pyrrolidin-2-one (6.0 g, 22.3 mmol), boc-L-valine (9.7 g, 44.6 mmol), and N,N-dimethylaminopyridine (272 mg, 2.23 mmol) in dichloromethane (100 mL) at 0° C. was added dicyclohexyl carbodiimide (17.5 g, 84.8 mmol). The reaction mixture stirred for 2 hours. The solid was filtered off, and the filtrate was poured into a solution of saturated sodium bicarbonate solution and extracted with dichloromethane. The organic layer was dried over magnesium sulfate, filtered and concentrated. The crude residue was purified by column chromatography (10 to 40% ethyl acetate in hexanes) to afford diastereomer 2 (Yield 4.9 g, 47%) and Diastereomer 1 (Yield: 4.3 g, 41%) Diastereomer 1 was used in the next step without further purification.

### Step 5: Preparation of 2,2-Difluoro-1S-[1-(1R-phenyl-ethyl)-pyrrolidin-3R-yl]-ethanol

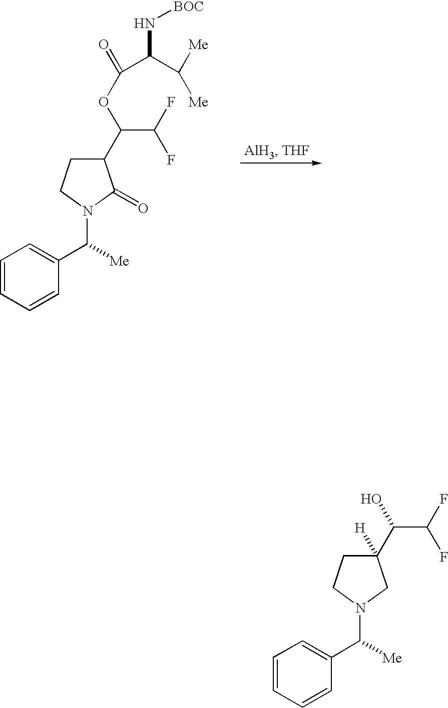

To aluminum chloride (7.3 g, 54.5 mmol) at 0° C. was added tetrahydrofuran (30 mL) dropwise. After 5 minutes, lithium aluminum hydride (163 mL of a 1M solution in tetrahydrofuran) was added. The solution of alane was warmed to room temperature and stirred for 20 minutes, cooled to −78° C., and a solution of 2,2-difluoro-1S-[1-(1R-phenyl-ethyl)-pyrrolidin-3R-yl]-ethanol (17 g, 36.3 mmol) in tetrahydrofuran (60 mL) was added. The reaction warmed to room temperature over 12 hours and was quenched with 1N HCl, then basified to pH 10 with 50% aqueous sodium hydroxide solution, filtered through celite, and extracted with ethyl acetate. The organic layer was dried over magnesium sulfate, filtered and concentrated. The crude residue was purified by column chromatography (2% methanol/0.2% ammonium hydroxide/98% dichloromethane to 5% methanol/0.5% ammonium hydroxide/95% dichloromethane) to afford the title compound (Yield: 1.9 g, 20%) which was used in the next step without further purification.

### Step 6: Preparation of 2,2-Difluoro-1S-[1-(1R-phenyl-ethyl)-pyrrolidin-3R-yl]-ethanol

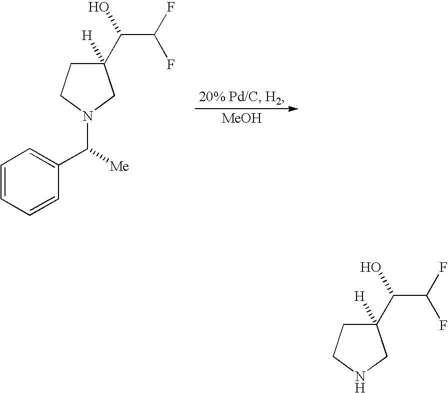

To a solution of 2,2-difluoro-1S-[1-(1R-phenyl-ethyl)-pyrrolidin-3R-yl]-ethanol (1.9 g, 7.5 mmol) in methanol (50 mL) was added 20% Pd/C (500 mg). The reaction ran under an atmosphere of hydrogen gas for 12 hours. The reaction mixture was filtered, and the filtrate concentrated to give the title compound (Yield: 1.1 g, quantitative). MS(APCI+): m/z 152 (M+H)+.

### EXAMPLE 6

### 2,2,2-Trifluoro-1S-pyrrolidin-3R-yl-ethanol

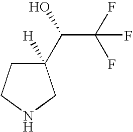

### Step 1: Preparation of 3-(2,2,2-Trifluoro-acetyl)-1-(1R-phenyl-ethyl)-pyrrolidin-2-one

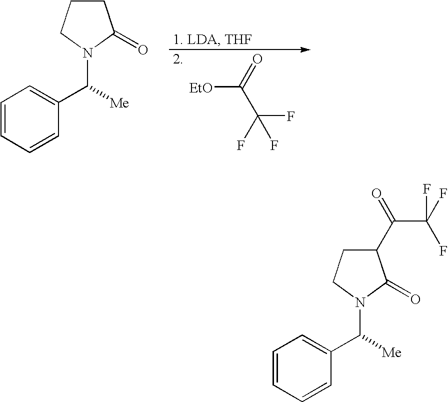

To a solution of 1-(1R-Phenyl-ethyl)-pyrrolidin-2-one (Experiment 5, step 1, 30 g, 159 mmol) in tetrahydrofuran (500 mL) at −78° C. was added lithium diisopropylamide (97 mL of a 1.8M solution in heptane/tetrahydrofuran/ethyl benzene). The reaction stirred for 30 minutes and ethyl trifluoroacetate (24.5 mL, 206 mmol) was added. After 30 minutes, the reaction was quenched by the addition of saturated aqueous ammonium chloride solution. The reaction was warmed to room temperature and concentrated to remove the tetrahydrofuran. The mixture was diluted with water and extracted with ethyl acetate. The organic layer was dried over magnesium sulfate, filtered and concentrated. The crude residue was purified by column chromatography (20 to 40% ethyl acetate in hexanes) to afford the title compound (Yield: 33.5 g, 74%) which was used in the next step without further purification.

### Step 2: Preparation of 3-(2,2,2-Trifluoro-1-hydroxy-ethyl)-1-(1R-phenyl-ethyl)-pyrrolidin-2-one

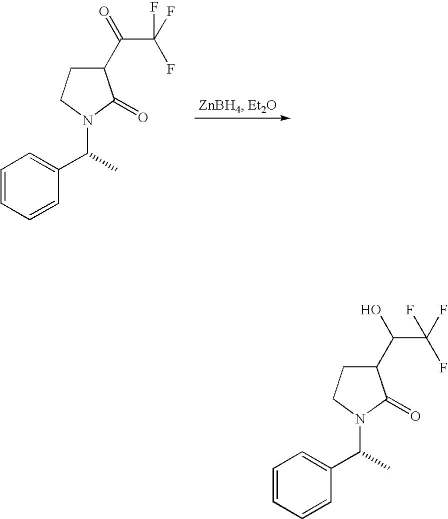

To a solution of 3-(2,2,2-Trifluoro-acetyl)-1-(1R-phenyl-ethyl)-pyrrolidin-2-one (5.2g, 18.2 mmol) in ether (50 mL) was added a solution of zinc borohydride (100 mmol) in ether (200 mL) via cannula. The reaction warmed to room temperature and stirred overnight. The reaction was quenched by the dropwise addition of saturated aqueous ammonium chloride solution. The mixture was extracted with ethyl acetate and the organic layer was dried over magnesium sulfate, filtered and concentrated. The crude residue was purified by column chromatography (10% ether in dichloromethane) and the least polar diastereomer was isolated as the title compound out of a mixture of four diastereomers (Yield: 597 mg) which was carried into the next step without further purification.

### Step 3: Preparation of 3-(2,2,2-Trifluoro-1S-hydroxy-ethyl)-1-(1R-phenyl-ethyl)-pyrrolidin-2-one

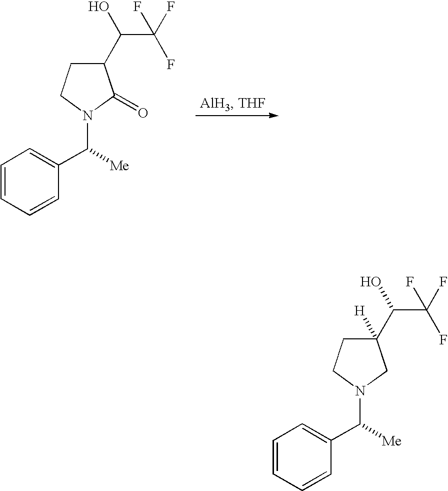

To aluminum chloride (1.0 g, 7.6 mmol) at 0° C. was added tetrahydrofuran (10 mL) dropwise. After 5 minutes, lithium aluminum hydride (23 mL of a 1.0 M solution in tetrahydrofuran) was added. The solution of alane was warmed to room temperature and stirred for 20 minutes, cooled to −78° C., and a solution of 3-(2,2,2-Trifluoro-1-hydroxy-ethyl)-1-(1R-phenyl-ethyl)-pyrrolidin-2-one (1.88 g, 6.6 mmol) in tetrahydrofuran (10 mL) was added. The reaction warmed to room temperature over 12 hours and was quenched with 1N HCl, then basified to pH 10 with 50% aqueous sodium hydroxide solution, filtered through celite, and extracted with ethyl acetate. The organic layer was dried over magnesium sulfate, filtered and concentrated. The crude residue was purified by column chromatography (5% methanol in dichloromethane) to afford the title compound (Yield: 1.6 g, 87%) which was used in the next step without further purification.

### Step 4: Preparation of 3-(2,2,2-Trifluoro-acetyl)-1-(1R-phenyl-ethyl)-pyrrolidin-2-one

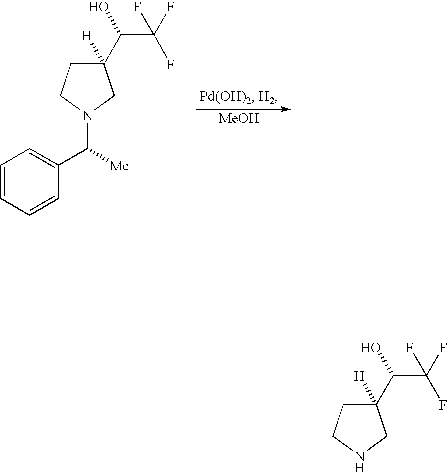

To a solution of 3-(2,2,2-Trifluoro-1S-hydroxy-ethyl)-1-(1R-phenyl-ethyl)-pyrrolidin-2-one (1.6 g, 5.8 mmol) in methanol (50 mL) was added 20% Pd(OH)₂/C (200 mg). The reaction ran under an atmosphere of hydrogen gas for 26 hours. The reaction mixture was filtered, and the filtrate concentrated to give the title compound (Yield: 967mg, quantitative). MS(APCI+): m/z 170 (M+H)+.

### EXAMPLE 7

### 1-Pyrrolidin-3R-yl-ethane-1R,2-diol

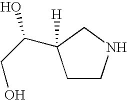

### Step 1: Prepaaration of 5-Hydroxymethyl-5H-furan-2-one

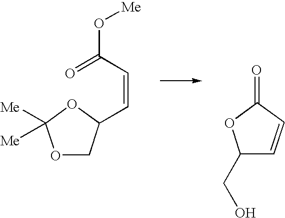

5-Hydroxymethyl-5H-furan-2-one was prepared according to Nagaoka, Iwashima, Abe, Yamada Tet. Lett. (1989), 30, 5911-5914.

### Step 2: Preparation of 5-Triisopropylsilanyloxymethyl-5H-furan-2-one

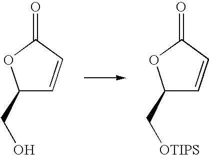

To a solution of crude 5-Hydroxymethyl-5H-furan-2-one (3.1 g, 27 mmol) in dichloromethane (100 mL) was added imidazole (4.7 g, 69 mmol) and then triisopropyl chloride (7.1 mL, 33 mmol) and the solution was stirred at ambient temperature for 18 hours. The mixture was concentrated in vacuo, taken up in ethyl acetate (100 mL), washed with satd. sodium bicarbonate (100 mL), satd. ammonium chloride (2×100 mL), brine (100 mL), dried with magnesium sulfate and concentrated in vacuo. The crude was run on a 100 g silica gel column eluted with 0 to 30% ethyl acetate in hexanes over 80 minutes to give 6.4 g of the title compound (yield: 86%). MS (APCI+): m/z 271 (M+H)+.

### Step 3: Preparation of 5-Triisopropylsilanyloxymethyl-4-nitromethyl-dihydro-furan-2-one

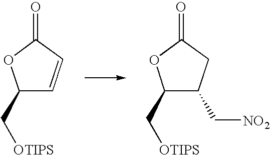

To a solution of 1,8-diazabicyclo[5.4.0]undec-7-ene (3.9 mL, 26 mmol) in nitromethane (50 mL) at 0° C. was added 5-Triisopropylsilanyloxymethyl-5H-furan-2-one and the solution stirred for 30 minutes. The solution was concentrated in vacuo to approximately 20 mL and diluted with ethyl acetate (225 mL) and washed with saturated. ammonium chloride (2×200 mL), brine (200 mL), dried with magnesium sulfate and concentrated in vacuo. The crude was run on a 110 g silica gel column eluted with 0 to 25% ethyl acetate in hexanes over 2 hours to give 4.8 g of the title compound (yield: 55%). MS (APCI−): m/z 330 (M−H)−.

### Step 4: Preparation of 4R-Aminomethyl-5S-triisopropylsilanyloxymethyl-dihydro-furan-2-one

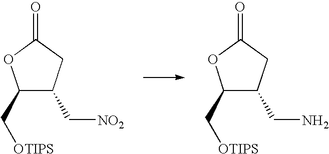

A solution of triisopropylsilanyloxymethyl-4-nitromethyl-dihydro-furan-2-one (13.9 g, 0.04 mol) in methanol (150 mL) was treated with Ra—Ni (12.0 g) and hydrogenated at ˜50 psi for ˜15 hours. The reaction mixture was filtered through celite (until the product was properly washed off the catalyst) and the filtrate was evaporated under vacuum to give 12 g of the crude amine (yield: 98%), which is directly used in the next step. MS (APCI+): m/z 302 (M+H)+.

### Step 5: Preparation of 4R-(1-Hydroxy-2-triisopropylsilanyloxymethyl-ethyl)-pyrrolidin-2-one

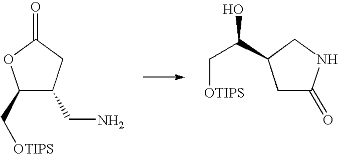

To a solution of (5S,4R)-4-(aminomethyl)-5-{[1,1-bis(methylethyl)-2-methyl-1-silapropoxy]methyl}-4R-Aminomethyl-5S-triisopropylsilanyloxy methyl-dihydro-furan-2-one (12.0 g, 0.039 mol) in absolute ethanol (200 mL) was added sodium tert-butoxide (3.81 g, 0.039 mol) at room temperature and after completion of addition the solution was heated to 50° C. for 3 hours. The reaction mixture was cooled to room temperature, quenched with acetic acid (5 mL) and concentrated under vacuum. The residue was diluted with ethyl acetate (500 mL), washed with saturated ammonium chloride (1×100 mL), saturated bicarbonate (1×100 mL) and then with brine (1×100 mL), dried over anhydrous sodium sulfate, filtered and concentrated under reduced pressure. The residue was purified by column chromatography over silica gel (50% ethyl acetate in hexane) to give 10 g of the title compound (yield: 83%). MS (APCI+): m/z 302 (M+H)+.

### Step 6: Preparation 4R-(1S-Benzyloxy-2-triisopropylsilanyloxy-ethyl)-pyrrolidin-2-one

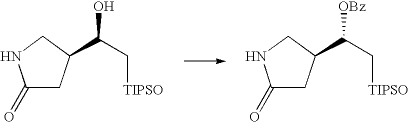

To a solution of benzoic acid (16.5 g, 0.135 mol) in diethyl ether (500 mL) was slowly added diethylazodicarboxylate (DEAD) (23.92 g, 21.62 mL, 0.137 mol) followed by a solution of triphenyl phosphine (35.5 g, 0.135 mol) and 4R-(1-Hydroxy-2-triisopropylsilanyloxymethyl-ethyl)-pyrrolidin-2-one (18.0 g, 0.059 mol) in diethyl ether at room temperature. The reaction mixture was stirred overnight at room temperature, the suspension (white precipitate) was filtered, and the filtrate was concentrated under reduced pressure. The thick residue was purified by column chromatography over silica gel to give 8.2 g of the title compound (yield: 34%). H¹ NMR (400 MHz, CDCl₃): δ 8.0 (d, 2H), 7.7-7.4 (m, 3H), 5.2 (m, 1H), 4.2 (m, 1H), 3.9 (d, 2H), 3.6 (m, 1H), 3.4 (m, 1H), 3.2 (m, 1H), 2.5 (m, 1H), 2.3 (m, 1H), 1.2-1.0 (m, 21H).

### Step 7: Preparation of 4R-(1S-Hydroxy-2-triisopropylsilanyloxy-ethyl)-pyrrolidin-2-one

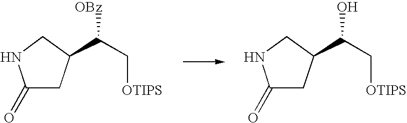

To a solution of 4R-(1S-Benzyloxy-2-triisopropylsilanyloxy-ethyl)-pyrrolidin-2-one (18.0 g, 0.044 mol) in methanol (2.1 L) was added sodium methoxide (3.0 g, 0.055 mol) and the solution was stirred overnight at room temperature. The solution was neutralized by Dowex 50WX8(H⁺), filtered and concentrated under reduced pressure to give a thick residue, which upon column chromatography gave 9.9 g of the title compound (yield: 74%). MS (APCI+): m/z 302 (M+H)+.

### Step 8: Preparation of N-Benzyl-4R-(1S,2-dibenzyloxyethyl)-pyrrolidin-2-one

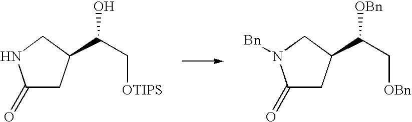

To a solution of 4R-(1S,2-dibenzyloxyethyl)-pyrrolidin-2-one (3.8 g, 13 mmol) in anhydrous tetrahydrofuran (100 mL) at 0° C. was added 1.0 M tetrabutylammonium fluoride in tetrahydrofuran (15 mL, 15 mmol). The solution was allowed to come to ambient temperature and stirred for 2 hours. To the solution was added benzyl bromide (9 mL, 76 mmol) and tetrabutylammonium iodide (0.94 g, 3 mmol) and the solution was cooled to 0° C. To the solution was slowly added 60 wt % sodium hydride in oil (3.6 g, 90 mmol). The ice bath was removed and the mixture stirred for 15 minutes. The mixture was then heated at 60° C. for 2.5 hours. The solution was concentrated in vacuo and taken up in ethyl acetate (200 mL), washed with saturated. ammonium chloride (200 mL), saturated sodium bicarbonate (200 mL), brine (200 mL), dried with magnesium sulfate and concentrated in vacuo. The crude product was purified on a 120 g silica gel column eluted with 10 to 100% ethyl acetate in hexanes over 1 hour to give 3.5 g of the title compound (yield: 66%). MS (APCI+): m/z 416 (M+H)+.

### Step 9: Preparation of N-Benzyl-4R-(1S,2-dibenzyloxyethyl)-pyrrolidine

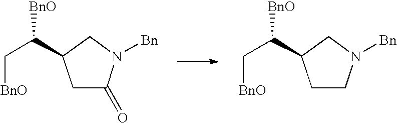

To a solution of N-Benzyl-4R-(1S,2-dibenzyloxyethyl)-pyrrolidin-2-one (3.5 g, 8 mmol) in tetrahydrofuran (80 mL) at 0° C. was slowly added 1.0 M solution of lithium aluminum hydride in tetrahydrofuran (17 mL, 17 mmol). The solution was heated at 70° C. for 1.5 hours. The solution was cooled to 0° C. and water (0.65 mL), 15% sodium hydroxide solution in water (0.65 mL) and water (2 mL) were added carefully. The mixture was stirred at ambient temperature for 1 hour and then filtered through a pad of celite and rinsed with tetrahydrofuran (50 mL). The filtrate was concentrated in vacuo to give 3.2 g of the title compound (yield: 95%). MS (APCI+): m/z 402 (M+H)+.

### Step 10: Preparation of 1-Pyrrolidin-3R-yl-ethane-1R,2-diol

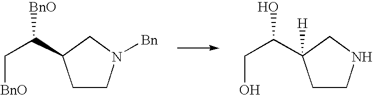

A mixture of N-Benzyl-4R-(1S,2-dibenzyloxyethyl)-pyrrolidine (3.3 g, 8 mmol) and 20 wt % palladium hydroxide on carbon (1.2 g) in methanol (100 mL) was hydrogenated. The catalyst was removed by filtration and the filtrate was concentrated in vacuo to give 1.1 g of the title compound (yield: 100%). MS (APCI+): m/z 132 (M+H)+.

### EXAMPLE 8

### Pyrrolidin-3-yl-thiazol-2-yl-methanol

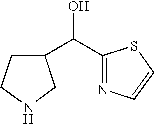

### Step 1: Preparation of N-Benzyloxycarbonyl Pyrrolidine-3-carboxylic acid ethyl ester

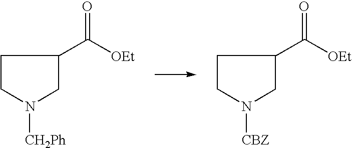

To a solution of N-Benzyl Pyrrolidine-3-carboxylic acid ethyl ester (4.0 g, 17.14 mmol) in chloroform (40 mL) was added benzyl chloroformate (4.94 mL, 34.6 mmol). The mixture was heated at reflux for 4 hours. After evaporation of the solvent, the residue was purified by column chromatography on silica gel (1:2 ethyl acetate:hexanes to give the title compound (Yield: 4.0 g, 85%). ¹H NMR (200 MHz, CDCl₃) δ 7.43-7.28 (m, 5H), 5.12 (s, 2H), 4.24-4.08 (dd, 2H), 3.75-3.35 (m, 4H), 3.15-2.95 (m, 1H), 2.22-2.06 (m, 2H), 1.32-1.20 (t, 3H).

### Step 2: Preparation of N-Benzyloxycarbonyl-Pyrrolidin-3-yl-methanol

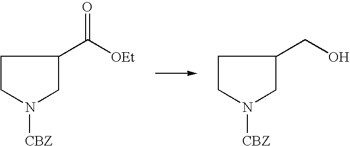

To a solution of N-Benzyloxycarbonyl Pyrrolidine-3-carboxylic acid ethyl ester (1.0 g, 3.6 mmol) in tetrahydrofuran (100 mL) was slowly added sodium borohydride (0.2 g, 5.4 mmol), followed by the slow addition of methanol (2 mL) under ice-water cooling. The mixture was stirred at room temperature for 2 hours. Solvent was evaporated and the residue was dissolved in ethyl acetate (100 mL), washed with water (100 mL) and brine (100 mL), and dried over Na₂SO₄. Evaporation of the solvent gave the title compound (Yield: 0.56 g, 56%). ¹H NMR (200 MHz, CDCl₃) δ 7.42-7.30 (m, 5H), 5.13 (s, 2H), 3.68-3.32 (m, 5H), 3.28-3.10 (m, 1H), 2.52-2.32 (m, 1H), 2.10-1.90 (m, 1H), 1.80-1.50 (m, 2H).

### Step 3: Preparation of N-Benzyloxycarbonyl Pyrrolidine-3-carbaldehyde

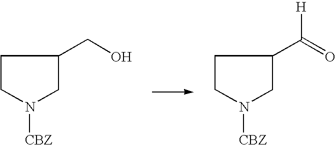

To a solution of oxalyl chloride (0.23 mL, 2.8 mmol) in dichloromethane (5 mL) at −75° C. was added dimethylsulfoxide (0.43 mL, 5.6 mmol) dissolved in dichloromethane (1 mL) and the solution stirred for 2 minutes. N-Benzyloxycarbonyl-Pyrrolidin-3-yl-methanol (0.56 g, 2.5 mmol) in dichloromethane (2 mL) was slowly added and stirred at the same temperature for 0.5 h. Triethylamine (1.8 mL, 12.8 mmol) was then added. The solution was stirred at the same temperature for 5 minutes and let warm to room temperature. Water (10 mL) was added and the products were extracted with dichloromethane (100 mL). The organic extract was washed with water (1×100 mL) and brine (1×100 mL), and dried over Na₂SO₄. After evaporation of the solvent the residue was purified by column chromatography on silica gel (1:1 ethyl acetate:hexanes) to obtain the title compound (Yield: 0.46 g, 78%). ¹H NMR (200 MHz, CDCl₃) δ 9.70 (s, 1H), 7.42-7.28 (m, 5H), 5.14 (s, 2H), 3.90-3.72 (m, 1H), 3.68-3.35 (m, 3H), 3.15-2.96 (m, 1H), 2.32-2.02 (m, 2H).

### Step 4: Preparation of N-Benzyloxycarbonyl Pyrrolidin-3-yl-thiazol-2-yl-methanol

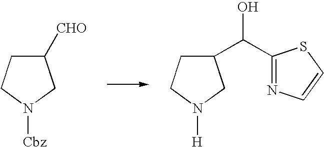

To a stirred solution of 2-bromothiazole (0.90 g, 5.5 mmol) in anhydrous diethyl ether (10 mL), cooled to −70° C. was added a solution of n-butyllithium (2.0 mL of 2.5 M solution in hexane, 5.0 mmol) under nitrogen by syringe and stirring was continued for 30 minutes at −70° C. A solution of N-benzyloxycarbonyl pyrrolidine-3-carbaldehyde (1.17 g, 5.0 mmol) in anhydrous tetrahydrofuran (2 mL) was added by syringe at −70° C., the mixture was stirred at −70° C. for 1 hour, and then it was allowed to warm to 0° C. The mixture was quenched with saturated aqueous NH₄Cl solution and extracted with ethyl acetate. The organic layer was separated, washed with saturated aqueous NH₄Cl solution, brine, dried over anhydrous Na₂SO₄ and concentrated under vacuum. Purification by flash chromatography over silica gel (hexane/ethyl acetate, 3:2 to 1:2) gave the title compound as pale yellow oil (Yield: 0.66 g, 42%). ¹H NMR (400 MHz, CDCl₃) δ 7.71 (m, 1H), 7.38-7.24 (m, 6H), 5.10 (m, 2H), 4.98-4.87 (m, 1H), 4.07-3.90 (m, 1H), 3.64-3.28 (m, 4H), 2.72 (m, 1H), 1.98-1.77 (m, 2H).

### Step 5: Preparation of Pyrrolidin-3-yl-thiazol-2-yl-methanol

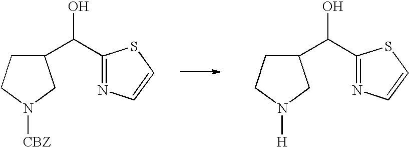

To a stirred solution of N-Benzyloxycarbonyl Pyrrolidin-3-yl-thiazol-2-yl-methanol (0.66 g, 2.0 mmol) in anhydrous dichloromethane (15 mL) cooled to 0° C. was added neat trimethylsilyl iodide (0.60 g, 3.0 mmol) under nitrogen by syringe and stirring was continued for 30 minutes at 0° C. An aqueous 2 N HCl solution (3 mL) was added followed by hexane (15 mL), and the mixture was concentrated under vacuum at room temperature. The residue was washed with diethyl ether and the ether extracts were discarded. The aqueous phase was concentrated under vacuum to dryness, dissolved in methanol (30 mL) and stirred with the basic form of Amberlite IRA-400 (1.5 g) for 30 minutes at room temperature. The solution was filtered through celite and concentrated under vacuum to give the title compound as pale yellow oil (Yield: 0.33 g, 89%). ¹H NMR (400 MHz, CDCl₃) δ 7.66 (m, 1H), 7.27 (m, 1H), 5.01 and 4.89 (d each, 1H), 3.35-2.90 (m, 7H), 2.86-2.70 (m, 1H), 2.04-1.78 (m, 2H).

### EXAMPLE 9

### 2,2-Difluoro-2-pyrrolidin-3-yl-ethanol

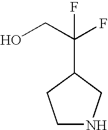

### Step 1: Preparation of 1-Benzyl-3-(2-benzyloxy-1,1-difluoro-ethyl)-pyrrolidine

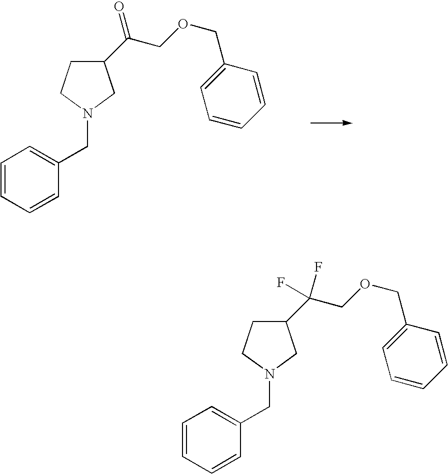

2-Benzyloxy-1-(1-benzyl-pyrrolidin-3-yl)-ethanone (prepared as indicated in Example 1, 587 mg, 1.90 mmol) was dissolved in dichloromethane (1.5 ml) under nitrogen and cooled to 0° C. Diethylaminosulfur trifluoride was added dropwise over 5 minutes and the reaction was heated at 60° C. for four hours. The reaction was poured onto an ice/water mixture (100 ml) and extracted with dichloromethane (3×30 ml). The solvent was removed under reduced pressure and the residue was purified on silica gel by flash chromatography (0-5% isopropanol in dichloromethane) to yield 166 mg (26%) of the title compound. LCMS: m/z 332.3 (M+1).

### Step 2: Preparation of 2,2-Difluoro-2-pyrrolidin-3-yl-ethanol

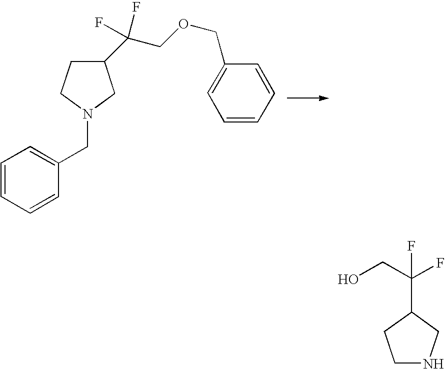

To a solution of 1-Benzyl-3-(2-benzyloxy-1,1-difluoro-ethyl)-pyrrolidine (166 mg, 0.50 mmol) in methanol was added 20% Pd/C. The reaction was conducted under an atmosphere of hydrogen gas for 12 hours. The mixture was filtered and the filtrate was concentrated to give the title compound (74 mg, 98%). LCMS: m/z 153.4 (M+1).

### EXAMPLE 10

### (1-Hydroxymethyl-cyclopropyl)-pyrrolidin-3-yl-methanol Acetate Salt

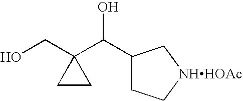

### Step 1: Preparation of 1-[(1-Benzyl-2-oxo-pyrrolidin-3-ylidene)-hydroxy-methyl]-cyclopropanecarboxylic acid ethyl ester

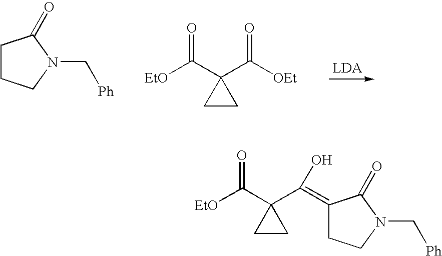

LDA (1M, 13 mL) was stirred in THF (25 mL) at −78° C., then 1-benzyl-pyrrolidine (2.0 g, in solution of THF, 3 mL) was added. After stirring for 30 minutes, cyclopropane-1,1-dicarboxylic acid diethyl ester (2.80 g, in solution of THF, 3 mL) was added. The reaction was stirred at −78° C. for another 15 minutes, then at 0° C. for 1 hour. The reaction mixture was quenched with saturated NH₄Cl, diluted with EtOAc, then washed with water and brine. The organic layer was dried over MgSO₄, filtered and the filtrate was concentrated at reduced pressure. The resulting residue was purified via flash column chromatography (Hexanes/EtOAc gradient) to afford the title compound (2.0 g, 55%). MS (APCI+): m/z 316 (M+H)+.

### Step 2: Preparation of (1-Benzyl-pyrrolidin-3-yl)-(1-hydroxymethyl-cyclopropyl)-methanol

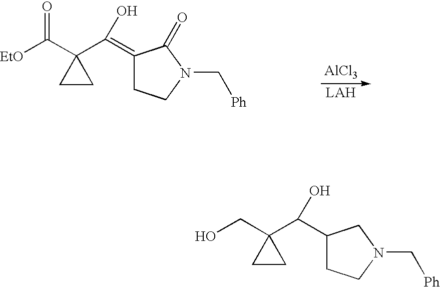

AlCl₃ (0.89 g) was cooled to 0° C., then THF (10 mL) was added slowly. After 15 minutes, LAH (1M, 18 mL) was added slowly over 5 minutes. The reaction mixuture was stirred at 0° C. for 15 minutes, warmed to room temperature for 30 minutes, then cooled to −78° C. Preparation of 1-[(1-Benzyl-2-oxo-pyrrolidin-3-ylidene)-hydroxy-methyl]-cyclopropanecarboxylic acid ethyl ester (2.10 g, in THF solution, 5 mL) was added. The reaction mixture was stirred at −78° C. for 15 minutes, then warmed to room temperature for 1 hour. 1.0 N HCl was added until bubbling ceased. The reaction mixture was diluted with EtOAc, washed with saturated Na₂CO₃, water, and brine. The organic layer was dried over MgSO₄, filtered and the filtrate was concentrated at reduced pressure to afford the title compound (1.75 g, 100%). MS (APCI+): m/Z 262 (M+H)+.

### Step 3: Preparation of (1-Hydroxymethyl-cyclopropyl)-pyrrolidin-3-yl-methanol Acetate Salt

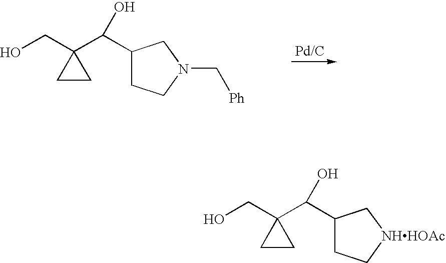

To a solution of (1-Benzyl-pyrrolidin-3-yl)-(1-hydroxymethyl-cyclopropyl)-methanol (1.70 g) in methanol (20 mL) was added 20% Pd/C (0.20 g) and HOAc (0.5 mL). The reaction mixture was hydrogenated for over 20 hours, then the catalyst was filtered, and filtrate concentrated to afford the title compound (1.75 g, 100%). MS (APCI+): m/z 172+ (M+H)+.

### EXAMPLE 11

### (5-Methanesulfonyl-furan-2-yl)-pyrrolidin-3-yl-methanol

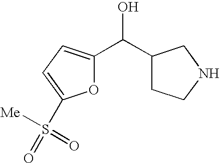

### Step 1: Preparation of 5-Nitro-furan-2-carboxylic acid ethyl ester

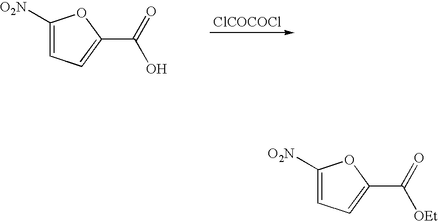

5-Nitro-furan-2-carboxylic acid was stirred in dichloromethane (50 mL), then oxalyl chloride (2.60 g) and DMF (1 mL) were added. After 45 minutes, the reaction mixture was concentrated, and the resulting residue was stirred in CH₂Cl₂ (50 mL) at 0° C. After the solution was sufficiently cooled, EtOH (4 mL) and triethylamine (4.40 mL) were added. After 15 minutes, the reaction mixture was warmed to room temperature and was allowed to stir for 1 hour. The reaction mixture was then washed with saturated NaHCO₃, water, and brine. The organic layer was dried over MgSO₄, filtered and the filtrate was concentrated at reduced pressure to afford the title compound (2.89 g, 98%). ¹H NMR (400 MHz, CDCl₃): 7.33 (1H, d, J=3.9 Hz), 7.26 (1H, d, J=6.8 Hz), 4.44 (2H, t, J=7.1 Hz), 1.41 (3H, q, J=7.1 Hz).

### Step 2: Preparation of 5-Methylsulfanyl-furan-2-carboxylic acid ethyl ester

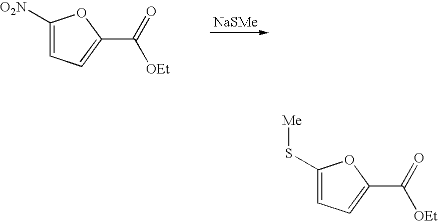

To a solution of 5-Nitro-furan-2-carboxylic acid ethyl ester (2.90 g) in DMSO (20 mL) was added NaSMe (1.20 g). The resulting reaction mixture was heated overnight to 100° C., then quenched with saturated NH₄Cl. The mixture was diluted with EtOAc and washed with saturated NaHCO₃, water, and brine. The organic layer was dried over MgSO₄, filtered and the filtrate was concentrated to at reduced pressure to afford the title compound (2.70 g, 92%). ¹H NMR (400 MHz, CDCl₃): 7.13 (1H, d, J=3.7 Hz), 6.36 (1H, d, J=3.7 Hz), 4.34 (2H, t, J=7.1 Hz), 2.50 (3H, s), 1.36 (3H, q, J=7.1 Hz).

### Step 3: Preparation of 5-Methanesulfonyl-furan-2-carboxylic acid ethyl ester

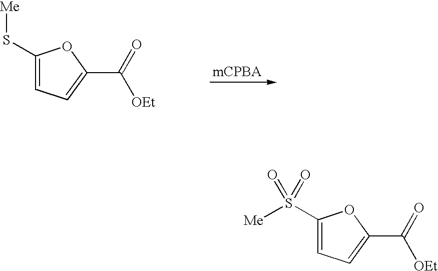

To a solution of 5-Methylsulfanyl-furan-2-carboxylic acid ethyl ester (5.30 g) in CH₂Cl₂ (50 mL) was added mCPBA (10.0 g). The resulting reaction mixture was heated to reflux. After 4 hours, the reaction mixture was washed with saturated Na₂CO₃, H₂O, and brine. The organic layer was dried over MgSO₄, filtered and the filtrate was concentrated at reduced pressure to afford the title compound (3.96 g, 64%). ¹H NMR (400 MHz, CDCl₃): 7.22-7.19 (1H, m), 5.29 (1H, s), 4.39 (2H, t, J=7.1 Hz), 3.21 (3H, s), 1.39 (3H, q, J=7.1 Hz).

### Step 4: Preparation of 1-Benzyl-3-[hydroxy-(5-methanesulfonyl-furan-2-yl)-methylene]-pyrrolidin-2-one

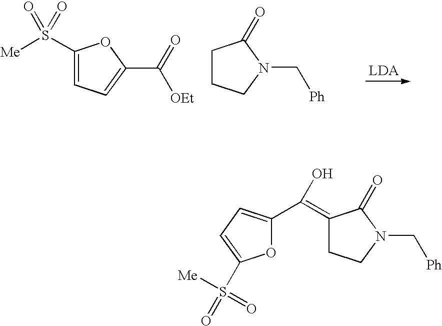

LDA was stirred in THF (50 mL) at −78° C., then 1-benzyl-2-pyrrolidinone (in solution of THF, 5 mL) was added dropwise over 2 minutes. After 45 minutes, 5-Methanesulfonyl-furan-2-carboxylic acid ethyl ester (in solution of THF, 3 mL) was added dropwise over 5 minutes. After additional 15 minutes at −78° C., reaction was warmed to room temperature. After 90 minutes, the reaction mixture was diluted with EtOAc and washed with saturated NH₄Cl, H₂O, and brine. Organic layer was dried over MgSO₄, filtered and filtrate concentrated. Resulting residue was purified via flash column chromatography (Hexanes/EtOAc gradient) to afford the title compound (5.02 g, 87%). MS (APCI+): m/z 348 (M+H)+.

### Step 5: Preparation of 1-Benzyl-3-[hydroxy-(5-methanesulfonyl-furan-2-yl)-methyl]-pyrrolidin-2-one

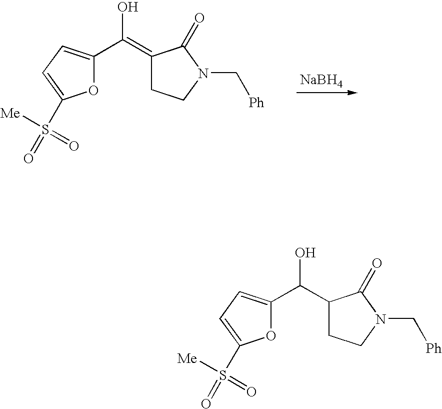

To a solution of 1-Benzyl-3-[hydroxy-(5-methanesulfonyl-furan-2-yl)-methylene]-pyrrolidin-2-one (5.02 g) in MeOH (20 mL) and THF (20 mL) was added sodium borohydride (NaBH₄) (1.65 g) portionwise. After 16 hours, the reaction mixture was concentrated. The resulting residue was dissolved in EtOAc, and the resulting solution was washed with water and brine. The organic layer was dried over MgSO₄, filtered, and the filtrate was concentrated at reduced pressure to afford the title compound (3.99 g, 79%). MS (APCI+): m/z 350 (M+H)+.

### Step 6: Preparation of (1-Benzyl-pyrrolidin-3-yl)-(5-methanesulfonyl-furan-2-yl)-methanol

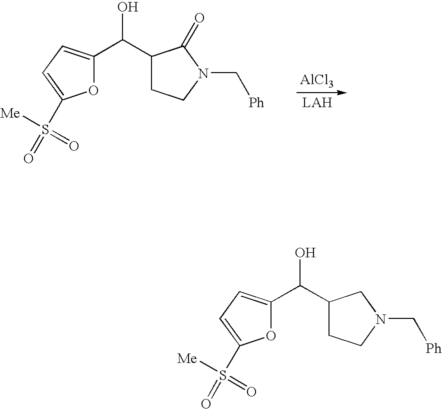

AlCl₃ (1.34 g) was cooled to 0° C., then THF (10 mL) was added slowly. After 15 minutes, LAH (1M, 30 mL) was added slowly over 5 minutes. The reaction mixture was stirred at 0° C. for 15 minutes, warmed to room temperature for 30 minutes, then cooled to −78° C. 1-Benzyl-3-[hydroxy-(5-methanesulfonyl-furan-2-yl)-methyl]-pyrrolidin-2-one (3.50 g, in THF solution, 5 mL) was then added. The reaction mixture was stirred at −78° C. for 15 minutes, then warmed to room temperature for 1 hour. 1N HCl was added until bubbling ceased. The reaction mixture was diluted with EtOAc, washed with saturated Na₂CO₃, H₂O, and brine. The organic layer was dried over MgSO₄, filtered and the filtrate was concentrated at reduced pressure to afford the title compound (3.02 g, 90%). MS (APCI+): m/z 336 (M+H)+.

### Step 7: Preparation of 1-Benzyl-3-[(5-methanesulfonyl-furan-2-yl)-methoxymethoxy-methyl]-pyrrolidine

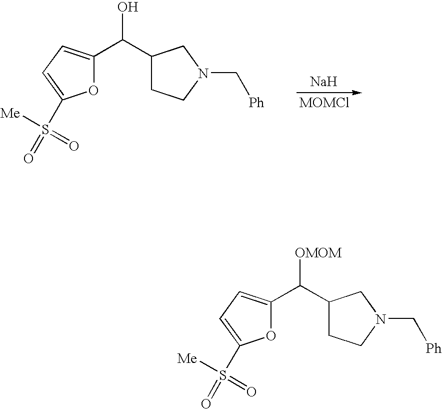

To a solution of (1-Benzyl-pyrrolidin-3-yl)-(5-methanesulfonyl-furan-2-yl)-methanol (2.80 g) in THF (20 mL) at 0° C. was added NaH in small portions. After 15 minutes, the reaction mixture was warmed to room temperature for 30 minutes. Not all the solid was soluble, so the reaction mixture was then heated to reflux for 14 minutes, then cooled to room temperature. Methoxymethyl chloride (MOMCl) (0.74 g) was then added. After 2 hours, the reaction mixture was diluted with EtOAc and washed with saturated NH₄Cl, H₂O, and brine. The organic layer was dried over MgSO₄, filtered and the filtrate was concentrated at reduced pressure. The resulting residue was purified via flash column chromatography (Hexanes/EtOAc gradient) to afford the title compound (0.98 g, 34%). MS (APCI+): n/z 380 (M+H)+.

### Step 8: Preparation of (5-Methanesulfonyl-furan-2-yl)-pyrrolidin-3-yl-methanol

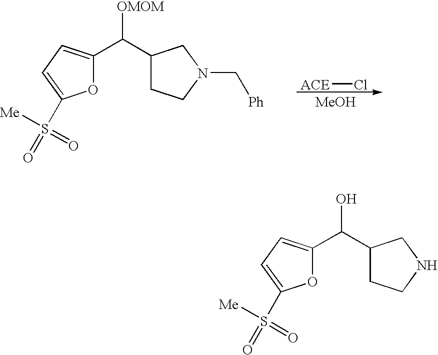

To a solution of 1-Benzyl-3-[(5-methanesulfonyl-furan-2-yl)-methoxymethoxy-methyl]-pyrrolidine (0.97 g) in CH₂Cl₂ (10 mL) at 0° C. was added 1-chloroethyl chloroformate (0.44 g). After 15 minutes, the reaction mixture was warmed to room temperature for 30 minutes. The reaction mixture was then concentrated and the resulting residue was dissolved in MeOH (20 mL) and heated to reflux. After 3 hours, the reaction mixture was cooled to room temperature and concentrated HCl (0.5 mL) was added. The reaction mixture was heated to reflux for another 4 hours, then concentrated. The resulting residue was dissolved in EtOAc and washed with saturated NaHCO₃ and H₂O. The aqueous layers were combined and concentrated. The resulting solids were stirred in MeOH/CH₂Cl₂, filtered and the filtrate concentrated at reduced pressure to afford the title compound as an HCl salt (0.29 g, 46%). MS (APCI+): m/z 246 (M+H)°.

### EXAMPLE 12

### 3-{(tert-Butyl-dimethyl-silanyloxy)-[1-(tert-butyl-dimethyl-silanyloxy)-cyclopropyl]-methyl}-pyrrolidine

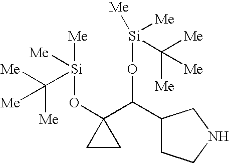

### Step 1: Preparation of 1-(tert-Butyl-dimethyl-silanyloxy)-cyclopropanecarboxylic acid ethyl ester

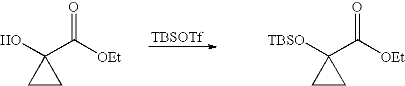

To a solution of 1-Hydroxy-cyclopropanecarboxylic acid ethyl ester (3.0 g) and 2,6-lutidine (2.72 g) in dichloromethane (20 mL) at 0° C. was added tert butyldimethyl silyltrifluoromethanesulfonate (6.09 g). After 15 minutes at 0° C., the reaction mixture was warmed to room temperature. After 30 minutes, the reaction mixture was concentrated to afford crude 1-(tert-Butyl-dimethyl-silanyloxy)-cyclopropanecarboxylic acid ethyl ester, which was used immediately without purification.

### Step 2: Preparation of 1-Benzyl-3-{[1-(tert-butyl-dimethyl-silanyloxy)-cyclopropyl]-hydroxy-methylene}-pyrrolidin-2-one

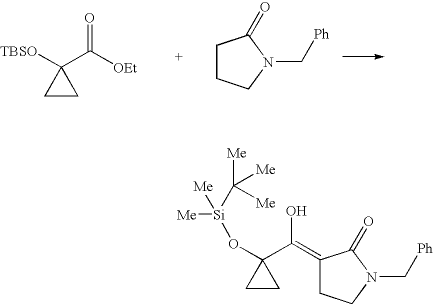

LDA (2M, 14 mL) was stirred in THF (50 mL) at −78° C., then 1-benzyl-2-pyrrolidinone (4.04 g) was added slowly as solution in THF (5 mL). After 30 minutes, crude 1-(tert-Butyl-dimethyl-silanyloxy)-cyclopropanecarboxylic acid ethyl ester was added as a solution in THF (5 mL). The reaction mixture remained at −78° C. for 30 minutes, and then was warmed to 0° C. After 1 hour, saturated NH₄Cl was added to quench excess base, then the reaction mixture was diluted with EtOAc and washed with saturated NaHCO₃, water, and brine. The organic layer was dried over MgSO₄, filtered, and the filtrate was concentrated. The resulting residue was purified via flash column chromatography (Hexanes/EtOAc gradient) to afford the title compound (3.23 g, 38%). MS (APCI+): m/z 374 (M+H)+.

### Step 3: Preparation of 1-Benzyl-3-{[1-(tert-butyl-dimethyl-silanyloxy)-cyclopropyl]-hydroxy-methylene}-pyrrolidine

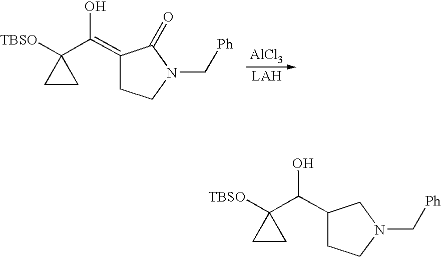

AlCl₃ (1.40 g) was cooled to 0° C., then THF (10 mL) was added slowly. After 15 minutes, LAH (1M, 31 mL) was added slowly over 5 minutes. The reaction mixture was stirred at 0° C. for 15 minutes, warmed to room temperature for 30 minutes, then cooled to −78° C. 1-Benzyl-3-{[1-(tert-butyl-dimethyl-silanyloxy)-cyclopropyl]-hydroxy-methylene}-pyrrolidin-2-one (3.23 g, in THF solution, 5 mL) was added. The reaction was stirred at −78° C. for 15 minutes, then warmed to room temperature for 1 hour. 1N HCl was added until bubbling ceased. Reaction mixture was diluted with EtOAc, washed with saturated Na₂CO₃, H₂O, and brine. The organic layer was dried over MgSO₄, filtered and the filtrate was concentrated at reduced pressure to afford the title compound (3.20 g, 100%). MS (APCI+): m/z 362 (M+H)+.

### Step 4: Preparation of 1-Benzyl-3-{[1-(tert-butyl-dimethyl-silanyloxy)-cyclopropyl]-(tert-butyl-dimethyl-silanyloxy)-methylene}-pyrrolidine

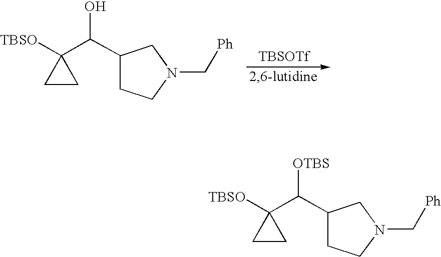

To a solution of 1-Benzyl-3-{[1-(tert-butyl-dimethyl-silanyloxy)-cyclopropyl]-hydroxy-methylene}-pyrrolidine (3.20 g) and 2,6-lutidine (0.95 g) in CH₂Cl₂ (20 mL) was added tert-butyldimethylsilyltrifluoromethanesulfonate (6.09 g). The reaction was stirred at 0° C. for 15 minutes, then warmed to room temperature. After 30 minutes, the reaction mixture was concentrated. Purification via flash column chromatography (Hexanes/EtOAc gradient) afforded the title compound (0.87 g, 21%). MS (APCI+): m/z 476 (M+H)+.

### Step 5: Preparation of 3-{[1-(tert-butyl-dimethyl-silanyloxy)-cyclopropyl]-(tert-butyl-dimethyl-silanyloxy)-methylene}-pyrrolidine

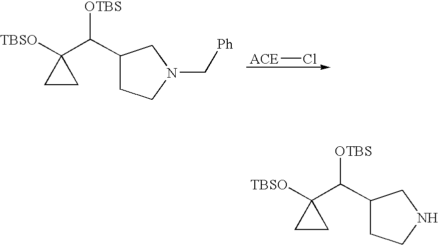

To a solution of 1-Benzyl-3-{[1-(tert-butyl-dimethyl-silanyloxy)-cyclopropyl]-(tert-butyl-dimethyl-silanyloxy)-methylene}-pyrrolidine (0.87 g) in CH₂Cl₂ (20 mL) at 0° C. was added 1-chloroethyl chloroformate (0.39 g). After 15 minutes, the reaction was warmed to room temp for 1 hour. The reaction mixture was concentrated and the resulting residue was dissolved in methanol (10 mL) and heated to reflux. After 3 hours, the reaction mixture was cooled to room temperature and concentrated to afford the title compound as the HCl salt (0.78 g, 100%). MS (APCI+): m/z 386 (M+H)+.

### EXAMPLE 13

### Preparation of Octahydro-cyclopenta[c]pyrrol-4-ol

### Step 1: Preparation of 2-Benzyl-hexahydro-cyclopenta[c]pyrrol-4-one

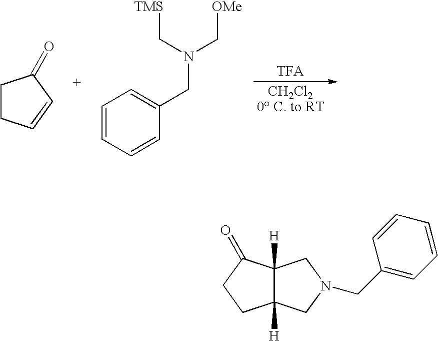

To a solution of 2-cyclopenten-1-one (10.2 mL, 122 mmol) and benzyl-1-methoxymethyl-1-trimethylsilylmethyl amine (34.8 g, 146 mmol) in dichloromethane (240 mL) was added trifluoroacetic acid (11 mL, 1 M in dichloromethane). The reaction mixture was allowed to warm to room temperature over 18 hours. The mixture was diluted with saturated aqueous sodium bicarbonate, and the organic phase was removed. The aqueous layer was extracted two times with dichloromethane, and the combined organic layers were washed with saturated aqueous sodium bicarbonate, dried over magnesium sulfate, filtered, and concentrated in vacuo to provide the title compound (26.3 g, 100%) as a dark orange liquid which was used without further purification. MS(APCI+): m/z 216.1 (M+H)⁺.

### Step 2: 4-Oxo-hexahydro-cyclopenta[c]pyrrole-2-carboxylic acid benzyl ester

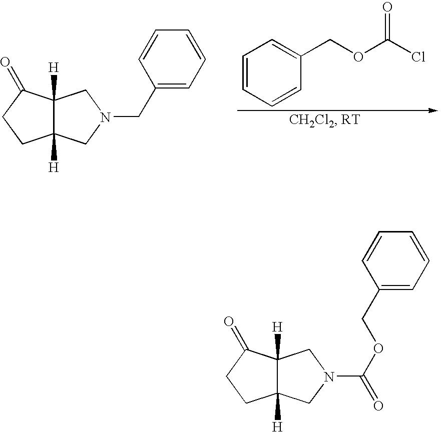

To a solution of 2-benzyl-hexahydro-cyclopenta[c]pyrrol-4-one (26.3 g, 122 mmol) in dichloromethane (1000 mL) was added benzyl chloroformate (30.0 mL, 207 mmol). The reaction mixture was allowed to stir at room temperature for 24 hours and was then concentrated in vacuo. The resulting residue was purified on a 65M Biotage column using an ethyl acetate/hexanes gradient to afford the title compound (16.0 g, 51%) as a yellow liquid. MS(APCI+): m/z 260.2 (M+H)⁺, 216.2 (M-CO₂+H)⁺.

### Step 3: Preparation of 4-Hydroxy-hexahydro-cyclopenta[c]pyrrole-2-carboxylic acid benzyl ester

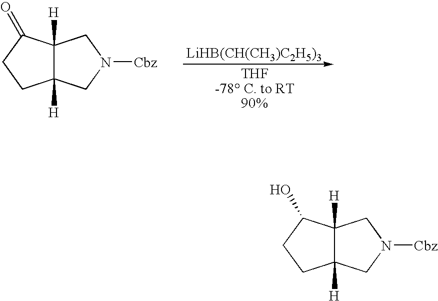

To a cooled (−78° C.) solution of 4-oxo-hexahydro-cyclopenta[c]pyrrole-2-carboxylic acid benzyl ester (7.5 g, 29 mmol) in tetrahydrofuran (70 mL) was added lithium tri-sec-butylborohydride (43 mL, 43 mmol, 1 M in tetrahydrofuran) dropwise by addition funnel. The reaction mixture was allowed to slowly warm to room temperature over 18 hours. The mixture was cooled to 0° C. and 30% hydrogen peroxide was added dropwise with caution until all gas evolution ceased. The quenched solution was poured into water and extracted three times with ethyl acetate. The combined organics were washed with brine, dried over magnesium sulfate, filtered, and concentrated in vacuo. The residue was purified on a 40L Biotage column using an ethyl acetate/dichloromethane gradient to afford the title compound (6.8 g, 90%) as a yellow oil. MS(APCI+): m/z 262.2 (M+H)⁺, 218.2 (M-CO₂+H)⁺.

### Step 4: Preparation of Octahydro-cyclopenta[c]pyrrol-4-ol

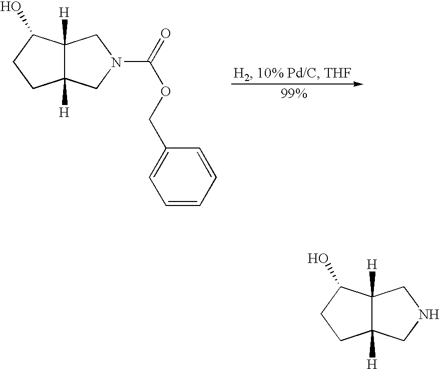

To a solution of 4-Hydroxy-hexahydro-cyclopenta[c]pyrrole-2-carboxylic acid benzyl ester (2.5 g, 9.6 mmol) in tetrahydrofuran (50 mL) in a Parr shaker was added 10% palladium on carbon (1.5 g), and hydrogen gas was introduced at 40 psi for 40 hours. The reaction mixture was diluted with methanol, filtered through diatomaceous earth (Celite®), and concentrated in vacuo to afford the title compound (1.2 g, 99%) as a yellow solid which was used without further purification. MS(APCI+): m/z 128.0 (M+H)⁺.

### EXAMPLE 14

### Preparation of Octahydro-cyclopenta[c]pyrrol-4-ol

### Step 1: Preparation of 4-Benzoyloxy-hexahydro-cyclopenta[c]pyrrole-2-carboxylic acid benzyl ester

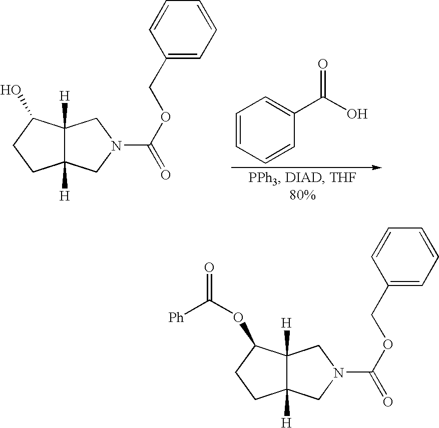

To a solution of 4-hydroxy-hexahydro-cyclopenta[c]pyrrole-2-carboxylic acid benzyl ester (4.3 g, 16 mmol) in tetrahydrofuran (50 mL) was added triphenylphosphine (5.6 g, 21 mmol), benzoic acid (2.6 g, 21 mmol), and diisopropyl azodicarboxylate (DIAD) (4.2 mL, 21 mmol), in that order. The resulting yellow solution was stirred at room temperature for 20 hours and concentrated in vacuo. The residue was purified on a 40L Biotage column using an ethyl acetate/hexanes gradient to afford the title compound (4.8 g, 80%) as a colorless oil. MS(APCI+): m/z 366.1 (M+H)⁺, 322.1 (M-CO₂+H)⁺.

### Step 2: Preparation of 4-Hydroxy-hexahydro-cyclopenta[c]pyrrole-2-carboxylic acid benzyl ester

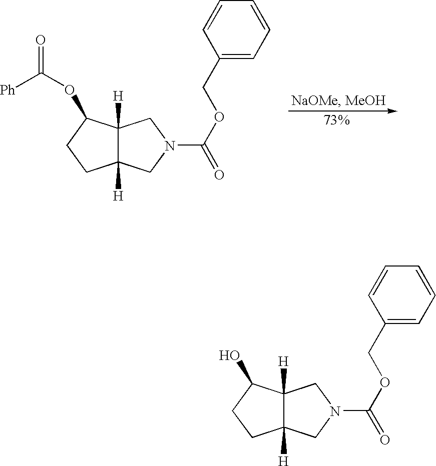

To a solution of 4-benzoyloxy-hexahydro-cyclopenta[c]pyrrole-2-carboxylic acid benzyl ester (4.8 g, 13 mmol) in methanol (65 mL) was added sodium methoxide (1.4 g, 26 mmol), and the reaction mixture was stirred at room temperature for 4 hours and concentrated in vacuo. The resulting white residue was partitioned between saturated aqueous ammonium chloride and dichloromethane. The aqueous phase was extracted two times with dichloromethane, and the combined organics were dried over magnesium sulfate, filtered, and concentrated in vacuo. The residue was purified on a 40M Biotage column using and ethyl acetate/dichloromethane gradient to afford the title compound (2.5 g, 73%) as a colorless oil. MS(APCI+): m/z 262.2 (M+H)⁺, 218.2 (M-CO₂+H)⁺.

### Step 3: Preparation of Octahydro-cyclopenta[c]pyrrol-4-ol

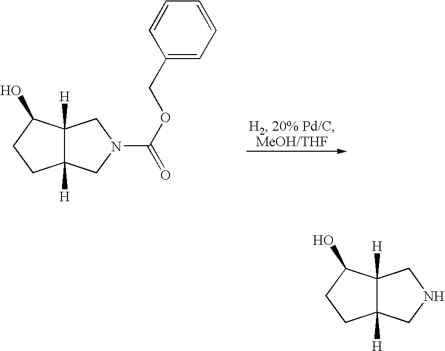

To a solution of 4-hydroxy-hexahydro-cyclopenta[c]pyrrole-2-carboxylic acid benzyl ester (2.5 g, 9.6 mmol) in tetrahydrofuran (50 mL) and methanol (50 mL) in a Parr shaker was added 20% palladium on carbon (0.40 g), and hydrogen gas was introduced at 40 psi for 15 hours. The reaction mixture was diluted with methanol, filtered through diatomaceous earth (Celite®), and concentrated in vacuo to afford the title compound (1.2 g, 99%) as a yellow solid which was used without further purification. MS(APCI+): m/z 128.0 (M+H)⁺.

### EXAMPLE 15

### Preparation of Octahydro-cyclopenta[c]pyrrole-4,5-diol

### Step 1: Preparation of 3,3a,4,6a-Tetrahydro-1H-cyclopenta[c]pyrrole-2-carboxylic acid benzyl ester

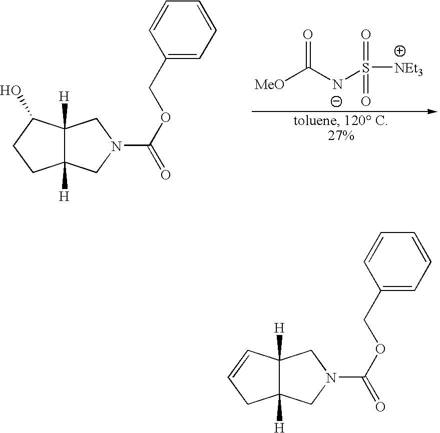

To a solution of 4-Hydroxy-hexahydro-cyclopenta[c]pyrrole-2-carboxylic acid benzyl ester (1.5 g, 5.7 mmol) in toluene (60 mL) was added (methoxycarbonylsulfamoyl)triethylammonium hydroxide, inner salt (1.8 g, 7.5 mmol). The reaction mixture was heated at 120° C. for 24 hours, concentrated in vacuo, and partitioned between ethyl acetate and saturated aqueous sodium bicarbonate. The aqueous phase was extracted two times with ethyl acetate, and the combined organics were washed with brine, dried over magnesium sulfate, filtered, and concentrated in vacuo. The residue was purified on a 40S Biotage column using an ethyl acetate/hexanes gradient to afford the title compound (0.38 g, 27%) as a colorless oil. MS(APCI+): m/z 244.2 (M+H)⁺, 200.1 (M-CO₂+H)⁺.

### Step 2: Preparation of 4,5-Dihydroxy-hexahydro-cyclopenta[c]pyrrole-2-carboxylic acid benzyl ester

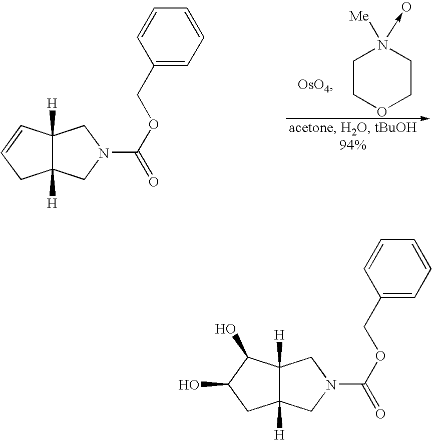

To a solution of 3,3a,4,6a-tetrahydro-1H-cyclopenta[c]pyrrole-2-carboxylic acid benzyl ester (0.53 g, 2.2 mmol) in acetone (6 mL), tert-butyl alcohol (1.3 mL), and water (6 mL) was added 4-methylmorpholine N-oxide (0.38 g, 3.2 mmol) followed by osmium tetroxide (0.13 mL, 0.011 mmol, 2.5 wt % in 2-methyl-2-propanol). The resulting yellow reaction mixture was stirred at room temperature for 3 hours and diluted with saturated aqueous sodium bisulfite and ethyl acetate. The aqueous phase was extracted three times with ethyl acetate, and the combined organics were washed with brine, dried over magnesium sulfate, filtered, and concentrated in vacuo. The residue was purified on a 10 g Isco column using a methanol/dichloromethane gradient to afford the title compound as a racemic mixture of syn diols (0.54 g, 90%). MS(APCI+): m/z 278.2 (M+H)⁺, 234.2 (M-CO₂+H)⁺.

### Step 3: Preparation of Octahydro-cyclopenta[c]pyrrole-4,5-diol

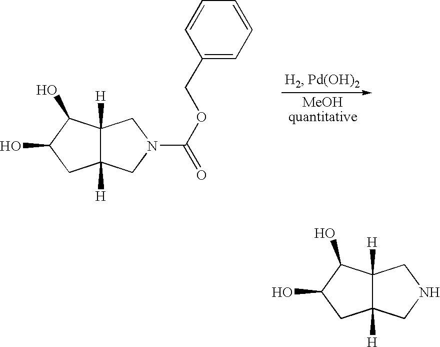

To a solution of 4,5-dihydroxy-hexahydro-cyclopenta[c]pyrrole-2-carboxylic acid benzyl ester (0.54 g, 1.9 mmol) in methanol (50 mL) in a Parr shaker was added 20% palladium hydroxide on carbon (0.050 g), and hydrogen gas was introduced at 37 psi for 28 hours. The reaction mixture was diluted with methanol, filtered through diatomaceous earth (Celite®), and concentrated in vacuo to afford the title compound (0.28 g, 100%) which was used without further purification. MS(APCI+): m/z 144.1 (M+H)⁺.

### EXAMPLE 16

### Preparation of 4-Hydroxymethyl-octahydro-cyclopenta[c]pyrrol-4-ol

### Step 1: 4-Benzyloxymethyl-4-hydroxy-hexahydro-cyclopenta[c]pyrrole-2-carboxylic acid benzyl ester

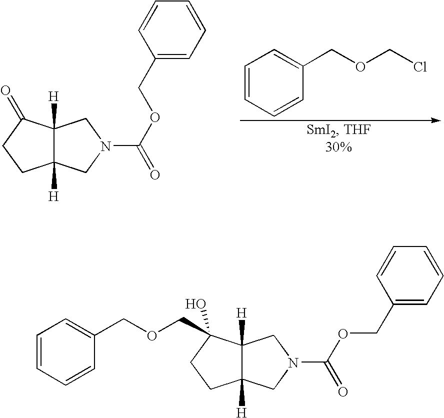

To a solution of 4-oxo-hexahydro-cyclopenta[c]pyrrole-2-carboxylic acid benzyl ester (1.5 g, 5.8 mmol) in tetrahydrofuran (60 mL) was added benzyl chloromethyl ether (1.6 mL, 12 mmol, 60% technical grade) followed by samarium iodide (6.34 g, 15.7 mmol). The dark blue reaction mixture was stirred at room temperature for 3.5 hours, during which time it turned bright yellow. The mixture was diluted with saturated aqueous ammonium chloride and ethyl acetate. The aqueous layer was extracted two times with ethyl acetate, and the combined organics were washed with brine, dried over magnesium sulfate, filtered, and concentrated in vacuo. The residue was purified on a 40L Biotage column (0:100 to 80:20 ethyl acetate/hexanes) to afford the title compound (0.66 g, 30%) as a colorless oil. MS(APCI+): m/z 382.4 (M+H)⁺, 338.2 (M-CO₂+H)⁺.

### Step 2: Preparation of 4-Hydroxymethyl-octahydro-cyclopenta[c]pyrrol-4-ol

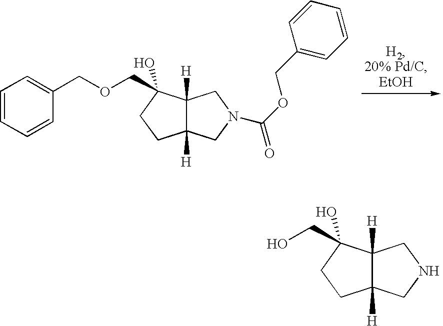

To a solution of 4-benzyloxymethyl-4-hydroxy-hexahydro-cyclopenta[c]pyrrole-2-carboxylic acid benzyl ester (1.15 g, 3.01 mmol) in ethanol (50 mL) in a Parr shaker was added 20% palladium on carbon (1.0 g), and hydrogen gas was introduced at 37 psi for 100 hours. The reaction mixture was diluted with methanol, filtered through diatomaceous earth (Celite®), and concentrated in vacuo to afford the title compound (0.43 g, 91%) which was used without further purification. MS(APCI+): m/z 158.1 (M+H)⁺.

### EXAMPLE 17

### Preparation of Preparation of 5-Fluoro-octahydro-cyclopenta[c]pyrrol-4-ol Isomers

### Step 1: Preparation of 6-(tert-Butyl-dimethyl-silanyloxy)-3,3a,4,6a-tetrahydro-1H-cyclopenta[c]pyrrole-2-carboxylic acid benzyl ester

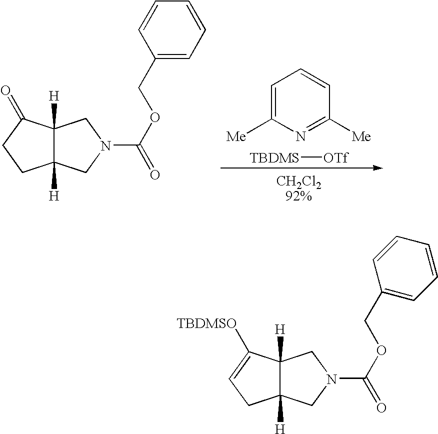

To a cooled (0° C.) solution of 4-oxo-hexahydro-cyclopenta[c]pyrrole-2-carboxylic acid benzyl ester product (5.2 g, 20 mmol) and 2,6-lutidine (4.6 mL, 40 mmol) in dichloromethane (150 mL) was added tert-butyldimethylsilyl trifluoromethanesulfonate (7.0 mL, 30 mmol). The reaction mixture was warmed to room temperature and after 20 hours, the mixture was diluted with saturated aqueous sodium bicarbonate and dichloromethane. The aqueous phase was extracted two times with dichloromethane, and the combined organics were dried over magnesium sulfate, filtered, and concentrated in vacuo. The residue was purified on a 40M Biotage column using an ethyl acetate/hexanes gradient to afford the title compound (6.9 g, 92%). MS(APCI+): m/z 374.1 (M+H)⁺.

### Step 2: Preparation of 5-Fluoro-4-oxo-hexahydro-cyclopenta[c]pyrrole-2-carboxylic acid benzyl ester (A) and 5-Fluoro-4-oxo-hexahydro-cyclopenta[c]pyrrole-2-carboxylic acid benzyl ester (B)

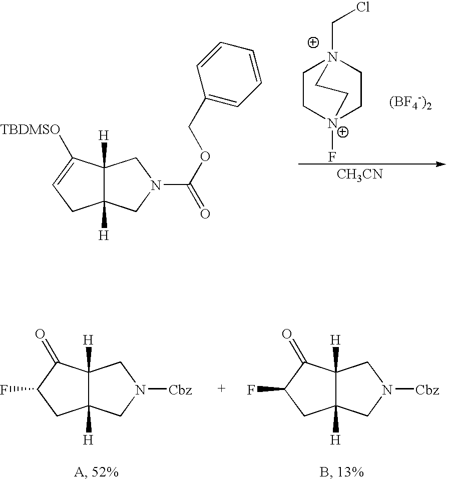

To a solution of example 23 product (16.2 g, 43.4 mmol) in acetonitrile (400 mL) was added Selectfluor™ (20.0 g, 56.4 mmol). After 2.5 hours, the solvent was removed in vacuo, and the resulting residue was partitioned between water and dichloromethane. The aqueous phase was extracted two times with dichloromethane, and the combined organics were washed with brine, dried over magnesium sulfate, filtered, and concentrated in vacuo. The residue was purified on a 40L Biotage column using an ethyl acetate/hexanes solvent system to afford the title compounds (A, 6.2 g, 52%; B, 1.6 g, 13%). MS(APCI+): m/z 278.1 (M+H)⁺.

### Step 3A: Preparation of 5-Fluoro-4-hydroxy-hexahydro-cyclopenta[c]pyrrole-2-carboxylic acid benzyl ester (A)

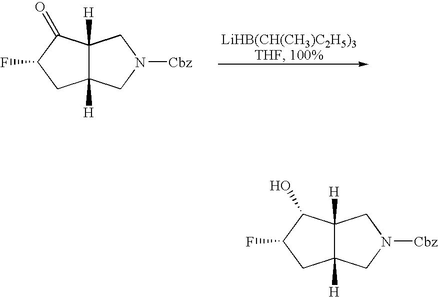

To a cooled (−78° C.) solution of 5-Fluoro-4-oxo-hexahydro-cyclopenta[c]pyrrole-2-carboxylic acid benzyl ester (A) (1.8 g, 6.5 mmol) in tetrahydrofuran (30 mL) was added lithium tri-sec-butylborohydride (7.8 mL, 7.8 mmol, 1 M in tetrahydrofuran) dropwise. After 45 minutes, the reaction mixture was warmed to room temperature, then cooled to 0° C., and 30% aqueous H₂O₂ was added dropwise with caution until all gas evolution ceased. The mixture was warmed to room temperature, poured into water, and extracted three times with ethyl acetate. The combined organics were washed with brine, dried over magnesium sulfate, filtered, and concentrated in vacuo to give the title compound (1.8 g, 100%) which was used without further purification. MS(APCI+): m/z 280.2 (M+H)⁺, 236.2 (M-CO₂+H)⁺.

### Step 4A: Preparation of 5-Fluoro-octahydro-cyclopenta[c]pyrrol-4-ol

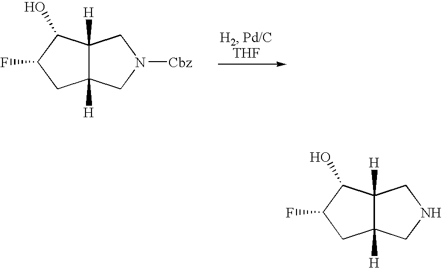

To a solution of 5-Fluoro-4-hydroxy-hexahydro-cyclopenta [c]pyrrole-2-carboxylic acid benzyl ester (A) (0.46 g, 1.6 mmol) in tetrahydrofuran (50 mL) in a Parr shaker was added 20% palladium on carbon (0.19 g), and hydrogen gas was introduced at 37 psi for 15 hours. The reaction mixture was diluted with methanol, filtered through diatomaceous earth (Celite®), and concentrated in vacuo to afford the title compound (0.24 g, 100%) which was used without further purification. MS(APCI+): m/z 146.1 (M+H)⁺.

### Step 3B: Preparation of 5-Fluoro-4-hydroxy-hexahydro-cyclopenta[c]pyrrole-2-carboxylic acid benzyl ester (B)

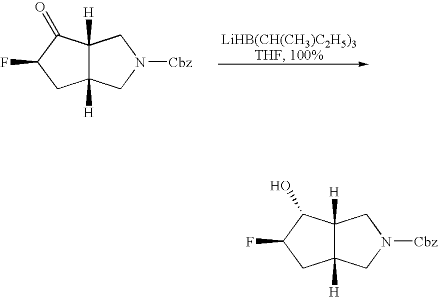

To a cooled (−78° C.) solution of 5-Fluoro-4-oxo-hexahydro-cyclopenta[c]pyrrole-2-carboxylic acid benzyl ester (B) (2.0 g, 7.2 mmol) in tetrahydrofuran (50 mL) was added lithium tri-sec-butylborohydride (8.6 mL, 8.6 mmol, 1 M in tetrahydrofuran) dropwise. After 1.5 hours, the reaction mixture was warmed to room temperature, then cooled to 0° C., and 30% aqueous H₂O₂ was added dropwise with caution until all gas evolution ceased. The mixture was warmed to room temperature, poured into water, and extracted three times with ethyl acetate. The combined organics were washed with brine, dried over magnesium sulfate, filtered, and concentrated in vacuo to give the title compound (2.0 g, 100%), which was used without further purification. MS(APCI+): m/z 280.2 (M+H)⁺, 236.1 (M-CO₂+H)⁺.

### Step 4B: Preparation of 5-Fluoro-octahydro-cyclopenta[c]pyrrol-4-ol

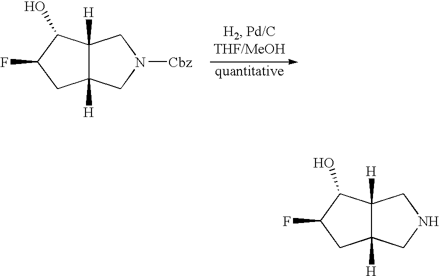

To a solution of 5-Fluoro-4-hydroxy-hexahydro-cyclopenta[c]pyrrole-2-carboxylic acid benzyl ester (B) (0.38 g, 1.4 mmol) in tetrahydrofuran (10 mL) and methanol (10 mL) in a Parr shaker was added 20% palladium hydroxide on carbon (0.050 g), and hydrogen gas was introduced at 37 psi for 15 hours. The reaction mixture was diluted with methanol, filtered through diatomaceous earth (Celite®), and concentrated in vacuo to afford the title compound (0.20 g, 100%) which was used without further purification. MS)(APCI+): m/z 146.1 (M+H)⁺.

### EXAMPLE 18

### Preparation of 5-Fluoro-octahydro-cyclopenta[c]pyrrol-4-ol

### Step 1: Preparation of 4-Benzoyloxy-5-fluoro-hexahydro-cyclopenta[c]pyrrole-2-carboxylic acid benzyl ester

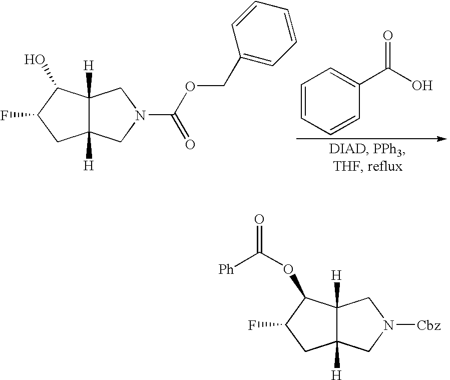

To a solution of triphenylphosphine (4.51 g, 17.2 mmol) in tetrahydrofuran (20 mL) was added diisopropyl azodicarboxylate (3.4 mL, 17 mmol). After 40 minutes, benzoic acid (2.10 g, 17.2 mmol) was added, followed by 5-Fluoro-4-hydroxy-hexahydro-cyclopenta[c]pyrrole-2-carboxylic acid benzyl ester (A) (1.6 g, 5.7 mmol) in tetrahydrofuran (2 mL). The transfer was completed with 2×2 mL of tetrahydrofuran. The reaction mixture was heated at 70° C. for 48 hours, cooled to room temperature, and concentrated in vacuo. The resulting residue was purified on a 40L Biotage column using an ethyl acetate/hexanes gradient to afford the title compound (2.0 g, 91%). MS(APCI+): m/z 384.1 (M+H)⁺, 340.2 (M-CO₂+H)⁺.

### Step 2: Preparation of 5-Fluoro-4-hydroxy-hexahydro-cyclopenta[c]pyrrole-2-carboxylic acid benzyl ester

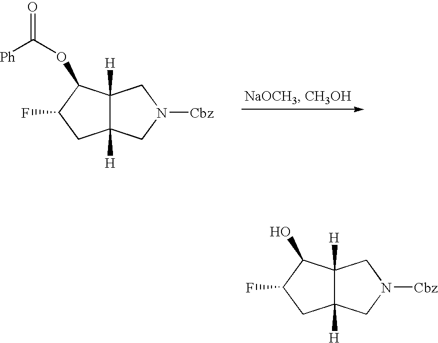

To a cooled (0° C.) solution of 4-benzoyloxy-5-fluoro-hexahydro-cyclopenta[c]pyrrole-2-carboxylic acid benzyl ester (2.15 g, 5.61 mmol) in methanol (25 mL) was added sodium methoxide (0.455 g, 8.42 mmol). After 2 hours, saturated aqueous ammonium chloride was added, and the reaction mixture was warmed to room temperature and concentrated in vacuo. The residue was partitioned between water and dichloromethane. The aqueous phase was extracted two times with dichloromethane, and the combined organics were dried over magnesium sulfate, filtered, and concentrated in vacuo. The residue was purified on a 40S Biotage column using an ethyl acetate/hexanes gradient to afford the title compound (0.33 g 21%). MS(APCI+): m/z 280.2 (M+H)⁺.

### Step 3: Preparation of 5-Fluoro-octahydro-cyclopenta[c]pyrrol-4-ol

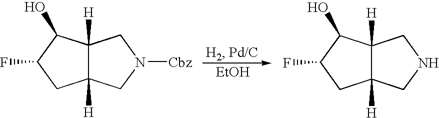

To a solution of 5-fluoro-4-hydroxy-hexahydro-cyclopenta[c]pyrrole-2-carboxylic acid benzyl ester (0.33 g, 1.2 mmol) in ethanol (50 mL) in a Parr shaker was added 20% palladium on carbon (0.10 g), and hydrogen gas was introduced at 37 psi for 19 hours. The reaction mixture was diluted with methanol, filtered through diatomaceous earth (Celite®), and concentrated in vacuo to afford the title compound (0.17 g, 100%) which was used without further purification. MS: m/z 146.1 (M+H)⁺.

### EXAMPLE 19

### Preparation of 5-Fluoro-octahydro-cyclopenta[c]pyrrol-4-ol

### Step 1: Preparation of 4-Benzoyloxy-5-fluoro-hexahydro-cyclopenta[c]pyrrole-2-carboxylic acid benzyl ester

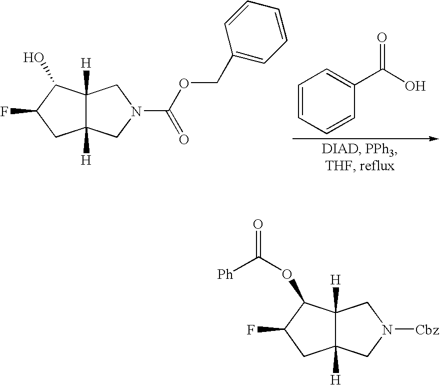

To a solution of triphenylphosphine (5.62 g, 21.4 mmol) in tetrahydrofuran (20 mL) was added diisopropyl azodicarboxylate (4.2 mL, 21 mmol). After 45 minutes, benzoic acid (2.62 g, 21.4 mmol) was added, followed by 5-fluoro-4-hydroxy-hexahydro-cyclopenta[c]pyrrole-2-carboxylic acid benzyl ester (B) (2.0 g, 7.2 mmol) in tetrahydrofuran (2 mL). The transfer was completed with 2×2 mL of tetrahydrofuran. The reaction mixture was heated at 70° C. for 48 hours, and additional triphenylphosphine (2.81 g, 10.7 mmol), diisopropyl azodicarboxylate (2.1 mL, 11 mmol), and benzoic acid (1.31 g, 10.7 mmol) were added. Heating was continued for 24 hours, and the mixture was cooled to room temperature, and concentrated in vacuo. The residue was purified on a 40 L Biotage column using an ethyl acetate/hexanes gradient to afford the title compound (2.7 g, 100%). MS (APCI+): m/z 384.0 (M+H)⁺.

### Step 2: Preparation of 5-Fluoro-4-hydroxy-hexahydro-cyclopenta[c]pyrrole-2-carboxylic acid benzyl ester

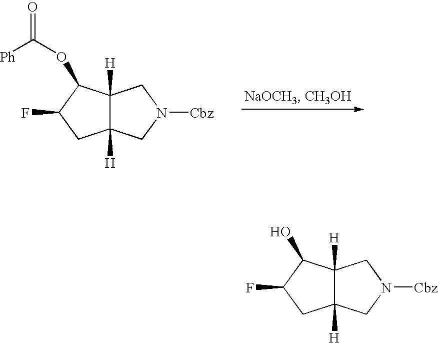

To a cooled (0° C.) solution of 4-benzoyloxy-5-fluoro-hexahydro-cyclopenta[c]pyrrole-2-carboxylic acid benzyl ester (2.7 g, 7.0 mmol) in methanol (40 mL) was added sodium methoxide (0.59 g, 11 mmol). After 2 hours, glacial acetic acid (0.4 mL) was added, and the reaction mixture was warmed to room temperature and concentrated in vacuo. The residue was partitioned between saturated aqueous ammonium chloride and dichloromethane. The aqueous phase was extracted two times with dichloromethane, and the combined organics were dried over magnesium sulfate, filtered, and concentrated in vacuo. The residue was purified on a 40S Biotage column using an ethyl acetate/hexanes gradient to afford the title compound (0.37 g 19%). MS(APCI+): m/z 280.2 (M+H)⁺, 236.2 (M-CO₂+H).

### Step 3: Preparation of 5-Fluoro-octahydro-cyclopenta[c]pyrrol-4-ol

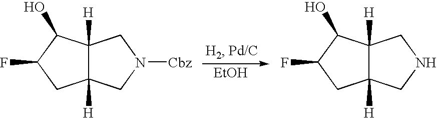

To a solution of 5-fluoro-4-hydroxy-hexahydro-cyclopenta[c]pyrrole-2-carboxylic acid benzyl ester (0.37 g, 1.3 mmol) in ethanol (20 mL) in a Parr shaker was added 5% palladium on carbon (0.50 g), and hydrogen gas was introduced at 37 psi for 16 hours. The reaction mixture was diluted with methanol, filtered through diatomaceous earth (Celite®), and concentrated in vacuo to afford the title compound (0.19 g, 100%) which was used without further purification. MS(APCI+): m/z 146.1 (M+H)⁺.

### EXAMPLE 20

### Preparation of 5,5-Difluoro-octahydro-cyclopenta[c]pyrrol-4-ol

### Step 1: Preparation of 5,5-Difluoro-4-oxo-hexahydro-cyclopenta[c]pyrrole-2-carboxylic acid benzyl ester

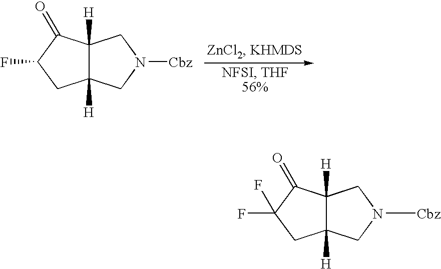

To a cooled (−78° C.) solution of 5-fluoro-4-oxo-hexahydro-cyclopenta[c]pyrrole-2-carboxylic acid benzyl ester (A) (5.38 g, 19.4 mmol) and zinc chloride (41 mL, 41 mmol, 1.0 M in diethyl ether) in tetrahydrofuran (100 mL) was added potassium bis(trimethylsilyl)amide (61 mL, 31 mmol, 0.5M in toluene), and the reaction mixture was stirred for 45 minutes. N-fluorobenzenesulfonimide (8.56 g, 27.1 mmol) in tetrahydrofuran (15 mL) was added, and the transfer was completed with 2×2 mL of tetrahydrofuran. The reaction mixture was allowed to slowly warm to room temperature over 20 hours, and the mixture was diluted with saturated aqueous sodium bicarbonate and ethyl acetate. The aqueous phase was extracted two times with ethyl acetate, and the combined organics were washed with brine, dried over magnesium sulfate, filtered, and concentrated in vacuo. The residue was absorbed onto diatomaceous earth (Celite®) and purified on a 40L Biotage column using an ethyl acetate/hexanes gradient to afford the title compound (3.2 g, 56%). MS(APCI+): m/z 296.1 (M+H)⁺.

### Step 2: Preparation of 5,5-Difluoro-4-hydroxy-hexahydro-cyclopenta[c]pyrrole-2-carboxylic acid benzyl ester

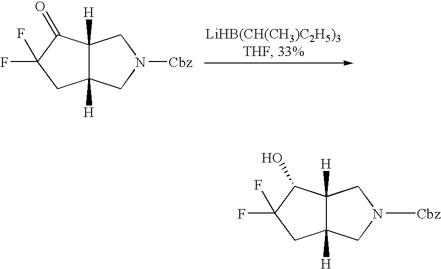

To a cooled (−78° C.) solution of 5,5-difluoro-4-oxo-hexahydro-cyclopenta[c]pyrrole-2-carboxylic acid benzyl ester (2.6 g, 8.7 mmol) in tetrahydrofuran (30 mL) was added lithium tri-sec-butylborohydride (10.5 mL, 10.5 mmol, 1 M in tetrahydrofuran) dropwise, and the reaction mixture was slowly warmed to room temperature over 20 hours. The mixture was cooled to 0° C., treated with 30% aqueous hydrogen peroxide dropwise with caution until all gas evolution ceased, warmed to room temperature and stirred for 30 minutes, and extracted three times with ethyl acetate. The combined organics were washed with brine, dried over magnesium sulfate, filtered, and concentrated in vacuo. The residue was purified on a 40S Biotage column using an ethyl acetate/hexanes gradient to afford the title compound (0.85 g, 33%). MS(APCI+): m/z 298.1 (M+H)⁺.

### Step 3: Preparation of 5,5-Difluoro-octahydro-cyclopenta[c]pyrrol-4-ol

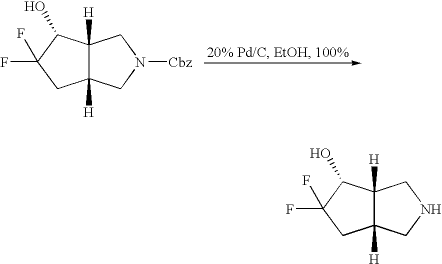

To a solution of 5,5-difluoro-4-hydroxy-hexahydro-cyclopenta[c]pyrrole-2-carboxylic acid benzyl ester (0.71 g, 2.4 mmol) in ethanol (50 ML) in a Parr shaker was added 20% palladium on carbon (0.05 g), and hydrogen gas was introduced at 37 psi for 16 hours. The reaction mixture was diluted with methanol, filtered through diatomaceous earth (Celite®), and concentrated in vacuo to afford the title compound (0.39 g, 100%) which was used without further purification. MS(APCI+): m/z 164.1 (M+H)⁺.

### EXAMPLE 21

### Preparation of (7a-Hydroxymethyl-octahydro-isoindol-3a-yl)-methanol

### Step 1: Preparation of (2-Benzyl-7a-hydroxymethyl-octahydro-isoindol-3a-yl)-methanol

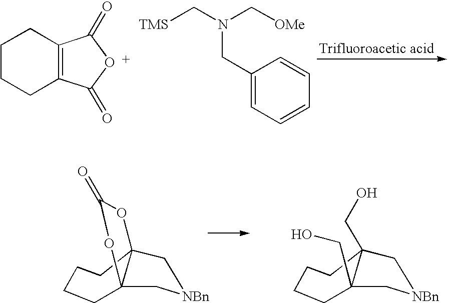

A solution of 4,5,6,7-Tetrahydro-isobenzofuran-1,3-dione (7.00 g, 46 mmol) and benzyl-methoxymethyl-trimethylsilanylmethyl-amine (13.7 g, 57.5 mmol) in dichloromethane (150 mL) was cooled to 0° C. and treated with catalytic trifluoroacetic acid (0.157 g, 1.38 mmol) and allowed to stir and slowly warm to ambient temperature. The mixture was stirred overnight then concentrated, diluted with ethylacetate, and the mixture washed with aqueous saturated sodium bicarbonate solution. The organic layer was then dried with sodium sulfate and filtered through a plug of silica with a 20/1 mixture of ethylacetate/triethylamine. The mixture was then concentrated in vacuo. The resulting residue was then dissolved in tetrahydrofuran (200 mL), under a nitrogen atomosphere (0° C.), and treated with a diethylether solution of lithium aluminum hydride (150 mL, 1.0 M) and allowed to slowly warm to ambient temperature and stirred overnight. The mixture was then treated with 5.6 mL of water and stirred for 15 minutes. The mixture was then treated with 5.6 mL of 15% aqueous sodium hydroxide and stirred for 15 minutes before being treated with 17 mL of water. The mixture was then stirred for 4 hr and filtered. The filtrate was then concentrated in vacuo to provide an oil (11.0 g). MS(APCI+): m/z 276 (M+H)⁺.

### Step 2: Preparation of (7a-Hydroxymethyl-octahydro-isoindol-3a-yl)-methanol

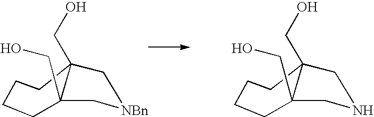

To a solution of (2-benzyl-7a-hydroxymethyl-octahydro-isoindol-3a-yl)-methanol (11.0 g, 39.9 mmol) in 100 mL of methanol in a Parr shaker was added 2.0 g of 20% palladium on carbon and hydrogen gas was introduced at 50 psi for 63 hrs. The mixture was then diluted with additional methanol, filtered through diatomaceous earth (Celite (R)), and concentrated in vacuo to afford the title compound as an oil that was used without further purification. MS(APCI+): m/z 186 (M+H)⁺

B. Coupling of Sidechain Precursors to Quinolone Cores

### EXAMPLE 22

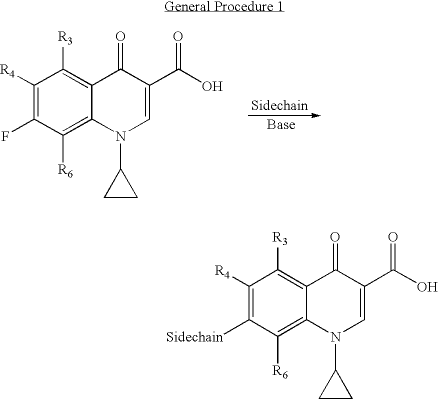

To a slurry of the side chain (1.3 equivalents) and the quinolone core (1 equivalent) in acetonitrile (0.20-0.25 mmol) is added triethylamine (4.9-5.1 equiv.). The resulting reaction mixture is heated at 80° C., upon which it becomes homogenous. After 2-5 hours, the reaction mixture is cooled to room temperature and concentrated in vacuo. The resulting residue is partitioned between ethyl acetate and 1.0 N hydrochloric acid. The aqueous phase is extracted two times with ethyl acetate, and the combined organic layers are washed with brine, dried over magnesium sulfate, filtered, and concentrated in vacuo to afford the coupled product.

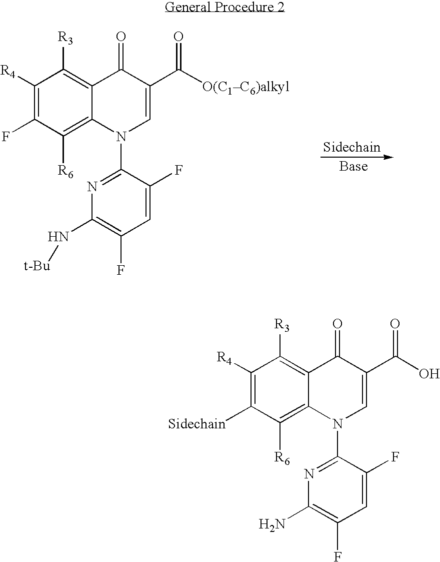

To a solution of the quinone ester (1 equiv.) and sidechain (1.3 equiv) in acetonitrile (0.20-0.25 mMol) is added triethylamine (4.9-5.1 equiv.). The reaction mixture 1 s heated at 80° C. for 12-36 hours. The mixture is then cooled to room temperature, diluted with ethyl acetate, and washed with 1.0 N hydrochloric acid. The aqueous phase is extracted with ethyl acetate, and the combined organics are dried over magnesium sulfate, filtered, and concentrated in vacuo. The residue is purified on a 40S Biotage column using an ethyl acetate/ dichloromethane gradient to afford the coupled product.

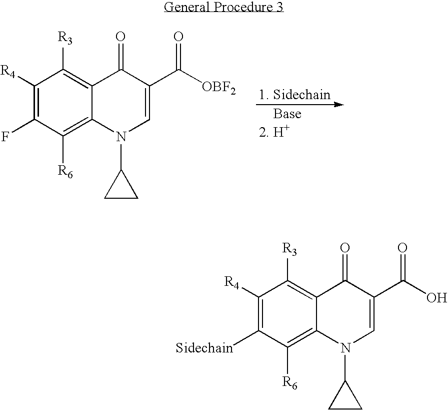

To a solution of the quinolone borate ester (1 equiv.) and sidechain (0.14 g, 1.3 equiv.) in acetonitrile (0.20-0.25 mMol) is added triethylamine ((4.9-5.1 equiv.). The reaction mixture is stirred at room temperature for 24-96 hours and concentrated in vacuo. The resulting residue is dissolved in ethanol, treated with triethylamine (0.20-0.25 mMol), and heated at 85° C. After 2-5 hours, the reaction mixture is cooled to room temperature and concentrated in vacuo. The residue is partitioned between chloroform and saturated aqueous ammonium chloride. The aqueous phase is extracted one time with chloroform, and the combined organics are dried over magnesium sulfate, filtered, and concentrated in vacuo to afford the coupled product.

Coupling Reactions

| Core | Sidechain | Product MS/MP |
|---|---|---|
| 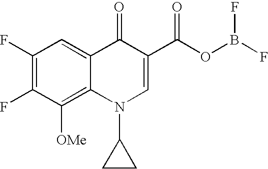 | 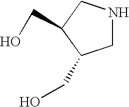 | 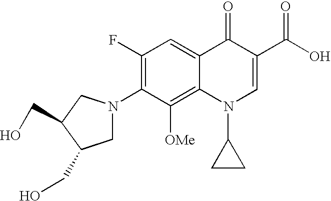 MS (APCI): m/z 410 (M + H)⁺ |
| 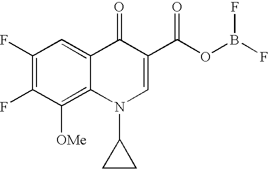 | 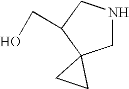 | 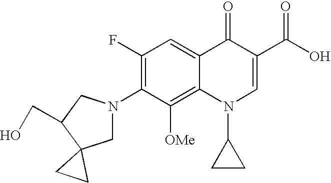 mp 185-187° C. |
| 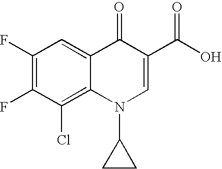 | 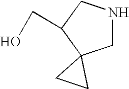 | 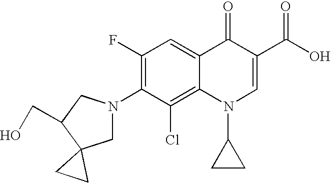 MS (APCI): m/z 407 (M + H)⁺ |
| 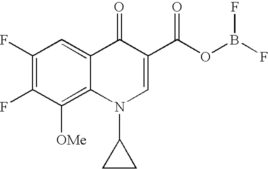 | 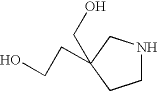 | 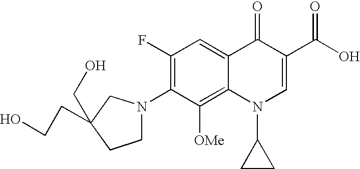 mp = 185-187° C. |
| 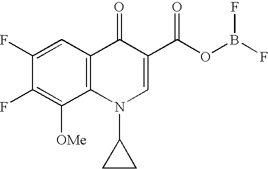 | 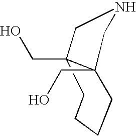 | 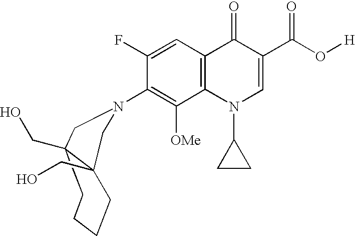 mp = >250° C. |
| 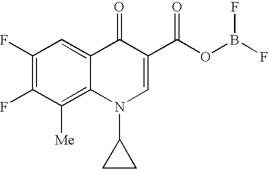 | 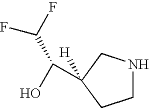 | 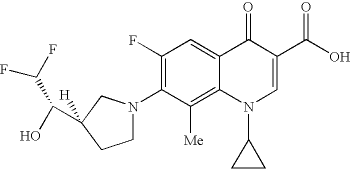 mp = 153-155° C. |
| 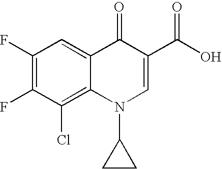 | 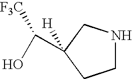 | 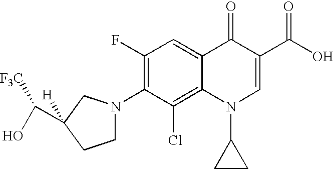 mp = 179-181° C. |
| 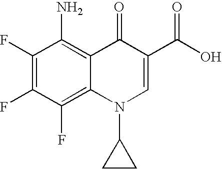 | 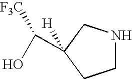 | 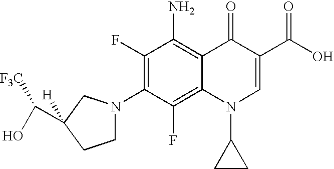 mp = 250-252° C. |
| 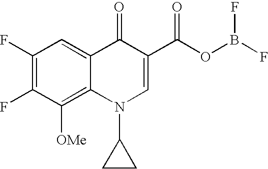 | 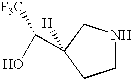 | 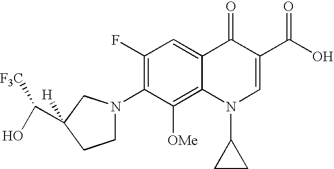 mp = 210-212° C. |
| 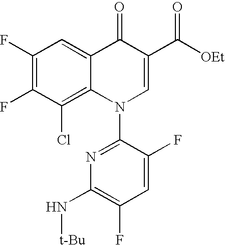 | 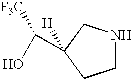 | 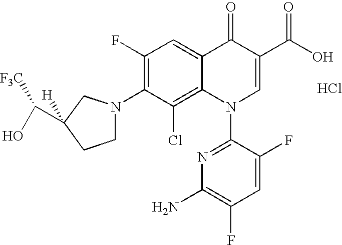 Two steps from quinone ethyl ester (second step deprotection) Mp = 226-228° C. |
| 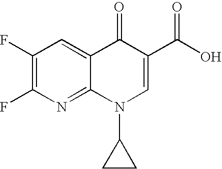 | 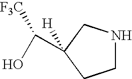 | 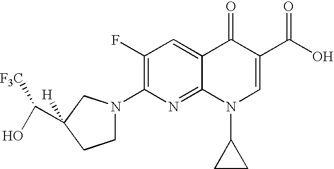 mp = 241-243° C. |
| 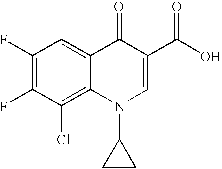 | 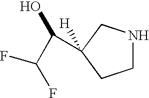 | 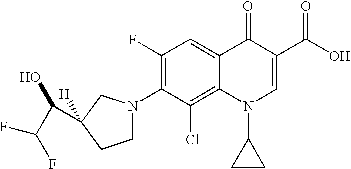 mp 154-156° C. |
| 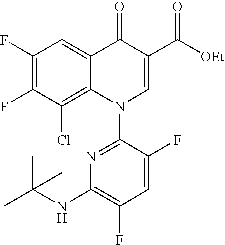 | 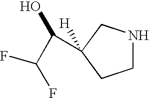 | 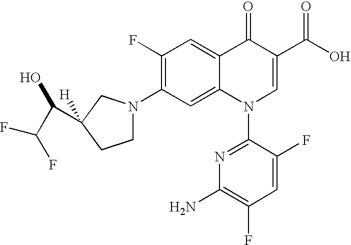 Two steps from quinone ethyl ester (second step deprotection) MS (APCI+): m/z 485 (M + H) |
| 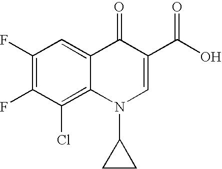 | 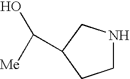 | 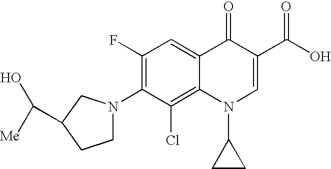 MS (APCI+): m/z 395 (M + H) |
| 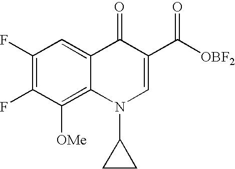 | 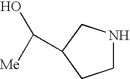 | 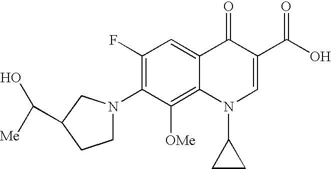 MS (APCI+): m/z 391 (M + H) |
| 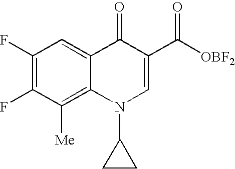 | 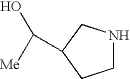 | 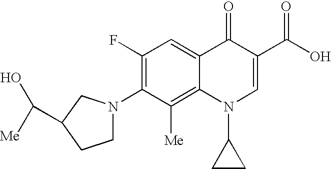 mp (136-140° C.) MS (APCI+) m/z 375 (M + H) |
| 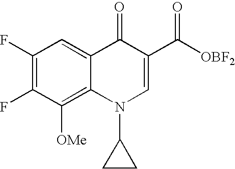 | 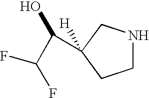 | 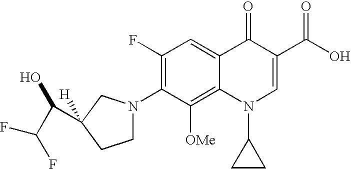 mp (220-222° C. |
| 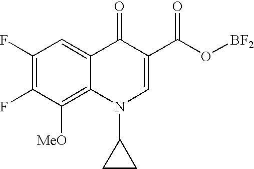 | 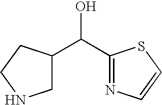 | 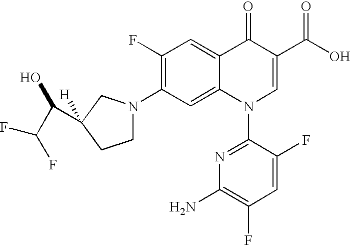 m.p. = 195-205 C. MS (APCI) (m + 1)/z 460.1 |
| 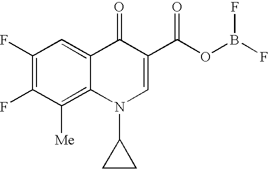 | 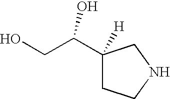 | 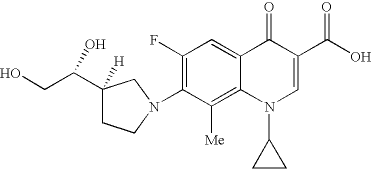 MS (APCI+): m/z 391 (M + H) |
| 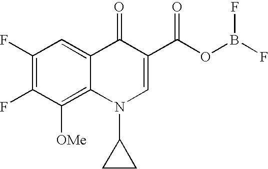 | 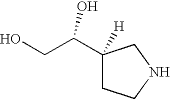 | 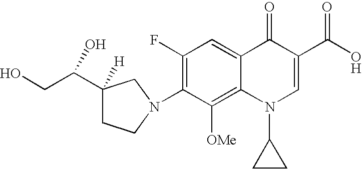 MS (APCI+): m/z 407 (M + H) |
| 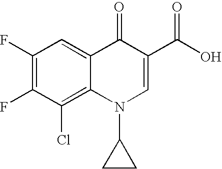 | 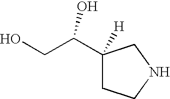 | 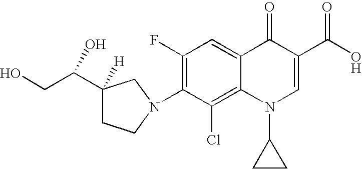 MS (APCI+): m/z 411 (M + H) |
| 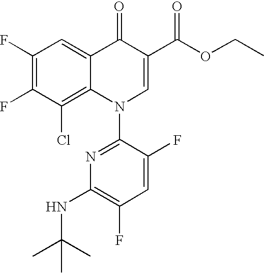 | 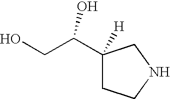 | 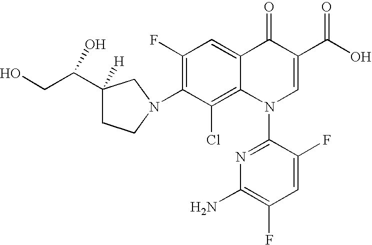 MS (APCI+): m/z 499 (M + H) |
| 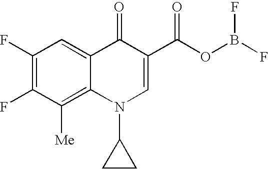 | 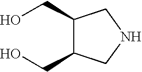 | 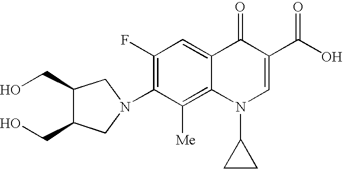 MS (APCI+): m/z 391 (M + H) |
| 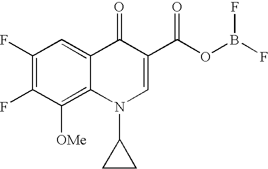 | 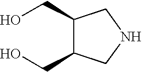 | 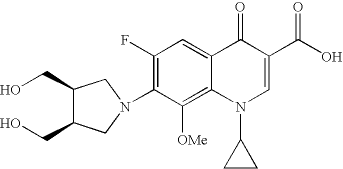 MS (APCI+): m/z 407 (M + H) |
| 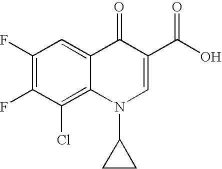 | 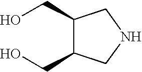 | 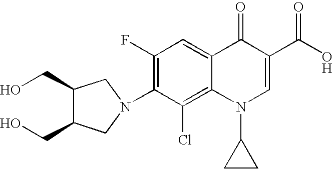 MS (APCI+): m/z 411 (M + H) |
| 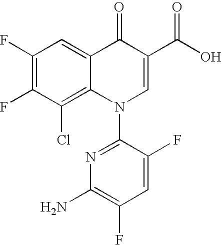 | 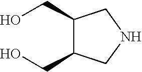 | 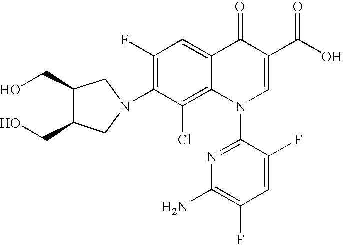 MS (APCI+): m/z 499 (M + H) |
| 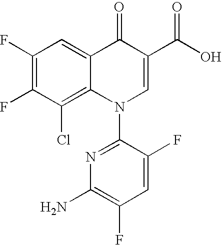 | 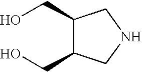 | 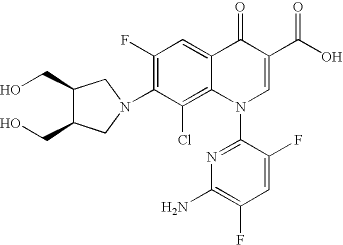 MS (APCI+): m/z 377 (M + H) |
| 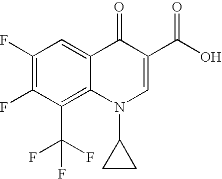 | 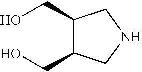 | 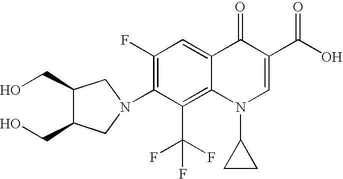 MS (APCI+): m/z 445 (M + H) |
| 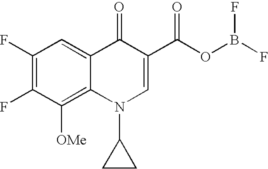 | 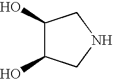 | 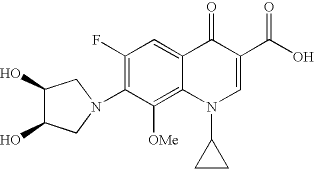 MS (APCI+): m/z 379 (M + H) |
| 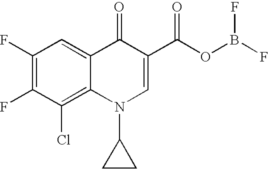 | 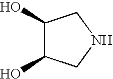 | 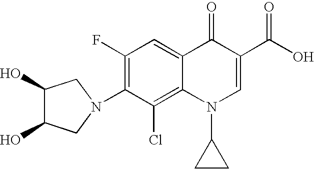 MS (APCI+): m/z 383 (M + H) |
| 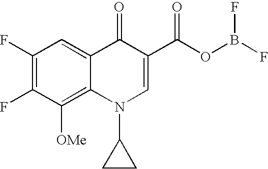 | 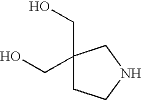 | 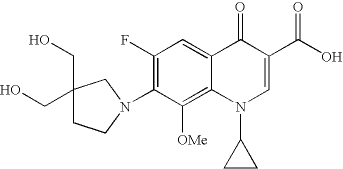 MS (APCI+): m/z 407 (M + H) |
| 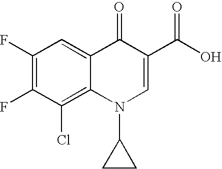 | 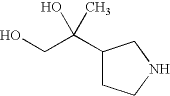 | 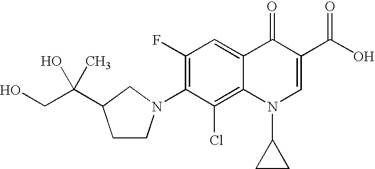 mp 170-172° C. |
| 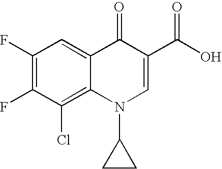 | 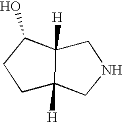 | 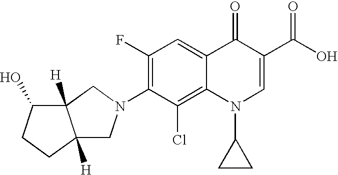 mp 174-176° C. |
| 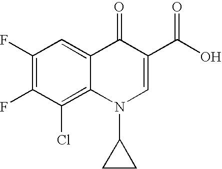 | 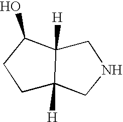 | 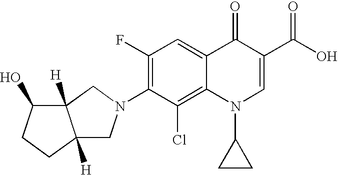 mp 223-225° C. (decomposed) |
| 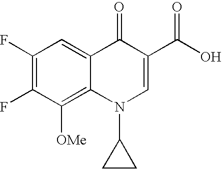 | 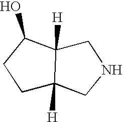 | 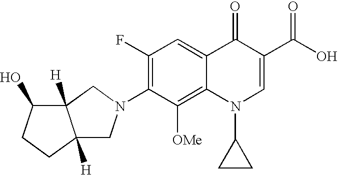 mp 195-200° C. (decomposed) |
| 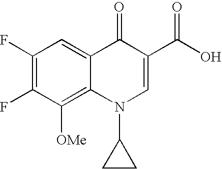 | 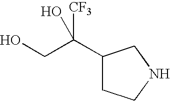 | 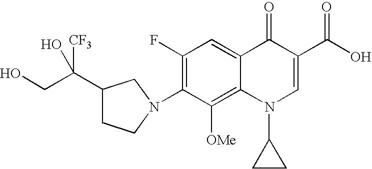 mp 245-248° C. |
| 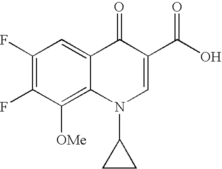 | 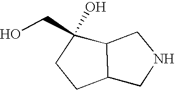 | 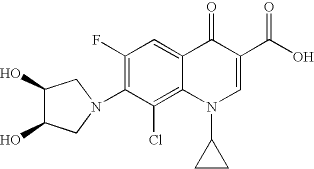 mp 212-215° C. |
| 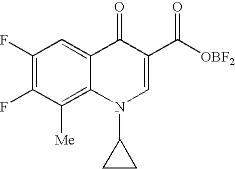 | 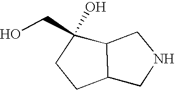 | 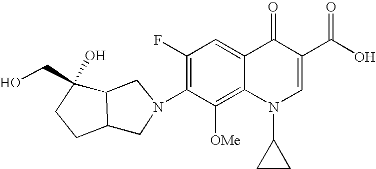 mp 193-195° C. |
| 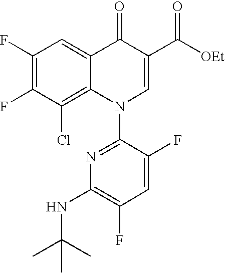 | 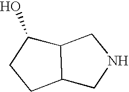 | 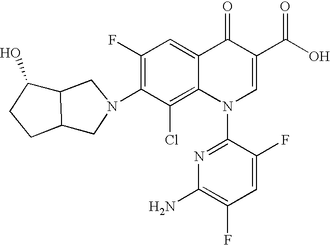 MS: m/z 579.1 (M + H)⁺, 578.1 (M −H)⁺ 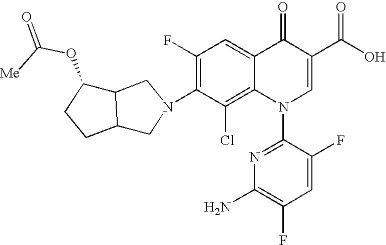 MS: m/z 537.0 (M + H)⁺ |
| 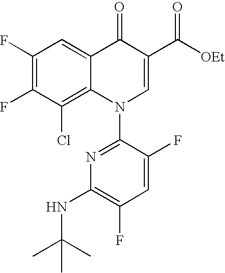 | 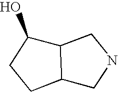 | 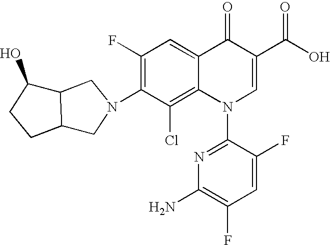 MS: MS: m/z 579.1 (M + H)⁺ 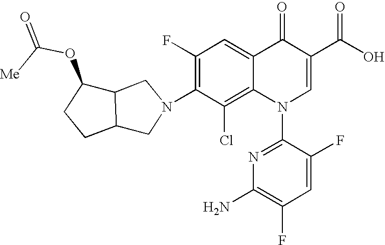 mp 150-153° C. (decomposed) |
| 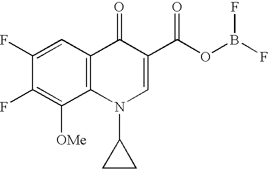 | 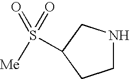 | 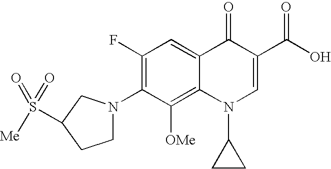 MS: m/z 425 (M + 1) |
| 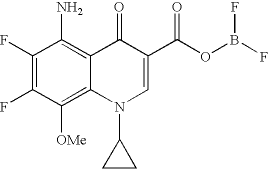 | 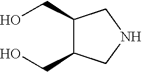 | 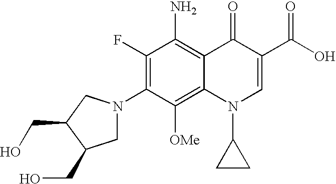 MS: m/z 422 (M + 1) |
| 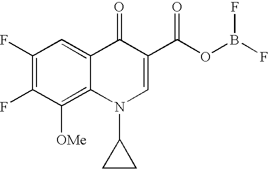 | 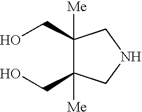 | 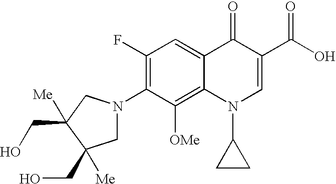 MS: m/z 435 (M + 1) |
| 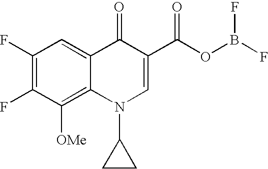 | 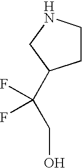 | 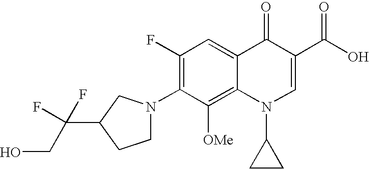 MS: m/z 427 (M + 1) |
| 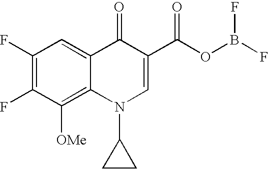 | 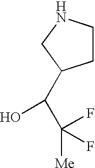 | 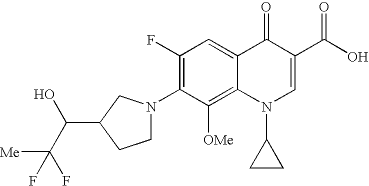 MS (EI): m/z = 441.1 (M + 1) |
| 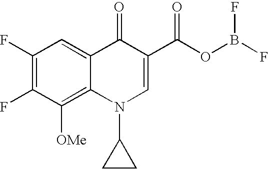 | 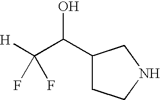 | 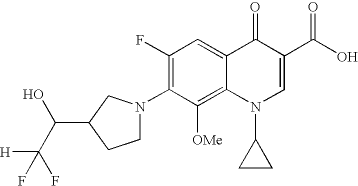 MS (APCI+): m/z 427 (M + H) |
| 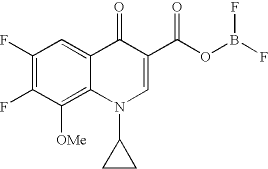 | 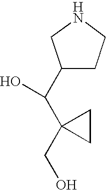 | 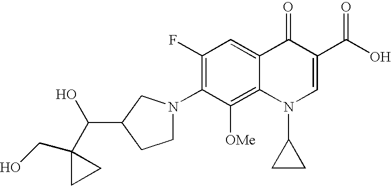 MS (APCI+): m/z 447 (M + H) |
| 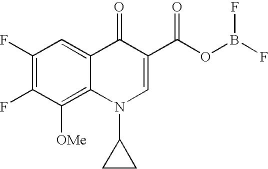 | 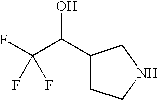 | 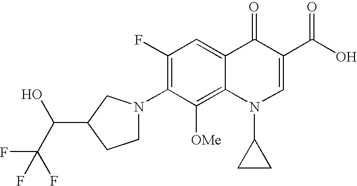 MS (APCI+): m/z 445 (M + H) |
| 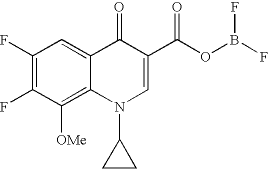 | 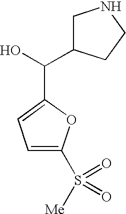 | 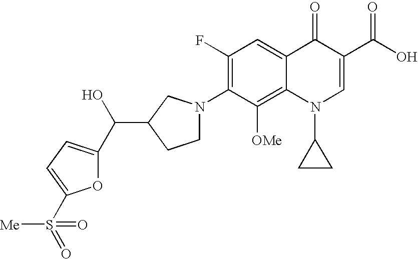 MS (APCI+): m/z 521 (M + H) |
| 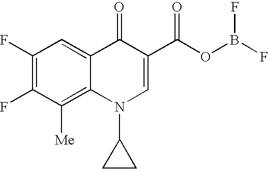 | 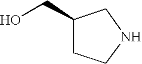 | 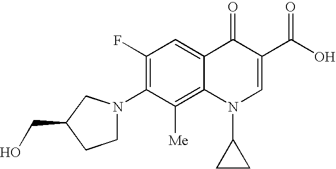 MS (APCI+): m/z 361 (M + H) |
| 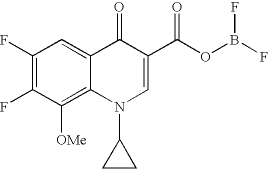 | 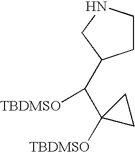 | 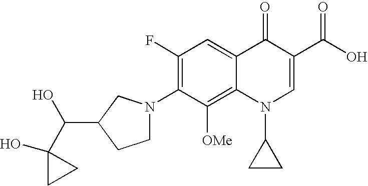 Desilylation occurred upon aqueous acidic workup MS (APCI+): m/z 433 (M + H) |
| 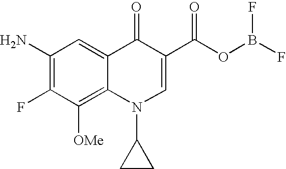 | 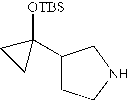 | 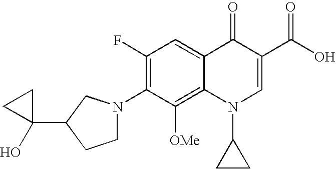 Desilylation occurred upon aqueous acidic workup MS (APCI+): m/z 403 (M + H) |

C. Elaboration of Product Quinolones

### EXAMPLE 23

### Preparation of 7-(3,4-Bis-acetoxymethyl-pyrrolidin-1-yl)-1-cyclopropyl-6-fluoro-8-methyl-4-oxo-1,4-dihydro-quinoline-3-carboxylic acid

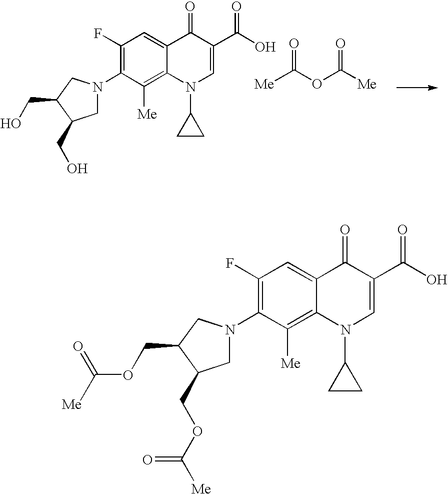

A mixture of 7-(3,4-Bis-hydroxymethyl-pyrrolidin-1-yl)-1-cyclopropyl-6-fluoro-8-methyl-4-oxo-1,4-dihydro-quinoline-3-carboxylic acid (0.140 g, 0.359 mmol), acetic anhydride (0.548 mL, 5.38 mmol), and pyridine (1.45 mL, 17.9 mmol) was heated to 50° C. for 5 hours. The mixture was then concentrated, diluted with ethyl acetate, and washed with 1.0 N hydrochloric acid. The organic layer was then dried with sodium sulfate and the solvent removed in vacuo The residue was then taken up in a minimum amount of dichloromethane and triturated with hexanes. Precipitate washed with hexanes. Product dried in vacuo (0.139 g). MS (APCI+): m/z 475 (M+H)⁺.

### EXAMPLE 24

### Preparation of 7-(3,4-Bis-isobutyryloxymethyl-pyrrolidin-1-yl)-1-cyclopropyl-6-fluoro-8-methyl-4-oxo-1,4-dihydro-quinoline-3-carboxylic acid

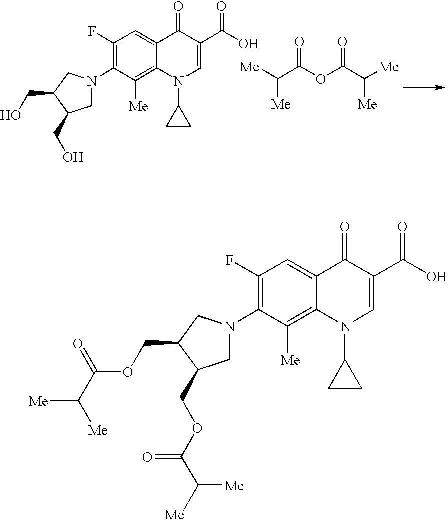

A mixture of 7-(3,4-Bis-hydroxymethyl-pyrrolidin-1-yl)-1-cyclopropyl-6-fluoro-8-methyl-4-oxo-1,4-dihydro-quinoline-3-carboxylic acid (0.140 g, 0.359 mmol), isobutyric anhydride (0.0.850 mL, 5.38 mmol) and pyridine (1.45 mL, 17.93 mmol) was heated to 50° C. for 5 hours. The mixture was then concentrated, diluted with ethyl acetate, and washed with 1 N hydrochloric acid. The organic layer was then dried with sodium sulfate and the solvent removed in vacuo. The residue was then submitted to column chromatography (SiO₂, dichloromethane to 10% methanol/dichloromethane). The residue was then recrystallized form ethanol. The product was dried in vacuo (0.095 g). mp=141-143° C.

D. Pharmaceutical Formulations

### EXAMPLE 25

The following illustrates representative pharmaceutical dosage forms, containing a compound of Formula I (“Invention Compound”), for therapeutic or prophylactic use in humans.

| | | |
|---|---|---|
| (i) | Tablet | mg/tablet |

| | |
|---|---|
| ‘Invention Compound’ | 25.0 |
| Lactose | 50.0 |
| Corn Starch (for mix) | 10.0 |
| Corn Starch (paste) | 10.0 |
| Magnesium Stearate (1%) | 3.0 |
| | 300.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| | | |
|---|---|---|
| (ii) | Tablet | Mg/capsule |

| | |
|---|---|
| ‘Invention Compound | 10.0 |
| Colloidal Silicon Dioxide | 1.5 |
| Lactose | 465.5 |
| Pregelatinized Starch | 120.0 |
| Magnesium Stearate (1%) | 3.0 |
| | 600.0 |

| | | |
|---|---|---|
| | Preparation for | |
| (iii) | Oral Solution | Amount |

| | | |
|---|---|---|
| ‘Invention Compound’ | 400 | mg |
| Sorbitol Solution (70% N.F.) | 40 | mL |
| Sodium Benzoate | 20 | mg |
| Saccharin | 5 | mg |
| Cherry Flavor | 20 | mg |
| Distilled Water q.s. | 100 | mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| | | |
|---|---|---|
| (v) | Injection 1 (1 mg/mL) | Amount |

| | |
|---|---|
| ‘Invention Compound’ | 1.0 |
| Dibasic Sodium Phosphate | 12.0 |
| Monobasic Sodium Phosphate | 0.7 |
| Sodium Chloride | 4.5 |
| N Sodium hydroxide solution | q.s. |
| (pH adjustment to 7.0-7.5) | |
| Water for injection | q.s. ad 1 mL |

| | | |
|---|---|---|
| (vi) | Injection 2 (10 mg/mL) | Amount |

| | |
|---|---|
| ‘Invention Compound’ | 10.0 |
| Dibasic Sodium Phosphate | 1.1 |
| Monobasic Sodium Phosphate | 0.3 |
| Polyethylene glyco 400 | 200.0 |
| N hydrochloric acid solution | q.s. |
| (pH adjustment to 7.0-7.5) | |
| Water for injection | q.s. ad 1 mL |

| | | |
|---|---|---|
| (vii) | Injection 2 (10 mg/mL) | Amount |

| | |
|---|---|
| ‘Invention Compound’ | 20.0 |
| Oleic Acid | 10.0 |
| Trichloromonofluoromethane | 5,000.0 |
| Dichlorodifluoromethane | 10,000.0 |
| Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound of formula I 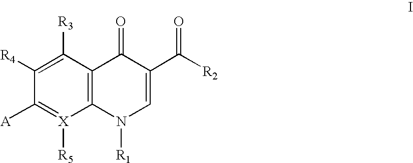
or a pharmaceutically acceptable salt thereof, wherein:
X is N or C, provided that when X is N, R₅ is absent at that position;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl,
O—(CHR₂ₐ)ₘ—OQR_{2b}, wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂ₐ)ₙ—Y wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 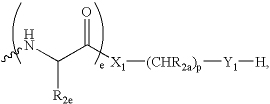
wherein “ [symbol] ” indicates the point of attachment, 2a is as defined above, R₂e is H or (C₁-C₆)alkyl, e is an integer of from 1 to 10, p is an integer of from 2 to 10, and X₁ and Y₁ are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R₁ and R₅, together with the carbons to which they are attached, form a substituted or unsubstituted 6-membered ring containing an additional heteroatom selected from O, S, NH, or N(C₁-C₆)alkyl;
with the proviso that when R₁ is (C₃-C₆)cycloalkyl or halo(C₃-C₆)cycloalkyl, R₅ is H, halo, NH₂, (C₁-C₆)alkoxy, or halo(C₁-C₆)alkoxy.
A is 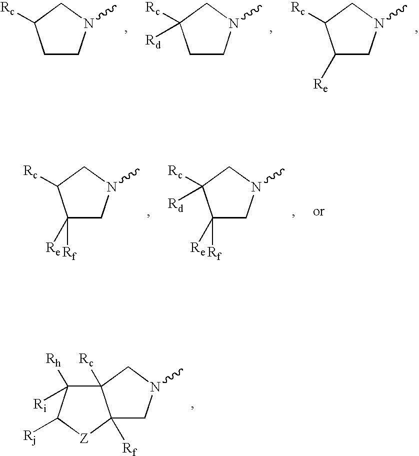
wherein R_{c} is OH,
OPO(OH)₂,
OPO(O(C₁-C₆)alkyl)₂, 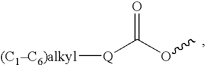
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 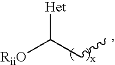
wherein “ [symbol] ” indicates the point of attachment, het is a 5- or 6-membered heterocyclo or heteroaryl group, and x is an integer of from 0 to 10; 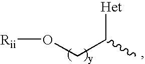
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, or 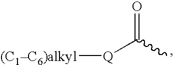
as defined above; and
R_{d}, Rₑ, and R_{f} are each independently (C₁-C₆)alkyl, 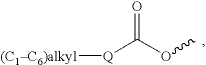
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 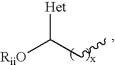
wherein “ [symbol] ” indicates the point of attachment, het is a 5- or 6-membered heterocyclo or heteroaryl group, and x is an integer of from 0 to 10; 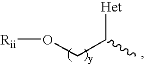
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, or 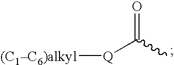
Z is absent or is a linker containing1, 2, or 3 substituted or unsubstituted carbon atoms;
Rₕ, Rᵢ, and Rⱼ are each independently H,
OH,
OPO(OH)₂,
OPO(O(C₁-C₆)alkyl)₂, 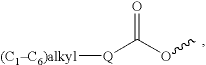
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 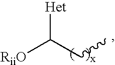
wherein “ [symbol] ” indicates the point of attachment, het is a 5- or 6-membered heterocyclo or heteroaryl group, and x is an integer of from 0 to 10; 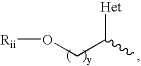
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 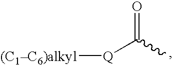
as defined above;
provided that not all of Rₕ Rᵢ, and Rⱼ are H.

2. The compound of claim 1, wherein
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy, difluoromethoxy, or trifluoromethoxy.

3. The compound of claim 1, wherein
R₁ is cyclopropyl or fluorocyclopropyl;
R₃ is H or NH_{2.};
R₄ is H or F.
X is C or N.
R₅ is halo or methoxy.

4. The compound of claim 1, wherein R₁, R₃, R₄, and R₅ are as provided in the following structures, wherein R₂ is OH, OBF₂, or O(C₁-C₆)alkyl, R₄ is H or F, and wherein A′ is

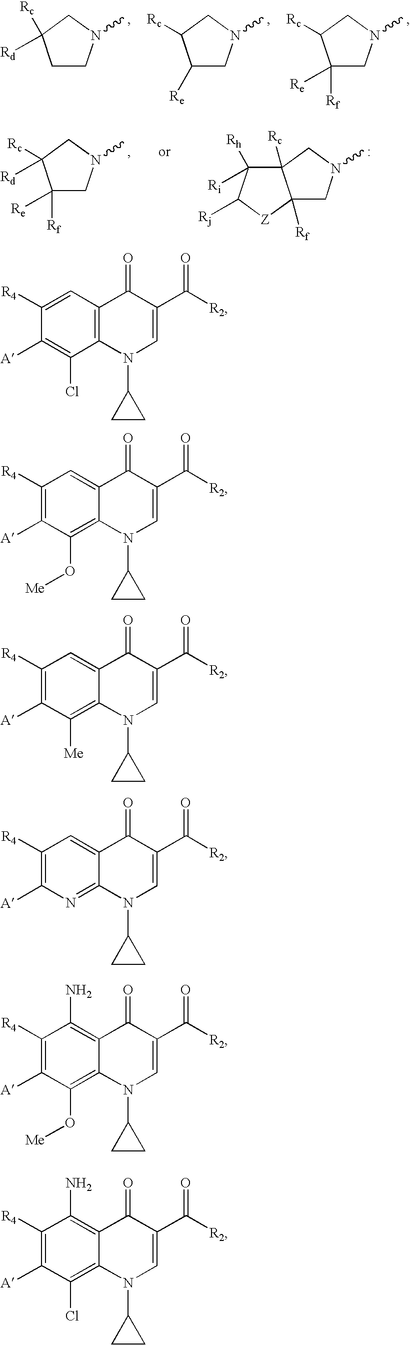

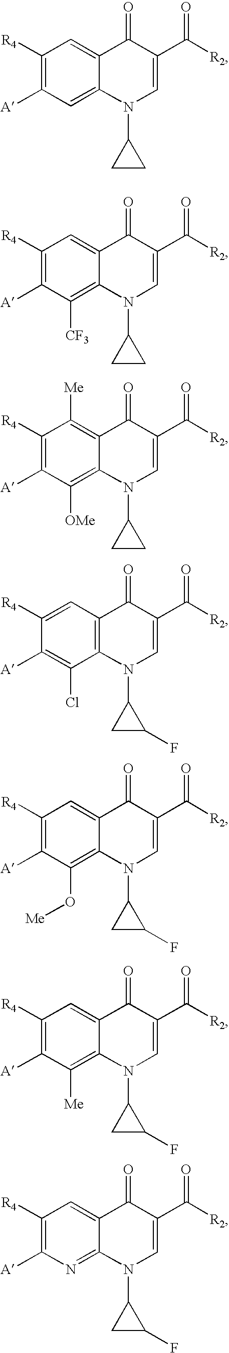

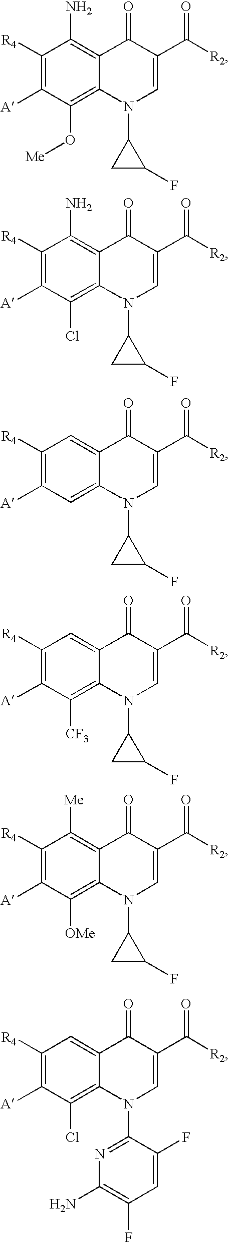

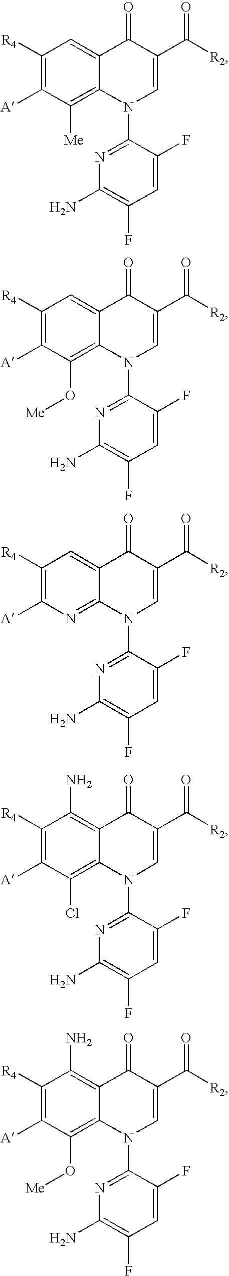

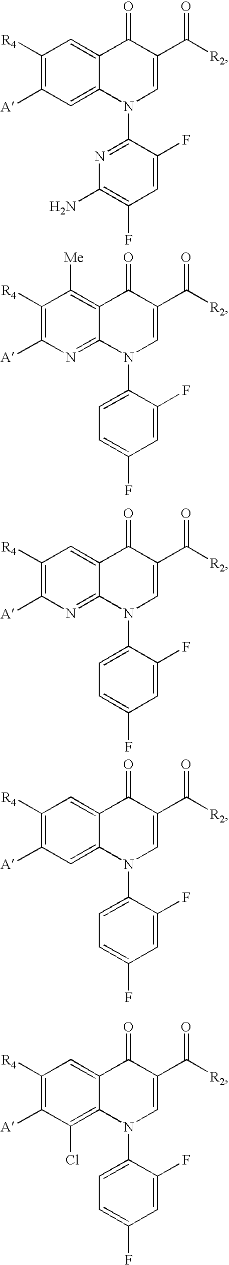

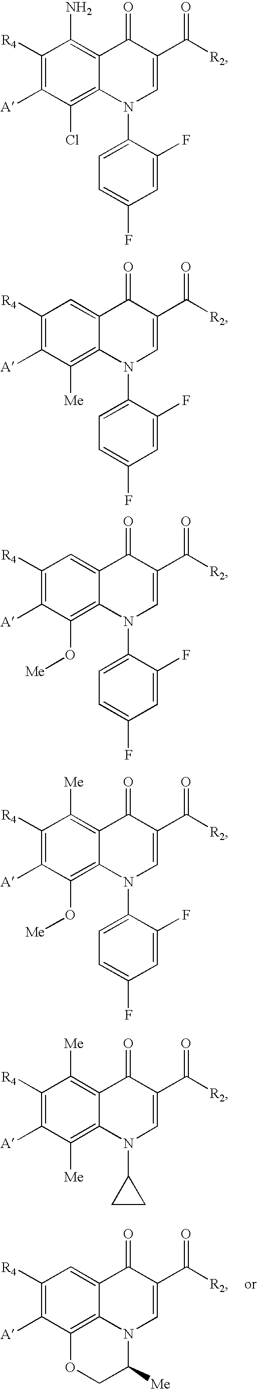

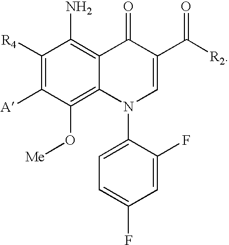

5. The compound of claim 4, wherein R₂ is OH and wherein A′ is

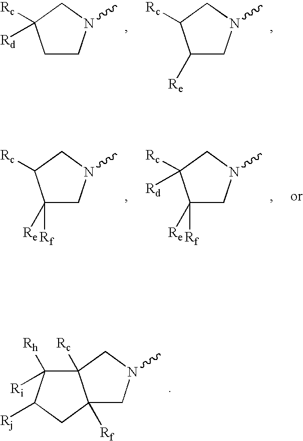

6. The compound of claim 5, wherein A′ is 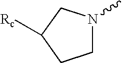
wherein R_{c} is OPO(OH)₂,
OPO(O(C₂-C₆)alkyl)₂, 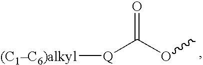
wherein “ [symbol] ” indicates the point of attachment and Q is O or is absent,
RᵢᵢO(C₂-C₆)alkyl,
RᵢᵢO(C₁-C₆)haloalkyl,
RᵢᵢO(C₃-C₆)cycloalkyl,
RᵢᵢO(Cl -C₆)alkyl-O—,
RᵢᵢO(C₁ -C₆)haloalkyl-O—,
RᵢᵢO(C₃-C₆)cycloalkyl-O—, 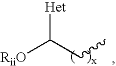
wherein “ [symbol] ” indicates the point of attachment, het is a 5- or 6-membered heterocyclo or heteroaryl group, and x is an integer of from 0 to 10; 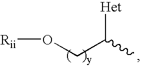
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₂-C₆)alkyl)₂, or 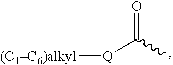
as defined above.

7. The compound of claim 6, wherein

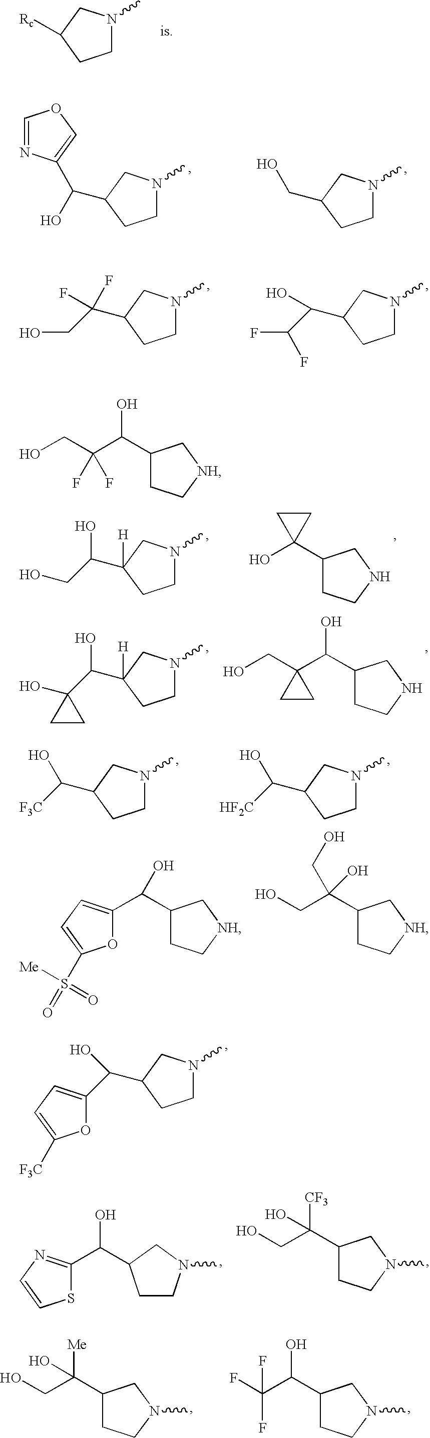

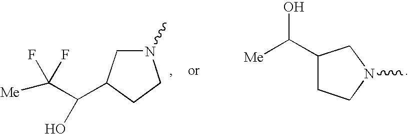

8. The compound of claim 5 wherein A′ is 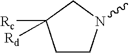
wherein R_{c} is OH,
OPO(OH)₂,
OPO(O(C₁-C₆)alkyl)₂, 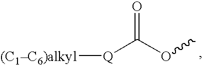
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)Cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 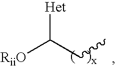
wherein “ [symbol] ” indicates the point of attachment, het is a 5- or 6-membered heterocyclo or heteroaryl group, and x is an integer of from 0 to 10; 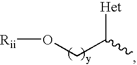
wherein “ [symbol] ” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, or 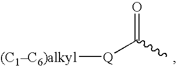
as defined above; and
R_{d} is halo,
(C₁-C₆)alkyl,
RᵢᵢO(C₁-C₆)alkyl,
RᵢᵢO(Cc-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, or 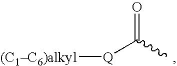
wherein “ [symbol] ” indicates the point of attachment and Q is O or is absent.

9. The compound of claim 8, wherein

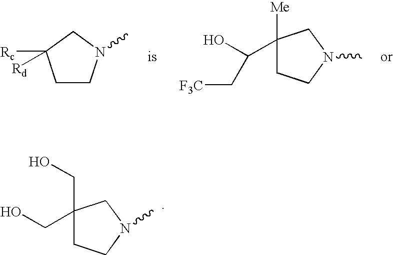

10. The compound of claim 5 wherein A′ is 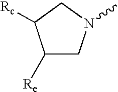
wherein R_{c} is OH,
OPO(OH)₂,
OPO(O(C₁-C₆)alkyl)₂, 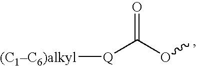
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 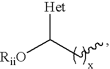
wherein “ [symbol] ” indicates the point of attachment, het is a 5- or 6-membered heterocyclo or heteroaryl group, and x is an integer of from 0 to 10; 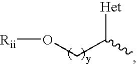
wherein “ [symbol] ” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 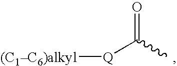
as defined above; and
Rₑ is halo 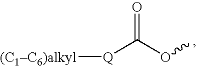
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 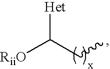
wherein “ [symbol] ” indicates the point of attachment, het is a 5- or 6-membered heterocyclo or heteroaryl group, and x is an integer of from 0 to 10; 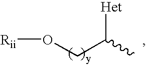
wherein “ [symbol] ” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, or 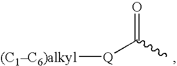
as defined above.

11. The compound of claim 10, wherein

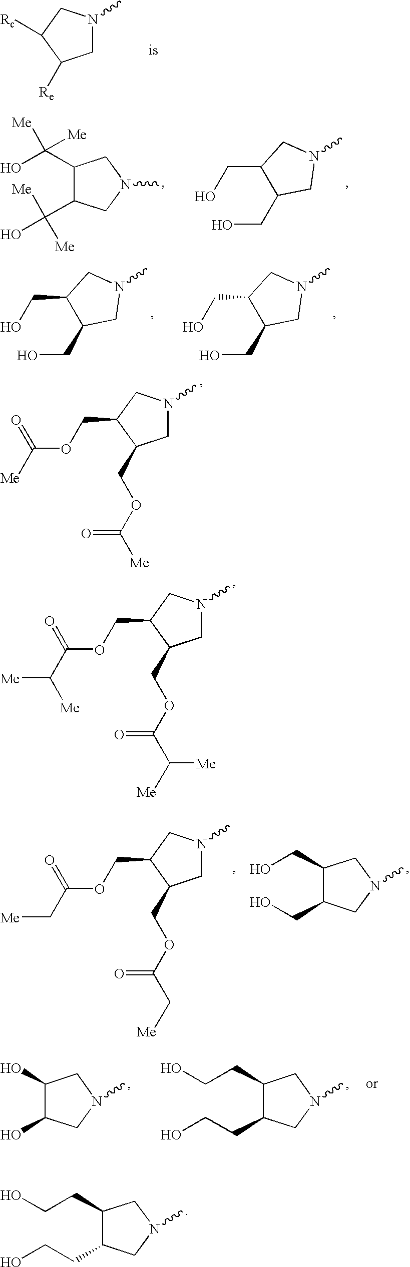

12. The compound of claim 5 wherein A′ is 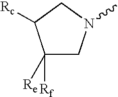
wherein R_{c} is
OPO(OH)₂i
OPO(O(C₁-C₆)alkyl)₂, 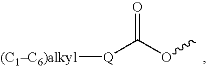
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 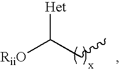
wherein “ [symbol] ” indicates the point of attachment, het is a 5- or 6-membered heterocyclo or heteroaryl group, and x is an integer of from 0 to 10; 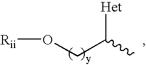
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 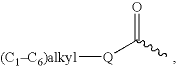
as defined above; and
Rₑ and R_{f} are each independently (C₁-C₆)alkyl or together with the carbon to which they are attached, form a substituted or unsubstituted 3, 4, 5, or 6-membered ring containing 0, 1, 2, or 3 heteroatoms selected from NH, N(C₁-C₆)alkyl, S, or O.

13. The compound of claim 12, wherein

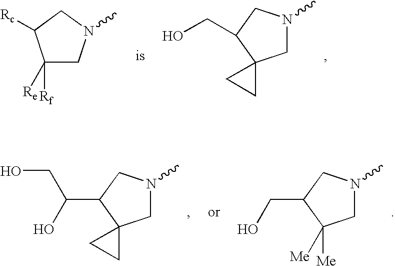

14. The compound of claim 5 wherein A is 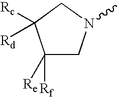
wherein R_{c} and Re are each independantly H,
OH,
OPO(OH)₂, 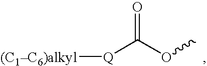
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 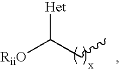
wherein “ [symbol] ” indicates the point of attachment, het is a 5- or 6-membered heterocyclo or heteroaryl group, and x is an integer of from 0 to 10; 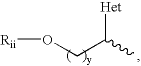
wherein “ [symbol] ” indicates the point of attachment, het is as defined above, and y is an integer of from 1 to 10,
wherein Rᵢᵢ is H,
(C₁-C₆)alkyl,
PO(OH)₂, 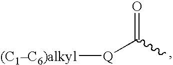
as defined above; and
R_{d} and R_{f} are each independently (C₁-C₆)alkyl, or taken together with the carbons to which they are attached form a substituted or unsubstituted 4, 5, or 6 membered ring, optionally containing one heteroatom selected from NH, N(C₁-C₆)alkyl, S, or O.

15. The compound of claim 14, wherein

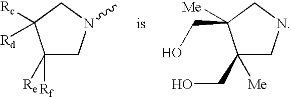

16. The compound of claim 5, wherein A′ is 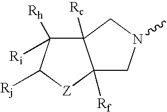
wherein
Z is absent or is a linker containing1, 2, or 3 substituted or unsubstituted carbon atoms;
R_{c} and R_{f} are each independently H, (C₁-C₆)alkyl, or HO—(C₁-C₆)alkyl;
Rₕ, Rᵢ, and Rⱼ are each independently H,
OH,
OPO(OH)₂, 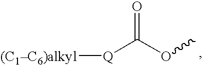
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 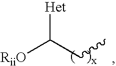
wherein “ [symbol] ” indicates the point of attachment, het is a 5- or 6-membered heterocyclo or heteroaryl group, and x is an integer of from 0 to 10; 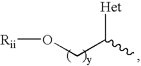
wherein “ [symbol] ” indicates the point of attachment, het is as defined above, and y is an integer of from 1 to 10,
wherein Rᵢᵢ is H,
(C₁-C₆)alkyl,
PO(OH)₂, 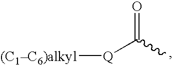
as defined above;
provided that not all of Rₕ Rᵢ, and Rⱼ are H.

17. The compound of claim 16, wherein

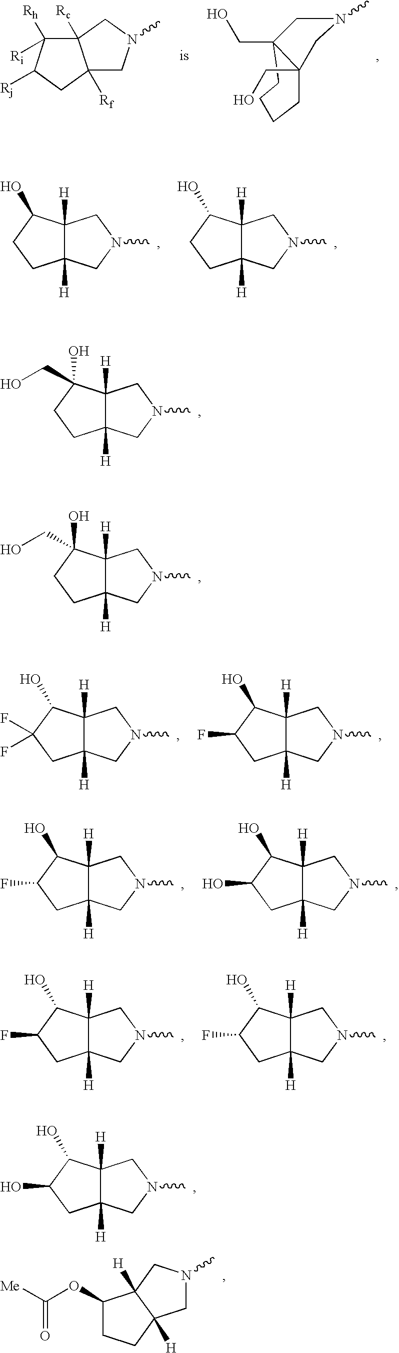

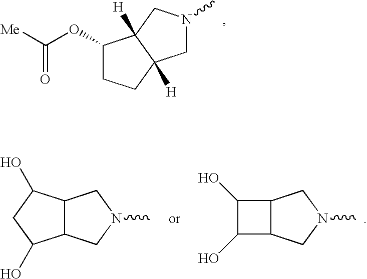

18. A compound of formula II 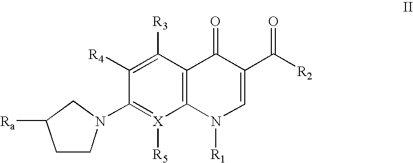
or a pharmaceutically acceptable salt thereof, wherein:
X is N or C, provided that when X is N, R₅ is absent at that position;
R₁ is (C₃-C₆)cycloalkyl,
halo(C₃-C₆)cycloalkyl,
(C₁-C₆)alkyl,
halo(C₁-C₆)alkyl,
aryl, and
heteroaryl;
R₂ is OH,
OBF₂,
O(C₁-C₆)alkyl,
O(C₃-C₆)cycloalkyl, 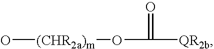
wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂ₐ)ₙ—Y wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 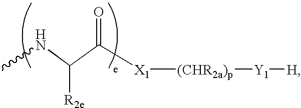
wherein “ [symbol] ” indicates the point of attachment, 2a is as defined above, R₂e is H or (C₁-C₆)alkyl, e is an integer of from 1 to 10, p is an integer of from 2 to 10, and X₁ and Y, are each independently NH or O;
R₃ and R₄ are each independently H,
halo,
NH₂,
(C₁-C₆)alkyl,
halo(C₁-C₆)alkyl,
(C₁-C₆)alkoxy, or
halo(C₁-C₆)alkoxy;
R₅ is H,
halo,
NH₂,
(C₁-C₆)alkyl,
halo(C₁-C₆)alkyl,
(C₁-C₆)alkoxy, or
halo(C₁-C₆)alkoxy;
Rₐ is OPO(OH)₂,
OPO(O(C₁-C₆)alkyl)₂, 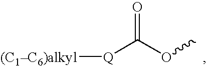
wherein “ [symbol] ” indicates the point of attachment and Q is O or is absent,
RᵢᵢO(C₂-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(Cl-C₆)haloalkyl-O—,
RᵢᵢO(C₃-C₆)cycloalkyl-O—, 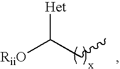
wherein “ [symbol] ” indicates the point of attachment, het is a 5- or 6-membered heterocyclo or heteroaryl group, and x is an integer of from 0 to 10; 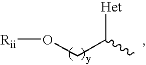
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, or 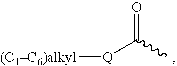
as defined above; or
R₁ and R₅, together with the carbons to which they are attached, form a substituted or unsubstituted 6-membered ring containing an additional heteroatom selected from O, S, NH, or N(C₁-C₆)alkyl;
with the proviso that when R₁ is (C₃-C₆)cycloalkyl or halo(C₃-C₆)cycloalkyl, R₅ is H, halo, NH₂, (C₁-C₆)alkoxy, or halo(C₁-C₆)alkoxy.

19. The compound of claim 18, wherein
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy, difluoromethoxy, or trifluoromethoxy.

20. The compound of claim 18, wherein
R₁ is cyclopropyl or fluorocyclopropyl;
R₃ is H or NH₂.;
R₄ is H or F.
X is C or N.
R₅ is halo or methoxy.

21. The compound of claim 18, wherein R₁, R₃, R₄, and R₅ are as provided in the following structures, wherein R₂ is OH, OBF₂, or O(C₁-C₆)alkyl, R₄ is H or F, and wherein A′ is

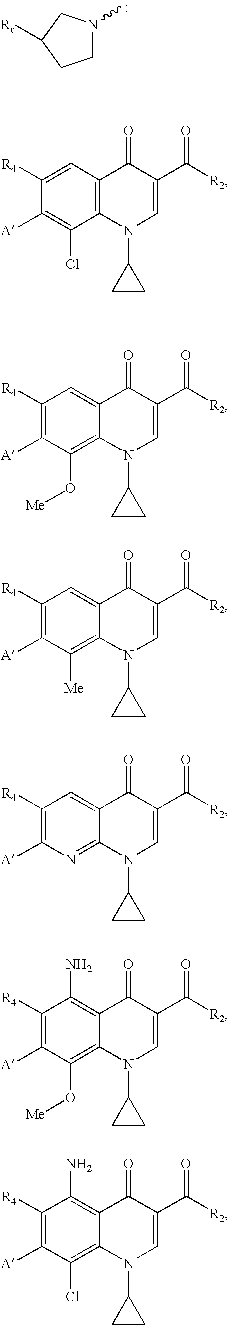

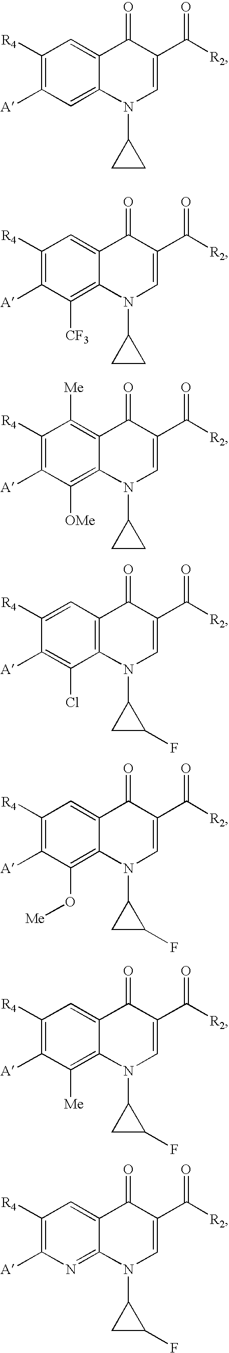

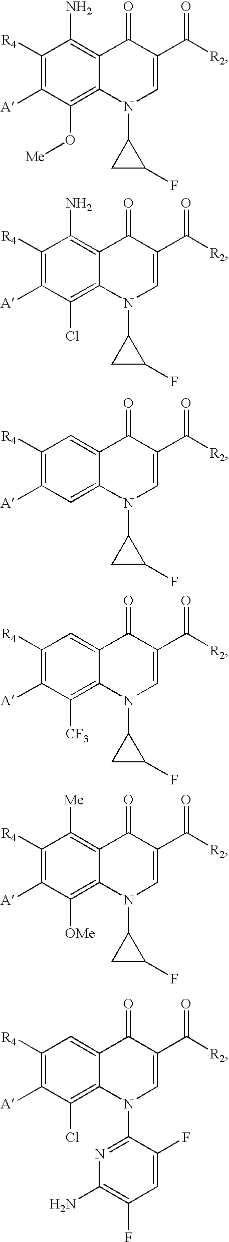

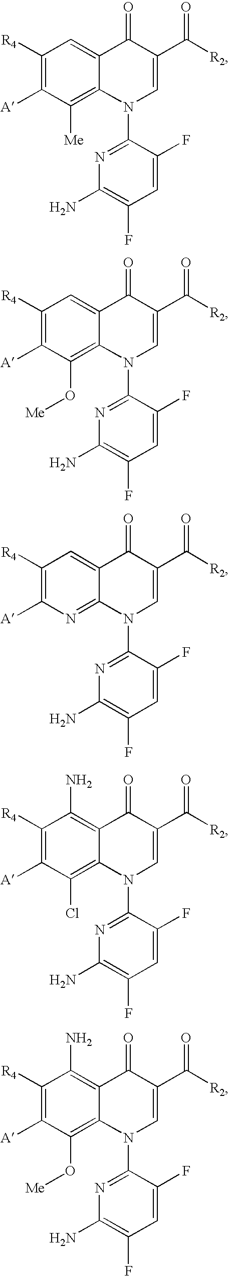

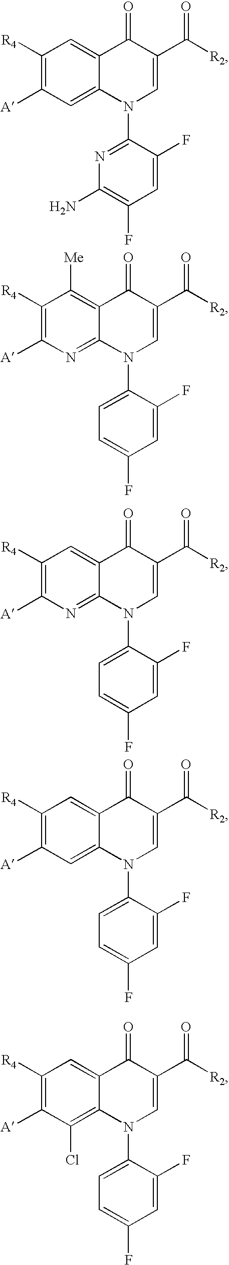

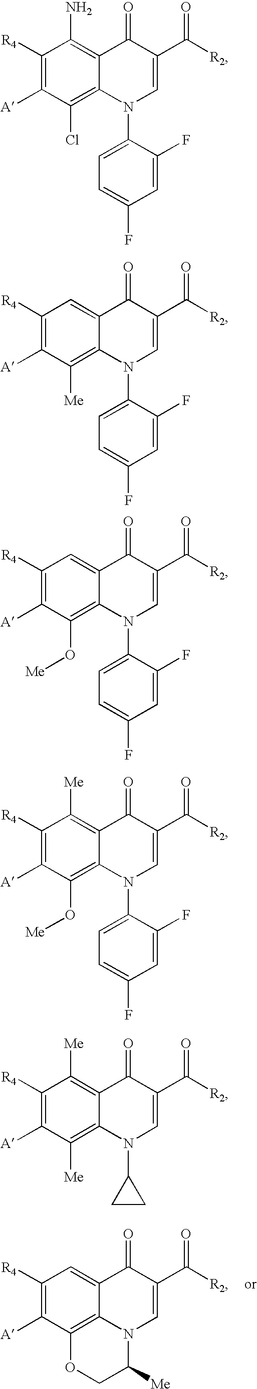

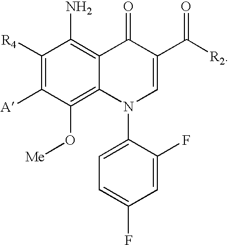

22. The compound of claim 21, wherein is

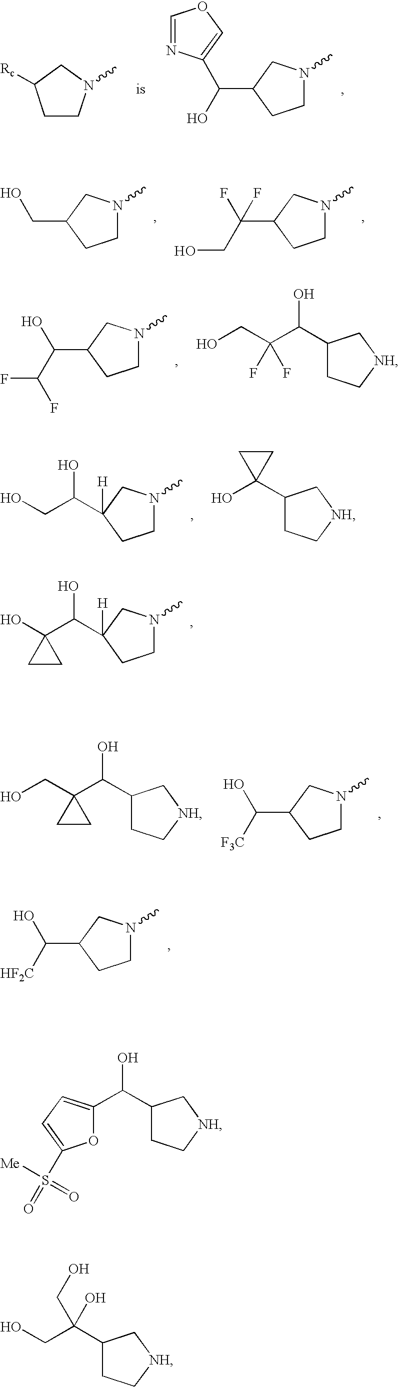

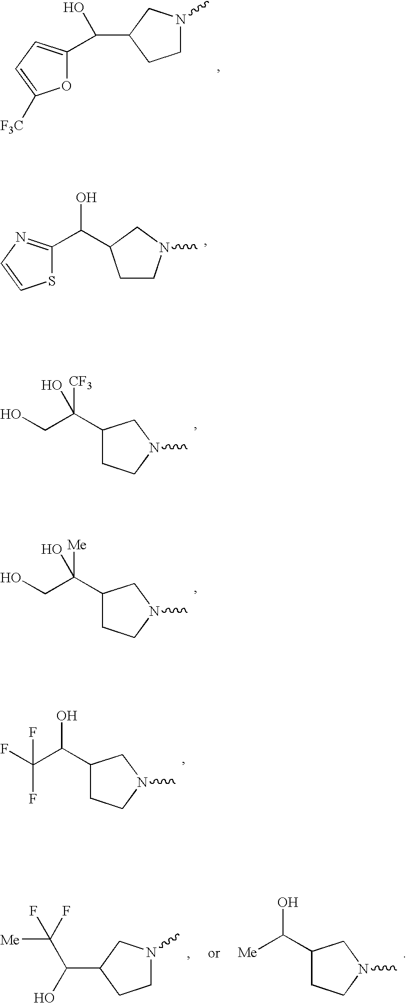

23. The compound of claim 21 which is
1-Cyclopropyl-6-fluoro-7-[3-(2-hydroxy-1-hydroxymethyl-ethyl)-pyrrolidin-1-yl]-8-methoxy-4-oxo-1,4-dihydro-quinoline-3-carboxylic acid;
1-Cyclopropyl-6-fluoro-7-(3-hydroxymethyl-pyrrolidin-1-yl)-8-methoxy-4-oxo-1,4-dihydro-quinoline-3-carboxylic acid;
1-Cyclopropyl-7-[3-(2,2-difluoro-1-hydroxy-ethyl)-pyrrolidin-1-yl]-6-fluoro-8-methoxy-4-oxo-1,4-dihydro-quinoline-3-carboxylic acid;
1-Cyclopropyl-7-[3-(2,2-difluoro-1-hydroxy-propyl)-pyrrolidin-1-yl]-6-fluoro-8-methoxy-4-oxo-1,4-dihydro-quinoline-3-carboxylic acid;
1-Cyclopropyl-6-fluoro-7-[3-(1-hydroxy-2-methoxy-ethyl)-pyrrolidin-1-yl]-8-methoxy-4-oxo-1,4-dihydro-quinoline-3-carboxylic acid;
1-Cyclopropyl-6-fluoro-8-methoxy-4-oxo-7-[3-(2,2,2-trifluoro-1-hydroxy-1-hydroxymethyl-ethyl)-pyrrolidin-1-yl]-1,4-dihydro-quinoline-3-carboxylic acid;
1-Cyclopropyl-7-[3R-(1R,2-dihydroxy-ethyl)-pyrrolidin-1-yl]-6-fluoro-8-methoxy-4-oxo-1,4-dihydro-quinoline-3-carboxylic acid;
8-Chloro-1-cyclopropyl-7-[3R-(1R,2-dihydroxy-ethyl)-pyrrolidin-1-yl]-6-fluoro-4-oxo-1,4,8,8a-tetrahydro-quinoline-3-carboxylic acid;
1-Cyclopropyl-6-fluoro-7-{3-[hydroxy-(1-hydroxymethyl-cyclopropyl)-methyl]-pyrrolidin-1-yl}-8-methoxy-4-oxo-1,4-dihydro-quinoline-3-carboxylic acid;
8-Chloro-1-cyclopropyl-6-fluoro-7-{3-[hydroxy-(1-hydroxymethyl-cyclopropyl)-methyl]-pyrrolidin-1-yl}-4-oxo-1,4-dihydro-quinoline-3-carboxylic acid;
1-Cyclopropyl-6-fluoro-7-[3-(1-hydroxy-cyclopropyl)-pyrrolidin-1-yl]-8-methoxy-4-oxo-1,4-dihydro-quinoline-3-carboxylic acid;
1-Cyclopropyl-6-fluoro-8-chloro-4-oxo-7-[3-(1-hydroxy-ethyl)-pyrrolidin-1-yl]-1,4-dihydro-quinoline-3-carboxylic acid;
1-Cyclopropyl-6-fluoro-8-methoxy-4-oxo-7-[3-(1-hydroxy-ethyl)-pyrrolidin-1-yl]-1,4-dihydro-quinoline-3-carboxylic acid;
1-Cyclopropyl-6-fluoro-8-methoxy-4-oxo-7-[3-(2,2,2-trifluoro-1-hydroxy-ethyl)-pyrrolidin-1-yl]-1,4-dihydro-quinoline-3-carboxylic acid;
1-Cyclopropyl-6-fluoro-8-methoxy-4-oxo-7-[3-(2,2,2-trifluoro-1-hydroxy-ethyl)-pyrrolidin-1-yl]-1,4-dihydro-quinoline-3-carboxylic acid;
5-Amino-1-cyclopropyl-6,8-difluoro-4-oxo-7-[3-(2,2,2-trifluoro-1-hydroxy-ethyl)-pyrrolidin-1-yl]-1,4-dihydro-quinoline-3-carboxylic acid;
8-Chloro-1-cyclopropyl-6-fluoro-4-oxo-7-[3-(2,2,2-trifluoro-1-hydroxy-ethyl)-pyrrolidin-1-yl]-1,4-dihydro-quinoline-3-carboxylic acid;
1-Cyclopropyl-6-fluoro-4-oxo-7-[3-(2,2,2-trifluoro-1-hydroxy-ethyl)-pyrrolidin-1-yl]-1,4-dihydro-[1,8]naphthyridine-3-carboxylic acid;
1-Cyclopropyl-6-fluoro-7-[3-(hydroxy-thiazol-4-yl-methyl)-pyrrolidin-1-yl]-8-methoxy-4-oxo-1,4-dihydro-quinoline-3-carboxylic acid;
1-Cyclopropyl-6-fluoro-7-[3-(hydroxy-thiazol-4-yl-methyl)-pyrrolidin-1-yl]-8-methoxy-4-oxo-1,4-dihydro-quinoline-3-carboxylic acid;
8-Chloro-1-cyclopropyl-6-fluoro-7-(3-hydroxymethyl-pyrrolidin-1-yl)-4-oxo-1,4-dihydro-quinoline-3-carboxylic acid;
1-Cyclopropyl-7-[3-(1,1 -difluoro-2-hydroxy-ethyl)-pyrrolidin-1-yl]-6-fluoro-8-methoxy-4-oxo-1,4-dihydro-quinoline-3-carboxylic acid;
1-Cyclopropyl-7-[3-(2,2-difluoro-1-hydroxy-ethyl)-pyrrolidin-1-yl]-6-fluoro-8-methoxy-4-oxo-1,4-dihydro-quinoline-3-carboxylic acid;
1-Cyclopropyl-7-[3-(2,2-difluoro-1-hydroxy-ethyl)-pyrrolidin-1-yl]-6-fluoro-8-chloro-4-oxo-1,4-dihydro-quinoline-3-carboxylic acid;
1-Cyclopropyl-7-[3-(2,2-difluoro-1-hydroxy-ethyl)-pyrrolidin-1-yl]-6-fluoro-4-oxo-1,4-dihydro-[1,8]naphthyridine-3-carboxylic acid;
1-Cyclopropyl-6-fluoro-4-oxo-7-[3-(2,2,2-trifluoro-1-hydroxy-ethyl)-pyrrolidin-1-yl]-1,4-dihydro-[1,8]naphthyridine-3-carboxylic acid;
1-Cyclopropyl-6-fluoro-8-methoxy-4-oxo-7-[3-(2,2,2-trifluoro-1-hydroxy-ethyl)-pyrrolidin-1-yl]-1,4-dihydro-quinoline-3-carboxylic acid;
1-Cyclopropyl-6-fluoro-7-{3-[hydroxy-(5-methanesulfonyl-furan-2-yl)-methyl]-pyrrolidin-1-yl}-8-methoxy-4-oxo-1,4-dihydro-quinoline-3-carboxylic acid;
1-Cyclopropyl-7-[3-(1,2-dihydroxy-1-hydroxymethyl-ethyl)-pyrrolidin-1-yl]-6-fluoro-8-methoxy-4-oxo-1,4-dihydro-quinoline-3-carboxylic acid;
1-Cyclopropyl-7-[3-(2,2-difluoro-1,3-dihydroxy-propyl)-pyrrolidin-1-yl]-6-fluoro-8-methoxy-4-oxo-1,4-dihydro-quinoline-3-carboxylic acid;
1-Cyclopropyl-6-fluoro-7-{3-[hydroxy-(1-hydroxy-cyclopropyl)-methyl]-pyrrolidin-1-yl}-8-methoxy-4-oxo-1,4-dihydro-quinoline-3-carboxylic acid;
1-Cyclopropyl-6-fluoro-7-[3-(hydroxy-thiazol-2-yl-methyl)-pyrrolidin-1-yl]-8-methoxy-4-oxo-1,4-dihydro-quinoline-3-carboxylic acid;
8-Chloro-1-cyclopropyl-7-[3-(1,2-dihydroxy-1-methyl-ethyl)-pyrrolidin-1-yl]-6-fluoro-4-oxo-1,4-dihydro-quinoline-3-carboxylic acid;
1-Cyclopropyl-6-fluoro-8-methoxy-4-oxo-7-[3-(2,2,2-trifluoro-1-hydroxy-1-hydroxymethyl-ethyl)-pyrrolidin-1-yl]-1,4-dihydro-quinoline-3-carboxylic acid;
1-(6-Amino-3,5-difluoro-pyridin-2-yl)-8-chloro-6-fluoro-4-oxo-7-[3-(2,2,2-trifluoro-1-hydroxy-ethyl)-pyrrolidin-1-yl]-1,4-dihydro-quinoline-3-carboxylic acid;
1-(6-Amino-3,5-difluoro-pyridin-2-yl)-8-chloro-7-[3-(2,2-difluoro-1-hydroxy-ethyl)-pyrrolidin-1-yl]-6-fluoro-4-oxo-1,4-dihydro-quinoline-3-carboxylic acid; or
1-(6-Amino-3,5-difluoro-pyridin-2-yl)-7-[3-(2,2-difluoro-1-hydroxy-ethyl)-pyrrolidin-1-yl]-6-fluoro-4-oxo-1,4-dihydro-quinoline-3-carboxylic acid.

24. A compound of formula III 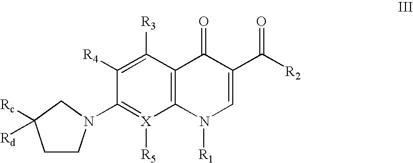
or a pharmaceutically acceptable salt thereof, wherein:
X is C or N, provided that when X is N, R₅ is absent;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl, 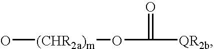
wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂ₐ)ₙ—Y wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 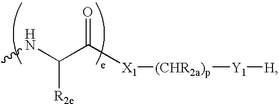
wherein “ [symbol] ” indicates the point of attachment, 2a is as defined above, R₂e is H or (C₁-C₆)alkyl, e is an integer of from 1 to 10, p is an integer of from 2 to 10, and X₁ and Y₁ are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R_{c} is OH,
OPO(OH)₂,
OPO(O(C₁-C₆)alkyl)₂, 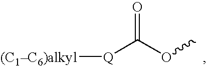
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 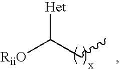
wherein “ [symbol] ” indicates the point of attachment, het is a 5- or 6-membered heterocyclo or heteroaryl group, and x is an integer of from 0 to 10; 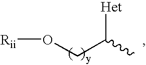
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 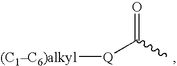
as defined above; and
R_{d} is (C₁-C₆)alkyl, 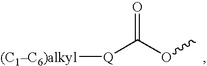
wherein “ [symbol] ˜ indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 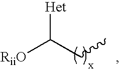
wherein “ [symbol] ” indicates the point of attachment, het is a 5- or 6-membered heterocyclo or heteroaryl group, and x is an integer of from 0 to 10; 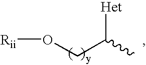
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂,

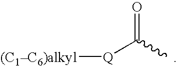

25. The compound of claim 24, wherein
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy, difluoromethoxy, or trifluoromethoxy.

26. The compound of claim 25, wherein
R₁ is cyclopropyl or fluorocyclopropyl;
R₃ is H or NH₂.;
R₄ is H or F.
X is C or N.
R₅ is halo or methoxy.

27. The compound of claim 1, wherein R₁, R₃, R₄, and R₅ are as provided in the following structures, wherein R₂ is OH, OBF₂, or O(C₁-C₆)alkyl, R₄ is H or F, and wherein A′ is

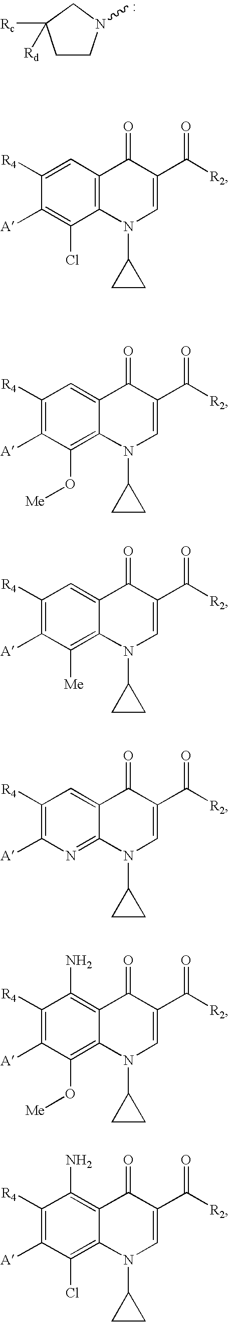

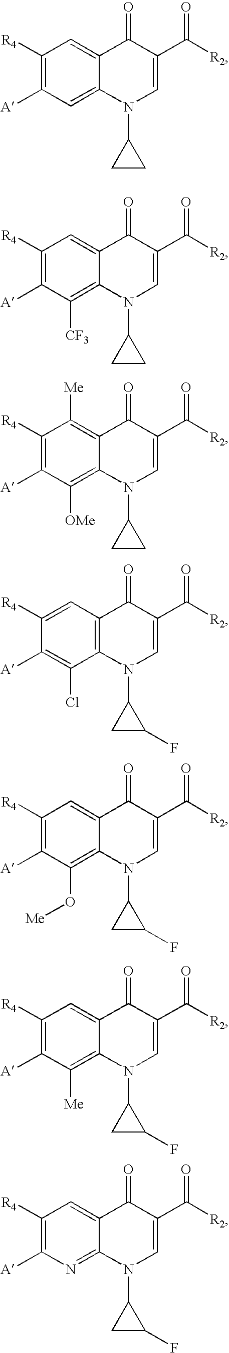

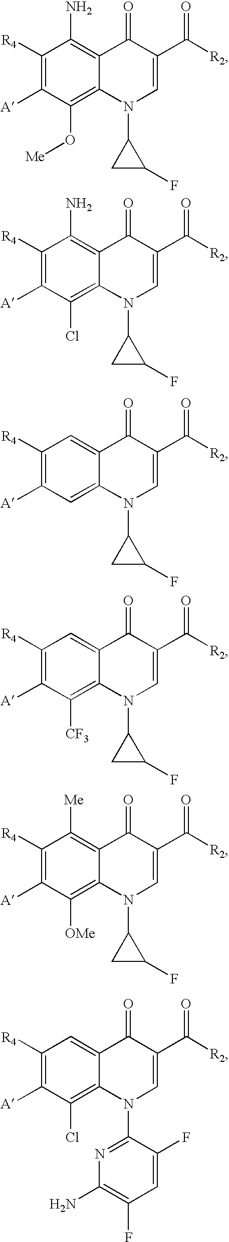

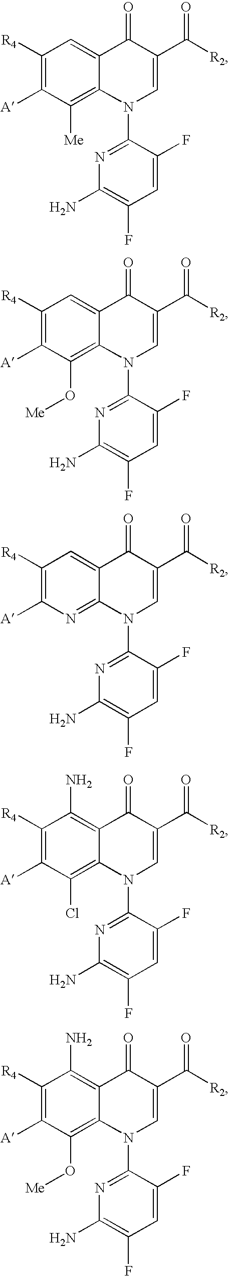

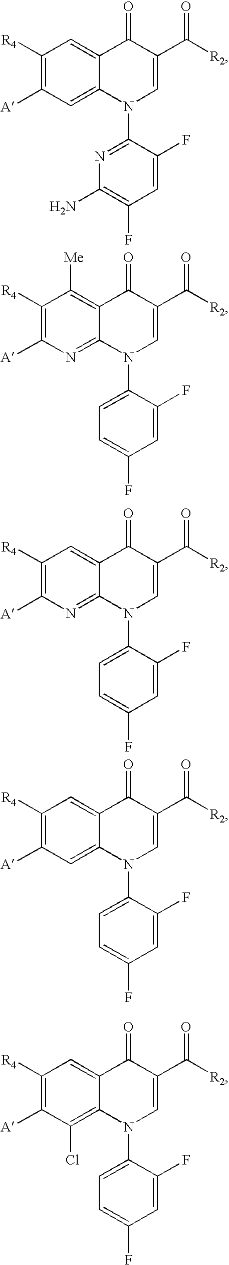

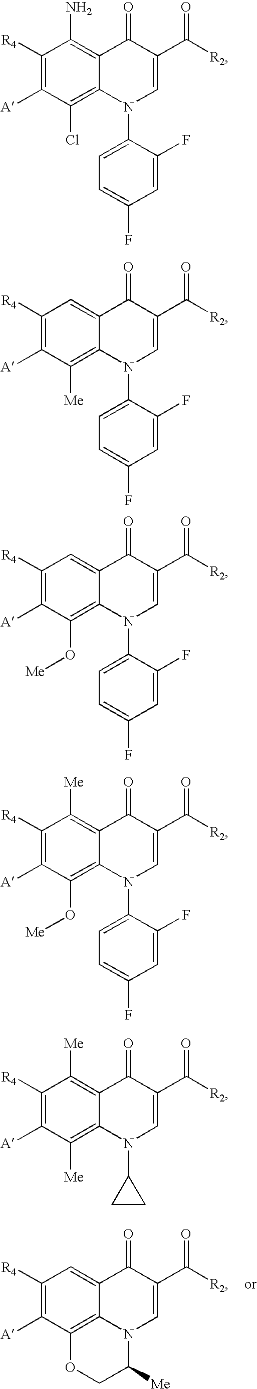

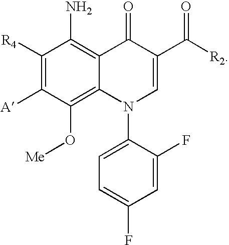

28. The compound of claim 27, wherein 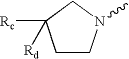
wherein R_{c} is OH,
OPO(OH)₂,
OPO(O(C₁-C₆)alkyl)₂, 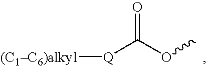
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 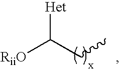
wherein “ [symbol] ” indicates the point of attachment, het is a 5- or 6-membered heterocyclo or heteroaryl group, and x is an integer of from 0 to 10; 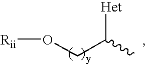
wherein “ [symbol] ” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, or 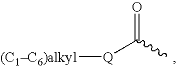
as defined above; and
R_{d} is halo,
(C₁-C₆)alkyl,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, or 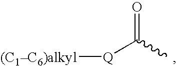
wherein “ [symbol] ” indicates the point of attachment and Q is O or is absent.

29. The compound of claim 27, wherein

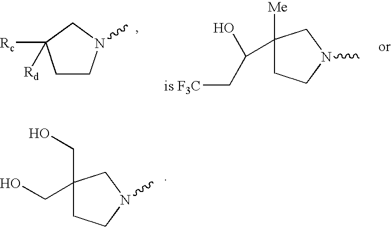

30. The compound of claim 29 which is
1-Cyclopropyl-6-fluoro-8-methoxy-7-[3-methyl-3-(3,3,3-trifluoro-1-hydroxy-propyl)-pyrrolidin-1-yl]-4-oxo-1,4-dihydro-quinoline-3-carboxylic acid;
7-(3,3-Bis-hydroxymethyl-pyrrolidin-1-yl)-1-cyclopropyl-6-fluoro-8-methyl-4-oxo-1,4-dihydro-quinoline-3-carboxylic acid; or
7-(3,3-Bis-hydroxymethyl-pyrrolidin-1-yl)-1-cyclopropyl-6-fluoro-8-methoxy-4-oxo-1,4-dihydro-quinoline-3-carboxylic acid.

31. A compound of formula IV 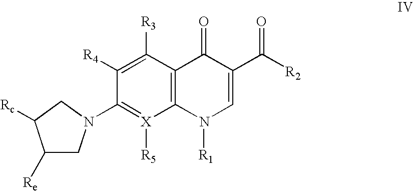
or a pharmaceutically acceptable salt thereof, wherein:
X is C or N, provided that when X is N, R₅ is absent;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl, 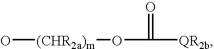
wherein m is an integer of from 1 to 10, Q is O or is absent, and R₂ₐ is H or (C₁-C₆)alkyl and R_{2b} is (C₁-C₆)alkyl, aryl, or heteroaryl,
O (CHR₂ₐ)n Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 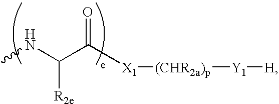
wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y, are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R_{c} is OH,
OPO(OH)₂,
OPO(O(C₁-C₆)alkyl)₂, 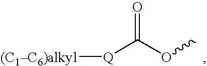
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 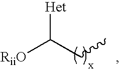
wherein “ [symbol] ” indicates the point of attachment, het is a 5- or 6-membered heterocyclo or heteroaryl group, and x is an integer of from 0 to 10; 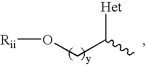
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 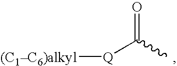
as defined above;
provided that 3 or fewer of R_{c} R_{d}, Rₑ, and R_{f} are H; and
Rₑ is OPO(OH)₂,
OPO(O(C₁-C₆)alkyl)₂, 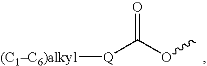
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 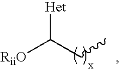
wherein “ [symbol] ” indicates the point of attachment, het is a 5- or 6-membered heterocyclo or heteroaryl group, and x is an integer of from 0 to 10; 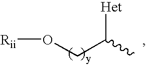
wherein “ [symbol] ” indicates the point of attachment, het is as defined above, and y is an integer of from 1 to 10, wherein Rᵢᵢ is H,
(C₁-C₆)alkyl,
PO(OH)₂,
PO(O(C₁-C₆)alkyl)₂, or 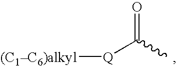
defined above.

32. The compound of claim 31, wherein
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy, difluoromethoxy, or trifluoromethoxy.

33. The compound of claim 31, wherein
R₁ is cyclopropyl or fluorocyclopropyl;
R₃ is H or NH₂.;
R₄ is H or F.
X is C or N.
R₅ is halo or methoxy.

34. The compound of claim 31, wherein R₁, R₃, R₄, and R₅ are as provided in the following structures, wherein R₂ is OH, OBF₂, or O(C₁-C₆)alkyl, R₄ is H or F, and wherein A′ is

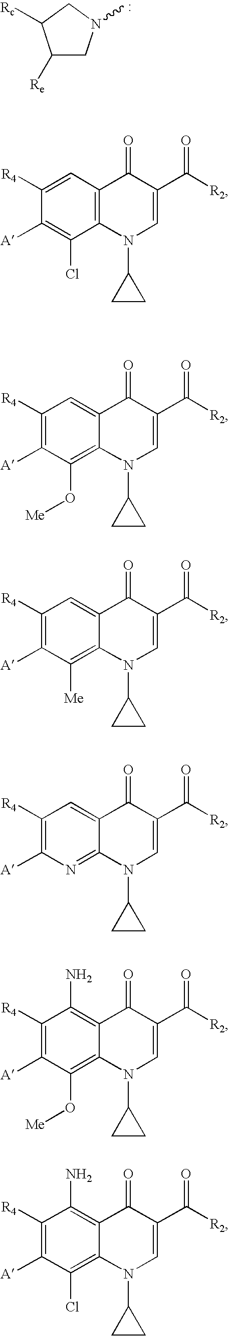

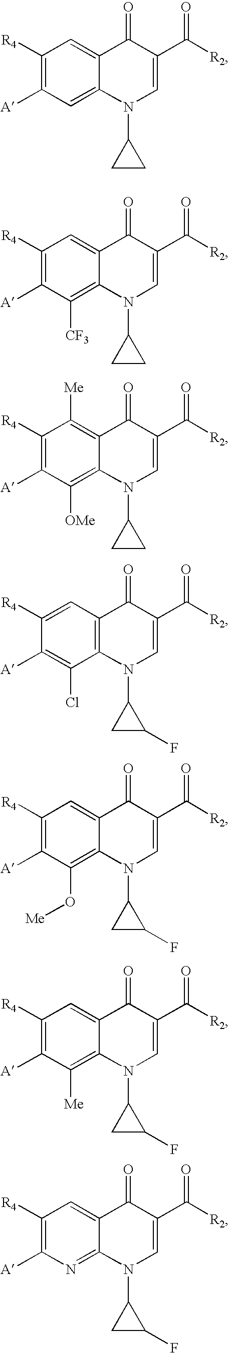

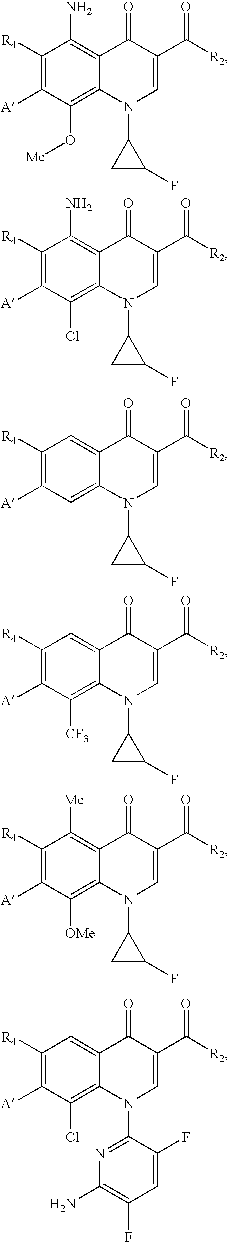

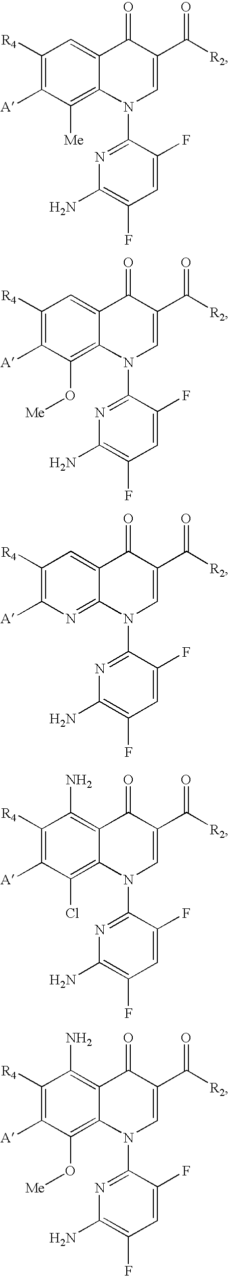

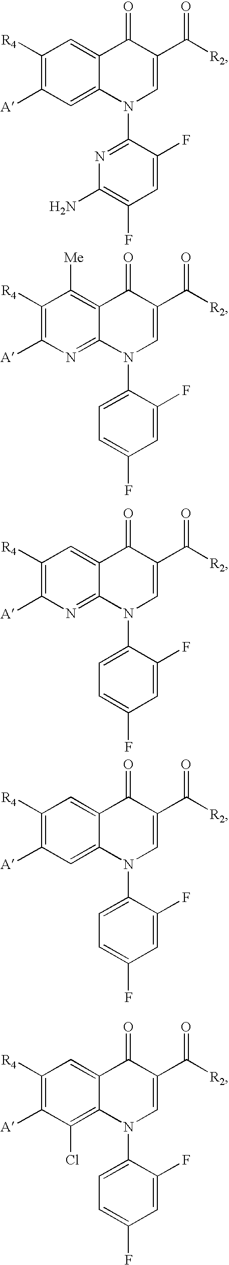

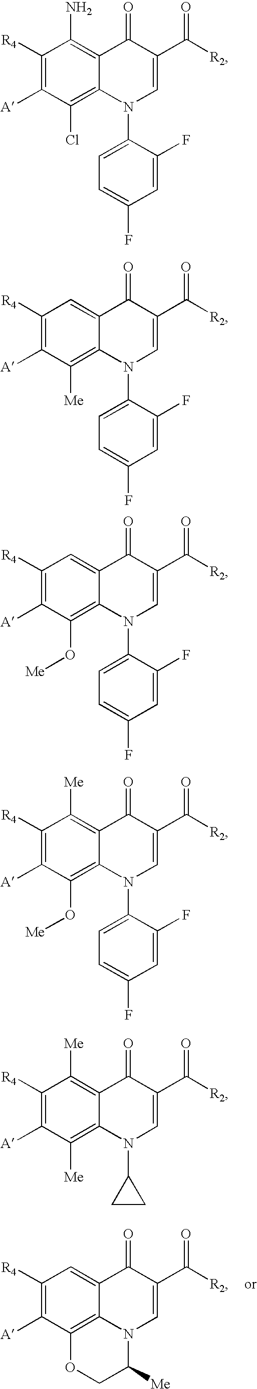

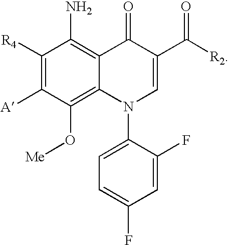

35. The compound of claim 34 wherein A is 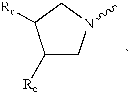
wherein
wherein R_{c} is OH,
OPO(OH)₂,
OPO(O(C₁-C₆)alkyl)₂, 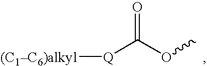
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 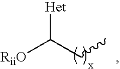
wherein “ [symbol] ” indicates the point of attachment, het is a 5- or 6-membered heterocyclo or heteroaryl group, and x is an integer of from 0 to 10; 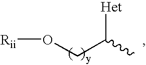
wherein “ [symbol] ” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 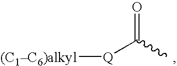
as defined above; and
Rₑ is halo 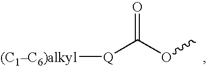
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 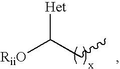
wherein “ [symbol] ” indicates the point of attachment, het is a 5- or 6-membered heterocyclo or heteroaryl group, and x is an integer of from 0 to 10; 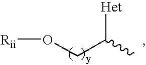
wherein “ [symbol] ” indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, or 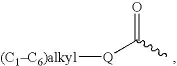
as defined above.

36. The compound of claim 35, wherein

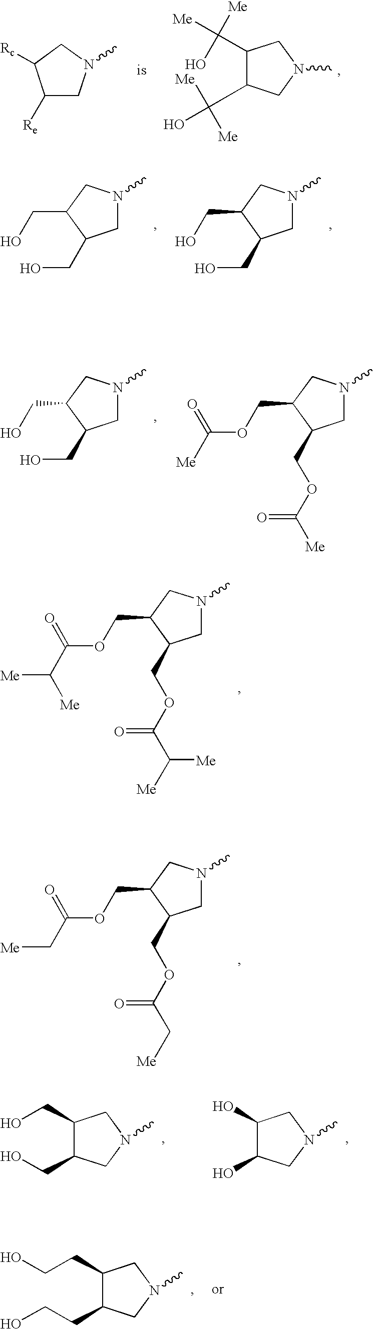

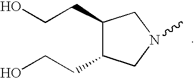

37. A compound which is
7-(3,4-Bis-hydroxymethyl-pyrrolidin-1-yl)-1-(2,4-difluoro-phenyl)-6-fluoro-4-oxo-1,4-dihydro-[1,8]naphthyridine-3-carboxylic acid;
7-(3R,4S-bis-hydroxymethyl-pyrrolidin-1-yl)-8-chloro-1-cyclopropyl-6-fluoro-4-oxo-1,4-dihydro-quinoline-3-carboxylic acid;
5-Amino-7-(3R,4S-bis-hydroxymethyl-pyrrolidin-1-yl)-8-chloro-1-cyclopropyl-6-fluoro-4-oxo-1,4-dihydro-quinoline-3-carboxylic acid;
1-(6-Amino-3,5-difluoro-pyridin-2-yl)-7-(3,4-bis-hydroxymethyl-pyrrolidin-1-yl)-8-chloro-6-fluoro-4-oxo-1,4-dihydro-quinoline-3-carboxylic acid;
7-(3,4-Bis-hydroxymethyl-pyrrolidin-1-yl)-1-cyclopropyl-6-fluoro-8-methoxy-4-oxo-1,4-dihydro-quinoline-3-carboxylic acid;
7-(3,4-Bis-hydroxymethyl-pyrrolidin-1-yl)-1-cyclopropyl-6-fluoro-8-methoxy-4-oxo-1,4-dihydro-quinoline-3-carboxylic acid;
7-(3,4-Bis-hydroxymethyl-pyrrolidin-1-yl)-1-cyclopropyl-6-fluoro-8-methyl-4-oxo-1,4-dihydro-quinoline-3-carboxylic acid;
7-(3,4-Bis-methoxymethyl-pyrrolidin-1-yl)-1-cyclopropyl-6-fluoro-8-methyl-4-oxo-1,4-dihydro-quinoline-3-carboxylic acid;
1-Cyclopropyl-7-(3-methoxymethyl-4-hydroxymethyl-pyrrolidin-1-yl)-6-fluoro-8-methyl-4-oxo-1,4-dihydro-quinoline-3-carboxylic acid;
1-Cyclopropyl-7-(3 -ethoxymethyl-4-hydroxymethyl-pyrrolidin-1-yl)-6-fluoro-8-methyl-4-oxo-1,4-dihydro-quinoline-3-carboxylic acid;
1-Cyclopropyl-7-(3-propoxymethyl-4-hydroxymethyl-pyrrolidin-1-yl)-6-fluoro-8-methyl-4-oxo-1,4-dihydro-quinoline-3-carboxylic acid;
7-(3,4-Bis-acetoxymethyl-pyrrolidin-1-yl)-1-cyclopropyl-6-fluoro-8-methyl-4-oxo-1,4-dihydro-quinoline-3-carboxylic acid;
7-(3,4-Bis-propionyloxymethyl-pyrrolidin-1-yl)-1-cyclopropyl-6-fluoro-8-methyl-4-oxo-1,4-dihydro-quinoline-3-carboxylic acid;
7-(3,4-Bis-isobutyryloxymethyl-pyrrolidin-1-yl)-1-cyclopropyl-6-fluoro-8-methyl-4-oxo-1,4-dihydro-quinoline-3-carboxylic acid;
7-[3S,4R-Bis-(2-hydroxy-ethyl)-pyrrolidin-1-yl]-1-cyclopropyl-6-fluoro-8-methyl-4-oxo-1,4-dihydro-quinoline-3-carboxylic acid;
7-[3S,4R-Bis-(2-hydroxy-ethyl)-pyrrolidin-1-yl]-1-cyclopropyl-6-fluoro-8-methoxy-4-oxo-1,4-dihydro-quinoline-3-carboxylic acid; or
7-[3,4-Bis-(2-hydroxy-ethyl)-pyrrolidin-1-yl]-1-cyclopropyl-8-difluoromethoxy-6-fluoro-4-oxo-1,4-dihydro-quinoline-3-carboxylic acid.

38. A compound of formula V 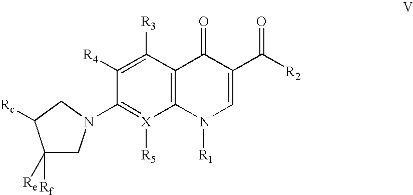
or a pharmaceutically acceptable salt thereof, wherein:
X is C or N, provided that when X is N, R₅ is absent;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl, 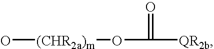
wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂ₐ)ₙ—Y wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 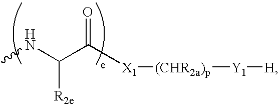
wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R_{c} is
OPO(OH)₂,
OPO(O(C₁-C₆)alkyl)₂, 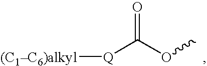
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C]-C₆)haloalkyl-O—,
RᵢᵢO(C₃-C₆)cycloalkyl-O—, 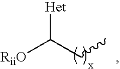
wherein “ [symbol] ” indicates the point of attachment, het is a 5- or 6-membered heterocyclo or heteroaryl group, and x is an integer of from 0 to 10; 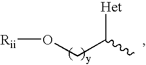
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 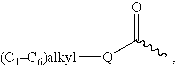
as defined above; and
Rₑ and R_{f} are each independently (C₁-C₆)alkyl or together with the carbon to which they are attached, form a substituted or unsubstituted 3,4, 5, or 6-membered ring containing 0, 1, 2, or 3 heteroatoms selected from NH, N(C₁-C₆)alkyl, S, or O.

39. The compound of claim 38, wherein
R₁ is (C₁-C₆)cycloalkyl and halo(C₁-C₆)cycloalkyl, aryl, or heteroaryl;
R₃ is H or NH₂;
R₄is H or halo;
R₅ is halo, methyl, trifluoromethyl, methoxy, fluoromethoxy, difluoromethoxy, or trifluoromethoxy.

40. The compound of claim 39, wherein
R₁ is cyclopropyl or fluorocyclopropyl;
R₃ is H or NH₂.;
R₄ is H or F.
X is C or N.
R₅ is halo or methoxy.

41. The compound of claim 39, wherein R₁, R₃, R₄, and R₅ are as provided in the following structures, wherein R₂ is OH, OBF₂, or O(C₁-C₆)alkyl, R₄ is H or F, and wherein A′ is

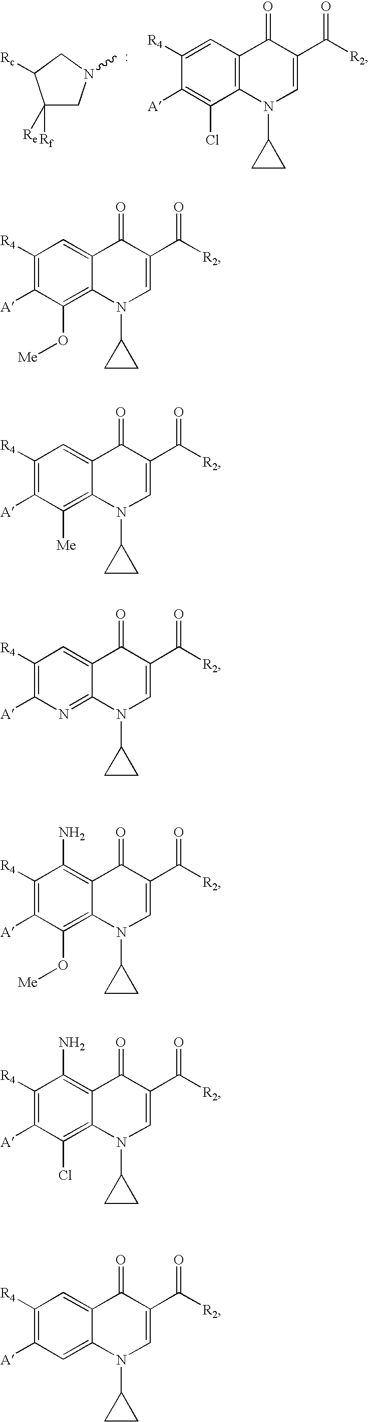

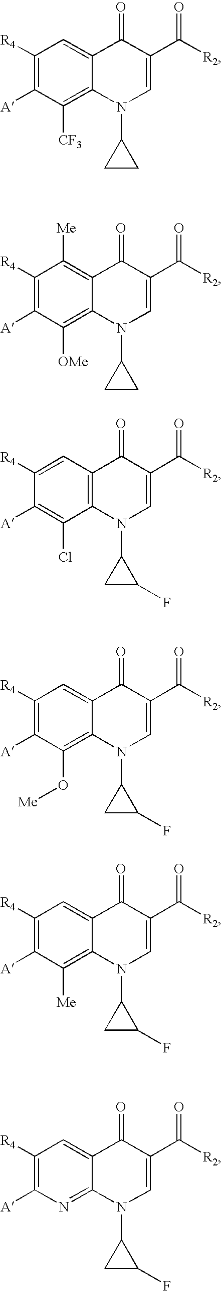

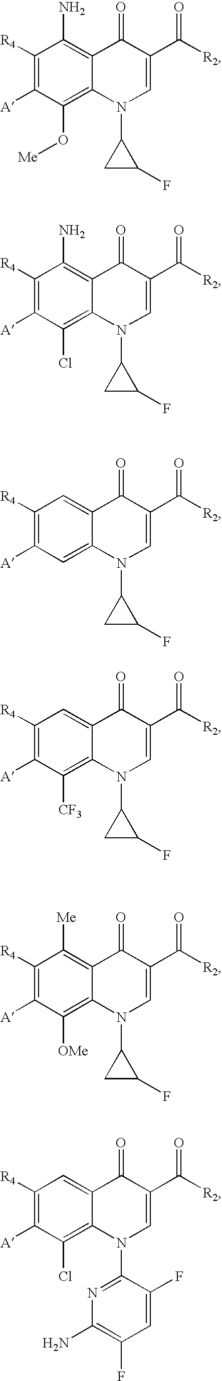

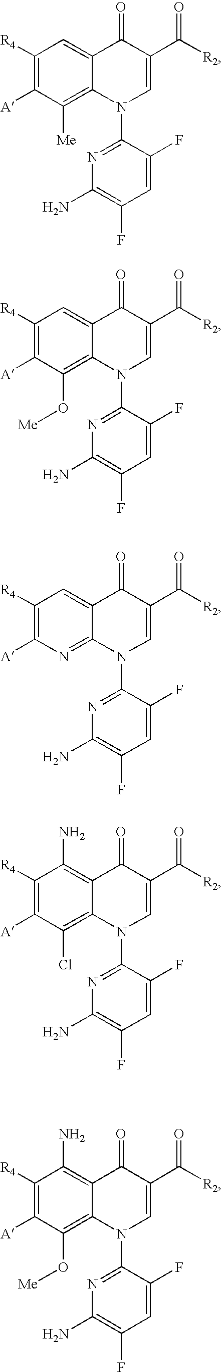

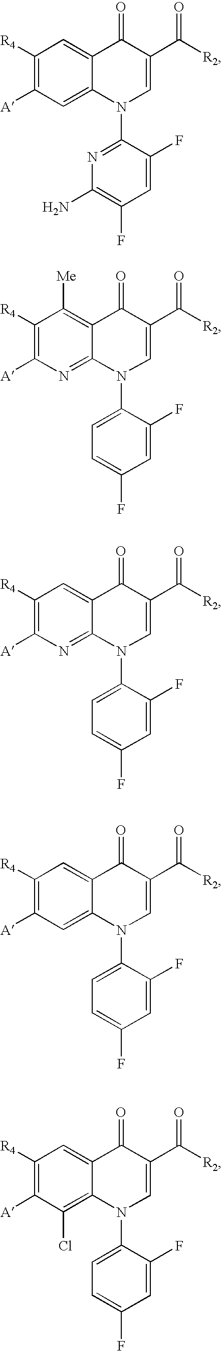

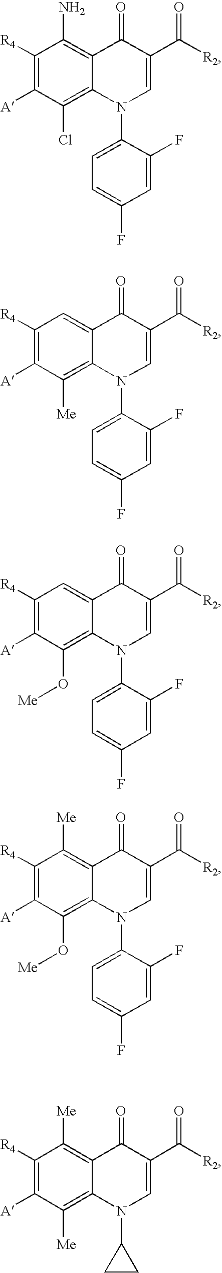

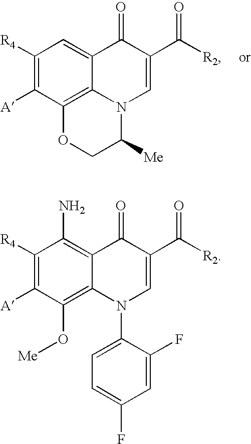

42. The compound of claim 41, wherein A′ is 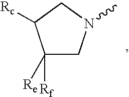
wherein R_{c} is
OPO(OH)₂,
OPO(O(C₁-C₆)alkyl)₂, 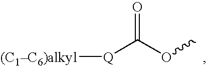
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 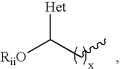
wherein “ [symbol] ” indicates the point of attachment, het is a 5- or 6-membered heterocyclo or heteroaryl group, and x is an integer of from 0 to 10; 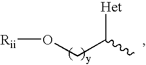
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 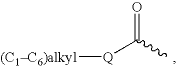
as defined above; and
Rₑ and R_{f} are each independently (C₁-C₆)alkyl or together with the carbon to which they are attached, form a substituted or unsubstituted 3, 4, 5, or 6-membered ring containing 0, 1, 2, or 3 heteroatoms selected from NH, N(C₁-C₆)alkyl, S, or O.

43. The compound of claim 42, wherein

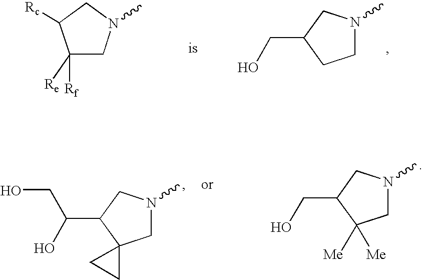

44. A compound which is
8-Methyl-1-cyclopropyl-6-fluoro-7-(4-hydroxymethyl-3,3-dimethyl-pyrrolidin-1-yl)-4-oxo-1,4-dihydro-quinoline-3-carboxylic acid;
8-Methyl-1-cyclopropyl-6-fluoro-7-(7-hydroxymethyl-5-aza-spiro[2.4]hept-5-yl)-4-oxo-1,4-dihydro-quinoline-3-carboxylic acid;
8-Methoxy-1-cyclopropyl-6-fluoro-7-(7-hydroxymethyl-5-aza-spiro[2.4]hept-5-yl)-4-oxo-1,4-dihydro-quinoline-3-carboxylic acid;
8-Chloro-1-cyclopropyl-6-fluoro-7-(7-hydroxymethyl-5-aza-spiro[2.4]hept-5-yl)-4-oxo-1,4-dihydro-quinoline-3-carboxylic acid;
1-Cyclopropyl-7-[7-(1,2-dihydroxy-ethyl)-5-aza-spiro[2.4]hept-5-yl]-6-fluoro-8-methyl-4-oxo-1,4-dihydro-quinoline-3-carboxylic acid;
1-Cyclopropyl-6-fluoro-7-[4-(1-hydroxy-ethyl)-3,3-dimethyl-pyrrolidin-1-yl]-8-methyl-4-oxo-1,4-dihydro-quinoline-3-carboxylic acid;
1-Cyclopropyl-6-fluoro-7-{4-[1-hydroxy-2-(2-methoxy-ethoxy)-ethyl]-3,3-dimethyl-pyrrolidin-1-yl}-8-methyl-4-oxo-1,4-dihydro-quinoline-3-carboxylic acid;
1-Cyclopropyl-6-fluoro-7-[4-(1-hydroxy-ethyl)-3,3-dimethyl-pyrrolidin-1-yl]-8-methyl-4-oxo-1,4-dihydro-quinoline-3-carboxylic acid; or
1-Cyclopropyl-6-fluoro-7-[4-(1-hydroxy-2-methoxy-ethyl)-3,3-dimethyl-pyrrolidin-1-yl]-8-methyl-4-oxo-1,4-dihydro-quinoline-3-carboxylic acid.

45. A compound of formula VI 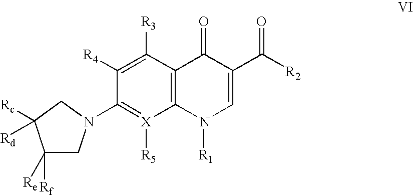
or a pharmaceutically acceptable salt thereof, wherein:
X is C or N, provided that whne X is N, R₅ is absent;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl, 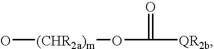
O—(CHR2,),-O 11 QR2b, wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂₁)ₙ—Y, wherein R₂ₐ is as defined above, n is an integer of from 2 to 10, Y is OH or NR_{2c}R_{2d}, wherein R_{2c} and R_{2d} are each independently H, (C₁-C₆)alkyl, or (C₃-C₆)cycloalkyl, or
NR_{2d}, wherein R_{2d} is as defined above, 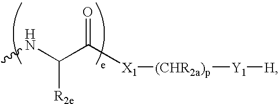
wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y₁ are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R_{c} and Rₑ are OH,
OPO(OH)₂,
OPO(O(C₁-C₆)alkyl)₂, 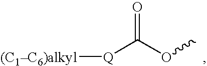
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 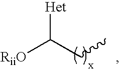
wherein “ [symbol] ” indicates the point of attachment, het is a 5- or 6-membered heterocyclo or heteroaryl group, that does not contain an NH, and x is an integer of from 0 to 10; 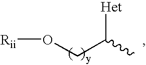
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 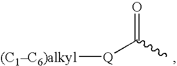
as defined above; and
R_{d} and R_{f} are each independently (C₁-C₆)alkyl.

46. The compound of claim 45, wherein
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy, difluoromethoxy, or trifluoromethoxy.

47. The compound of claim 46, wherein
R₁ is cyclopropyl or fluorocyclopropyl;
R₃is H or NH₂.;
R₄ is H or F.
X is C or N.
R₅ is halo or methoxy.

48. The compound of claim 47, wherein R₁, R₃, R₄, and R₅ are as provided in the following structures, wherein R₂ is OH, OBF₂, or O(C₁-C₆)alkyl, R₄ is H or F, and wherein A′ is

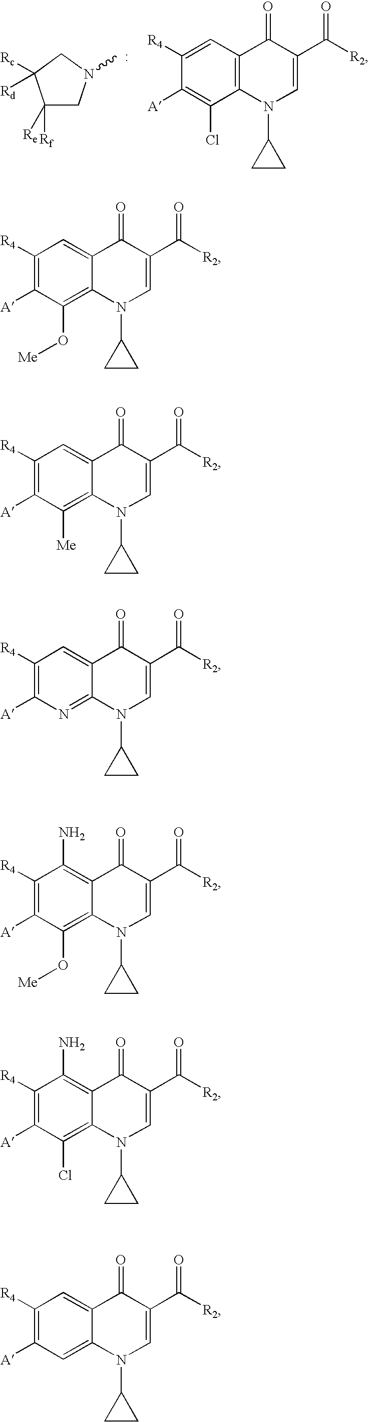

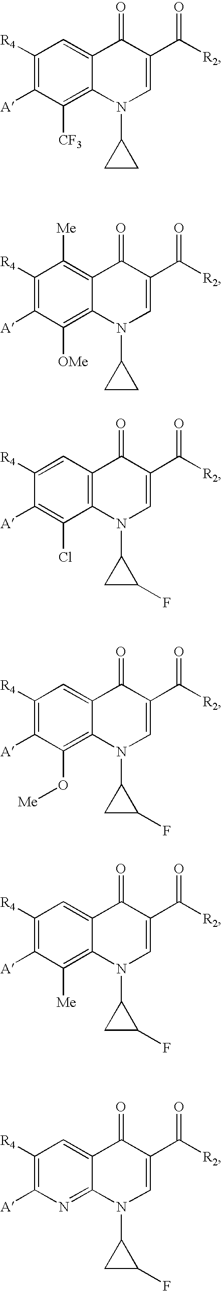

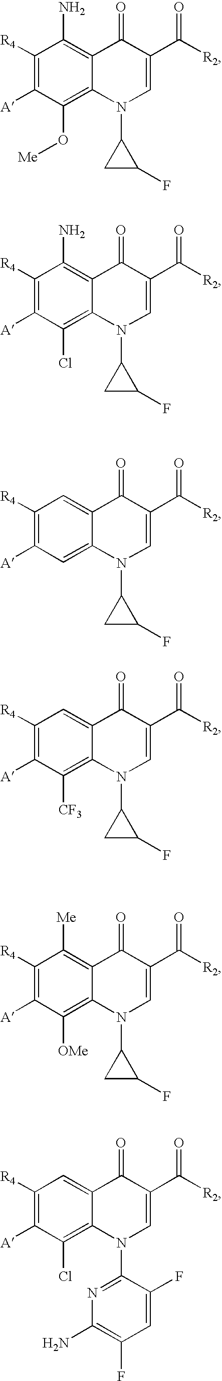

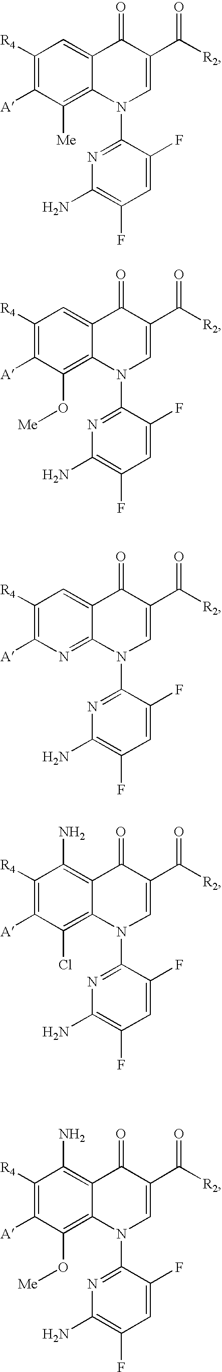

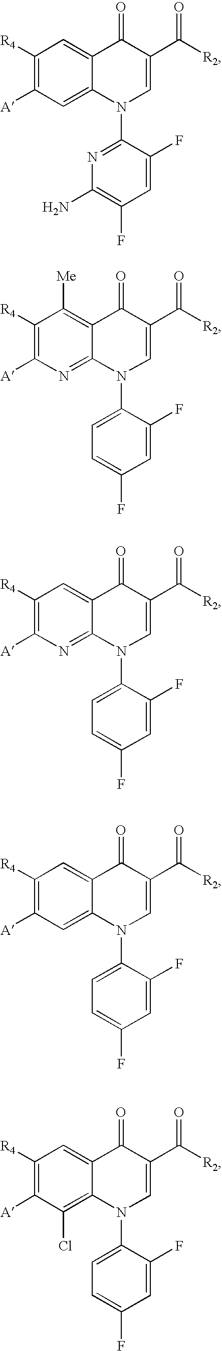

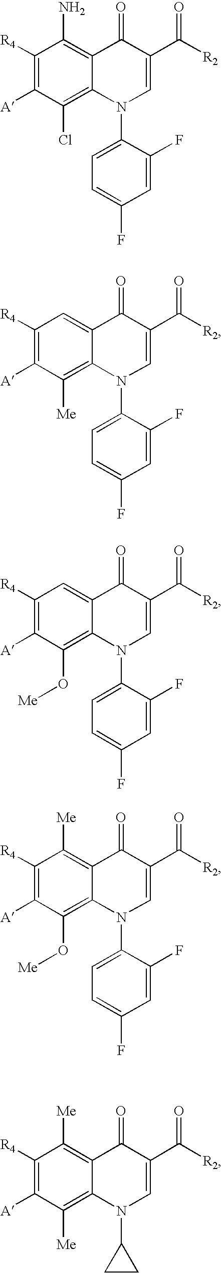

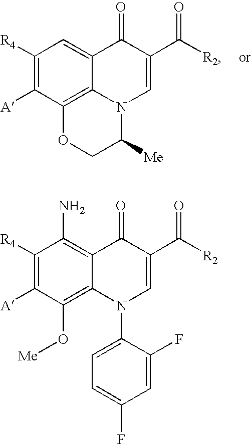

49. The compound of claim 48, wherein A′ is 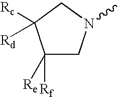
wherein R_{c} and Rₑ are each independantly H,
OH,
OPO(OH)₂, 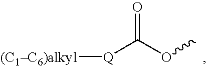
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 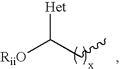
wherein “ [symbol] ” indicates the point of attachment, het is a 5- or 6-membered heterocyclo or heteroaryl group, and x is an integer of from 0 to 10; 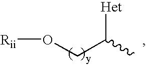
wherein “ [symbol] ” indicates the point of attachment, het is as defined above, and y is an integer of from 1 to 10,
wherein Rᵢᵢ is H,
(C₁-C₆)alkyl,
PO(OH)₂, 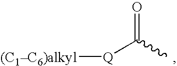
as defined above; and
R_{d} and R_{f} are each independently (C₁-C₆)alkyl, or taken together with the carbons to which they are attached form a substituted or
unsubstituted 4, 5, or 6 membered ring, optionally containing one heteroatom selected from NH, N(C₁-C₆)alkyl, S, or O.

50. The compound of claim 49, wherein

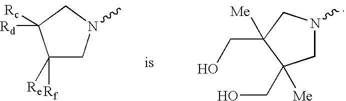

51. A compound which is
7-(3 ,4-Bis-hydroxymethyl-3,4-dimethyl-pyrrolidin-1-yl)-1-cyclopropyl-6-fluoro-8-methyl-4-oxo-1,4-dihydro-quinoline-3-carboxylic acid; or
7-(3,4-Bis-hydroxymethyl-3,4-dimethyl-pyrrolidin-1-yl)-1-cyclopropyl-6-fluoro-8-methoxy-4-oxo-1,4-dihydro-quinoline-3-carboxylic acid.

52. A compound of formula VII 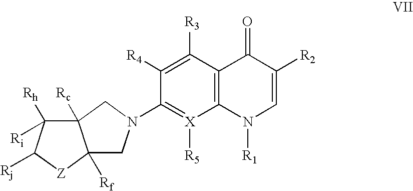
or a pharmaceutically acceptable salt thereof, wherein:
X is C or N, provided that whne X is N, R₅ is absent;
Z is absent or is a linker containing1, 2, or 3 substituted or unsubstituted carbon atoms;
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is OH,
OBF₂,
O(C₁-C₆)alkyl,
O(C₃-C₆)cycloalkyl, 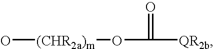
wherein m is an integer of from 1 to 10, Q is O or is absent, and R₂ₐ is H or (C₁-C₆)alkyl and R_{2b} is (C₁-C₆)alkyl, aryl, or heteroaryl,
O—(CHR₂ₐ)ₙ—Y wherein R₂ₐ is as defined above, n is an integer of from 2 to 10, Y is OH or NR_{2c}R_{2d}, wherein R₂C and R_{2d} are each independently H, (C₁-C₆)alkyl, or (C₃-C₆)cycloalkyl, or
NR_{2d}, wherein R_{2d} is as defined above, 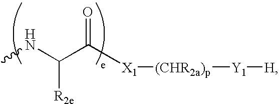
wherein “ [symbol] ” indicates the point of attachment, 2a is as defined above, R₂ₑ is H or (C₁-C₆)alkyl, e is an integer of from 1 to 10, p is an integer of from 2 to 10, and X₁ and Y, are each independently NH or O;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
R_{c} and R_{f} are each independently H, (C₁-C₆)alkyl, or HO—(C₁-C₆)alkyl;
Rₕ, Rᵢ, and Rⱼ are each independently H,
OH,
OPO(OH)₂,
OPO(O(C₁-C₆)alkyl)₂, 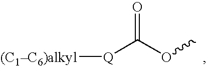
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 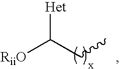
wherein “ [symbol] ” indicates the point of attachment, het is a 5- or 6-membered heterocyclo or heteroaryl group that does not contain an NH, and x is an integer of from 0 to 10; 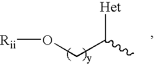
wherein “ [symbol] ” indicates the point of attachment, het is as defined above, and y is an integer of from 1 to 10,
wherein Rᵢᵢ is H,
(C₁-C₆)alkyl,
PO(OH)₂,
PO(O(C₁-C₆)alkyl)₂, 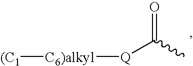
as defined above;
provided that not all of Rₕ Rᵢ, and Rⱼ are H.

53. The compound of claim 52, wherein
R₁ is (C₁-C₆)cycloalkyl and halo(C₁-C₆)cycloalkyl, aryl, or heteroaryl;
R₃ is H or NH₂;
R₄ is H or halo;
R₅ is halo, methyl, trifluoromethyl, methoxy, fluoromethoxy, difluoromethoxy, or trifluoromethoxy.

54. The compound of claim 53, wherein
R₁ is cyclopropyl or fluorocyclopropyl;
R₃ is H or NH₂.;
R₄ is H or F.
X is C or N.
R₅ is halo or methoxy.

56. The compound of claim 53, wherein R₁, R₃, R₄, and R₅ are as provided in the following structures, wherein R₂ is OH, OBF₂, or O(C₁-C₆)alkyl, R₄ is H or F, and wherein A′ is

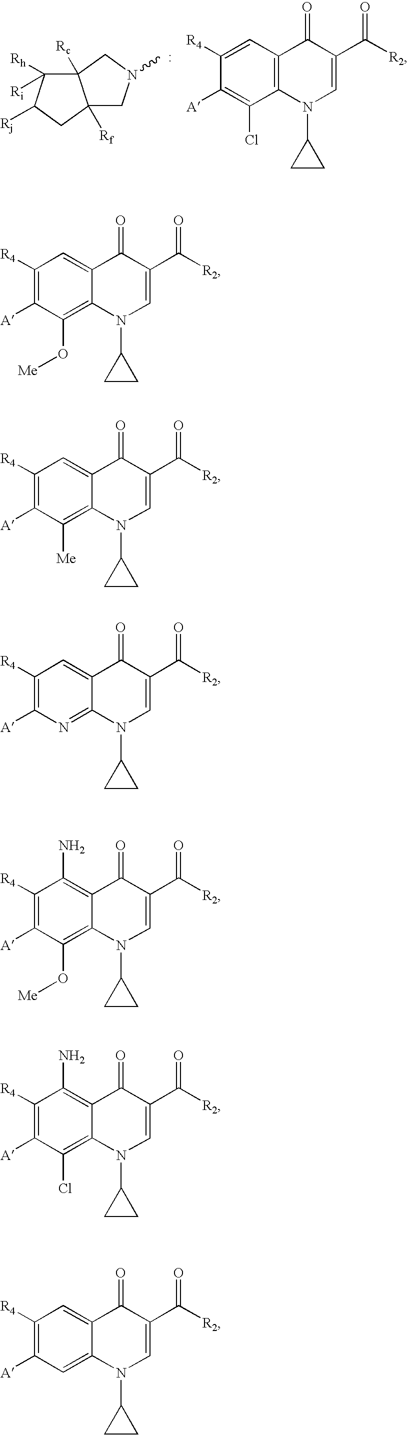

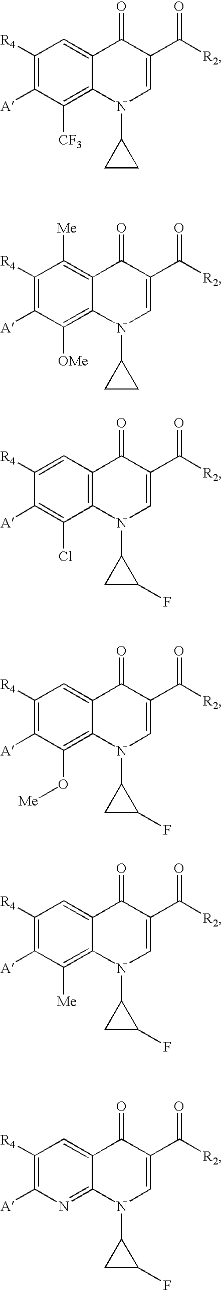

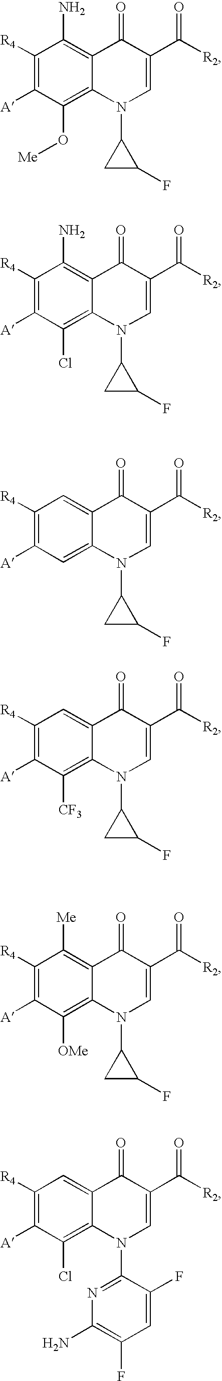

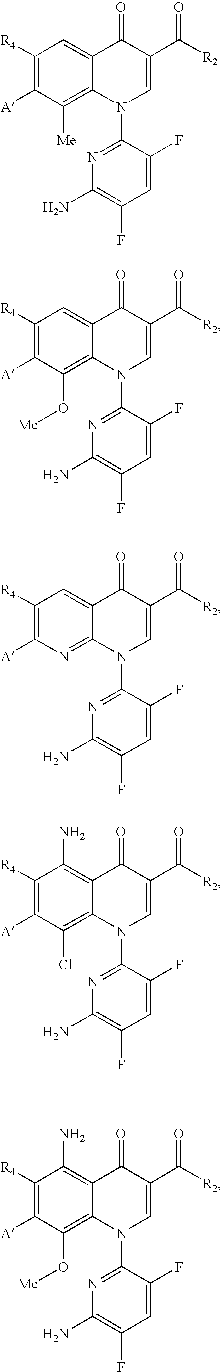

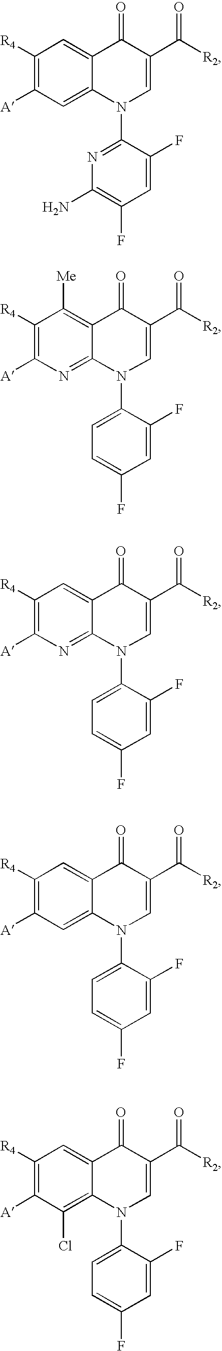

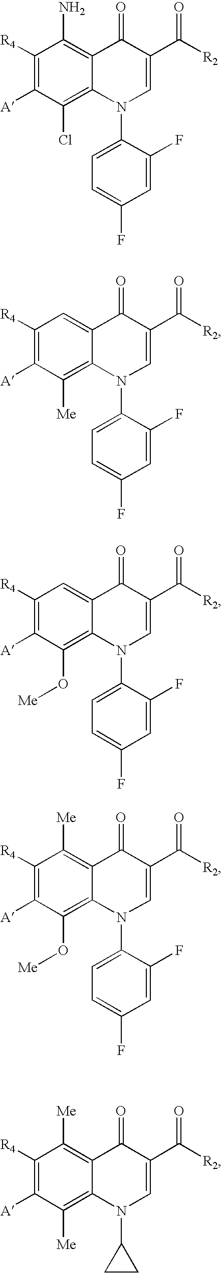

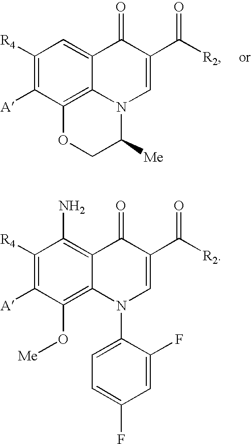

56. The compound of claim 55, wherein A′ is 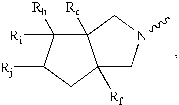
wherein
Z is absent or is a linker containing1, 2, or 3 substituted or unsubstituted carbon atoms;
R_{c} and R_{f} are each independently H, (C₁-C₆)alkyl, or HO—(C₁-C₆)alkyl;
Rₕ, Rᵢ, and Rⱼ are each independently H,
OH,
OPO(OH)₂, 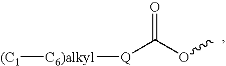
wherein “ [symbol] ” indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 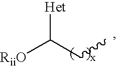
wherein “ [symbol] ” indicates the point of attachment, het is a 5- or 6-membered heterocyclo or heteroaryl group, and x is an integer 5 of from 0 to 10; 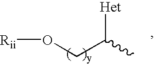
wherein “ [symbol] ” indicates the point of attachment, het is as defined above, and y is an integer of from 1 to 10,
wherein Rᵢᵢ is H,
(C₁-C₆)alkyl,
PO(OH)₂, 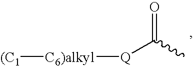
as defined above;
provided that not all of Rₕ Rᵢ, and Rⱼ are H.

57. The compound of claim 56, wherein

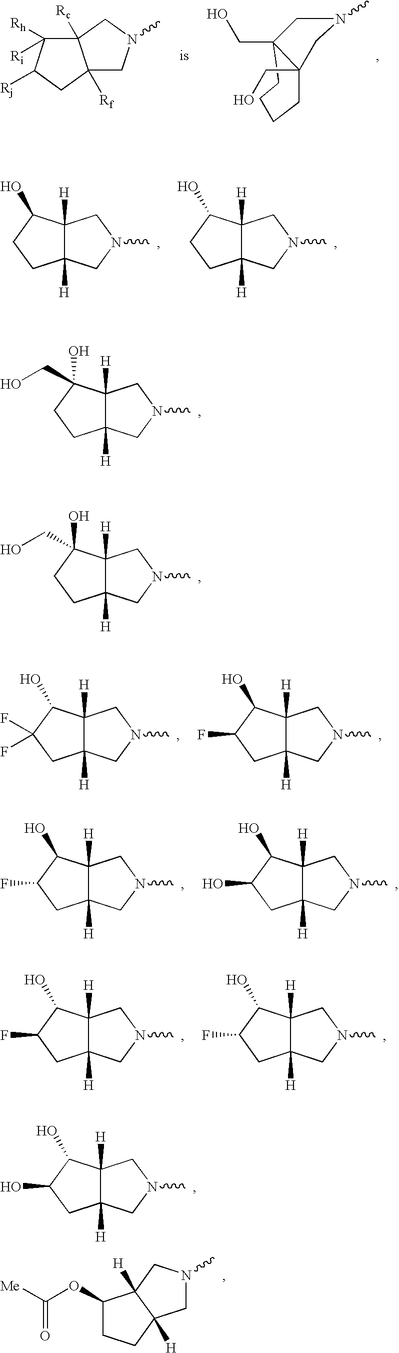

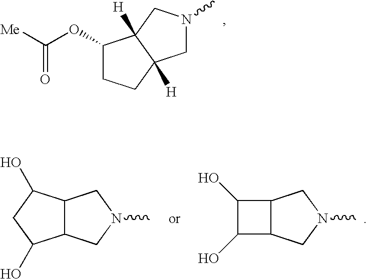

58. A compound which is
1-Cyclopropyl-6-fluoro-7-(4-hydroxy-hexahydro-cyclopenta[c]pyrrol-2-yl)-8-methyl-4-oxo-1,4-dihydro-quinoline-3-carboxylic acid;
1-Cyclopropyl-6-fluoro-7-(4-hydroxy-hexahydro-cyclopenta[c]pyrrol-2-yl)-8-methoxy-4-oxo-1,4-dihydro-quinoline-3-carboxylic acid;
1-(6-Amino-3,5-difluoro-pyridin-2-yl)-8-chloro-6-fluoro-7-(4-hydroxy-hexahydro-cyclopenta[c]pyrrol-2-yl)-4-oxo-1,4-dihydro-quinoline-3-carboxylic acid;
7-(4-Acetoxy-hexahydro-cyclopenta[c]pyrrol-2-yl)-1-(6-amino-3,5-difluoro-pyridin-2-yl)-8-chloro-6-fluoro-4-oxo-1,4-dihydro-quinoline-3-carboxylic acid;
1-Cyclopropyl-7-(4,5-dihydroxy-hexahydro-cyclopenta[c]pyrrol-2-yl)-6-fluoro-8-methyl-4-oxo-1,4-dihydro-quinoline-3-carboxylic acid;
1-Cyclopropyl-7-(4,5-dihydroxy-hexahydro-cyclopenta[c]pyrrol-2-yl)-6-fluoro-8-methoxy-4-oxo-1,4-dihydro-quinoline-3-carboxylic acid;
1-Cyclopropyl-8-difluoromethoxy-7-(4,5-dihydroxy-hexahydro-cyclopenta [c]pyrrol-2-yl)-6-fluoro-4-oxo-1,4-dihydro-quinoline-3-carboxylic acid;
1-Cyclopropyl-6-fluoro-7-(4-hydroxy-4-hydroxymethyl-hexahydro-cyclopenta [c]pyrrol-2-yl)-8-methoxy-4-oxo-1,4-dihydro-quinoline-3-carboxylic acid;
1-Cyclopropyl-6-fluoro-7-(4-hydroxy-4-hydroxymethyl-hexahydro-cyclopenta [c]pyrrol-2-yl)-8-methyl-4-oxo-1,4-dihydro-quinoline-3-carboxylic acid;
7-(3aR,7aS-Bis-hydroxymethyl-octahydro-isoindol-2-yl)-1-cyclopropyl-6-fluoro-8-methoxy-4-oxo-1,4-dihydro-quinoline-3-carboxylic acid; or
7-(3aR,7aS-Bis-hydroxymethyl-octahydro-isoindol-2-yl)-1-cyclopropyl-6-fluoro-8-methyl-4-oxo-1,4-dihydro-quinoline-3-carboxylic acid.

59. A pharmaceutical formulation comprising a compound of formula I, II, III, IV, V, VI, or VII admixed with a pharmaceutically acceptable diluent, carrier, or excipient.

60. A method of treating a bacterial infection in a mammal, comprising administering to a mammal in need thereof an effective amount of a compound of formula I, II, III, IV, V, VI, or VII.
